SEQUENCE LISTING (914 sequences)

SEQ ID NO: 1 - RNA, 22 nt, Homo sapiens
uggagugugacaaugguguuug

SEQ ID NO: 2 - RNA, 20 nt, Homo sapiens
uaaggcacgcggugaaugcc

SEQ ID NO: 3 - RNA, 24 nt, Homo sapiens
ucccugagacccuuuaaccuguga

SEQ ID NO: 4 - RNA, 22 nt, Homo sapiens
ucguaccgugaguaauaaugcg

SEQ ID NO: 5 - RNA, 22 nt, Homo sapiens
ucggauccgucugagcuuggcu

SEQ ID NO: 6 - RNA, 21 nt, Homo sapiens
ucacagugaaccggucucuuu

SEQ ID NO: 7 - RNA, 22 nt, Homo sapiens
aagcccuuaccccaaaaaguau

SEQ ID NO: 8 - RNA, 21 nt, Homo sapiens
cuuuuugcggucugggcuugc

SEQ ID NO: 9 - RNA, 22 nt, Homo sapiens
cagugcaaugaugaaagggcau

SEQ ID NO: 10 - RNA, 21 nt, Homo sapiens
acucuuucccuguugcacuac

SEQ ID NO: 11 - RNA, 22 nt, Homo sapiens
uuugguccccuucaaccagcug

SEQ ID NO: 12 - RNA, 22 nt, Homo sapiens
uuugguccccuucaaccagcua

SEQ ID NO: 13 - RNA, 23 nt, Homo sapiens
ccugugggccaccuagucaccaa

SEQ ID NO: 14 - RNA, 23 nt, Homo sapiens
uuauugcuuaagaauacgcguag

SEQ ID NO: 15 - RNA, 22 nt, Homo sapiens
uggaauguaaagaaguauguau

SEQ ID NO: 16 - RNA, 21 nt, Homo sapiens
ugagaugaagcacuguagcuc

SEQ ID NO: 17 - RNA, 22 nt, Homo sapiens
ggauuccuggaaauacuguucu

SEQ ID NO: 18 - RNA, 23 nt, Homo sapiens
guccaguuuucccaggaaucccu

SEQ ID NO: 19 - RNA, 22 nt, Homo sapiens
uggacggagaacugauaagggu

SEQ ID NO: 20 - RNA, 22 nt, Homo sapiens
acaguagucugcacauugguua

SEQ ID NO: 21 - RNA, 23 nt, Homo sapiens
cccaguguucagacuaccuguuc

SEQ ID NO: 22 - RNA, 22 nt, Homo sapiens
uucccuuugucauccuaugccu

SEQ ID NO: 23 - RNA, 22 nt, Homo sapiens
auaagacgaacaaaagguuugu

SEQ ID NO: 24 - RNA, 22 nt, Homo sapiens
acagcaggcacagacaggcagu

SEQ ID NO: 25 - RNA, 23 nt, Homo sapiens
uacugcaucaggaacugauugga

SEQ ID NO: 26 - RNA, 21 nt, Homo sapiens
ugauuguccaaacgcaauucu

SEQ ID NO: 27 - RNA, 22 nt, Homo sapiens
ugucaguuugucaaauacccca

SEQ ID NO: 28 - RNA, 22 nt, Homo sapiens
uggcagugucuuagcugguugu

SEQ ID NO: 29 - RNA, 22 nt, Homo sapiens
aaaccguuaccauuacugaguu

SEQ ID NO: 30 - RNA, 21 nt, Homo sapiens
uaaaguaaauaugcaccaaaa

SEQ ID NO: 31 - RNA, 22 nt, Homo sapiens
ugagguaguagguuguauaguu

SEQ ID NO: 32 - RNA, 23 nt, Homo sapiens
ucuuugguuaucuagcuguauga

SEQ ID NO: 33 - RNA, 22 nt, Homo sapiens
ugagguaguagguugugugguu

SEQ ID NO: 34 - RNA, 22 nt, Homo sapiens
ugagguaguagguuguaugguu

SEQ ID NO: 35 - RNA, 22 nt, Homo sapiens
agagguaguagguugcauaguu

SEQ ID NO: 36 - RNA, 22 nt, Homo sapiens
ugagguaggagguuguauaguu

SEQ ID NO: 37 - RNA, 22 nt, Homo sapiens
ugagguaguagauuguauaguu

SEQ ID NO: 38 - RNA, 22 nt, Homo sapiens
ugagguaguaguuuguacaguu

SEQ ID NO: 39 - RNA, 22 nt, Homo sapiens
ugagguaguaguuugugcuguu

SEQ ID NO: 40 - RNA, 22 nt, Homo sapiens
aacccguagauccgaacuugug

SEQ ID NO: 41 - RNA, 21 nt, Homo sapiens
uacaguacugugauaacugaa

SEQ ID NO: 42 - RNA, 23 nt, Homo sapiens
agcagcauuguacagggcuauga

SEQ ID NO: 43 - RNA, 23 nt, Homo sapiens
ucaaaugcucagacuccuguggu

SEQ ID NO: 44 - RNA, 23 nt, Homo sapiens
aaaagugcuuacagugcagguag

SEQ ID NO: 45 - RNA, 21 nt, Homo sapiens
uaaagugcugacagugcagau

SEQ ID NO: 46 - RNA, 23 nt, Homo sapiens
agcagcauuguacagggcuauca

SEQ ID NO: 47 - RNA, 23 nt, Homo sapiens
uacccuguagauccgaauuugug

SEQ ID NO: 48 - RNA, 23 nt, Homo sapiens
uacccuguagaaccgaauuugug

SEQ ID NO: 49 - RNA, 21 nt, Homo sapiens
uugcucacuguucuucccuag

SEQ ID NO: 50 - RNA, 22 nt, Homo sapiens
uuuccggcucgcgugggugugu

SEQ ID NO: 51 - RNA, 27 nt, Homo sapiens
cacuguaggugauggugagagugggca

SEQ ID NO: 52 - RNA, 22 nt, Homo sapiens
auauacagggggagacucuuau

SEQ ID NO: 53 - RNA, 22 nt, Homo sapiens
auauacagggggagacucucau

SEQ ID NO: 54 - RNA, 21 nt, Homo sapiens
agaggauacccuuuguauguu

SEQ ID NO: 55 - RNA, 23 nt, Homo sapiens
gggaugguagaccggugacgugc

SEQ ID NO: 56 - RNA, 21 nt, Homo sapiens
uaggacacauggucuacuucu

SEQ ID NO: 57 - RNA, 22 nt, Homo sapiens
cuccugagccauucugagccuc

SEQ ID NO: 58 - RNA, 21 nt, Homo sapiens
gugccagcugcagugggggag

SEQ ID NO: 59 - RNA, 21 nt, Homo sapiens
cccggagccaggaugcagcuc

SEQ ID NO: 60 - RNA, 21 nt, Homo sapiens
ucguggccuggucuccauuau

SEQ ID NO: 61 - RNA, 20 nt, Homo sapiens
ucugcaggguuugcuuugag

SEQ ID NO: 62 - RNA, 21 nt, Homo sapiens
uguucauguagauguuuaagc

SEQ ID NO: 63 - RNA, 21 nt, Homo sapiens
ccccaccuccucucuccucag

SEQ ID NO: 64 - RNA, 19 nt, Homo sapiens
gugaggacucgggaggugg

SEQ ID NO: 65 - RNA, 22 nt, Homo sapiens
ucaccagcccuguguucccuag

SEQ ID NO: 66 - RNA, 20 nt, Homo sapiens
ucacaccugccucgcccccc

SEQ ID NO: 67 - RNA, 20 nt, Homo sapiens
ugagcccuguccucccgcag

SEQ ID NO: 68 - RNA, 22 nt, Homo sapiens
ucggccugaccacccaccccac

SEQ ID NO: 69 - RNA, 22 nt, Homo sapiens
ccucuuccccuugucucuccag

SEQ ID NO: 70 - RNA, 26 nt, Homo sapiens
aaguaguugguuuguaugagaugguu

SEQ ID NO: 71 - RNA, 21 nt, Homo sapiens
aagugaucuaaaggccuacau

SEQ ID NO: 72 - RNA, 22 nt, Homo sapiens
ucagaugaucuaaaggccuaua

SEQ ID NO: 73 - RNA, 21 nt, Homo sapiens
uaggccuuuagaucacuuaaa

SEQ ID NO: 74 - RNA, 19 nt, Homo sapiens
aauggauuuuuggagcagg

SEQ ID NO: 75 - RNA, 22 nt, Homo sapiens
acccgucccguucguccccgga

SEQ ID NO: 76 - RNA, 27 nt, Homo sapiens
accuucuuguauaagcacugugcuaaa

SEQ ID NO: 77 - RNA, 22 nt, Homo sapiens
acgcccuucccccccuucuuca

SEQ ID NO: 78 - RNA, 24 nt, Homo sapiens
aggagggaggagaugggccaaguu

SEQ ID NO: 79 - RNA, 21 nt, Homo sapiens
acggugcuggauguggccuuu

SEQ ID NO: 80 - RNA, 22 nt, Homo sapiens
agaaggaaauugaauucauuua

SEQ ID NO: 81 - RNA, 21 nt, Homo sapiens
agagaagaagaucagccugca

SEQ ID NO: 82 - RNA, 24 nt, Homo sapiens
agccuggaagcuggagccugcagu

SEQ ID NO: 83 - RNA, 23 nt, Homo sapiens
aggaugagcaaagaaaguagauu

SEQ ID NO: 84 - RNA, 22 nt, Homo sapiens
cggaugagcaaagaaagugguu

SEQ ID NO: 85 - RNA, 21 nt, Homo sapiens
agugaaugauggguucugacc

SEQ ID NO: 86 - RNA, 21 nt, Homo sapiens
aguuaggauuaggucguggaa

SEQ ID NO: 87 - RNA, 22 nt, Homo sapiens
acaggugagguucuugggagcc

SEQ ID NO: 88 - RNA, 22 nt, Homo sapiens
ucccugagacccuaacuuguga

SEQ ID NO: 89 - RNA, 18 nt, Homo sapiens
aucccaccucugccacca

SEQ ID NO: 90 - RNA, 19 nt, Homo sapiens
aucccaccacugccaccau

SEQ ID NO: 91 - RNA, 19 nt, Homo sapiens
auggauaaggcuuuggcuu

SEQ ID NO: 92 - RNA, 22 nt, Homo sapiens
augggugaauuuguagaaggau

SEQ ID NO: 93 - RNA, 23 nt, Homo sapiens
caagucuuauuugagcaccuguu

SEQ ID NO: 94 - RNA, 23 nt, Homo sapiens
ccucagggcuguagaacagggcu

SEQ ID NO: 95 - RNA, 18 nt, Homo sapiens
cgggcguggugguggggg

SEQ ID NO: 96 - RNA, 20 nt, Homo sapiens
cgggcgugguggugggggug

SEQ ID NO: 97 - RNA, 22 nt, Homo sapiens
cuggacugagccgugcuacugg

SEQ ID NO: 98 - RNA, 22 nt, Homo sapiens
cuggacugagccaugcuacugg

SEQ ID NO: 99 - RNA, 23 nt, Homo sapiens
cuggagauauggaagagcugugu

SEQ ID NO: 100 - RNA, 22 nt, Homo sapiens
agugccugcuaugugccaggca

SEQ ID NO: 101 - RNA, 22 nt, Homo sapiens
cuuggcaccuagcaagcacuca

SEQ ID NO: 102 - RNA, 26 nt, Homo sapiens
gaugaugauggcagcaaauucugaaa

SEQ ID NO: 103 - RNA, 22 nt, Homo sapiens
ggcgacaaaacgagacccuguc

SEQ ID NO: 104 - RNA, 17 nt, Homo sapiens
gugggggagaggcuguc

SEQ ID NO: 105 - RNA, 22 nt, Homo sapiens
cugaagcucagagggcucugau

SEQ ID NO: 106 - RNA, 20 nt, Homo sapiens
uaaagagcccuguggagaca

SEQ ID NO: 107 - RNA, 22 nt, Homo sapiens
uacguagauauauauguauuuu

SEQ ID NO: 108 - RNA, 22 nt, Homo sapiens
uaguacugugcauaucaucuau

SEQ ID NO: 109 - RNA, 17 nt, Homo sapiens
ucauauugcuucuuucu

SEQ ID NO: 110 - RNA, 17 nt, Homo sapiens
ucgccuccuccucuccc

SEQ ID NO: 111 - RNA, 23 nt, Homo sapiens
cggggccguagcacugucugaga

SEQ ID NO: 112 - RNA, 23 nt, Homo sapiens
gggggccgauacacuguacgaga

SEQ ID NO: 113 - RNA, 22 nt, Homo sapiens
ucuacaaaggaaagcgcuuucu

SEQ ID NO: 114 - RNA, 22 nt, Homo sapiens
ucugggcaacaaagugagaccu

SEQ ID NO: 115 - RNA, 21 nt, Homo sapiens
gaucucacuuuguugcccagg

SEQ ID NO: 116 - RNA, 21 nt, Homo sapiens
ugcaggaccaagaugagcccu

SEQ ID NO: 117 - RNA, 22 nt, Homo sapiens
cucuagccacagaugcagugau

SEQ ID NO: 118 - RNA, 22 nt, Homo sapiens
ugcuggaucagugguucgaguc

SEQ ID NO: 119 - RNA, 21 nt, Homo sapiens
uggacugcccugaucuggaga

SEQ ID NO: 120 - RNA, 23 nt, Homo sapiens
uggaguccaggaaucugcauuuu

SEQ ID NO: 121 - RNA, 19 nt, Homo sapiens
uggauuuuuggaucaggga

SEQ ID NO: 122 - RNA, 24 nt, Homo sapiens
uggcccugacugaagaccagcagu

SEQ ID NO: 123 - RNA, 22 nt, Homo sapiens
aagcccuuaccccaaaaagcau

SEQ ID NO: 124 - RNA, 22 nt, Homo sapiens
uggguggucuggagauuugugc

SEQ ID NO: 125 - RNA, 21 nt, Homo sapiens
uuaggccgcagaucuggguga

SEQ ID NO: 126 - RNA, 22 nt, Homo sapiens
gaguggggcuucgacccuaacc

SEQ ID NO: 127 - RNA, 22 nt, Homo sapiens
uuagggcccuggcuccaucucc

SEQ ID NO: 128 - RNA, 17 nt, Homo sapiens
uucaaguaauucaggug

SEQ ID NO: 129 - RNA, 22 nt, Homo sapiens
uucauucggcuguccagaugua

SEQ ID NO: 130 - RNA, 22 nt, Homo sapiens
uucuggaauucugugugaggga

SEQ ID NO: 131 - RNA, 24 nt, Homo sapiens
uugcagcugccugggagugacuuc

SEQ ID NO: 132 - RNA, 21 nt, Homo sapiens
uugggacauacuuaugcuaaa

SEQ ID NO: 133 - RNA, 22 nt, Homo sapiens
uuuagagacggggucuugcucu

SEQ ID NO: 134 - RNA, 22 nt, Homo sapiens
ucucacuguagccucgaacccc

SEQ ID NO: 135 - RNA, 22 nt, Homo sapiens
uuugaggcuacagugagaugug

SEQ ID NO: 136 - RNA, 22 nt, Homo sapiens
uuuucaacucuaaugggagaga

SEQ ID NO: 137 - RNA, 18 nt, Homo sapiens
acguuggcucugguggug

SEQ ID NO: 138 - RNA, 22 nt, Homo sapiens
ccaccuccccugcaaacgucca

SEQ ID NO: 139 - RNA, 22 nt, Homo sapiens
acucggcguggcgucggucgug

SEQ ID NO: 140 - RNA, 21 nt, Homo sapiens
ucgaccggaccucgaccggcu

SEQ ID NO: 141 - RNA, 22 nt, Homo sapiens
cagugcaauguuaaaagggcau

SEQ ID NO: 142 - RNA, 22 nt, Homo sapiens
cagugcaaugaugaaagggcau

SEQ ID NO: 143 - RNA, 19 nt, Homo sapiens
gaugaugcugcugaugcug

SEQ ID NO: 144 - RNA, 22 nt, Homo sapiens
ucaaaacugaggggcauuuucu

SEQ ID NO: 145 - RNA, 22 nt, Homo sapiens
uaacagucuacagccauggucg

SEQ ID NO: 146 - RNA, 22 nt, Homo sapiens
agcugguaaaauggaaccaaau

SEQ ID NO: 147 - RNA, 22 nt, Homo sapiens
ugugacugguugaccagagggg

SEQ ID NO: 148 - RNA, 23 nt, Homo sapiens
uauggcuuuuuauuccuauguga

SEQ ID NO: 149 - RNA, 23 nt, Homo sapiens
uauggcuuuucauuccuauguga

SEQ ID NO: 150 - RNA, 23 nt, Homo sapiens
acuccauuuguuuugaugaugga

SEQ ID NO: 151 - RNA, 23 nt, Homo sapiens
agcugguguugugaaucaggccg

SEQ ID NO: 152 - RNA, 23 nt, Homo sapiens
uggagacgcggcccuguuggagu

SEQ ID NO: 153 - RNA, 23 nt, Homo sapiens
ucuacagugcacgugucuccagu

SEQ ID NO: 154 - RNA, 21 nt, Homo sapiens
uaccacaggguagaaccacgg

SEQ ID NO: 155 - RNA, 22 nt, Homo sapiens
cagugguuuuacccuaugguag

SEQ ID NO: 156 - RNA, 22 nt, Homo sapiens
uaacacugucugguaaagaugg

SEQ ID NO: 157 - RNA, 23 nt, Homo sapiens
uguaguguuuccuacuuuaugga

SEQ ID NO: 158 - RNA, 21 nt, Homo sapiens
cauaaaguagaaagcacuacu

SEQ ID NO: 159 - RNA, 20 nt, Homo sapiens
uacaguauagaugauguacu

SEQ ID NO: 160 - RNA, 22 nt, Homo sapiens
cucggcgcggggcgcgggcucc

SEQ ID NO: 161 - RNA, 22 nt, Homo sapiens
ugagaacugaauuccauggguu

SEQ ID NO: 162 - RNA, 22 nt, Homo sapiens
ugcccuguggacucaguucugg

SEQ ID NO: 163 - RNA, 22 nt, Homo sapiens
ugagaacugaauuccauaggcu

SEQ ID NO: 164 - RNA, 20 nt, Homo sapiens
guguguggaaaugcuucugc

SEQ ID NO: 165 - RNA, 22 nt, Homo sapiens
gugugcggaaaugcuucugcua

SEQ ID NO: 166 - RNA, 22 nt, Homo sapiens
ucagugcacuacagaacuuugu

SEQ ID NO: 167 - RNA, 22 nt, Homo sapiens
ucagugcaucacagaacuuugu

SEQ ID NO: 168 - RNA, 23 nt, Homo sapiens
ucuggcuccgugucuucacuccc

SEQ ID NO: 169 - RNA, 22 nt, Homo sapiens
ucucccaacccuuguaccagug

SEQ ID NO: 170 - RNA, 21 nt, Homo sapiens
cuagacugaagcuccuugagg

SEQ ID NO: 171 - RNA, 21 nt, Homo sapiens
ucgaggagcucacagucuagu

SEQ ID NO: 172 - RNA, 18 nt, Homo sapiens
ucgaggagcucacagucu

SEQ ID NO: 173 - RNA, 21 nt, Homo sapiens
ucagugcaugacagaacuugg

SEQ ID NO: 174 - RNA, 23 nt, Homo sapiens
agguucugugauacacuccgacu

SEQ ID NO: 175 - RNA, 22 nt, Homo sapiens
uugcauagucacaaaagugauc

SEQ ID NO: 176 - RNA, 22 nt, Homo sapiens
aaaaccgucuaguuacaguugu

SEQ ID NO: 177 - RNA, 22 nt, Homo sapiens
uagguuauccguguugccuucg

SEQ ID NO: 178 - RNA, 23 nt, Homo sapiens
uuaaugcuaaucgugauaggggu

SEQ ID NO: 179 - RNA, 22 nt, Homo sapiens
uagcagcacauaaugguuugug

SEQ ID NO: 180 - RNA, 22 nt, Homo sapiens
uagcagcacaucaugguuuaca

SEQ ID NO: 181 - RNA, 22 nt, Homo sapiens
acauacuucuuuauaugcccau

SEQ ID NO: 182 - RNA, 22 nt, Homo sapiens
uagcagcacguaaauauuggcg

SEQ ID NO: 183 - RNA, 22 nt, Homo sapiens
accacugaccguugacuguacc

SEQ ID NO: 184 - RNA, 22 nt, Homo sapiens
accaucgaccguugauuguacc

SEQ ID NO: 185 - RNA, 23 nt, Homo sapiens
aacauucaacgcugucggugagu

SEQ ID NO: 186 - RNA, 20 nt, Homo sapiens
cucacugaucaaugaaugca

SEQ ID NO: 187 - RNA, 23 nt, Homo sapiens
aacauucauugcugucggugggu

SEQ ID NO: 188 - RNA, 22 nt, Homo sapiens
aacauucaaccugucggugagu

SEQ ID NO: 189 - RNA, 21 nt, Homo sapiens
ccaccgggggaugaaugucac

SEQ ID NO: 190 - RNA, 21 nt, Homo sapiens
ugguucuagacuugccaacua

SEQ ID NO: 191 - RNA, 24 nt, Homo sapiens
uuuggcaaugguagaacucacacu

SEQ ID NO: 192 - RNA, 18 nt, Homo sapiens
ugaggcaguagauugaau

SEQ ID NO: 193 - RNA, 22 nt, Homo sapiens
uauggcacugguagaauucacu

SEQ ID NO: 194 - RNA, 22 nt, Homo sapiens
uggagagaaaggcaguuccuga

SEQ ID NO: 195 - RNA, 22 nt, Homo sapiens
caaagaauucuccuuuugggcu

SEQ ID NO: 196 - RNA, 22 nt, Homo sapiens
ucgugucuuguguugcagccgg

SEQ ID NO: 197 - RNA, 21 nt, Homo sapiens
cucccacaugcaggguuugca

SEQ ID NO: 198 - RNA, 21 nt, Homo sapiens
caucccuugcaugguggaggg

SEQ ID NO: 199 - RNA, 23 nt, Homo sapiens
uaaggugcaucuagugcagauag

SEQ ID NO: 200 - RNA, 23 nt, Homo sapiens
uaaggugcaucuagugcaguuag

SEQ ID NO: 201 - RNA, 21 nt, Homo sapiens
ccggccgccggcuccgccccg

SEQ ID NO: 202 - RNA, 21 nt, Homo sapiens
cggcggggacggcgauugguc

SEQ ID NO: 203 - RNA, 22 nt, Homo sapiens
cgcaggggccgggugcucaccg

SEQ ID NO: 204 - RNA, 21 nt, Homo sapiens
cuauauaucaaacauauuccu

SEQ ID NO: 205 - RNA, 22 nt, Homo sapiens
ugauauguuugauauauuaggu

SEQ ID NO: 206 - RNA, 21 nt, Homo sapiens
ugauauguuugauauuggguu

SEQ ID NO: 207 - RNA, 20 nt, Homo sapiens
gaggcagaagcaggaugaca

SEQ ID NO: 208 - RNA, 21 nt, Homo sapiens
ccaguccugugccugccgccu

SEQ ID NO: 209 - RNA, 20 nt, Homo sapiens
ccccagggcgacgcggcggg

SEQ ID NO: 210 - RNA, 23 nt, Homo sapiens
caacggaaucccaaaagcagcug

SEQ ID NO: 211 - RNA, 21 nt, Homo sapiens
cugaccuaugaauugacagcc

SEQ ID NO: 212 - RNA, 22 nt, Homo sapiens
aacuggccuacaaagucccagu

SEQ ID NO: 213 - RNA, 22 nt, Homo sapiens
ugggucuuugcgggcgagauga

SEQ ID NO: 214 - RNA, 22 nt, Homo sapiens
aacuggcccucaaagucccgcu

SEQ ID NO: 215 - RNA, 22 nt, Homo sapiens
uguaacagcaacuccaugugga

SEQ ID NO: 216 - RNA, 21 nt, Homo sapiens
uagcagcacagaaauauuggc

SEQ ID NO: 217 - RNA, 22 nt, Homo sapiens
cggcaacaagaaacugccugag

SEQ ID NO: 218 - RNA, 22 nt, Homo sapiens
uagguaguuucauguuguuggg

SEQ ID NO: 219 - RNA, 22 nt, Homo sapiens
uagguaguuuccuguuguuggg

SEQ ID NO: 220 - RNA, 22 nt, Homo sapiens
ucaggccaggcacaguggcuca

SEQ ID NO: 221 - RNA, 19 nt, Homo sapiens
accgugcaaagguagcaua

SEQ ID NO: 222 - RNA, 22 nt, Homo sapiens
uucaccaccuucuccacccagc

SEQ ID NO: 223 - RNA, 23 nt, Homo sapiens
cggguagagagggcagugggagg

SEQ ID NO: 224 - RNA, 20 nt, Homo sapiens
ccuccugcccuccuugcugu

SEQ ID NO: 225 - RNA, 22 nt, Homo sapiens
gguccagaggggagauagguuc

SEQ ID NO: 226 - RNA, 23 nt, Homo sapiens
cccaguguuuagacuaucuguuc

SEQ ID NO: 227 - RNA, 23 nt, Homo sapiens
ugugcaaaucuaugcaaaacuga

SEQ ID NO: 228 - RNA, 23 nt, Homo sapiens
ugugcaaauccaugcaaaacuga

SEQ ID NO: 229 - RNA, 22 nt, Homo sapiens
uaacacugucugguaacgaugu

SEQ ID NO: 230 - RNA, 22 nt, Homo sapiens
uaauacugccugguaaugauga

SEQ ID NO: 231 - RNA, 23 nt, Homo sapiens
uaauacugccggguaaugaugga

SEQ ID NO: 232 - RNA, 20 nt, Homo sapiens
agagguauagggcaugggaa

SEQ ID NO: 233 - RNA, 22 nt, Homo sapiens
gugaaauguuuaggaccacuag

SEQ ID NO: 234 - RNA, 25 nt, Homo sapiens
agugguucuuaacaguucaacaguu

SEQ ID NO: 235 - RNA, 23 nt, Homo sapiens
guguuaauuaaaccucuauuuac

SEQ ID NO: 236 - RNA, 22 nt, Homo sapiens
uccuucauuccaccggagucug

SEQ ID NO: 237 - RNA, 22 nt, Homo sapiens
uggaauguaaggaagugugugg

SEQ ID NO: 238 - RNA, 22 nt, Homo sapiens
auaagacgagcaaaaagcuugu

SEQ ID NO: 239 - RNA, 22 nt, Homo sapiens
aagcuuuuugcucgaauuaugu

SEQ ID NO: 240 - RNA, 23 nt, Homo sapiens
uaaagugcuuauagugcagguag

SEQ ID NO: 241 - RNA, 22 nt, Homo sapiens
cugugcgugugacagcggcuga

SEQ ID NO: 242 - RNA, 22 nt, Homo sapiens
agccccugcccaccgcacacug

SEQ ID NO: 243 - RNA, 22 nt, Homo sapiens
uuggggaaacggccgcugagug

SEQ ID NO: 244 - RNA, 21 nt, Homo sapiens
auuugugcuuggcucugucac

SEQ ID NO: 245 - RNA, 21 nt, Homo sapiens
gcagggacagcaaaggggugc

SEQ ID NO: 246 - RNA, 22 nt, Homo sapiens
uucccuuugucauccuucgccu

SEQ ID NO: 247 - RNA, 21 nt, Homo sapiens
gguucuuagcauaggaggucu

SEQ ID NO: 248 - RNA, 21 nt, Homo sapiens
uguucucuuugccaaggacag

SEQ ID NO: 249 - RNA, 21 nt, Homo sapiens
uaacagucuccagucacggcc

SEQ ID NO: 250 - RNA, 21 nt, Homo sapiens
augaccuaugaauugacagac

SEQ ID NO: 251 - RNA, 22 nt, Homo sapiens
uagcuuaucagacugauguuga

SEQ ID NO: 252 - RNA, 22 nt, Homo sapiens
uaaucucagcuggcaacuguga

SEQ ID NO: 253 - RNA, 22 nt, Homo sapiens
aaaucucugcaggcaaauguga

SEQ ID NO: 254 - RNA, 21 nt, Homo sapiens
uugugcuugaucuaaccaugu

SEQ ID NO: 255 - RNA, 22 nt, Homo sapiens
agaguugagucuggacgucccg

SEQ ID NO: 256 - RNA, 22 nt, Homo sapiens
agaauuguggcuggacaucugu

SEQ ID NO: 257 - RNA, 23 nt, Homo sapiens
agaauugcguuuggacaaucagu

SEQ ID NO: 258 - RNA, 23 nt, Homo sapiens
agcuacauugucugcuggguuuc

SEQ ID NO: 259 - RNA, 22 nt, Homo sapiens
accuggcauacaauguagauuu

SEQ ID NO: 260 - RNA, 21 nt, Homo sapiens
agcuacaucuggcuacugggu

SEQ ID NO: 261 - RNA, 22 nt, Homo sapiens
aagcugccaguugaagaacugu

SEQ ID NO: 262 - RNA, 21 nt, Homo sapiens
caagucacuagugguuccguu

SEQ ID NO: 263 - RNA, 22 nt, Homo sapiens
gagagcaguguguguugccugg

SEQ ID NO: 264 - RNA, 21 nt, Homo sapiens
aucacauugccagggauuucc

SEQ ID NO: 265 - RNA, 21 nt, Homo sapiens
aucacauugccagggauuacc

SEQ ID NO: 266 - RNA, 22 nt, Homo sapiens
aucacauugccagugauuaccc

SEQ ID NO: 267 - RNA, 22 nt, Homo sapiens
uggcucaguucagcaggaacag

SEQ ID NO: 268 - RNA, 22 nt, Homo sapiens
cauugcacuugucucggucuga

SEQ ID NO: 269 - RNA, 21 nt, Homo sapiens
aggcggagacuugggcaauug

SEQ ID NO: 270 - RNA, 23 nt, Homo sapiens
caggcagugacuguucagacguc

SEQ ID NO: 271 - RNA, 22 nt, Homo sapiens
uucaaguaauccaggauaggcu

SEQ ID NO: 272 - RNA, 21 nt, Homo sapiens
uucaaguaauucaggauaggu

SEQ ID NO: 273 - RNA, 21 nt, Homo sapiens
uucacaguggcuaaguuccgc

SEQ ID NO: 274 - RNA, 21 nt, Homo sapiens
uucacaguggcuaaguucugc

SEQ ID NO: 275 - RNA, 22 nt, Homo sapiens
cacuagauugugagcuccugga

SEQ ID NO: 276 - RNA, 22 nt, Homo sapiens
aaggagcucacagucuauugag

SEQ ID NO: 277 - RNA, 22 nt, Homo sapiens
gaggguuggguggaggcucucc

SEQ ID NO: 278 - RNA, 21 nt, Homo sapiens
agggcccccccucaauccugu

SEQ ID NO: 279 - RNA, 21 nt, Homo sapiens
auguaugugugcaugugcaug

SEQ ID NO: 280 - RNA, 24 nt, Homo sapiens
agcagaagcagggagguucuccca

SEQ ID NO: 281 - RNA, 22 nt, Homo sapiens
uaugugggaugguaaaccgcuu

SEQ ID NO: 282 - RNA, 22 nt, Homo sapiens
ugguuuaccgucccacauacau

SEQ ID NO: 283 - RNA, 22 nt, Homo sapiens
uagcaccaucugaaaucgguua

SEQ ID NO: 284 - RNA, 23 nt, Homo sapiens
uagcaccauuugaaaucaguguu

SEQ ID NO: 285 - RNA, 22 nt, Homo sapiens
uagcaccauuugaaaucgguua

SEQ ID NO: 286 - RNA, 22 nt, Homo sapiens
uauacaagggcagacucucucu

SEQ ID NO: 287 - RNA, 23 nt, Homo sapiens
cagugcaauaguauugucaaagc

SEQ ID NO: 288 - RNA, 22 nt, Homo sapiens
gcucugacuuuauugcacuacu

SEQ ID NO: 289 - RNA, 23 nt, Homo sapiens
cagugcaaugauauugucaaagc

SEQ ID NO: 290 - RNA, 22 nt, Homo sapiens
gcucugacgagguugcacuacu

SEQ ID NO: 291 - RNA, 23 nt, Homo sapiens
uaagugcuuccauguuuugguga

SEQ ID NO: 292 - RNA, 23 nt, Homo sapiens
acuuaaacguggauguacuugcu

SEQ ID NO: 293 - RNA, 23 nt, Homo sapiens
uaagugcuuccauguuuuaguag

SEQ ID NO: 294 - RNA, 23 nt, Homo sapiens
uaagugcuuccauguuucagugg

SEQ ID NO: 295 - RNA, 23 nt, Homo sapiens
uaagugcuuccauguuugagugu

SEQ ID NO: 296 - RNA, 17 nt, Homo sapiens
uaagugcuuccaugcuu

SEQ ID NO: 297 - RNA, 17 nt, Homo sapiens
uaauugcuuccauguuu

SEQ ID NO: 298 - RNA, 23 nt, Homo sapiens
ucagcaccaggauauuguuggag

SEQ ID NO: 299 - RNA, 23 nt, Homo sapiens
ucaacaaaaucacugaugcugga

SEQ ID NO: 300 - RNA, 22 nt, Homo sapiens
gauaucagcucaguaggcaccg

SEQ ID NO: 301 - RNA, 22 nt, Homo sapiens
cuuucagucggauguuugcagc

SEQ ID NO: 302 - RNA, 22 nt, Homo sapiens
uguaaacauccucgacuggaag

SEQ ID NO: 303 - RNA, 22 nt, Homo sapiens
uguaaacauccuacacucagcu

SEQ ID NO: 304 - RNA, 23 nt, Homo sapiens
uguaaacauccuacacucucagc

SEQ ID NO: 305 - RNA, 22 nt, Homo sapiens
uguaaacauccccgacuggaag

SEQ ID NO: 306 - RNA, 22 nt, Homo sapiens
cuuucagucggauguuuacagc

SEQ ID NO: 307 - RNA, 22 nt, Homo sapiens
uguaaacauccuugacuggaag

SEQ ID NO: 308 - RNA, 23 nt, Homo sapiens
aucagggcuuguggaaugggaag

SEQ ID NO: 309 - RNA, 21 nt, Homo sapiens
gcugcaccggagacuggguaa

SEQ ID NO: 310 - RNA, 23 nt, Homo sapiens
ucgaggacugguggaagggccuu

SEQ ID NO: 311 - RNA, 23 nt, Homo sapiens
cugacugaauagguagggucauu

SEQ ID NO: 312 - RNA, 22 nt, Homo sapiens
agcuuuugggaauucagguagu

SEQ ID NO: 313 - RNA, 21 nt, Homo sapiens
accugaauuaccaaaagcuuu

SEQ ID NO: 314 - RNA, 22 nt, Homo sapiens
auauaccuguucggucucuuua

SEQ ID NO: 315 - RNA, 22 nt, Homo sapiens
aggggaccaaagagauauauag

SEQ ID NO: 316 - RNA, 24 nt, Homo sapiens
gguugggcagugaggaggguguga

SEQ ID NO: 317 - RNA, 21 nt, Homo sapiens
ugaggagaucgucgagguugg

SEQ ID NO: 318 - RNA, 21 nt, Homo sapiens
gguggggcaaugggaucaggu

SEQ ID NO: 319 - RNA, 22 nt, Homo sapiens
aagggcuuccucucugcaggac

SEQ ID NO: 320 - RNA, 21 nt, Homo sapiens
aggcaagaugcuggcauagcu

SEQ ID NO: 321 - RNA, 23 nt, Homo sapiens
cugauaagaacagaggcccagau

SEQ ID NO: 322 - RNA, 22 nt, Homo sapiens
ugugacuuuaagggaaauggcg

SEQ ID NO: 323 - RNA, 17 nt, Homo sapiens
gaguucuacagucagac

SEQ ID NO: 324 - RNA, 22 nt, Homo sapiens
agaaggggugaaauuuaaacgu

SEQ ID NO: 325 - RNA, 19 nt, Homo sapiens
uggggcggagcuuccggag

SEQ ID NO: 326 - RNA, 22 nt, Homo sapiens
uggggcggagcuuccggaggcc

SEQ ID NO: 327 - RNA, 25 nt, Homo sapiens
cuuccagacgcuccgccccacgucg

SEQ ID NO: 328 - RNA, 17 nt, Homo sapiens
gcuucuguaguguaguc

SEQ ID NO: 329 - RNA, 23 nt, Homo sapiens
agaagaaggcggucggucugcgg

SEQ ID NO: 330 - RNA, 23 nt, Homo sapiens
uguggaagguagacggccagaga

SEQ ID NO: 331 - RNA, 23 nt, Homo sapiens
ucugggagguuguagcaguggaa

SEQ ID NO: 332 - RNA, 17 nt, Homo sapiens
cgcgccgggcccggguu

SEQ ID NO: 333 - RNA, 18 nt, Homo sapiens
cggggcggcaggggccuc

SEQ ID NO: 334 - RNA, 22 nt, Homo sapiens
uggaagggagaagagcuuuaau

SEQ ID NO: 335 - RNA, 22 nt, Homo sapiens
aaaagcuggguugagagggcga

SEQ ID NO: 336 - RNA, 22 nt, Homo sapiens
aaaagcuggguugagagggcaa

SEQ ID NO: 337 - RNA, 20 nt, Homo sapiens
aaaagcuggguugagagggu

SEQ ID NO: 338 - RNA, 19 nt, Homo sapiens
aaaagcuggguugagagga

SEQ ID NO: 339 - RNA, 18 nt, Homo sapiens
aaagcuggguugagaagg

SEQ ID NO: 340 - RNA, 21 nt, Homo sapiens
cacauuacacggucgaccucu

SEQ ID NO: 341 - RNA, 22 nt, Homo sapiens
aggugguccguggcgcguucgc

SEQ ID NO: 342 - RNA, 22 nt, Homo sapiens
cccaauacacggucgaccucuu

SEQ ID NO: 343 - RNA, 23 nt, Homo sapiens
agguuguccguggugaguucgca

SEQ ID NO: 344 - RNA, 20 nt, Homo sapiens
acugccccaggugcugcugg

SEQ ID NO: 345 - RNA, 23 nt, Homo sapiens
cgcauccccuagggcauuggugu

SEQ ID NO: 346 - RNA, 23 nt, Homo sapiens
ccuaguagguguccaguaagugu

SEQ ID NO: 347 - RNA, 22 nt, Homo sapiens
uauugcacauuacuaaguugca

SEQ ID NO: 348 - RNA, 20 nt, Homo sapiens
ccucugggcccuuccuccag

SEQ ID NO: 349 - RNA, 22 nt, Homo sapiens
cuggcccucucugcccuuccgu

SEQ ID NO: 350 - RNA, 23 nt, Homo sapiens
gggggggcaggaggggcucaggg

SEQ ID NO: 351 - RNA, 22 nt, Homo sapiens
aacacaccugguuaaccucuuu

SEQ ID NO: 352 - RNA, 23 nt, Homo sapiens
gagguuuucuggguuucuguuuc

SEQ ID NO: 353 - RNA, 23 nt, Homo sapiens
gcaaagcacacggccugcagaga

SEQ ID NO: 354 - RNA, 22 nt, Homo sapiens
ucucugggccugugucuuaggc

SEQ ID NO: 355 - RNA, 21 nt, Homo sapiens
gccccugggccuauccuagaa

SEQ ID NO: 356 - RNA, 22 nt, Homo sapiens
cuagguauggucccagggaucc

SEQ ID NO: 357 - RNA, 23 nt, Homo sapiens
ucaagagcaauaacgaaaaaugu

SEQ ID NO: 358 - RNA, 22 nt, Homo sapiens
cuccuauaugaugccuuucuuc

SEQ ID NO: 359 - RNA, 21 nt, Homo sapiens
gaacggcuucauacaggaguu

SEQ ID NO: 360 - RNA, 22 nt, Homo sapiens
aacaauauccuggugcugagug

SEQ ID NO: 361 - RNA, 23 nt, Homo sapiens
ugagcgccucgacgacagagccg

SEQ ID NO: 362 - RNA, 23 nt, Homo sapiens
ucccuguccuccaggagcucacg

SEQ ID NO: 363 - RNA, 21 nt, Homo sapiens
gugcauuguaguugcauugca

SEQ ID NO: 364 - RNA, 20 nt, Homo sapiens
gugcauugcuguugcauugc

SEQ ID NO: 365 - RNA, 22 nt, Homo sapiens
uuauaaagcaaugagacugauu

SEQ ID NO: 366 - RNA, 23 nt, Homo sapiens
ucucacacagaaaucgcacccgu

SEQ ID NO: 367 - RNA, 21 nt, Homo sapiens
aggggugcuaucugugauuga

SEQ ID NO: 368 - RNA, 22 nt, Homo sapiens
gcccugaacgaggggucuggag

SEQ ID NO: 369 - RNA, 22 nt, Homo sapiens
gcugacuccuaguccagggcuc

SEQ ID NO: 370 - RNA, 23 nt, Homo sapiens
ugucugcccgcaugccugccucu

SEQ ID NO: 371 - RNA, 22 nt, Homo sapiens
caaucacuaacuccacugccau

SEQ ID NO: 372 - RNA, 22 nt, Homo sapiens
aaucacuaaccacacggccagg

SEQ ID NO: 373 - RNA, 23 nt, Homo sapiens
aggcaguguaguuagcugauugc

SEQ ID NO: 374 - RNA, 23 nt, Homo sapiens
ccuccguguuaccuguccucuag

SEQ ID NO: 375 - RNA, 23 nt, Homo sapiens
ugaggauggauagcaaggaagcc

SEQ ID NO: 376 - RNA, 24 nt, Homo sapiens
acaaaaaaaaaagcccaacccuuc

SEQ ID NO: 377 - RNA, 22 nt, Homo sapiens
uguuguacuuuuuuuuuuguuc

SEQ ID NO: 378 - RNA, 23 nt, Homo sapiens
ucccccaggugugauucugauuu

SEQ ID NO: 379 - RNA, 23 nt, Homo sapiens
uagccuucagaucuugguguuuu

SEQ ID NO: 380 - RNA, 23 nt, Homo sapiens
ccacuuggaucugaaggcugccc

SEQ ID NO: 381 - RNA, 21 nt, Homo sapiens
ucucucggcuccucgcggcuc

SEQ ID NO: 382 - RNA, 22 nt, Homo sapiens
uuaucagaaucuccagggguac

SEQ ID NO: 383 - RNA, 22 nt, Homo sapiens
aacacaccuauucaaggauuca

SEQ ID NO: 384 - RNA, 24 nt, Homo sapiens
aauccuuggaaccuaggugugagu

SEQ ID NO: 385 - RNA, 22 nt, Homo sapiens
aauugcacgguauccaucugua

SEQ ID NO: 386 - RNA, 22 nt, Homo sapiens
cggguggaucacgaugcaauuu

SEQ ID NO: 387 - RNA, 22 nt, Homo sapiens
uaaugccccuaaaaauccuuau

SEQ ID NO: 388 - RNA, 22 nt, Homo sapiens
agggacuuucaggggcagcugu

SEQ ID NO: 389 - RNA, 22 nt, Homo sapiens
aauugcacuuuagcaaugguga

SEQ ID NO: 390 - RNA, 23 nt, Homo sapiens
accuggacccagcguagacaaag

SEQ ID NO: 391 - RNA, 21 nt, Homo sapiens
aauaauacaugguugaucuuu

SEQ ID NO: 392 - RNA, 22 nt, Homo sapiens
agaucgaccguguuauauucgc

SEQ ID NO: 393 - RNA, 22 nt, Homo sapiens
gccugcugggguggaaccuggu

SEQ ID NO: 394 - RNA, 22 nt, Homo sapiens
caggucacgucucugcaguuac

SEQ ID NO: 395 - RNA, 20 nt, Homo sapiens
acucaaacugugggggcacu

SEQ ID NO: 396 - RNA, 22 nt, Homo sapiens
acucaaaagauggcggcacuuu

SEQ ID NO: 397 - RNA, 23 nt, Homo sapiens
aaagugcugcgacauuugagcgu

SEQ ID NO: 398 - RNA, 23 nt, Homo sapiens
gaagugcuucgauuuuggggugu

SEQ ID NO: 399 - RNA, 22 nt, Homo sapiens
cuuaucagauuguauuguaauu

SEQ ID NO: 400 - RNA, 22 nt, Homo sapiens
uuauaauacaaccugauaagug

SEQ ID NO: 401 - RNA, 22 nt, Homo sapiens
auauaauacaaccugcuaagug

SEQ ID NO: 402 - RNA, 22 nt, Homo sapiens
auaauacaaccugcuaagugcu

SEQ ID NO: 403 - RNA, 22 nt, Homo sapiens
uuuguucguucggcucgcguga

SEQ ID NO: 404 - RNA, 21 nt, Homo sapiens
gguagauuuuccuucuauggu

SEQ ID NO: 405 - RNA, 21 nt, Homo sapiens
aucauagaggaaaauccacgu

SEQ ID NO: 406 - RNA, 22 nt, Homo sapiens
aucauagaggaaaauccauguu

SEQ ID NO: 407 - RNA, 21 nt, Homo sapiens
aacauagaggaaauuccacgu

SEQ ID NO: 408 - RNA, 21 nt, Homo sapiens
gguggauauuccuucuauguu

SEQ ID NO: 409 - RNA, 22 nt, Homo sapiens
aucacacaaaggcaacuuuugu

SEQ ID NO: 410 - RNA, 19 nt, Homo sapiens
acuggacuuggaggcagaa

SEQ ID NO: 411 - RNA, 25 nt, Homo sapiens
acuggacuuggagucagaagagugg

SEQ ID NO: 412 - RNA, 20 nt, Homo sapiens
acuggacuuggagucagaaa

SEQ ID NO: 413 - RNA, 19 nt, Homo sapiens
acuggacuuggagucagga

SEQ ID NO: 414 - RNA, 20 nt, Homo sapiens
acuggacuuggagccagaag

SEQ ID NO: 415 - RNA, 20 nt, Homo sapiens
acugggcuuggagucagaag

SEQ ID NO: 416 - RNA, 21 nt, Homo sapiens
acuggacuuggugucagaugg

SEQ ID NO: 417 - RNA, 21 nt, Homo sapiens
acuggacuaggagucagaagg

SEQ ID NO: 418 - RNA, 21 nt, Homo sapiens
ugguagacuauggaacguagg

SEQ ID NO: 419 - RNA, 22 nt, Homo sapiens
uauguaauaugguccacaucuu

SEQ ID NO: 420 - RNA, 22 nt, Homo sapiens
uauacaagggcaagcucucugu

SEQ ID NO: 421 - RNA, 22 nt, Homo sapiens
agcgagguugcccuuuguauau

SEQ ID NO: 422 - RNA, 21 nt, Homo sapiens
aaucauucacggacaacacuu

SEQ ID NO: 423 - RNA, 22 nt, Homo sapiens
gaaguuguucgugguggauucg

SEQ ID NO: 424 - RNA, 22 nt, Homo sapiens
agaucagaaggugauuguggcu

SEQ ID NO: 425 - RNA, 20 nt, Homo sapiens
auuccuagaaauuguucaua

SEQ ID NO: 426 - RNA, 26 nt, Homo sapiens
aagaggaagaaauggcugguucucag

SEQ ID NO: 427 - RNA, 21 nt, Homo sapiens
acagggccgcagauggagacu

SEQ ID NO: 428 - RNA, 22 nt, Homo sapiens
ggaggaaccuuggagcuucggc

SEQ ID NO: 429 - RNA, 22 nt, Homo sapiens
ucagguguggaaacugaggcag

SEQ ID NO: 430 - RNA, 22 nt, Homo sapiens
gaauguugcucggugaaccccu

SEQ ID NO: 431 - RNA, 23 nt, Homo sapiens
agguuacccgagcaacuuugcau

SEQ ID NO: 432 - RNA, 21 nt, Homo sapiens
aauauaacacagauggccugu

SEQ ID NO: 433 - RNA, 21 nt, Homo sapiens
uaguagaccguauagcguacg

SEQ ID NO: 434 - RNA, 23 nt, Homo sapiens
acuucaccugguccacuagccgu

SEQ ID NO: 435 - RNA, 23 nt, Homo sapiens
aucaacagacauuaauugggcgc

SEQ ID NO: 436 - RNA, 22 nt, Homo sapiens
acuggacuuagggucagaaggc

SEQ ID NO: 437 - RNA, 23 nt, Homo sapiens
agcucggucugaggccccucagu

SEQ ID NO: 438 - RNA, 23 nt, Homo sapiens
ugaggggcagagagcgagacuuu

SEQ ID NO: 439 - RNA, 22 nt, Homo sapiens
cagcagcaauucauguuuugaa

SEQ ID NO: 440 - RNA, 23 nt, Homo sapiens
aaugacacgaucacucccguuga

SEQ ID NO: 441 - RNA, 18 nt, Homo sapiens
gggcucacaucaccccau

SEQ ID NO: 442 - RNA, 17 nt, Homo sapiens
accccacuccugguacc

SEQ ID NO: 443 - RNA, 22 nt, Homo sapiens
uaauacugucugguaaaaccgu

SEQ ID NO: 444 - RNA, 21 nt, Homo sapiens
ugucuugcaggccgucaugca

SEQ ID NO: 445 - RNA, 23 nt, Homo sapiens
ucuuggaguaggucauugggugg

SEQ ID NO: 446 - RNA, 22 nt, Homo sapiens
aucaugaugggcuccucggugu

SEQ ID NO: 447 - RNA, 22 nt, Homo sapiens
uacggugagccugucauuauuc

SEQ ID NO: 448 - RNA, 22 nt, Homo sapiens
accugucuguggaaaggagcua

SEQ ID NO: 449 - RNA, 22 nt, Homo sapiens
uguugggauucagcaggaccau

SEQ ID NO: 450 - RNA, 22 nt, Homo sapiens
gcgacucugaaaacuagaaggu

SEQ ID NO: 451 - RNA, 22 nt, Homo sapiens
auggccagagcucacacagagg

SEQ ID NO: 452 - RNA, 17 nt, Homo sapiens
uuggaggcguggguuuu

SEQ ID NO: 453 - RNA, 20 nt, Homo sapiens
ggcuccuuggucuaggggua

SEQ ID NO: 454 - RNA, 17 nt, Homo sapiens
ugguagagcugaggaca

SEQ ID NO: 455 - RNA, 20 nt, Homo sapiens
ggauccgagucacggcacca

SEQ ID NO: 456 - RNA, 17 nt, Homo sapiens
aggguguguguguuuuu

SEQ ID NO: 457 - RNA, 19 nt, Homo sapiens
agagguagguguggaagaa

SEQ ID NO: 458 - RNA, 23 nt, Homo sapiens
gauugagacuaguagggcuaggc

SEQ ID NO: 459 - RNA, 22 nt, Homo sapiens
uugcauauguaggaugucccau

SEQ ID NO: 460 - RNA, 20 nt, Homo sapiens
uaacggccgcgguacccuaa

SEQ ID NO: 461 - RNA, 18 nt, Homo sapiens
agggggcgggcuccggcg

SEQ ID NO: 462 - RNA, 22 nt, Homo sapiens
uggcaguguauuguuagcuggu

SEQ ID NO: 463 - RNA, 22 nt, Homo sapiens
aggcaguguauuguuagcuggc

SEQ ID NO: 464 - RNA, 25 nt, Homo sapiens
uaggcaguguauugcuagcggcugu

SEQ ID NO: 465 - RNA, 22 nt, Homo sapiens
auugggaacauuuugcauguau

SEQ ID NO: 466 - RNA, 22 nt, Homo sapiens
auuggggacauuuugcauucau

SEQ ID NO: 467 - RNA, 22 nt, Homo sapiens
uuuugcgauguguuccuaauau

SEQ ID NO: 468 - RNA, 22 nt, Homo sapiens
uugggaucauuuugcauccaua

SEQ ID NO: 469 - RNA, 22 nt, Homo sapiens
uuuugcaauauguuccugaaua

SEQ ID NO: 470 - RNA, 17 nt, Homo sapiens
gggagaagggucggggc

SEQ ID NO: 471 - RNA, 22 nt, Homo sapiens
gcuaaggaaguccugugcucag

SEQ ID NO: 472 - RNA, 22 nt, Homo sapiens
auagcagcaugaaccugucuca

SEQ ID NO: 473 - RNA, 22 nt, Homo sapiens
aacuguuugcagaggaaacuga

SEQ ID NO: 474 - RNA, 17 nt, Homo sapiens
auggagaaggcuucuga

SEQ ID NO: 475 - RNA, 17 nt, Homo sapiens
ccccggggagcccggcg

SEQ ID NO: 476 - RNA, 21 nt, Homo sapiens
ucgugcauauaucuaccacau

SEQ ID NO: 477 - RNA, 21 nt, Homo sapiens
ugugguagauauaugcacgau

SEQ ID NO: 478 - RNA, 23 nt, Homo sapiens
uagugcaauauugcuuauagggu

SEQ ID NO: 479 - RNA, 21 nt, Homo sapiens
gcaguccaugggcauauacac

SEQ ID NO: 480 - RNA, 22 nt, Homo sapiens
uaugugccuuuggacuacaucg

SEQ ID NO: 481 - RNA, 23 nt, Homo sapiens
gaagauggugcugugcugaggaa

SEQ ID NO: 482 - RNA, 22 nt, Homo sapiens
agcccgccccagccgagguucu

SEQ ID NO: 483 - RNA, 23 nt, Homo sapiens
gccccggcgcgggcggguucugg

SEQ ID NO: 484 - RNA, 23 nt, Homo sapiens
cgggcuguccggaggggucggcu

SEQ ID NO: 485 - RNA, 22 nt, Homo sapiens
uuucccuucagagccuggcuuu

SEQ ID NO: 486 - RNA, 24 nt, Homo sapiens
gaugcgccgcccacugccccgcgc

SEQ ID NO: 487 - RNA, 22 nt, Homo sapiens
gcggggguggcggcggcauccc

SEQ ID NO: 488 - RNA, 18 nt, Homo sapiens
cggugagcgcucgcuggc

SEQ ID NO: 489 - RNA, 21 nt, Homo sapiens
ucacuccucuccucccgucuu

SEQ ID NO: 490 - RNA, 22 nt, Homo sapiens
aagacgggaggaaagaagggag

SEQ ID NO: 491 - RNA, 22 nt, Homo sapiens
ucaggcucaguccccucccgau

SEQ ID NO: 492 - RNA, 22 nt, Homo sapiens
gucauacacggcucuccucucu

SEQ ID NO: 493 - RNA, 22 nt, Homo sapiens
agaggcuggccgugaugaauuc

SEQ ID NO: 494 - RNA, 21 nt, Homo sapiens
cggggcagcucaguacaggau

SEQ ID NO: 495 - RNA, 22 nt, Homo sapiens
aaucauacagggacauccaguu

SEQ ID NO: 496 - RNA, 22 nt, Homo sapiens
aaucguacagggucauccacuu

SEQ ID NO: 497 - RNA, 22 nt, Homo sapiens
gugguuaucccuguccuguucg

SEQ ID NO: 498 - RNA, 21 nt, Homo sapiens
uugaaaggcuauuucuugguc

SEQ ID NO: 499 - RNA, 22 nt, Homo sapiens
gugacaucacauauacggcagc

SEQ ID NO: 500 - RNA, 22 nt, Homo sapiens
caaccuggaggacuccaugcug

SEQ ID NO: 501 - RNA, 20 nt, Homo sapiens
ccauggaucuccaggugggu

SEQ ID NO: 502 - RNA, 22 nt, Homo sapiens
cuuaugcaagauucccuucuac

SEQ ID NO: 503 - RNA, 22 nt, Homo sapiens
aguggggaacccuuccaugagg

SEQ ID NO: 504 - RNA, 23 nt, Homo sapiens
aggaccugcgggacaagauucuu

SEQ ID NO: 505 - RNA, 22 nt, Homo sapiens
ugaaggucuacugugugccagg

SEQ ID NO: 506 - RNA, 22 nt, Homo sapiens
ugaaacauacacgggaaaccuc

SEQ ID NO: 507 - RNA, 23 nt, Homo sapiens
agguuguccguguugucuucucu

SEQ ID NO: 508 - RNA, 22 nt, Homo sapiens
aaacaaacauggugcacuucuu

SEQ ID NO: 509 - RNA, 22 nt, Homo sapiens
gaaguugcccauguuauuuucg

SEQ ID NO: 510 - RNA, 22 nt, Homo sapiens
ugaguauuacauggccaaucuc

SEQ ID NO: 511 - RNA, 21 nt, Homo sapiens
cagcagcacacugugguuugu

SEQ ID NO: 512 - RNA, 23 nt, Homo sapiens
uuucaagccagggggcguuuuuc

SEQ ID NO: 513 - RNA, 22 nt, Homo sapiens
aacaucacagcaagucugugcu

SEQ ID NO: 514 - RNA, 21 nt, Homo sapiens
uuaagacuugcagugauguuu

SEQ ID NO: 515 - RNA, 22 nt, Homo sapiens
aacaucacugcaagucuuaaca

SEQ ID NO: 516 - RNA, 21 nt, Homo sapiens
acagacuugcugugauguuca

SEQ ID NO: 517 - RNA, 22 nt, Homo sapiens
uucugccucuguccagguccuu

SEQ ID NO: 518 - RNA, 24 nt, Homo sapiens
agggcuggacucagcggcggagcu

SEQ ID NO: 519 - RNA, 23 nt, Homo sapiens
uaauccuugcuaccugggugaga

SEQ ID NO: 520 - RNA, 22 nt, Homo sapiens
uuuugugucucccauuccccag

SEQ ID NO: 521 - RNA, 22 nt, Homo sapiens
agggggauggcagagcaaaauu

SEQ ID NO: 522 - RNA, 22 nt, Homo sapiens
aaugcacccgggcaaggauucu

SEQ ID NO: 523 - RNA, 22 nt, Homo sapiens
aaugcaccugggcaaggauuca

SEQ ID NO: 524 - RNA, 21 nt, Homo sapiens
auccuugcuaucugggugcua

SEQ ID NO: 525 - RNA, 23 nt, Homo sapiens
gggguauuguuuccgcugccagg

SEQ ID NO: 526 - RNA, 23 nt, Homo sapiens
uagcagcgggaacaguucugcag

SEQ ID NO: 527 - RNA, 21 nt, Homo sapiens
gggagugcagggcaggguuuc

SEQ ID NO: 528 - RNA, 22 nt, Homo sapiens
agacccuggucugcacucuauc

SEQ ID NO: 529 - RNA, 22 nt, Homo sapiens
cgucaacacuugcugguuuccu

SEQ ID NO: 530 - RNA, 21 nt, Homo sapiens
uaaggcacccuucugaguaga

SEQ ID NO: 531 - RNA, 22 nt, Homo sapiens
uauucaggaagguguuacuuaa

SEQ ID NO: 532 - RNA, 21 nt, Homo sapiens
uuuugcaccuuuuggagugaa

SEQ ID NO: 533 - RNA, 23 nt, Homo sapiens
ugauuguagccuuuuggaguaga

SEQ ID NO: 534 - RNA, 23 nt, Homo sapiens
uacuccagagggcgucacucaug

SEQ ID NO: 535 - RNA, 22 nt, Homo sapiens
uacugcagacguggcaaucaug

SEQ ID NO: 536 - RNA, 22 nt, Homo sapiens
ugauugguacgucuguggguag

SEQ ID NO: 537 - RNA, 21 nt, Homo sapiens
uacugcagacaguggcaauca

SEQ ID NO: 538 - RNA, 21 nt, Homo sapiens
auugaaaccucuaagagugga

SEQ ID NO: 539 - RNA, 22 nt, Homo sapiens
uacucaggagaguggcaaucac

SEQ ID NO: 540 - RNA, 21 nt, Homo sapiens
gugucuuuugcucugcaguca

SEQ ID NO: 541 - RNA, 22 nt, Homo sapiens
aagugcugucauagcugagguc

SEQ ID NO: 542 - RNA, 23 nt, Homo sapiens
cacucagccuugagggcacuuuc

SEQ ID NO: 543 - RNA, 23 nt, Homo sapiens
uaaauuucaccuuucugagaagg

SEQ ID NO: 544 - RNA, 18 nt, Homo sapiens
uucacagggaggugucau

SEQ ID NO: 545 - RNA, 22 nt, Homo sapiens
uucacaaggaggugucauuuau

SEQ ID NO: 546 - RNA, 23 nt, Homo sapiens
uaaauuucaccuuucugagaaga

SEQ ID NO: 547 - RNA, 22 nt, Homo sapiens
uucucaaggaggugucguuuau

SEQ ID NO: 548 - RNA, 21 nt, Homo sapiens
auugacacuucugugaguaga

SEQ ID NO: 549 - RNA, 23 nt, Homo sapiens
uacucuggagagugacaaucaug

SEQ ID NO: 550 - RNA, 21 nt, Homo sapiens
auugacaccucugugagugga

SEQ ID NO: 551 - RNA, 22 nt, Homo sapiens
uucucaagagggaggcaaucau

SEQ ID NO: 552 - RNA, 22 nt, Homo sapiens
gagugccuucuuuuggagcguu

SEQ ID NO: 553 - RNA, 24 nt, Homo sapiens
uucuccaaaagaaagcacuuucug

SEQ ID NO: 554 - RNA, 18 nt, Homo sapiens
ugcuuccuuucagagggu

SEQ ID NO: 555 - RNA, 23 nt, Homo sapiens
uucucgaggaaagaagcacuuuc

SEQ ID NO: 556 - RNA, 22 nt, Homo sapiens
aucuggagguaagaagcacuuu

SEQ ID NO: 557 - RNA, 22 nt, Homo sapiens
aucgugcaucccuuuagagugu

SEQ ID NO: 558 - RNA, 22 nt, Homo sapiens
aucgugcaucccuuuagagugu

SEQ ID NO: 559 - RNA, 22 nt, Homo sapiens
aucgugcauccuuuuagagugu

SEQ ID NO: 560 - RNA, 22 nt, Homo sapiens
caaagcgcuccccuuuagaggu

SEQ ID NO: 561 - RNA, 23 nt, Homo sapiens
caaagcgcuucucuuuagagugu

SEQ ID NO: 562 - RNA, 21 nt, Homo sapiens
caaagcgcuucccuuuggagc

SEQ ID NO: 563 - RNA, 21 nt, Homo sapiens
aaagcgcuucccuucagagug

SEQ ID NO: 564 - RNA, 21 nt, Homo sapiens
gaaagcgcuucucuuuagagg

SEQ ID NO: 565 - RNA, 21 nt, Homo sapiens
ucauccucgucucccucccag

SEQ ID NO: 566 - RNA, 22 nt, Homo sapiens
agggaaggggacgaggguuggg

SEQ ID NO: 567 - RNA, 22 nt, Homo sapiens
aaagugcauccuuuuagagguu

SEQ ID NO: 568 - RNA, 22 nt, Homo sapiens
cucuagagggaagcgcuuucug

SEQ ID NO: 569 - RNA, 22 nt, Homo sapiens
aaagugcaucuuuuuagaggau

SEQ ID NO: 570 - RNA, 22 nt, Homo sapiens
caaagugccucccuuuagagug

SEQ ID NO: 571 - RNA, 22 nt, Homo sapiens
aagugccuccuuuuagaguguu

SEQ ID NO: 572 - RNA, 22 nt, Homo sapiens
aaagugcuucccuuuggacugu

SEQ ID NO: 573 - RNA, 21 nt, Homo sapiens
cuccagagggaaguacuuucu

SEQ ID NO: 574 - RNA, 21 nt, Homo sapiens
aaagugcuuccuuuuagaggg

SEQ ID NO: 575 - RNA, 22 nt, Homo sapiens
aaagugcuuccuuuuagagggu

SEQ ID NO: 576 - RNA, 22 nt, Homo sapiens
aaagugcuucucuuuggugggu

SEQ ID NO: 577 - RNA, 20 nt, Homo sapiens
cuacaaagggaagcccuuuc

SEQ ID NO: 578 - RNA, 21 nt, Homo sapiens
aaagugcuuccuuuuugaggg

SEQ ID NO: 579 - RNA, 22 nt, Homo sapiens
aagugcuuccuuuuagaggguu

SEQ ID NO: 580 - RNA, 24 nt, Homo sapiens
acaaagugcuucccuuuagagugu

SEQ ID NO: 581 - RNA, 22 nt, Homo sapiens
acaaagugcuucccuuuagagu

SEQ ID NO: 582 - RNA, 22 nt, Homo sapiens
aacgcacuucccuuuagagugu

SEQ ID NO: 583 - RNA, 22 nt, Homo sapiens
aaaaugguucccuuuagagugu

SEQ ID NO: 584 - RNA, 23 nt, Homo sapiens
gaacgcgcuucccuauagagggu

SEQ ID NO: 585 - RNA, 21 nt, Homo sapiens
gaaggcgcuucccuuuggagu

SEQ ID NO: 586 - RNA, 22 nt, Homo sapiens
gaaggcgcuucccuuuagagcg

SEQ ID NO: 587 - RNA, 21 nt, Homo sapiens
cuccagagggaugcacuuucu

SEQ ID NO: 588 - RNA, 22 nt, Homo sapiens
cucuagagggaagcacuuucug

SEQ ID NO: 589 - RNA, 23 nt, Homo sapiens
cucuugagggaagcacuuucugu

SEQ ID NO: 590 - RNA, 22 nt, Homo sapiens
ccucccacacccaaggcuugca

SEQ ID NO: 591 - RNA, 22 nt, Homo sapiens
caugccuugaguguaggaccgu

SEQ ID NO: 592 - RNA, 22 nt, Homo sapiens
aucauacaaggacaauuucuuu

SEQ ID NO: 593 - RNA, 22 nt, Homo sapiens
ggagaaauuauccuuggugugu

SEQ ID NO: 594 - RNA, 22 nt, Homo sapiens
uggugggcacagaaucuggacu

SEQ ID NO: 595 - RNA, 22 nt, Homo sapiens
ugugacagauugauaacugaaa

SEQ ID NO: 596 - RNA, 23 nt, Homo sapiens
ucggggaucaucaugucacgaga

SEQ ID NO: 597 - RNA, 22 nt, Homo sapiens
aaacauucgcggugcacuucuu

SEQ ID NO: 598 - RNA, 22 nt, Homo sapiens
auucugcauuuuuagcaaguuc

SEQ ID NO: 599 - RNA, 22 nt, Homo sapiens
ucagcaaacauuuauugugugc

SEQ ID NO: 600 - RNA, 22 nt, Homo sapiens
caaaacuggcaauuacuuuugc

SEQ ID NO: 601 - RNA, 22 nt, Homo sapiens
aaaaguaauugcgaguuuuacc

SEQ ID NO: 602 - RNA, 25 nt, Homo sapiens
aaaaaccacaauuacuuuugcacca

SEQ ID NO: 603 - RNA, 22 nt, Homo sapiens
gaaaacgacaaugacuuuugca

SEQ ID NO: 604 - RNA, 20 nt, Homo sapiens
aaaagugauugcaguguuug

SEQ ID NO: 605 - RNA, 22 nt, Homo sapiens
aaagguaauugcaguuuuuccc

SEQ ID NO: 606 - RNA, 21 nt, Homo sapiens
aaaaguaacugcgguuuuuga

SEQ ID NO: 607 - RNA, 22 nt, Homo sapiens
aacggcaaugacuuuuguacca

SEQ ID NO: 608 - RNA, 21 nt, Homo sapiens
uaaaacugcaguuauuuuugc

SEQ ID NO: 609 - RNA, 21 nt, Homo sapiens
aaaaguaauugcaguuuuugc

SEQ ID NO: 610 - RNA, 22 nt, Homo sapiens
caagaaccucaguugcuuuugu

SEQ ID NO: 611 - RNA, 22 nt, Homo sapiens
aaaaguaauugcgguuuuugcc

SEQ ID NO: 612 - RNA, 22 nt, Homo sapiens
caaaaaccacaguuucuuuugc

SEQ ID NO: 613 - RNA, 22 nt, Homo sapiens
aaaaguaauugugguuuuugcc

SEQ ID NO: 614 - RNA, 22 nt, Homo sapiens
aaaaacugagacuacuuuugca

SEQ ID NO: 615 - RNA, 22 nt, Homo sapiens
caaaagcaaucgcgguuuuugc

SEQ ID NO: 616 - RNA, 22 nt, Homo sapiens
aaaacuguaauuacuuuuguac

SEQ ID NO: 617 - RNA, 22 nt, Homo sapiens
aaaaguaaucgcgguuuuuguc

SEQ ID NO: 618 - RNA, 22 nt, Homo sapiens
aaaaguaauugcggauuuugcc

SEQ ID NO: 619 - RNA, 21 nt, Homo sapiens
caaaaacugcauuacuuuugc

SEQ ID NO: 620 - RNA, 22 nt, Homo sapiens
aaaaguaauugcggucuuuggu

SEQ ID NO: 621 - RNA, 22 nt, Homo sapiens
aaaaguacuugcggauuuugcu

SEQ ID NO: 622 - RNA, 22 nt, Homo sapiens
aaaaguauuugcggguuuuguc

SEQ ID NO: 623 - RNA, 21 nt, Homo sapiens
caaagguauuugugguuuuug

SEQ ID NO: 624 - RNA, 22 nt, Homo sapiens
caaaaguaauuguggauuuugu

SEQ ID NO: 625 - RNA, 22 nt, Homo sapiens
ccaaaacugcaguuacuuuugc

SEQ ID NO: 626 - RNA, 22 nt, Homo sapiens
gcuggugcaaaaguaauggcgg

SEQ ID NO: 627 - RNA, 22 nt, Homo sapiens
agcuacaguuacuuuugcacca

SEQ ID NO: 628 - RNA, 22 nt, Homo sapiens
aaaaguaaucacuguuuuugcc

SEQ ID NO: 629 - RNA, 23 nt, Homo sapiens
caaaaaccgcaauuacuuuugca

SEQ ID NO: 630 - RNA, 21 nt, Homo sapiens
ugacaacuauggaugagcucu

SEQ ID NO: 631 - RNA, 23 nt, Homo sapiens
agugccugagggaguaagagccc

SEQ ID NO: 632 - RNA, 21 nt, Homo sapiens
gcgacccacucuugguuucca

SEQ ID NO: 633 - RNA, 21 nt, Homo sapiens
gcgacccauacuugguuucag

SEQ ID NO: 634 - RNA, 21 nt, Homo sapiens
aacaggugacugguuagacaa

SEQ ID NO: 635 - RNA, 21 nt, Homo sapiens
aaaacggugagauuuuguuuu

SEQ ID NO: 636 - RNA, 21 nt, Homo sapiens
gcuaguccugacucagccagu

SEQ ID NO: 637 - RNA, 21 nt, Homo sapiens
aggguaagcugaaccucugau

SEQ ID NO: 638 - RNA, 22 nt, Homo sapiens
auauuaccauuagcucaucuuu

SEQ ID NO: 639 - RNA, 22 nt, Homo sapiens
gaugagcucauuguaauaugag

SEQ ID NO: 640 - RNA, 22 nt, Homo sapiens
caaaguuuaagauccuugaagu

SEQ ID NO: 641 - RNA, 22 nt, Homo sapiens
aucaaggaucuuaaacuuugcc

SEQ ID NO: 642 - RNA, 20 nt, Homo sapiens
aaaguagcuguaccauuugc

SEQ ID NO: 643 - RNA, 19 nt, Homo sapiens
agguugacauacguuuccc

SEQ ID NO: 644 - RNA, 19 nt, Homo sapiens
aggcacggugucagcaggc

SEQ ID NO: 645 - RNA, 19 nt, Homo sapiens
gggcgccugugaucccaac

SEQ ID NO: 646 - RNA, 23 nt, Homo sapiens
aguauguucuuccaggacagaac

SEQ ID NO: 647 - RNA, 20 nt, Homo sapiens
auguauaaauguauacacac

SEQ ID NO: 648 - RNA, 22 nt, Homo sapiens
cgaaaacagcaauuaccuuugc

SEQ ID NO: 649 - RNA, 21 nt, Homo sapiens
ugaguuggccaucugagugag

SEQ ID NO: 650 - RNA, 20 nt, Homo sapiens
guccgcucggcgguggccca

SEQ ID NO: 651 - RNA, 24 nt, Homo sapiens
cugaagugauguguaacugaucag

SEQ ID NO: 652 - RNA, 22 nt, Homo sapiens
cacgcucaugcacacacccaca

SEQ ID NO: 653 - RNA, 23 nt, Homo sapiens
ugagugugugugugugagugugu

SEQ ID NO: 654 - RNA, 19 nt, Homo sapiens
gagccaguuggacaggagc

SEQ ID NO: 655 - RNA, 22 nt, Homo sapiens
aagauguggaaaaauuggaauc

SEQ ID NO: 656 - RNA, 22 nt, Homo sapiens
auucuaauuucuccacgucuuu

SEQ ID NO: 657 - RNA, 21 nt, Homo sapiens
uagauaaaauauugguaccug

SEQ ID NO: 658 - RNA, 21 nt, Homo sapiens
cuucuugugcucuaggauugu

SEQ ID NO: 659 - RNA, 23 nt, Homo sapiens
uucauuugguauaaaccgcgauu

SEQ ID NO: 660 - RNA, 22 nt, Homo sapiens
ucgcgguuugugccagaugacg

SEQ ID NO: 661 - RNA, 22 nt, Homo sapiens
uugagaaugaugaaucauuagg

SEQ ID NO: 662 - RNA, 22 nt, Homo sapiens
uaacugguugaacaacugaacc

SEQ ID NO: 663 - RNA, 23 nt, Homo sapiens
uuacaguuguucaaccaguuacu

SEQ ID NO: 664 - RNA, 22 nt, Homo sapiens
ucaguuccaggccaaccaggcu

SEQ ID NO: 665 - RNA, 22 nt, Homo sapiens
uuaugguuugccugggacugag

SEQ ID NO: 666 - RNA, 19 nt, Homo sapiens
ugggcguaucuguaugcua

SEQ ID NO: 667 - RNA, 21 nt, Homo sapiens
uuuccauaggugaugagucac

SEQ ID NO: 668 - RNA, 22 nt, Homo sapiens
ugagaaccacgucugcucugag

SEQ ID NO: 669 - RNA, 21 nt, Homo sapiens
uaauuuuauguauaagcuagu

SEQ ID NO: 670 - RNA, 22 nt, Homo sapiens
gagcuuauucauaaaagugcag

SEQ ID NO: 671 - RNA, 20 nt, Homo sapiens
agaccauggguucucauugu

SEQ ID NO: 672 - RNA, 22 nt, Homo sapiens
uugugucaauaugcgaugaugu

SEQ ID NO: 673 - RNA, 19 nt, Homo sapiens
ugucucugcugggguuucu

SEQ ID NO: 674 - RNA, 21 nt, Homo sapiens
gaagugugccguggugugucu

SEQ ID NO: 675 - RNA, 21 nt, Homo sapiens
aagccugcccggcuccucggg

SEQ ID NO: 676 - RNA, 22 nt, Homo sapiens
ugugucacucgaugaccacugu

SEQ ID NO: 677 - RNA, 22 nt, Homo sapiens
uacgucaucguugucaucguca

SEQ ID NO: 678 - RNA, 20 nt, Homo sapiens
guugugucaguuuaucaaac

SEQ ID NO: 679 - RNA, 23 nt, Homo sapiens
acuuacagacaagagccuugcuc

SEQ ID NO: 680 - RNA, 22 nt, Homo sapiens
uggucuaggauuguuggaggag

SEQ ID NO: 681 - RNA, 22 nt, Homo sapiens
cacacacugcaauuacuuuugc

SEQ ID NO: 682 - RNA, 19 nt, Homo sapiens
aggcugcggaauucaggac

SEQ ID NO: 683 - RNA, 23 nt, Homo sapiens
uaaaucccauggugccuucuccu

SEQ ID NO: 684 - RNA, 21 nt, Homo sapiens
aaacuacugaaaaucaaagau

SEQ ID NO: 685 - RNA, 21 nt, Homo sapiens
guucaaauccagaucuauaac

SEQ ID NO: 686 - RNA, 25 nt, Homo sapiens
aggggugguguugggacagcuccgu

SEQ ID NO: 687 - RNA, 21 nt, Homo sapiens
ugagcuaaaugugugcuggga

SEQ ID NO: 688 - RNA, 25 nt, Homo sapiens
gcugggcagggcuucugagcuccuu

SEQ ID NO: 689 - RNA, 20 nt, Homo sapiens
aggaauguuccuucuuugcc

SEQ ID NO: 690 - RNA, 23 nt, Homo sapiens
gaacgccuguucuugccaggugg

SEQ ID NO: 691 - RNA, 22 nt, Homo sapiens
uccgagccugggucucccucuu

SEQ ID NO: 692 - RNA, 22 nt, Homo sapiens
ggggguccccggugcucggauc

SEQ ID NO: 693 - RNA, 22 nt, Homo sapiens
agucauuggaggguuugagcag

SEQ ID NO: 694 - RNA, 22 nt, Homo sapiens
agacuucccauuugaagguggc

SEQ ID NO: 695 - RNA, 24 nt, Homo sapiens
gaccuggacauguuugugcccagu

SEQ ID NO: 696 - RNA, 20 nt, Homo sapiens
auggagauagauauagaaau

SEQ ID NO: 697 - RNA, 21 nt, Homo sapiens
cacaagguauugguauuaccu

SEQ ID NO: 698 - RNA, 21 nt, Homo sapiens
agggggaaaguucuauagucc

SEQ ID NO: 699 - RNA, 19 nt, Homo sapiens
agcugucugaaaaugucuu

SEQ ID NO: 700 - RNA, 20 nt, Homo sapiens
ucuuuucuuugagacucacu

SEQ ID NO: 701 - RNA, 22 nt, Homo sapiens
gugagucucuaagaaaagagga

SEQ ID NO: 702 - RNA, 21 nt, Homo sapiens
ucuaguaagaguggcagucga

SEQ ID NO: 703 - RNA, 22 nt, Homo sapiens
augcugacauauuuacuagagg

SEQ ID NO: 704 - RNA, 21 nt, Homo sapiens
uggguuuacguugggagaacu

SEQ ID NO: 705 - RNA, 22 nt, Homo sapiens
aguauucuguaccagggaaggu

SEQ ID NO: 706 - RNA, 21 nt, Homo sapiens
agaccuggcccagaccucagc

SEQ ID NO: 707 - RNA, 24 nt, Homo sapiens
acugggggcuuucgggcucugcgu

SEQ ID NO: 708 - RNA, 25 nt, Homo sapiens
agggaucgcgggcggguggcggccu

SEQ ID NO: 709 - RNA, 23 nt, Homo sapiens
aucgcugcgguugcgagcgcugu

SEQ ID NO: 710 - RNA, 21 nt, Homo sapiens
augauccaggaaccugccucu

SEQ ID NO: 711 - RNA, 24 nt, Homo sapiens
aaagacauaggauagagucaccuc

SEQ ID NO: 712 - RNA, 22 nt, Homo sapiens
agacacauuuggagagggaacc

SEQ ID NO: 713 - RNA, 22 nt, Homo sapiens
gucccucuccaaaugugucuug

SEQ ID NO: 714 - RNA, 22 nt, Homo sapiens
acuuguaugcuagcucagguag

SEQ ID NO: 715 - RNA, 19 nt, Homo sapiens
aguguggcuuucuuagagc

SEQ ID NO: 716 - RNA, 19 nt, Homo sapiens
aagugugcagggcacuggu

SEQ ID NO: 717 - RNA, 22 nt, Homo sapiens
aaaccuguguuguucaagaguc

SEQ ID NO: 718 - RNA, 21 nt, Homo sapiens
aggaggcagcgcucucaggac

SEQ ID NO: 719 - RNA, 21 nt, Homo sapiens
gggacuaggaugcagaccucc

SEQ ID NO: 720 - RNA, 23 nt, Homo sapiens
aggucugcauucaaauccccaga

SEQ ID NO: 721 - RNA, 22 nt, Homo sapiens
ccucaccaucccuucugccugc

SEQ ID NO: 722 - RNA, 23 nt, Homo sapiens
caggcagaaguggggcugacagg

SEQ ID NO: 723 - RNA, 22 nt, Homo sapiens
aaaggaaaguguauccuaaaag

SEQ ID NO: 724 - RNA, 22 nt, Homo sapiens
uuuaggauaagcuugacuuuug

SEQ ID NO: 725 - RNA, 21 nt, Homo sapiens
aauggcgccacuaggguugug

SEQ ID NO: 726 - RNA, 25 nt, Homo sapiens
caacccuaggagagggugccauuca

SEQ ID NO: 727 - RNA, 22 nt, Homo sapiens
uaugucugcugaccaucaccuu

SEQ ID NO: 728 - RNA, 22 nt, Homo sapiens
uggugggccgcagaacaugugc

SEQ ID NO: 729 - RNA, 22 nt, Homo sapiens
auaauacaugguuaaccucuuu

SEQ ID NO: 730 - RNA, 21 nt, Homo sapiens
aauauuauacagucaaccucu

SEQ ID NO: 731 - RNA, 21 nt, Homo sapiens
accuccugugugcauggauua

SEQ ID NO: 732 - RNA, 22 nt, Homo sapiens
uacccauugcauaucggaguug

SEQ ID NO: 733 - RNA, 24 nt, Homo sapiens
ugccugggucucuggccugcgcgu

SEQ ID NO: 734 - RNA, 22 nt, Homo sapiens
aggcggggcgccgcgggaccgc

SEQ ID NO: 735 - RNA, 23 nt, Homo sapiens
uauucauuuauccccagccuaca

SEQ ID NO: 736 - RNA, 22 nt, Homo sapiens
uucauuugccucccagccuaca

SEQ ID NO: 737 - RNA, 24 nt, Homo sapiens
ugggcuaagggagaugauugggua

SEQ ID NO: 738 - RNA, 20 nt, Homo sapiens
accaggaggcugaggccccu

SEQ ID NO: 739 - RNA, 21 nt, Homo sapiens
uccgguucucagggcuccacc

SEQ ID NO: 740 - RNA, 23 nt, Homo sapiens
aggaagcccuggaggggcuggag

SEQ ID NO: 741 - RNA, 22 nt, Homo sapiens
cgcgccugcaggaacugguaga

SEQ ID NO: 742 - RNA, 23 nt, Homo sapiens
ugggcaggggcuuauuguaggag

SEQ ID NO: 743 - RNA, 23 nt, Homo sapiens
cugggcccgcggcgggcgugggg

SEQ ID NO: 744 - RNA, 23 nt, Homo sapiens
uggugcggagagggcccacagug

SEQ ID NO: 745 - RNA, 23 nt, Homo sapiens
aaggagcuuacaaucuagcuggg

SEQ ID NO: 746 - RNA, 22 nt, Homo sapiens
ugcggggcuagggcuaacagca

SEQ ID NO: 747 - RNA, 22 nt, Homo sapiens
uuugugaccugguccacuaacc

SEQ ID NO: 748 - RNA, 22 nt, Homo sapiens
gaugguugaccagagagcacac

SEQ ID NO: 749 - RNA, 23 nt, Homo sapiens
uggaagacuagugauuuuguugu

SEQ ID NO: 750 - RNA, 20 nt, Homo sapiens
cggcucugggucugugggga

SEQ ID NO: 751 - RNA, 22 nt, Homo sapiens
gcagcagggugaaacugacaca

SEQ ID NO: 752 - RNA, 22 nt, Homo sapiens
gcaggugcucacuuguccuccu

SEQ ID NO: 753 - RNA, 21 nt, Homo sapiens
uggaggagaaggaaggugaug

SEQ ID NO: 754 - RNA, 22 nt, Homo sapiens
acuccagccccacagccucagc

SEQ ID NO: 755 - RNA, 22 nt, Homo sapiens
aggaggaauuggugcuggucuu

SEQ ID NO: 756 - RNA, 23 nt, Homo sapiens
ucugcucauaccccaugguuucu

SEQ ID NO: 757 - RNA, 23 nt, Homo sapiens
ugcaccaugguugucugagcaug

SEQ ID NO: 758 - RNA, 23 nt, Homo sapiens
cugggaucuccggggucuugguu

SEQ ID NO: 759 - RNA, 22 nt, Homo sapiens
ugagaccucuggguucugagcu

SEQ ID NO: 760 - RNA, 23 nt, Homo sapiens
uccaguaccacgugucagggcca

SEQ ID NO: 761 - RNA, 23 nt, Homo sapiens
caguaacaaagauucauccuugu

SEQ ID NO: 762 - RNA, 22 nt, Homo sapiens
ggagacugaugaguucccggga

SEQ ID NO: 763 - RNA, 21 nt, Homo sapiens
gcaggaacuugugagucuccu

SEQ ID NO: 764 - RNA, 22 nt, Homo sapiens
cugcccuggcccgagggaccga

SEQ ID NO: 765 - RNA, 23 nt, Homo sapiens
cggccccacgcaccaggguaaga

SEQ ID NO: 766 - RNA, 21 nt, Homo sapiens
ccuggaaacacugagguugug

SEQ ID NO: 767 - RNA, 22 nt, Homo sapiens
uggugguuuacaaaguaauuca

SEQ ID NO: 768 - RNA, 22 nt, Homo sapiens
uggauuucuuugugaaucacca

SEQ ID NO: 769 - RNA, 20 nt, Homo sapiens
guagaggagauggcgcaggg

SEQ ID NO: 770 - RNA, 22 nt, Homo sapiens
aggcagcgggguguaguggaua

SEQ ID NO: 771 - RNA, 22 nt, Homo sapiens
uccauuacacuacccugccucu

SEQ ID NO: 772 - RNA, 22 nt, Homo sapiens
gugaacgggcgccaucccgagg

SEQ ID NO: 773 - RNA, 21 nt, Homo sapiens
cuugggagcccuguuagacuc

SEQ ID NO: 774 - RNA, 21 nt, Homo sapiens
uacucaaaaagcugucaguca

SEQ ID NO: 775 - RNA, 21 nt, Homo sapiens
uuaauaucggacaaccauugu

SEQ ID NO: 776 - RNA, 21 nt, Homo sapiens
uacuuggaaaggcaucaguug

SEQ ID NO: 777 - RNA, 22 nt, Homo sapiens
ugcaacgaaccugagccacuga

SEQ ID NO: 778 - RNA, 22 nt, Homo sapiens
ugcaacuuaccugagucauuga

SEQ ID NO: 779 - RNA, 21 nt, Homo sapiens
cacuguguccuuucugcguag

SEQ ID NO: 780 - RNA, 22 nt, Homo sapiens
cacuggcuccuuucuggguaga

SEQ ID NO: 781 - RNA, 23 nt, Homo sapiens
gcagcagagaauaggacuacguc

SEQ ID NO: 782 - RNA, 20 nt, Homo sapiens
agagucuugugaugucuugc

SEQ ID NO: 783 - RNA, 23 nt, Homo sapiens
agguugggaucgguugcaaugcu

SEQ ID NO: 784 - RNA, 22 nt, Homo sapiens
uauugcacuugucccggccugu

SEQ ID NO: 785 - RNA, 22 nt, Homo sapiens
uauugcacucgucccggccucc

SEQ ID NO: 786 - RNA, 22 nt, Homo sapiens
ugugcgcagggagaccucuccc

SEQ ID NO: 787 - RNA, 22 nt, Homo sapiens
ugucuacuacuggagacacugg

SEQ ID NO: 788 - RNA, 23 nt, Homo sapiens
ccaguuaccgcuuccgcuaccgc

SEQ ID NO: 789 - RNA, 23 nt, Homo sapiens
caaagugcuguucgugcagguag

SEQ ID NO: 790 - RNA, 22 nt, Homo sapiens
acaguagagggaggaaucgcag

SEQ ID NO: 791 - RNA, 22 nt, Homo sapiens
auccgcgcucugacucucugcc

SEQ ID NO: 792 - RNA, 24 nt, Homo sapiens
uggggagcugaggcucugggggug

SEQ ID NO: 793 - RNA, 21 nt, Homo sapiens
aaggcagggcccccgcucccc

SEQ ID NO: 794 - RNA, 23 nt, Homo sapiens
cacccggcugugugcacaugugc

SEQ ID NO: 795 - RNA, 22 nt, Homo sapiens
cacauggccgaaacagagaagu

SEQ ID NO: 796 - RNA, 22 nt, Homo sapiens
ucuucucuguuuuggccaugug

SEQ ID NO: 797 - RNA, 22 nt, Homo sapiens
aaauuauuguacaucggaugag

SEQ ID NO: 798 - RNA, 22 nt, Homo sapiens
uucaacggguauuuauugagca

SEQ ID NO: 799 - RNA, 23 nt, Homo sapiens
uuuggcacuagcacauuuuugcu

SEQ ID NO: 800 - RNA, 22 nt, Homo sapiens
cuauacaacuuacuacuuuccc

SEQ ID NO: 801 - RNA, 22 nt, Homo sapiens
ugagguaguaaguuguauuguu

SEQ ID NO: 802 - RNA, 22 nt, Homo sapiens
aacccguagauccgaucuugug

SEQ ID NO: 803 - RNA, 22 nt, Homo sapiens
cacccguagaaccgaccuugcg

SEQ ID NO: 804 - DNA, 22 bp, Homo sapiens
caaacaccattgtcacactcca

SEQ ID NO: 805 - DNA, 20 bp, Homo sapiens
ggcattcaccgcgtgcctta

SEQ ID NO: 806 - DNA, 24 bp, Homo sapiens
tcacaggttaaagggtctcaggga

SEQ ID NO: 807 - DNA, 22 bp, Homo sapiens
cgcattattactcacggtacga

SEQ ID NO: 808 - DNA, 22 bp, Homo sapiens
cacattattactcacggtacga

SEQ ID NO: 809 - DNA, 22 bp, Homo sapiens
agccaagctcagacggatccga

SEQ ID NO: 810 - DNA, 21 bp, Homo sapiens
aaagagaccggttcactgtga

SEQ ID NO: 811 - DNA, 21 bp, Homo sapiens
aaagagaccggttcactgtgg

SEQ ID NO: 812 - DNA, 22 bp, Homo sapiens
atactttttggggtaagggctt

SEQ ID NO: 813 - DNA, 21 bp, Homo sapiens
gcaagcccagaccgcaaaaag

SEQ ID NO: 814 - DNA, 22 bp, Homo sapiens
atgccctttcatcattgcactg

SEQ ID NO: 815 - DNA, 21 bp, Homo sapiens
gtagtgcaacagggaaagagt

SEQ ID NO: 816 - DNA, 22 bp, Homo sapiens
cagctggttgaaggggaccaaa

SEQ ID NO: 817 - DNA, 22 bp, Homo sapiens
tagctggttgaaggggaccaaa

SEQ ID NO: 818 - DNA, 23 bp, Homo sapiens
ttggtgactaggtggcccacagg

SEQ ID NO: 819 - DNA, 23 bp, Homo sapiens
ctacgcgtattcttaagcaataa

SEQ ID NO: 820 - DNA, 22 bp, Homo sapiens
atacatacttctttacattcca

SEQ ID NO: 821 - DNA, 21 bp, Homo sapiens
gagctacagtgcttcatctca

SEQ ID NO: 822 - DNA, 22 bp, Homo sapiens
agaacagtatttccaggaatcc

SEQ ID NO: 823 - DNA, 23 bp, Homo sapiens
agggattcctgggaaaactggac

SEQ ID NO: 824 - DNA, 22 bp, Homo sapiens
acccttatcagttctccgtcca

SEQ ID NO: 825 - DNA, 22 bp, Homo sapiens
taaccaatgtgcagactactgt

SEQ ID NO: 826 - DNA, 23 bp, Homo sapiens
gaacaggtagtctgaacactggg

SEQ ID NO: 827 - DNA, 22 bp, Homo sapiens
aggcataggatgacaaagggaa

SEQ ID NO: 828 - DNA, 22 bp, Homo sapiens
acaaaccttttgttcgtcttat

SEQ ID NO: 829 - DNA, 22 bp, Homo sapiens
actgcctgtctgtgcctgctgt

SEQ ID NO: 830 - DNA, 23 bp, Homo sapiens
tccaatcagttcctgatgcagta

SEQ ID NO: 831 - DNA, 21 bp, Homo sapiens
agaattgcgtttggacaatca

SEQ ID NO: 832 - DNA, 22 bp, Homo sapiens
tggggtatttgacaaactgaca

SEQ ID NO: 833 - DNA, 22 bp, Homo sapiens
acaaccagctaagacactgcca

SEQ ID NO: 834 - DNA, 22 bp, Homo sapiens
aactcagtaatggtaacggttt

SEQ ID NO: 835 - DNA, 21 bp, Homo sapiens
ttttggtgcatatttacttta

SEQ ID NO: 836 - DNA, 22 bp, Homo sapiens
aactatacaacctactacctca

SEQ ID NO: 837 - DNA, 23 bp, Homo sapiens
tcatacagctagataaccaaaga

SEQ ID NO: 838 - DNA, 208 bp, Artificial Sequence
  miRT-1-143_1736
ccatatacatacttctttacattccatcctgagctacagtgcttcatctcattgcatacatacttctttacattccaacgtgagctacagtgcttcatctcatccgatacatacttctttacattccacggcgagctacagtgcttcatctcaccttatacatacttctttacattccaaaaagagctacagtgcttcatctcaccat

SEQ ID NO: 839 - DNA, 308 bp, Artificial Sequence
  miRT-128m-122-219_6793
cacgagaattgcgtttggacaatcagacacaaacaccattgtcacactccatcttaaagagaccggttcactgtggatgtcaaacaccattgtcacactccaacttagaattgcgtttggacaatcaagggaaagagaccggttcactgtggccagcaaacaccattgtcacactccaaaacaaagagaccggttcactgtggtacgagaattgcgtttggacaatcagaaaaaagagaccggttcactgtggaatacaaacaccattgtcacactccaacaaagaattgcgtttggacaatcaggtt

SEQ ID NO: 840 - DNA, 308 bp, Artificial Sequence
  miRT-128m-204-219_9304
aagtaaagagaccggttcactgtggaataagaattgcgtttggacaatcaaggtaggcataggatgacaaagggaacagcaaagagaccggttcactgtggggctagaattgcgtttggacaatcacgtaaggcataggatgacaaagggaacgagaaagagaccggttcactgtgggggaagaattgcgtttggacaatcatactaggcataggatgacaaagggaattagaaagagaccggttcactgtggatttagaattgcgtttggacaatcatagaaggcataggatgacaaagggaattgt

SEQ ID NO: 841 - DNA, 324 bp, Artificial Sequence
  miRT-217-137-126m_3163
tatgctacgcgtattcttaagcaataagacttccaatcagttcctgatgcagtacgaccacattattactcacggtacgaaagcctacgcgtattcttaagcaataaccgccacattattactcacggtacgataaatccaatcagttcctgatgcagtaattactacgcgtattcttaagcaataactattccaatcagttcctgatgcagtacccccacattattactcacggtacgagaattccaatcagttcctgatgcagtacagtcacattattactcacggtacgatcaactacgcgtattcttaagcaataaccaa

SEQ ID NO: 842 - RNA, 7310 nt, Senecavirus Senecavirus A
uuugaaauggggggcugggcccugaugcccaguccuuccuuuccccuuccgggggguuaaccggcuguguuugcuagaggcacagaggggcaacauccaaccugcuuuugcggggaacggugcggcuccgauuccugcgucgccaaagguguuagcgcacccaaacggcgcaccuaccaauguuauugguguggucugcgaguucuagccuacucguuucucccccgaccauucacucacccacgaaaaguguguuguaaccauaagauuuaacccccgcacgggaugugcgauaaccguaagacuggcucaagcgcggaaagcgcuguaaccacaugcuguuagucccuuuauggcugcaagauggcuacccaccucggaucacugaacuggagcucgacccuccuuaguaagggaaccgagaggccuucgugcaacaagcuccgacacagaguccacgugacugcuaccaccaugaguacaugguucuccccucucgacccaggacuucuuuuugaauauccacggcucgauccagaggguggggcaugaccccuagcauagcgagcuacagcgggaacuguagcuaggccuuagcgugccuuggauacugccugauagggcgacggccuagucgugucgguucuauagguagcacauacaaauaugcagaacucucauuuuucuuucgauacagccucuggcaccuuugaagauguaaccggaacaaaagucaagaucguugaauaccccagaucggugaacaaugguguuuacgauucgucuacucauuuggagauacugaaccuacagggugaaauugaaauuuuaaggucuuucaaugaauaccaaauucgcgccgccaaacaacaacucggacuggacaucguguacgaacuacaggguaauguucagacaacgucaaagaaugauuuugauucccguggcaauaaugguaacaugaccuucaauuacuacgcaaacacuuaucagaauucaguagacuucucgaccuccucgucggcgucaggcgccggaccugggaacucucggggcggauuagcgggucuccucacaaauuucaguggaaucuugaacccucuuggcuaccucaaagaucacaacaccgaagaaauggaaaacucugcugaucgagucacaacgcaaacggcgggcaacacugccauaaacacgcaaucaucauuggguguguugugugccuacguugaagacccgaccaaaucugauccuccguccagcagcacagaucaacccaccaccacuuucacugccaucgacaggugguacacuggacgucucaauucuuggacaaaagcuguaaaaaccuucucuuuucaggccgucccgcuucccggugccuuucugucuaggcagggaggccucaacggaggggccuucacagcuacccuacauagacacuuuuugaugaagugcggguggcaggugcagguccaauguaauuugacacaauuccaccaaggcgcucuucuuguugccaugguuccugaaaccacccuugaugucaagcccgacgguaaggcaaagagcuuacaggagcugaaugaagaacaguggguggaaaugucugacgauuaccggaccgggaaaaacaugccuuuucagucucuuggcacauacuaucggcccccuaacuggacuuggggucccaauuucaucaaccccuaucaaguaacgguuuucccacaccaaauucugaacgcgagaaccucuaccucgguagacauaaacgucccauacaucggggagacccccacgcaauccucagagacacagaacuccuggacccuccucguuauggugcucguuccccuagacuauaaggaaggagccacaacugacccagaaauuacauuuucuguaaggccuacaagucccuacuucaaugggcuucgcaaccgcuacacggccgggacggacgaagaacaggggcccauuccuacggcacccagagaaaauucgcuuauguuucucucaacccucccugacgacacugucccugcuuacgggaaugugcguaccccuccugucaauuaccucccuggugaaauaaccgaccuuuugcaacuggcccgcauacccacucucauggcauuugagcgggugccugaacccgugccugccucagacacauaugugcccuacguugccguucccacccaguucgaugacaggccucucaucuccuucccgaucacccuuucagaucccgucuaucagaacacccugguuggcgccaucaguucaaauuucgccaauuaccguggguguauccaaaucacucugacauuuuguggacccaugauggcgagagggaaauuccugcucucguauucucccccaaauggaacgcaaccacagacucuuuccgaagcuaugcagugcacauacucuauuugggacauaggcuugaacucuaguuggaccuucgucguccccuacaucucgcccagugacuaccgugaaacucgagccauuaccaacucgguuuacuccgcugaugguugguuuagccugcacaaguugaccaaaauuacucuaccaccugacuguccgcaaagucccugcauucucuuuuucgcuucugcuggugaggauuacacucuccgucuccccguugauuguaauccuuccuauguguuccacuccaccgacaacgccgagaccgggguuauugaggcggguaacacugacaccgauuucucuggugaacuggcggcuccuggcucuaaccacacuaaugucaaguuccuguuugaucgaucucgauuauugaauguaaucaagguacuggagaaggacgccguuuucccccgcccuuucccuacacaagaaggugcgcagcaggaugaugguuacuuuugucuucugaccccccgcccaacagucgcuucccgacccgccacucguuucggccuguacgccaauccguccggcagugguguucuugcuaacacuucacuggacuucaauuuuuauagcuuggccuguuucacuuacuuuagaucggaccuugagguuacgguggucucacuagagccggaucuggaauuugcuguagggugguuuccuucuggcagugaauaccaggcuuccagcuuugucuacgaccagcugcaugugcccuuccacuuuacugggcgcacuccccgcgcuuucgcuagcaagggugggaagguaucuuucgugcucccuuggaacucugucucgucugugcuccccgugcgcugggggggggcuuccaagcucucuucugcuacgcggggucuaccggcgcaugcugauugggggacuauuuacgccuuugucccccguccuaaugagaagaaaagcaccgcuguaaaacacguggccguguacauucgguacaagaacgcacgugccuggugccccagcaugcuucccuuucgcagcuacaagcagaagaugcugaugcaaucuggcgauaucgagaccaaucccgggccugcuucugacaacccaauuuuggaguuucuugaagcagaaaaugaucuagucacucuggccucucucuggaagauggugcacucuguucaacagaccuggagaaaguaugugaagaacgaugauuuuuggcccaauuuacucagcgagcuagugggggaaggcucugucgccuuggccgccacgcuauccaaccaagcuucaguaaaggcucuuuugggccugcacuuucucucucgggggcucaauuacacugacuuuuacucuuuacugauagagaaaugcucuaguuucuuuaccguagaaccaccuccuccaccagcugaaaaccugaugaccaagcccucagugaagucgaaauuccgaaaacuguuuaagaugcaaggacccauggacaaagucaaagacuggaaccaaauagcugccggcuugaagaauuuucaauuuguucgugaccuagucaaagagguggucgauuggcugcaggccuggaucaacaaagagaaagccagcccuguccuccaguaccaguuggagaugaagaagcucgggccuguggccuuggcucaugacgcuuucauggcugguuccgggcccccucuuagcgacgaccagauugaauaccuccagaaccucaaaucucuugcccuaacacuggggaagacuaauuuggcccaaagucucaccacuaugaucaaugccaaacaaaguucagcccaacgaguugaacccguuguggugguccuuagaggcaagccgggaugcggcaagagcuuggccucuacguugauugcccaggcuguguccaagcgccucuauggcucccaaaguguauauucucuucccccagauccagauuucuucgauggauacaaaggacaguucgugaccuugauggaugauuugggacaaaacccggauggacaagauuucuccaccuuuugucagauggugucgaccgcccaauuucuccccaacauggcggaccuugcagagaaagggcgucccuuuaccuccaaucucaucauugcaacuacaaaucucccccacuucaguccugucaccauugcugauccuucugcagucucucgccguaucaacuacgaucugacucuagaaguaucugaggccuacaagaaacacacacggcugaauuuugacuuggcuuucaggcgcacagacgccccccccauuuauccuuuugcugcccaugugcccuuuguggacguagcugugcgcuucaaaaauggucaccagaauuuuaaucuccuagaguuggucgauuccauuuguacagacauucgagccaagcaacaaggugcccgaaacaugcagacucugguucuacagagccccaacgagaaugaugacacccccgucgacgaggcguuggguagaguucucucccccgcugcggucgaugaggcgcuugucgaccucacuccagaggccgacccgguuggccguuuggcuauucuugccaagcuaggucuugcccuagcugcggucaccccuggucugauaaucuuggcagugggacucuacagguacuucucuggcucugaugcagaccaagaagaaacagaaagugagggaucugucaaggcacccaggagcgaaaaugcuuaugacggcccgaagaaaaacucuaagcccccuggagcacucucucucauggaaaugcaacagcccaacguggacaugggcuuugaggcugcggucgcuaagaaaguggucguccccauuaccuucaugguucccaacagaccuucugggcuuacacaguccgcucuucuggugaccggccggaccuuccuaaucaaugaacauacaugguccaaucccuccuggaccagcuucacaauccgcggugagguacacacucgugaugagcccuuccaaacgguucauuucacucaccacgguauucccacagaucugaugaugguacgucucggaccgggcaauucuuucccuaacaaucuagacaaguuuggacuugaccagaugccggcacgcaacucccgugugguuggcguuucguccaguuacggaaacuucuucuucucuggaaauuuccucggauuuguugauuccaucaccucugaacaaggaacuuacgcaagacucuuuagguacagggugacgaccuacaaaggauggugcggcucggcccuggucugugaggccgguggcguccgacgcaucauuggccugcauucugcuggcgccgccgguaucggcgccgggaccuauaucucaaaauuaggacuaaucaaagcccugaaacaccucggugaaccuuuggccacaaugcaaggacugaugacugaauuagagccuggaaucaccguacauguaccccggaaauccaaauugagaaagacgaccgcacacgcgguguacaaaccggaguuugagccugcuguguugucaaaauuugaucccagacugaacaaggauguugacuuggaugaaguaauuuggucuaaacacacugccaaugucccuuaccaaccuccuuuguucuacacauacaugucagaguacgcucaucgagucuucuccuucuuggggaaagacaaugacauucugaccgucaaagaagcaauucugggcauccccggacuagaccccauggauccccacacagcuccgggucugccuuacgccaucaacggccuucgacguacugaucucgucgauuuugugaacgguacaguagaugcggcgcuggcuguacaaauccagaaauucuuagacggugacuacucugaccaugucuuccaaacuuuucugaaagaugagaucagacccucagagaaaguccgagcgggaaaaacccgcauuguugaugugcccucccuggcgcauugcauugugggcagaauguugcuugggcgcuuugcugccaaguuucaaucccauccuggcuuucuccucggcucugcuaucgggucugacccugauguuuucuggaccgucauaggggcucaacucgaggggagaaagaacacguaugacguggacuacagugccuuugacucuucacacggcacuggcuccuucgaggcucucaucucucacuuuuucaccguggacaaugguuuuagcccugcgcugggaccguaucucagaucccuggcugucucggugcacgcuuacggcgagcgucgcaucaagauuaccgguggccuccccuccgguugugccgcgaccagccugcugaacacagugcucaacaaugugaucaucaggacugcucuggcauugacuuacaaggaauuugaauaugacaugguugauaucaucgccuacggugacgaccuucugguuggcacggauuacgaucuggacuucaaugagguggcacgacgcgcugccaaguugggguauaagaugacuccugccaacaaggguucugucuucccuccgacuuccucucuuuccgaugcuguuuuucuaaagcgcaaauucguccaaaacaacgacggcuuauacaaaccaguuauggauuuaaagaauuuggaagccaugcucuccuacuucaaaccaggaacacuacucgagaagcugcaaucuguuucuauguuggcucaacauucuggaaaagaagaauaugauagauugaugcaccccuucgcugacuacggugccguaccgagucacgaguaccugcaggcaagauggagggccuuguucgacugacccagauagcccaaggcgcuucggugcugccggcgauucugggagaacucagucggaacagaaaagggaaaaaaaaaaaaaaaaaaaaaaaaaaaaaa

SEQ ID NO: 843 - DNA, 7435 bp, Enterovirus Enterovirus A
ttaaaacagctctggggttgttcccaccccagaggcccacgtggcggctagtactctggtattacggtacctttgtacgcctgttttgtatcccttcccccgtaactttagaagcttatcaaaagttcaatagcaggggtacaaaccagtacctctacgaacaagcacttctgtttccccggtgatatcacatagactgtacccacggtcaaaagtgattgatccgttatccgcttgagtacttcgagaagcctagtatcaccttggaatcttcgatgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaaccgctcacaacccagtgagtaggttgtcgtaatgcgtaagtctgtggcggaaccgactactttgggtgtccgtgtttccctttatattcatactggctgcttatggtgacaatttacaaattgttaccatatagctattggattggccacccagtattgtgcaatatatttgagtgtttctttcataagccttattaacatcacatttttaatcacaataaacagtgcaaatgggggctcaagtttcaacgcaaaagaccggtgcgcacgagaatcaaaacgtggcagccaatggatccaccattaattacactactatcaactattacaaagacagtgcgagtaattccgctactagacaagacctctcccaagatccatcaaaattcacagaaccggttaaggacttaatgttgaaaacagcaccagctctaaactcgcctaacgtggaagcatgtgggtacagtgaccgtgtgaggcaaatcactttaggcaactcgactattactacacaagaagcagccaatgctattgttgcttacggtgaatggcccacttacataaatgattcagaagctaatccggtagatgcacccactgagccagacgttagtagcaaccggttttacaccctagaatcggtgtcttggaagaccacttcaaggggatggtggtggaagttaccagattgtttgaaggacatgggaatgtttggtcagaatatgtactatcactacttggggcgctctggttacaccattcatgtccagtgcaacgcttcaaaatttcaccaaggggcgttaggagtttttctgataccagagtttgtcatggcttgcaacactgagagtaaaacgtcatacgtttcatacatcaatgcaaatcctggtgagagaggcggtgagtttacgaacacctacaatccgtcaaatacagacgccagtgagggcagaaagtttgcagcattggattatttgctgggttctggtgttctagcaggaaacgcctttgtgtacccgcaccagatcatcaacctacgtaccaacaacagtgcaacaattgtggtgccatacgtaaactcacttgtgattgattgtatggcaaaacacaataactggggcattgtcatattaccactggcacccttggcctttgccgcaacatcgtcaccacaggtgcctattacagtgaccattgcacccatgtgtacagaattcaatgggttgagaaacatcaccgtcccagtacatcaagggttgccgacaatgaacacacctggttccaatcaattccttacatctgatgacttccagtcgccctgtgccttacctaattttgatgttactccaccaatacacatacccggggaagtaaagaatatgatggaactagctgaaattgacacattgatcccaatgaacgcagtggacgggaaggtgaacacaatggagatgtatcaaataccattgaatgacaatttgagcaaggcacctatattctgtttatccctatcacctgcttctgataaacgactgagccgcaccatgttgggtgaaatcctaaattattacacccattggacggggtccatcaggttcacctttctattttgtggtagtatgatggccactggtaaactgctcctcagctattccccaccgggagctaaaccaccaaccaatcgcaaggatgcaatgctaggcacacacatcatctgggacctagggttacaatccagttgttccatggttgcaccgtggatctccaacacagtgtacagacggtgtgcacgtgatgacttcactgagggcggatttataacttgcttctatcaaactagaattgtggtacctgcttcaacccctaccagtatgttcatgttaggctttgttagtgcgtgtccagacttcagtgtcagactgcttagggacactccccatattagtcaatcgaaactaataggacgtacacaaggcattgaagacctcattgacacagcgataaagaatgccttaagagtgtcccaaccaccctcgacccagtcaactgaagcaactagtggagtgaatagccaggaggtgccagctctaactgctgtggaaacaggagcatctggtcaagcaatccccagtgatgtggtggaaactaggcacgtggtaaattacaaaaccaggtctgaatcgtgtcttgagtcattctttgggagagctgcgtgtgtcacaatcctatccttgaccaactcctccaagagcggagaggagaaaaagcatttcaacatatggaatattacatacaccgacactgtccagttacgcagaaaattagagtttttcacgtattccaggtttgatcttgaaatgacttttgtattcacagagaactatcctagtacagccagtggagaagtgcgaaaccaggtgtaccagatcatgtatattccaccaggggcaccccgcccatcatcctgggatgactacacatggcaatcctcttcaaacccttccatcttctacatgtatggaaatgcacctccacggatgtcaattccttacgtagggattgccaatgcctattcacacttctacgatggctttgcacgggtgccacttgagggtgagaacaccgatgctggcgacacgttttacggtttagtgtccataaatgattttggagttttagcagttagagcagtaaaccgcagtaatccacatacaatacacacatctgtgagagtgtacatgaaaccaaaacacattcggtgttggtgccccagacctcctcgagctgtattatacaggggagagggagtggacatgatatccagtgcaattctacctctgaccaaggtagactcaattaccacttttgggtttggtcatcagaacaaagcagtgtacgttgccggttacaagatttgcaactaccacctagcaaccccaagtgatcacttgaatgcaattagtatgttatgggacagggatttaatggtggtggaatctagagcccagggaactgataccatcgccagatgtagttgcaggtgtggagtttactattgtgaatctaggaggaagtactaccctgtcacttttactggcccaacgtttcgattcatggaagcaaacgactactatccagcaagataccagtctcacatgctgatagggtgcggatttgcagaacccggggactgcggtgggatactgaggtgcactcatggggtaattggtatcattactgcaggaggtgaaggggtagtagcctttgctgacattagagacctctgggtgtatgaagaggaggccatggaacagggaataacaagctacatcgaatctctcggcacagcctttggcgcagggttcacccacacaatcagtgagaaagtgactgaattgacaacaatggttaccagcactatcacagaaaaactactgaaaaacttggtgaaaatagtgtcggctctagtgattgttgtgagaaattatgaggacactaccacgatccttgcaacactagcactactcgggtgtgatatatctccttggcaatggttgaagaagaaggcatgtgacttactagagattccttatgtgatgcgccaaggtgatgggtggatgaagaaattcacagaggcgtgcaatgcagctaaaggcttagagtggattagcaacaaaatttccaagtttatagattggttgaagtgtaaaattatcccagacgctaaggacaaggtggaatttctcaccaagttgaaacagctagacatgttggaaaatcaaattgcaaccatccaccaatcttgccccagccaagaacaacaagagattcttttcaacaatgtgagatggctagcagtccagtcccgtcggtttgcaccattatacgctgtggaggcacgccgaattaacaaaatggagagcacaataaacaattatatacagttcaagagcaaacaccgtattgaaccagtatgtatgctcattcatgggtcaccagggacgggtaaatctatagctacttcattaataggtagagcaatagcagagaaggaaagcacatcagtctattcaatgccacctgacccatctcactttgatggctataaacaacaaggggtagtgattatggacgacctaaaccaaaaccccgatggtatggacatgaaactgttttgccaaatggtatcaacagtggagtttattcctccaatggcctcattagaggagaagggcattttgtttacatctgattatgtcctggcttctaccaactctcattcaattgtaccacccacagtggctcacagtgatgccttaaccagacgatttgcatttgatgtggaggtttacacgatgtctgaacattcagtcaaaggcaaactgaatatggccacggccactcaattgtgtaaggattgtccaacacctgcaaattttaaaaagtgttgccctctcgtttgtggaaaggccttgcaattaatggacaggtacaccagacaaaggttcactgtagatgagattaccacattaatcatgaatgagaaaaacagaagggccaatatcggcaattgcatggaagccttgtttcaaggaccattaaggtataaagatttgaagatcgatgtgaagacagttcccccccctgagtgcatcagtgatttgttacaagcagtggattctcaagaggttagggattactgtgagaagaaaggctggatcgttaacgttactagccagattcaactagaaaggaacatcaatagggccatgactatactccaagctgttaccacattcgcagcagtcgcaggagtagtgtatgtaatgtacaaactcttcgccggtcaacagggtgcatacactggcttgccaaacaaaaaacccaatgtccctactatcagagtcgctaaagtccaggggccaggatttgactacgcagtggcaatggcaaaaagaaacatagttactgcaaccaccaccaagggtgaatttaccatgctaggggtgcatgataatgtagcaatattgccaacccatgccgctccaggagaaaccattattattgatgggaaagaagtagagatcctagatgccagagccttagaagatcaagcgggaaccaatcttgagatcaccattattactctaaaaagaaatgagaagtttagagacatcagatcacatattcccacccaaattactgaaactaacgatggagtgttgatcgtgaacactagcaagtaccccaatatgtatgtccccgttggtgctgtgaccgaacagggatatcttaatctcagtggacgtcaaactgctcgcactttaatgtacaactttccaacaagggcaggccagtgcggaggaatcatcacttgtactggcaaagtcattgggatgcatgttggcgggaacggttcacatgggtttgcagcagccctcaagcgatcatacttcactcaaaatcagggcgaaatccagtggatgaggtcatcaaaagaagtggggtaccccattataaatgccccatccaagacaaagttagaacccagtgctttccactatgtttttgaaggtgttaaggaaccagctgtactcactaagaatgaccccagactaaaaacagattttgaagaagccatcttttctaaatatgtggggaacaaaattactgaagtggacgagtacatgaaagaagcagtggatcactatgcaggacagttaatgtcactggatatcaacacagaacagatgtgcctggaggatgccatgtacggcaccgatggtcttgaggccctggatcttagcactagtgctggatatccttatgttgcaatggggaaaaagaaaagagacattctagataaacagaccagagatactaaggagatgcagagacttttagatacctatggaatcaatctaccattagtcacgtacgtgaaagatgaactcaggtcaaagactaaagtggaacaaggaaagtcaagattgattgaagcttccagccttaatgattcagttgcaatgagaatggcctttggcaatctttacgcagctttccacaagaatccaggtgtggtgacaggatcagcagttggttgtgacccagatttgttttggagtaagataccagtgctaatggaagaaaaactcttcgcttttgactacacagggtatgatgcctcactcagccctgcttggtttgaagctcttaaaatggtgttagaaaaaattggatttggcagtagagtagactatatagactacctgaaccactctcaccacctttacaaaaacaagacttattgtgtcaaaggcggcatgccatccggctgctctggcacctcaattttcaactcaatgattaacaacctgatcattaggacgcttttactgagaacctacaagggcatagacttggaccatttaaaaatgattgcctatggtgatgacgtgatagcttcctacccccatgaggttgacgctagtctcctagcccaatcaggaaaagactatggactaaccatgactccagcagataaatcagtaacctttgaaacagtcacatgggagaatgtaacatttctgaaaagatttttcagagcagatgagaagtatccattcctggtgcatccagtgatgccaatgaaagaaattcacgaatcaatcagatggaccaaggaccctagaaacacacaggatcacgtacgctcgttgtgcctattagcttggcacaacggtgaagaagaatacaataaatttttagctaaaatcagaagtgtgccaatcggaagagctttattgctcccagagtactctacattgtaccgccgatggctcgactcattttagtaaccctacctcagtcggattggattgggttatactgttgtaggggtaaatttttctttaattcggagaaaaaaaaaaaaaaaaaaaaaaaaaaaaaa

SEQ ID NO: 844 - DNA, 315 bp, Artificial Sequence
  TetON regulatory element
gagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 845 - DNA, 315 bp, Artificial Sequence; identical to SEQ ID NO: 844
  TetOff regulatory element

SEQ ID NO: 846 - DNA, 2220 bp, Artificial Sequence
  FLP-RGS-ERT2
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctgcgtacgcggatccgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 847 - DNA, 2253 bp, Artificial Sequence
  FLP-XTEN-ERT2
atgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatcagcggcagcgagacccccggcaccagcgagagcgccacccccgagagcgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 848 - DNA, 24 bp, Artificial Sequence
  SV40 NLS
atgcctaagaagaagaggaaggtg

SEQ ID NO: 849 - DNA, 132 bp, Artificial Sequence
  LYL1 PEST
atgtgcccacctcaggcacaggcagaggtgggccctaccatgactgagaaggcagagatggtgtgtgcccctagcccagcgcctgccccaccccctaagcctgcctcgcctgggcccccgcaggtggaggag

SEQ ID NO: 850 - DNA, 34 bp, Artificial Sequence
  FRT-1 site
gaagttcctattctctagaaagtataggaacttc

SEQ ID NO: 851 - DNA, 34 bp, Artificial Sequence
  FRT-14 site
gaagttcctattctatcagaagtataggaacttc

SEQ ID NO: 852 - DNA, 705 bp, Artificial Sequence
  rtTA
atgtctaggctggacaagagcaaagtcataaacggagctctggaattactcaatggtgtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgattttgaccttgacatgctccccggg

SEQ ID NO: 853 - DNA, 1008 bp, Artificial Sequence
  tTA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 854 - DNA, 49 bp, Artificial Sequence
  Stop1
aataaaatatctttattttcattacatctgtgtgttggttttttgtgtg

SEQ ID NO: 855 - DNA, 137 bp, Artificial Sequence
  Stop2
aataaaatatctttattttcattacatctgtgtgttggttttttgtgtggaataaaatgcatttgtataaaaaatgctttaaatgatggatatgttactttagcaagaacttttaggtcaggtttctcctttgtttt

SEQ ID NO: 856 - DNA, 214 bp, Artificial Sequence
  Stop3
aataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatgggg

SEQ ID NO: 857 - DNA, 150 bp, Artificial Sequence
  FRT-1-Stop1-FRT-1
gaagttcctattctctagaaagtataggaacttctaataataaataataataattagtagtaggaataaaatatctttattttcattacatctgtgtgttggttttttgtgtgggggaagttcctattctctagaaagtataggaacttc

SEQ ID NO: 858 - DNA, 235 bp, Artificial Sequence
  FRT-1-Stop2-FRT-1
gaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaagttcctattctctagaaagtataggaacttc

SEQ ID NO: 859 - DNA, 313 bp, Artificial Sequence
  FRT-1-Stop3-FRT-1
gaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatggggtgaagttcctattctctagaaagtataggaacttc

SEQ ID NO: 860 - DNA, 3579 bp, Artificial Sequence
  Promoter Inversion with STOP
gaagttcctatacttctgatagaataggaacttcaccccatgcctccatcagccagccgtgtttccctaaataagcacacatctcgcagcttttttcaattcccatttattcaaaacaaaggagaaacctgacctaaaagttcttgctaaagtaacatatccatcatttaaagcattttttatacaaatgcattttattccacacaaaaaaccaacacacagatgtaatgaaaataaagatattttattcctactactaattattattatttattattagaagttcctatactttctagagaataggaacttcggtaccgatctctatcactgatagggagatctctatcactgatagggagagctctgcttggtatatgtacactttacctgggggcgtgccggaccgccccgactgcatctgcgtgttcgaattcgccaatgacaagacgctgggcggggacgcgtgaagttcctattctatcagaagtataggaacttctaataataaataataataattagtagtaggaataaatgcagtttataaaagtgttagattgttgttataccttgtaagagtcatgtgatcatactgttttctacaaagttgtattttagatataatgcctgaaaccattttggtgtttgcttcagtcagtatttcattgtatgctggaataaagtccaaagtctgatctggtctagtttacctagaagtatttttgtctcttagaaatacttgtgatttttataatacaaaagggtcttgactctaaatgcagttttaagaattgtttttgaatttaaataaagttacttgaatttcaaagatcacagggcagtgtcttcatttgaccaggactgttgaaagaataaaaaagctcttttaatattgatatactgtcctttttaacgctttaaaaacagattttgaggggagaaaagtattgtttcagttttgcttttgataaaaatataatttgacttctttgaactggatttttctttaaggctttgccagttgtggaagataagctattttgggggtctttcagtatttaattgtgaagttcctattctctagaaagtataggaacttcgcggccgcgagctcggatccactagtccagtgtggtggaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaggaggaagagagaaggcagggggagccttctcacttgcggcgatgtcgaggaaaatccggggcctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 861 - DNA, 3591 bp, Artificial Sequence
  Payload Inversion with STOP
ccccgcccagcgtcttgtcattggcgaattcgaacacgcagatgcagtcggggcggtccggcacgcccccaggtaaagtgtacatataccaagcagagctctccctatcagtgatagagatctccctatcagtgatagagatcggtaccgaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatggggtgaagttcctattctatcagaagtataggaacttcgcggccgctgcagaattcttacacggcgatcttgccgcccttcttggccttaatgagaatctcgcggatcttgcgggcgtccaacttgccggtcagtcctttaggcacctcgtccacgaacacaacaccaccgcgcagcttcttggcggttgtaacctggctggccacatagtccacgatctccttctcggtcatggttttaccgtgttccagcacgacgactgcggcgggcagctcgccggcatcgtcgtcgggcaggccggcgaccccggcgtcgaagatgttggggtgttgcagcaggatgctctccagttcggctggggctacctggtagcccttgtatttgatcaggctcttcagccggtccacgatgaagaagtgctcgtcctcgtcccagtaggcgatgtcgccgctgtgcagccagccgtccttgtcgatgagagcgtttgtagcctcggggttgttaacgtagccgctcatgatcatggggccacggacgcacagctcgccgcgctggttcacacccagtgtcttaccggtgtccaagtccaccaccttagcctcgaagaagggcaccaccttgcctactgcgccaggcttgtcgtccccttcgggggtgatcagaatggcgctggttgtttctgtcaggccgtagccctggcggatgcctggtaggtggaagcgtttggccacggcctcacctacctccttgctgagcggcgccccgccgctggcgatctcgtgcaagttgcttaggtcgtacttgtcgatgagagtgctcttagcgaagaagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgatctggttgccgaagatggggtcgcgggcatgactgaatcggacacaagcggtgcggtgcggtagggctacgcccttgggcaatccggtactgccactactgttcatgatcagggcgatggttttgtcccggtcgaagctctcgggcacgaagtcgtactcgttgaagccgggtggcaaatgggaagtcacgaaggtgtacatgctttggaagccctggtagtcggtcttgctatccatgatgatgatcttttgtatgatcggtagcttcttttgcacgttgaggatcttttgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcaagctattctcgctgcacaccacgatccgatggtttgtattcagcccatagcgcttcatagcttctgccagccgaacgctcatctcgaagtactcggcgtaggtaatgtccacctcgatatgtgcgtcggtaaaggcgatggtgccgggcaccagggcgtagcgcttcatggctttgtgcagctgctcgccggcggtcccgtcttcgagtgggtagaatggcgctgggcccttcttaatgtttttggcatcttccataggccccggattttcctcgacatcgccgcaagtgagaaggctccccctgccttctctcttcctcctcttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcgaattccaccacactggactagtggatccgagctcgcggccgcgaagttcctatactttctagagaataggaacttcacaattaaatactgaaagacccccaaaatagcttatcttccacaactggcaaagccttaaagaaaaatccagttcaaagaagtcaaattatatttttatcaaaagcaaaactgaaacaatacttttctcccctcaaaatctgtttttaaagcgttaaaaaggacagtatatcaatattaaaagagcttttttattctttcaacagtcctggtcaaatgaagacactgccctgtgatctttgaaattcaagtaactttatttaaattcaaaaacaattcttaaaactgcatttagagtcaagacccttttgtattataaaaatcacaagtatttctaagagacaaaaatacttctaggtaaactagaccagatcagactttggactttattccagcatacaatgaaatactgactgaagcaaacaccaaaatggtttcaggcattatatctaaaatacaactttgtagaaaacagtatgatcacatgactcttacaaggtataacaacaatctaacacttttataaactgcatttattcctactactaattattattatttattattagaagttcctatacttctgatagaataggaacttc

SEQ ID NO: 862 - DNA, 4017 bp, Artificial Sequence
  Promoter with Split Intron Inversion and STOP
gaagttcctatacttctgatagaataggaacttcaccccatgcctccatcagccagccgtgtttccctaaataagcacacatctcgcagcttttttcaattcccatttattcaaaacaaaggagaaacctgacctaaaagttcttgctaaagtaacatatccatcatttaaagcattttttatacaaatgcattttattccacacaaaaaaccaacacacagatgtaatgaaaataaagatattttattcctatcactagtcatcactagctatcactagaagttcctatactttctagagaataggaacttcggatccaggaaggagggaggcggccaccagaagaggtagcgggccactcacctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggctggccaccacactggactagtgagctcggtaccgatctctatcactgatagggagatctctatcactgatagggagagctctgcttggtatatgtacactttacctgggggcgtgccggaccgccccgactgcatctgcgtgttcgaattcgccaatgacaagacgctgggcggggaacgcgtgaagttcctattctatcagaagtataggaacttctaataataaataataataattagtagtaggaataaatgcagtttataaaagtgttagattgttgttataccttgtaagagtcatgtgatcatactgttttctacaaagttgtattttagatataatgcctgaaaccattttggtgtttgcttcagtcagtatttcattgtatgctggaataaagtccaaagtctgatctggtctagtttacctagaagtatttttgtctcttagaaatacttgtgatttttataatacaaaagggtcttgactctaaatgcagttttaagaattgtttttgaatttaaataaagttacttgaatttcaaagatcacagggcagtgtcttcatttgaccaggactgttgaaagaataaaaaagctcttttaatattgatatactgtcctttttaacgctttaaaaacagattttgaggggagaaaagtattgtttcagttttgcttttgataaaaatataatttgacttctttgaactggatttttctttaaggctttgccagttgtggaagataagctattttgggggtctttcagtatttaattgtgaagttcctattctctagaaagtataggaacttcgatatcggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaggaggaagagagaaggcagggggagccttctcacttgcggcgatgtcgaggaaaatccggggcctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 863 - DNA, 846 bp, Artificial Sequence
  Intron 3 of ACTB
gtgagtggcccgctacctcttctggtggccgcctccctccttcctggatccgatatcggcctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacagctcccggagctgcgccctttctcactggttctctcttctgccgttttccgtaggactctcttctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacag

SEQ ID NO: 864 - DNA, 696 bp, Artificial Sequence
  NLS-TetR-NLS
atggccccaaagaagaagcggaaggtcatgtctagactggacaagtccaaggtcatcaattctgccctggaactcttgaacgaagtgggcattgagggcctgaccacaaggaagttggctcaaaaattgggcgtggaacaacccaccctctactggcacgtgaagaacaaaagagccctcttggatgctctggctatcgaaatgctggaccgccatcacacccatttctgtcccctggagggcgagtcttggcaggacttcctgaggaacaatgccaagtccttccggtgcgccctgttgtcccacagggacggcgccaaggtgcacctgggcaccagacccaccgagaagcaatacgagactttggagaaccagctggctttcctgtgtcagcagggcttcagcttggaaaacgccctgtacgccttgtctgccgtcggccacttcaccctgggctgtgtgctggaggaccaggaacaccaggtggccaaggaggagcgagagacccccaccaccgactccatgcccccactgctgaggcaggccattgagctgttcgaccatcagggcgccgaacccgcttttctgtttgggttggagctcattatctgtggcctggagaagcagttgaagtgcgagtccggcagtgcctattctggcagtagagagtttcccaagaagaagaggaaggtgtaa

SEQ ID NO: 865 - DNA, 127 bp, Artificial Sequence
  HBP1-TO
ccacgcccaccggctgatgacgcgcggggtgtgggaggggctggggcggcgcggtcccaggtccacttcgcatattaagcagagctctccctatcagtgatagagatctccctatcagtgatagaga

SEQ ID NO: 866 - DNA, 141 bp, Artificial Sequence
  HBP2-TO
ccccgcccagcgtcttgtcattggcgaattcgaacacgcagatgcagtcggggcggtccggcacgcccccaggtaaagtgtacatataccaagcagagctctccctatcagtgatagagatctccctatcagtgatagaga

SEQ ID NO: 867 - DNA, 2385 bp, Artificial Sequence
  FEXP nucleotide sequence
atgtgcccacctcaggcacaggcagaggtgggccctaccatgactgagaaggcagagatggtgtgtgcccctagcccagcgcctgccccaccccctaagcctgcctcgcctgggcccccgcaggtggaggagatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatcagcggcagcgagacccccggcaccagcgagagcgccacccccgagagcgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 868 - DNA, 2519 bp, Artificial Sequence
  FEXPi2 nucleotide sequence
atgtgcccacctcaggcacaggcagaggtgggccctaccatgactgagaaggcagagatggtgtgtgcccctagcccagcgcctgccccaccccctaagcctgcctcgcctgggcccccgcaggtggaggagatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggtaggggagctggctgggtggggcagccccgggagcgggcgggaggcaagggcgctttctctgcacaggagcctcccggtttccggggtgggggctgcgcccgtgctcagggcttcttgtcctttccttcccaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatcagcggcagcgagacccccggcaccagcgagagcgccacccccgagagcgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 869 - DNA, 2695 bp, Artificial Sequence
  HBP1-TO-FEXPi2
ccacgcccaccggctgatgacgcgcggggtgtgggaggggctggggcggcgcggtcccaggtccacttcgcatattaagcagagctctccctatcagtgatagagatctccctatcagtgatagagatcggtaccgagctcggatccactagtccagtgtggtggaattcgccaccatgtgcccacctcaggcacaggcagaggtgggccctaccatgactgagaaggcagagatggtgtgtgcccctagcccagcgcctgccccaccccctaagcctgcctcgcctgggcccccgcaggtggaggagatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggtaggggagctggctgggtggggcagccccgggagcgggcgggaggcaagggcgctttctctgcacaggagcctcccggtttccggggtgggggctgcgcccgtgctcagggcttcttgtcctttccttcccaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatcagcggcagcgagacccccggcaccagcgagagcgccacccccgagagcgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 870 - DNA, 2932 bp, Artificial Sequence
  HBP2-TO-STOP3-mCherry-fLuc
ccccgcccagcgtcttgtcattggcgaattcgaacacgcagatgcagtcggggcggtccggcacgcccccaggtaaagtgtacatataccaagcagagctctccctatcagtgatagagatctccctatcagtgatagagatcggtaccgagctcggatccactagtccagtgtggtggaattcgccaccatgtggaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatggggtgaagttcctattctctagaaagtataggaacttcgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaggaggaagagagaaggcagggggagccttctcacttgcggcgatgtcgaggaaaatccggggcctatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggcaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 871 - DNA, 8648 bp, Artificial Sequence
  HBP2-TO-STOP3-SVV-mCherry
ccccgcccagcgtcttgtcattggcgaattcgaacacgcagatgcagtcggggcggtccggcacgcccccaggtaaagtgtacatataccaagcagagctctccctatcagtgatagagatctccctatcagtgatagagatcggtaccgaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatggggtgaagttcctattctctagaaagtataggaacttcgcggccgcttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatggga

SEQ ID NO: 872 - DNA, 14108 bp, Artificial Sequence
  Full 14.1 kbp insertion sequence
gctttgacattgattattgactagtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtttctcgagtttactagtgaccggtttacaccttcctcttcttcttgggaaactctctactgccagaataggcactgccggactcgcacttcaactgcttctccaggccacagataatgagctccaacccaaacagaaaagcgggttcggcgccctgatggtcgaacagctcaatggcctgcctcagcagtgggggcatggagtcggtggtgggggtctctcgctcctccttggccacctggtgttcctggtcctccagcacacagcccagggtgaagtggccgacggcagacaaggcgtacagggcgttttccaagctgaagccctgctgacacaggaaagccagctggttctccaaagtctcgtattgcttctcggtgggtctggtgcccaggtgcaccttggcgccgtccctgtgggacaacagggcgcaccggaaggacttggcattgttcctcaggaagtcctgccaagactcgccctccaggggacagaaatgggtgtgatggcggtccagcatttcgatagccagagcatccaagagggctcttttgttcttcacgtgccagtagagggtgggttgttccacgcccaatttttgagccaacttccttgtggtcaggccctcaatgcccacttcgttcaagagttccagggcagaattgatgaccttggacttgtccagtctagacatgaccttccgcttcttctttggggccatggtggcaccggtggatcctctagagtcggtgtcttctatggaggtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctgctgtctctctgttcttattaatagtaatcaattacggggcatgatcaaagcttccacgcccaccggctgatgacgcgcggggtgtgggaggggctggggcggcgcggtcccaggtccacttcgcatattaagcagagctctccctatcagtgatagagatctccctatcagtgatagagatcggtaccgagctcggatccactagtccagtgtggtggaattcgccaccatgtgcccacctcaggcacaggcagaggtgggccctaccatgactgagaaggcagagatggtgtgtgcccctagcccagcgcctgccccaccccctaagcctgcctcgcctgggcccccgcaggtggaggagatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggtaggggagctggctgggtggggcagccccgggagcgggcgggaggcaagggcgctttctctgcacaggagcctcccggtttccggggtgggggctgcgcccgtgctcagggcttcttgtcctttccttcccaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatcagcggcagcgagacccccggcaccagcgagagcgccacccccgagagcgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacatgagaattctgcagatatccagcacagtggcggccgctcgagtctagagggcccgtttaacccgctgatcagcctaataaaagtgcacaccttaaaatgaggccaagtgtgactttgtggtgtggctgggttgggggcagcagagggtgaaccctgcaggagggtgaaccctgcaaaagggtggggcagtgggggggcctaggcttttgcaaaaagctaacttgtttattgcagcttaatctagacccaactttgtatacaaaagttggctttgaccgcggattgattattgactagccccagaccctagaataagacaggacaagtaactggttgagcacagggtactttatttttctcgagtttactagtgaccggttcatgatcagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtaagcttaccccgcccagcgtcttgtcattggcgaattcgaacacgcagatgcagtcggggcggtccggcacgcccccaggtaaagtgtacatataccaagcagagctctccctatcagtgatagagatctccctatcagtgatagagatcggtaccgaagttcctattctctagaaagtataggaacttctaataataaataataataattagtagtaggaataaaatatctttattttcattacatctgtgtgttggttttttgtgtggaataaaatgcatttgtataaaaaatgctttaaatgatggatatgttactttagcaagaacttttaggtcaggtttctcctttgttttgaataaatgggaattgaaaaaagctgcgagatgtgtgcttatttagggaaacacggctggctgatggaggcatggggtgaagttcctattctctagaaagtataggaacttcgcggccgcttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcctaggcttttgcaaaaagctaacttgtttattgcagcttaatctagacc

SEQ ID NO: 873 - DNA, 8480 bp, Artificial Sequence
  SVV expression cassette for ONCR-189
gctttgacattgattattgactagacattgattattgactagtacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccttatcaaactgatgagtccgtgaggacgaaacgagtaagctcgtctttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaaggccggcatggtcccagcctcctcgctggcgccggctgggcaacatgcttcggcatggcgaatgggacgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcctaggcttttgcaaaaagctaacttgtttattgcagcttaatctagacc

SEQ ID NO: 874 - DNA, 9100 bp, Artificial Sequence
  SVV expression cassette for ONCR-190
gctttgacattgattattgactagacattgattattgactagtacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccttaattaatttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacctgggaactctcggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggtgcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcccgggccgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcccgggcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagactagtgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggcctaggcttttgcaaaaagctaacttgtttattgcagcttaatctagacc

SEQ ID NO: 875 - DNA, 61 bp, Artificial Sequence
  KD_KDRT
aaacgatatcagacatttgtctgataatgcttcattatcagacaaatgtctgatatcgttt

SEQ ID NO: 876 - DNA, 39 bp, Artificial Sequence
  B2_B2RT
gagtttcattaaggaataactaattccctaatgaaactc

SEQ ID NO: 877 - DNA, 33 bp, Artificial Sequence
  B3_B3RT
ggttgcttaagaataagtaattcttaagcaacc

SEQ ID NO: 878 - DNA, 31 bp, Artificial Sequence
  R_RSRT
ttgatgaaagaataacgtattctttcatcaa

SEQ ID NO: 879 - DNA, 34 bp, Artificial Sequence
  Cre_loxP
ataacttcgtatagcatacattatacgaagttat

SEQ ID NO: 880 - DNA, 34 bp, Artificial Sequence
  VCre_VloxP
tcaatttctgagaactgtcattctcggaaattga

SEQ ID NO: 881 - DNA, 34 bp, Artificial Sequence
  SCre_SloxP
ctcgtgtccgataactgtaattatcggacatgat

SEQ ID NO: 882 - DNA, 34 bp, Artificial Sequence
  Vika_vox
aataggtctgagaacgcccattctcagacgtatt

SEQ ID NO: 883 - DNA, 32 bp, Artificial Sequence
  Dre_rox
taactttaaataatgccaattatttaaagtta

SEQ ID NO: 884 - DNA, 21 bp, Artificial Sequence
  Lambda-Int_attP
cagcttttttatactaagttg

SEQ ID NO: 885 - DNA, 21 bp, Artificial Sequence
  Lambda-Int_attB
ctgcttttttatactaacttg

SEQ ID NO: 886 - DNA, 21 bp, Artificial Sequence
  HK022_attP
atcctttaggtgaataagttg

SEQ ID NO: 887 - DNA, 21 bp, Artificial Sequence
  HK022_attB
gcactttaggtgaaaaaggtt

SEQ ID NO: 888 - DNA, 38 bp, Artificial Sequence
  phiC31_attP
ccccaactggggtaacctttgagttctctcagttgggg

SEQ ID NO: 889 - DNA, 34 bp, Artificial Sequence
  phiC31_attB
gtgccagggcgtgcccttgggctccccgggcgcg

SEQ ID NO: 890 - DNA, 48 bp, Artificial Sequence
  Bxb1_attP
ggtttgtctggtcaaccaccgcggtctcagtggtgtacggtacaaacc

SEQ ID NO: 891 - DNA, 38 bp, Artificial Sequence
  Bxb1_attB
ggcttgtcgacgacggcggtctccgtcgtcaggatcat

SEQ ID NO: 892 - DNA, 26 bp, Artificial Sequence
  Gin_gix
ttatccaaaacctcggtttacaggaa

SEQ ID NO: 893 - DNA, 28 bp, Artificial Sequence
  Tn3_res site I
cgttcgaaatattataaattatcagaca

SEQ ID NO: 894 - DNA, 316 bp, Homo sapiens
atctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggag

SEQ ID NO: 895 - DNA, 69 bp, Homo sapiens
ttttattgtgtttttaatttatttattaagatggattctcagatatttatatttttattttattttttt

SEQ ID NO: 896 - DNA, 385 bp, Homo sapiens
atctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggagttttattgtgtttttaatttatttattaagatggattctcagatatttatatttttattttattttttt

SEQ ID NO: 897 - DNA, 558 bp, Artificial Sequence
  CMV-TO
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagaga

SEQ ID NO: 898 - DNA, 209 bp, Artificial Sequence
  TK-TO
cccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaagcagagctctccctatcagtgatagagatctccctatcagtgatagaga

SEQ ID NO: 899 - DNA, 165 bp, Artificial Sequence
  ICP8-TO
tttttttataatagggccacgcccaccggctgatgacgcgcggggtgtgggaggggctggggcggtccggcacgcccccaggtaaagtgtacatataccaaccgcatatcagacgcgcagagctctccctatcagtgatagagatctccctatcagtgatagaga

SEQ ID NO: 900 - DNA, 306 bp, Artificial Sequence
  gB-TO
tcgcttcttcgagctcgtcaacgggcccctctttgatcactccacccacagcttcgcccagccccccaacaccgcgctgtattacagcgtcgagaacgtggggctcctgccgcacctgaaggaggagctcgcccggttcatcatgggggcggggggctcgggtgctgattgggccgtcagcgaatttcagaggttttactgttttgacggcatttccggaataacgcccactcagcgcgccgcctggcgatatattcgcagagctctccctatcagtgatagagatctccctatcagtgatagaga

SEQ ID NO: 901 - DNA, 99 bp, Artificial Sequence
  gC-TO
gtgatgatttcgccataacacccaaaccccggatggggcccgggtataaagcagagctctccctatcagtgatagagatctccctatcagtgatagaga

SEQ ID NO: 902 - DNA, 140 bp, Artificial Sequence
  miRNA-TS
  misc_feature (21)..(40): n is any naturally-occurring nucleotide and up to nineteen of them may be absent
  misc_feature (61)..(80): n is any naturally-occurring nucleotide and up to nineteen of them may be absent
  misc_feature (101)..(120): n is any naturally-occurring nucleotide and up to nineteen of them may be absent
ggcattcaccgcgtgccttannnnnnnnnnnnnnnnnnnnggcattcaccgcgtgccttannnnnnnnnnnnnnnnnnnnggcattcaccgcgtgccttannnnnnnnnnnnnnnnnnnnggcattcaccgcgtgcctta

SEQ ID NO: 903 - DNA, 312 bp, Artificial Sequence
  miRNA-TS
  misc_feature (23)..(42): n is any naturally-occurring nucleotide and up to nineteen of them may be absent
  misc_feature (64)..(83): n is any naturally-occurring nucleotide and up to nineteen of them may be absent
  misc_feature (106)..(125): n is any naturally-occurring nucleotide and up to nineteen of them may be absent
  misc_feature (147)..(166): n is any naturally-occurring nucleotide and up to nineteen of them may be absent
  misc_feature (189)..(208): n is any naturally-occurring nucleotide and up to nineteen of them may be absent
  misc_feature (230)..(249): n is any naturally-occurring nucleotide and up to nineteen of them may be absent
  misc_feature (272)..(291): n is any naturally-occurring nucleotide and up to nineteen of them may be absent
atacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctca

SEQ ID NO: 904 - DNA, 472 bp, Artificial Sequence
  miRNA-TS
  misc_feature (21)..(40): n is any naturally-occurring nucleotide and up to nineteen of them may be absent
  misc_feature (61)..(80): n is any naturally-occurring nucleotide and up to nineteen of them may be absent
  misc_feature (101)..(120): n is any naturally-occurring nucleotide and up to nineteen of them may be absent
  misc_feature (141)..(160): n is any naturally-occurring nucleotide and up to nineteen of them may be absent
  misc_feature (183)..(202): n is any naturally-occurring nucleotide and up to nineteen of them may be absent
  misc_feature (224)..(243): n is any naturally-occurring nucleotide and up to nineteen of them may be absent
  misc_feature (266)..(285): n is any naturally-occurring nucleotide and up to nineteen of them may be absent
  misc_feature (307)..(326): n is any naturally-occurring nucleotide and up to nineteen of them may be absent
  misc_feature (349)..(368): n is any naturally-occurring nucleotide and up to nineteen of them may be absent
  misc_feature (390)..(409): n is any naturally-occurring nucleotide and up to nineteen of them may be absent
  misc_feature (432)..(451): n is any naturally-occurring nucleotide and up to nineteen of them may be absent
ggcattcaccgcgtgccttannnnnnnnnnnnnnnnnnnnggcattcaccgcgtgccttannnnnnnnnnnnnnnnnnnnggcattcaccgcgtgccttannnnnnnnnnnnnnnnnnnnggcattcaccgcgtgccttannnnnnnnnnnnnnnnnnnnatacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctcannnnnnnnnnnnnnnnnnnnatacatacttctttacattccannnnnnnnnnnnnnnnnnnngagctacagtgcttcatctca

SEQ ID NO: 905 - DNA, 476 bp, Artificial Sequence
  miRNA-TS
  misc_feature (22)..(41): n is any naturally-occurring nucleotide and up to nineteen of them may be absent
  misc_feature (64)..(83): n is any naturally-occurring nucleotide and up to nineteen of them may be absent
  misc_feature (105)..(124): n is any naturally-occurring nucleotide and up to nineteen of them may be absent
  misc_feature (147)..(166): n is any naturally-occurring nucleotide and up to nineteen of them may be absent
  misc_feature (188)..(207): n is any naturally-occurring nucleotide and up to nineteen of them may be absent
  misc_feature (229)..(248): n is any naturally-occurring nucleotide and up to nineteen of them may be absent
  misc_feature (271)..(290): n is any naturally-occurring nucleotide and up to nineteen of them may be absent
  misc_feature (312)..(331): n is any naturally-occurring nucleotide and up to nineteen of them may be absent
  misc_feature (353)..(372): n is any naturally-occurring nucleotide and up to nineteen of them may be absent
  misc_feature (394)..(413): n is any naturally-occurring nucleotide and up to nineteen of them may be absent
  misc_feature (436)..(455): n is any naturally-occurring nucleotide and up to nineteen of them may be absent
agaattgcgtttggacaatcannnnnnnnnnnnnnnnnnnncaaacaccattgtcacactccannnnnnnnnnnnnnnnnnnnaaagagaccggttcactgtgrnnnnnnnnnnnnnnnnnnnncaaacaccattgtcacactccannnnnnnnnnnnnnnnnnnnagaattgcgtttggacaatcannnnnnnnnnnnnnnnnnnnaaagagaccggttcactgtgrnnnnnnnnnnnnnnnnnnnncaaacaccattgtcacactccannnnnnnnnnnnnnnnnnnnaaagagaccggttcactgtgrnnnnnnnnnnnnnnnnnnnnagaattgcgtttggacaatcannnnnnnnnnnnnnnnnnnnaaagagaccggttcactgtgrnnnnnnnnnnnnnnnnnnnncaaacaccattgtcacactccannnnnnnnnnnnnnnnnnnnagaattgcgtttggacaatca

SEQ ID NO: 906 - DNA, 476 bp, Artificial Sequence
  miRNA-TS
  misc_feature (22)..(41): n is any naturally-occurring nucleotide and up to nineteen of them may be absent
  misc_feature (63)..(82): n is any naturally-occurring nucleotide and up to nineteen of them may be absent
  misc_feature (105)..(124): n is any naturally-occurring nucleotide and up to nineteen of them may be absent
  misc_feature (146)..(165): n is any naturally-occurring nucleotide and up to nineteen of them may be absent
  misc_feature (187)..(206): n is any naturally-occurring nucleotide and up to nineteen of them may be absent
  misc_feature (229)..(248): n is any naturally-occurring nucleotide and up to nineteen of them may be absent
  misc_feature (270)..(289): n is any naturally-occurring nucleotide and up to nineteen of them may be absent
  misc_feature (311)..(330): n is any naturally-occurring nucleotide and up to nineteen of them may be absent
  misc_feature (353)..(372): n is any naturally-occurring nucleotide and up to nineteen of them may be absent
  misc_feature (394)..(413): n is any naturally-occurring nucleotide and up to nineteen of them may be absent
  misc_feature (435)..(454): n is any naturally-occurring nucleotide and up to nineteen of them may be absent
aaagagaccggttcactgtgrnnnnnnnnnnnnnnnnnnnnagaattgcgtttggacaatcannnnnnnnnnnnnnnnnnnnaggcataggatgacaaagggaannnnnnnnnnnnnnnnnnnnaaagagaccggttcactgtgrnnnnnnnnnnnnnnnnnnnnagaattgcgtttggacaatcannnnnnnnnnnnnnnnnnnnaggcataggatgacaaagggaannnnnnnnnnnnnnnnnnnnaaagagaccggttcactgtgrnnnnnnnnnnnnnnnnnnnnagaattgcgtttggacaatcannnnnnnnnnnnnnnnnnnnaggcataggatgacaaagggaannnnnnnnnnnnnnnnnnnnaaagagaccggttcactgtgrnnnnnnnnnnnnnnnnnnnnagaattgcgtttggacaatcannnnnnnnnnnnnnnnnnnnaggcataggatgacaaagggaa

SEQ ID NO: 907 - DNA, 488 bp, Artificial Sequence
  miRNA-TS
  misc_feature (23)..(42): n is any naturally-occurring nucleotide and up to nineteen of them may be absent
  misc_feature (65)..(84): n is any naturally-occurring nucleotide and up to nineteen of them may be absent
  misc_feature (108)..(127): n is any naturally-occurring nucleotide and up to nineteen of them may be absent
  misc_feature (150)..(169): n is any naturally-occurring nucleotide and up to nineteen of them may be absent
  misc_feature (193)..(212): n is any naturally-occurring nucleotide and up to nineteen of them may be absent
  misc_feature (235)..(254): n is any naturally-occurring nucleotide and up to nineteen of them may be absent
  misc_feature (277)..(296): n is any naturally-occurring nucleotide and up to nineteen of them may be absent
  misc_feature (320)..(339): n is any naturally-occurring nucleotide and up to nineteen of them may be absent
  misc_feature (362)..(381): n is any naturally-occurring nucleotide and up to nineteen of them may be absent
  misc_feature (405)..(424): n is any naturally-occurring nucleotide and up to nineteen of them may be absent
  misc_feature (447)..(466): n is any naturally-occurring nucleotide and up to nineteen of them may be absent
acaaaccttttgttcgtcttatnnnnnnnnnnnnnnnnnnnncrcattattactcacggtacgannnnnnnnnnnnnnnnnnnnctacgcgtattcttaagcaataannnnnnnnnnnnnnnnnnnnacaaaccttttgttcgtcttatnnnnnnnnnnnnnnnnnnnnctacgcgtattcttaagcaataannnnnnnnnnnnnnnnnnnncrcattattactcacggtacgannnnnnnnnnnnnnnnnnnnacaaaccttttgttcgtcttatnnnnnnnnnnnnnnnnnnnnctacgcgtattcttaagcaataannnnnnnnnnnnnnnnnnnncrcattattactcacggtacgannnnnnnnnnnnnnnnnnnnctacgcgtattcttaagcaataannnnnnnnnnnnnnnnnnnncrcattattactcacggtacgannnnnnnnnnnnnnnnnnnnacaaaccttttgttcgtcttat

SEQ ID NO: 908 - DNA, 492 bp, Artificial Sequence
  miRNA-TS
  misc_feature (24)..(43): n is any naturally-occurring nucleotide and up to nineteen of them may be absent
  misc_feature (66)..(85): n is any naturally-occurring nucleotide and up to nineteen of them may be absent
  misc_feature (109)..(128): n is any naturally-occurring nucleotide and up to nineteen of them may be absent
  misc_feature (151)..(170): n is any naturally-occurring nucleotide and up to nineteen of them may be absent
  misc_feature (194)..(213): n is any naturally-occurring nucleotide and up to nineteen of them may be absent
  misc_feature (237)..(256): n is any naturally-occurring nucleotide and up to nineteen of them may be absent
  misc_feature (280)..(299): n is any naturally-occurring nucleotide and up to nineteen of them may be absent
  misc_feature (322)..(341): n is any naturally-occurring nucleotide and up to nineteen of them may be absent
  misc_feature (365)..(384): n is any naturally-occurring nucleotide and up to nineteen of them may be absent
  misc_feature (407)..(426): n is any naturally-occurring nucleotide and up to nineteen of them may be absent
  misc_feature (450)..(469): n is any naturally-occurring nucleotide and up to nineteen of them may be absent
ctacgcgtattcttaagcaataannnnnnnnnnnnnnnnnnnncrcattattactcacggtacgannnnnnnnnnnnnnnnnnnntccaatcagttcctgatgcagtannnnnnnnnnnnnnnnnnnncrcattattactcacggtacgannnnnnnnnnnnnnnnnnnntccaatcagttcctgatgcagtannnnnnnnnnnnnnnnnnnnctacgcgtattcttaagcaataannnnnnnnnnnnnnnnnnnntccaatcagttcctgatgcagtannnnnnnnnnnnnnnnnnnncrcattattactcacggtacgannnnnnnnnnnnnnnnnnnnctacgcgtattcttaagcaataannnnnnnnnnnnnnnnnnnncrcattattactcacggtacgannnnnnnnnnnnnnnnnnnntccaatcagttcctgatgcagtannnnnnnnnnnnnnnnnnnnctacgcgtattcttaagcaataa

SEQ ID NO: 909 - DNA, 492 bp, Artificial Sequence
  miRNA-TS
  misc_feature (24)..(43): n is any naturally-occurring nucleotide and up to nineteen of them may be absent
  misc_feature (66)..(85): n is any naturally-occurring nucleotide and up to nineteen of them may be absent
  misc_feature (109)..(128): n is any naturally-occurring nucleotide and up to nineteen of them may be absent
  misc_feature (151)..(170): n is any naturally-occurring nucleotide and up to nineteen of them may be absent
  misc_feature (194)..(213): n is any naturally-occurring nucleotide and up to nineteen of them may be absent
  misc_feature (237)..(256): n is any naturally-occurring nucleotide and up to nineteen of them may be absent
  misc_feature (280)..(299): n is any naturally-occurring nucleotide and up to nineteen of them may be absent
  misc_feature (322)..(341): n is any naturally-occurring nucleotide and up to nineteen of them may be absent
  misc_feature (365)..(384): n is any naturally-occurring nucleotide and up to nineteen of them may be absent
  misc_feature (407)..(426): n is any naturally-occurring nucleotide and up to nineteen of them may be absent
  misc_feature (450)..(469): n is any naturally-occurring nucleotide and up to nineteen of them may be absent
ctacgcgtattcttaagcaataannnnnnnnnnnnnnnnnnnnagccaagctcagacggatccgannnnnnnnnnnnnnnnnnnntccaatcagttcctgatgcagtannnnnnnnnnnnnnnnnnnnagccaagctcagacggatccgannnnnnnnnnnnnnnnnnnntccaatcagttcctgatgcagtannnnnnnnnnnnnnnnnnnnctacgcgtattcttaagcaataannnnnnnnnnnnnnnnnnnntccaatcagttcctgatgcagtannnnnnnnnnnnnnnnnnnnagccaagctcagacggatccgannnnnnnnnnnnnnnnnnnnctacgcgtattcttaagcaataannnnnnnnnnnnnnnnnnnnagccaagctcagacggatccgannnnnnnnnnnnnnnnnnnntccaatcagttcctgatgcagtannnnnnnnnnnnnnnnnnnnctacgcgtattcttaagcaataa

SEQ ID NO: 910 - DNA, 488 bp, Artificial Sequence
  miRNA-TS
  misc_feature (24)..(43): n is any naturally-occurring nucleotide and up to nineteen of them may be absent
  misc_feature (65)..(84): n is any naturally-occurring nucleotide and up to nineteen of them may be absent
  misc_feature (108)..(127): n is any naturally-occurring nucleotide and up to nineteen of them may be absent
  misc_feature (149)..(168): n is any naturally-occurring nucleotide and up to nineteen of them may be absent
  misc_feature (192)..(211): n is any naturally-occurring nucleotide and up to nineteen of them may be absent
  misc_feature (235)..(254): n is any naturally-occurring nucleotide and up to nineteen of them may be absent
  misc_feature (278)..(297): n is any naturally-occurring nucleotide and up to nineteen of them may be absent
  misc_feature (319)..(338): n is any naturally-occurring nucleotide and up to nineteen of them may be absent
  misc_feature (362)..(381): n is any naturally-occurring nucleotide and up to nineteen of them may be absent
  misc_feature (403)..(422): n is any naturally-occurring nucleotide and up to nineteen of them may be absent
  misc_feature (446)..(465): n is any naturally-occurring nucleotide and up to nineteen of them may be absent
ctacgcgtattcttaagcaataannnnnnnnnnnnnnnnnnnnaaagagaccggttcactgtgrnnnnnnnnnnnnnnnnnnnntccaatcagttcctgatgcagtannnnnnnnnnnnnnnnnnnnaaagagaccggttcactgtgrnnnnnnnnnnnnnnnnnnnntccaatcagttcctgatgcagtannnnnnnnnnnnnnnnnnnnctacgcgtattcttaagcaataannnnnnnnnnnnnnnnnnnntccaatcagttcctgatgcagtannnnnnnnnnnnnnnnnnnnaaagagaccggttcactgtgrnnnnnnnnnnnnnnnnnnnnctacgcgtattcttaagcaataannnnnnnnnnnnnnnnnnnnaaagagaccggttcactgtgrnnnnnnnnnnnnnnnnnnnntccaatcagttcctgatgcagtannnnnnnnnnnnnnnnnnnnctacgcgtattcttaagcaataa

SEQ ID NO: 911 - DNA, 492 bp, Artificial Sequence
  miRNA-TS
  misc_feature (24)..(43): n is any naturally-occurring nucleotide and up to nineteen of them may be absent
  misc_feature (66)..(85): n is any naturally-occurring nucleotide and up to nineteen of them may be absent
  misc_feature (109)..(128): n is any naturally-occurring nucleotide and up to nineteen of them may be absent
  misc_feature (151)..(170): n is any naturally-occurring nucleotide and up to nineteen of them may be absent
  misc_feature (194)..(213): n is any naturally-occurring nucleotide and up to nineteen of them may be absent
  misc_feature (237)..(256): n is any naturally-occurring nucleotide and up to nineteen of them may be absent
  misc_feature (280)..(299): n is any naturally-occurring nucleotide and up to nineteen of them may be absent
  misc_feature (322)..(341): n is any naturally-occurring nucleotide and up to nineteen of them may be absent
  misc_feature (365)..(384): n is any naturally-occurring nucleotide and up to nineteen of them may be absent
  misc_feature (407)..(426): n is any naturally-occurring nucleotide and up to nineteen of them may be absent
  misc_feature (450)..(469): n is any naturally-occurring nucleotide and up to nineteen of them may be absent
ctacgcgtattcttaagcaataannnnnnnnnnnnnnnnnnnnatactttttggggtaagggcttnnnnnnnnnnnnnnnnnnnntccaatcagttcctgatgcagtannnnnnnnnnnnnnnnnnnnatactttttggggtaagggcttnnnnnnnnnnnnnnnnnnnntccaatcagttcctgatgcagtannnnnnnnnnnnnnnnnnnnctacgcgtattcttaagcaataannnnnnnnnnnnnnnnnnnntccaatcagttcctgatgcagtannnnnnnnnnnnnnnnnnnnatactttttggggtaagggcttnnnnnnnnnnnnnnnnnnnnctacgcgtattcttaagcaataannnnnnnnnnnnnnnnnnnnatactttttggggtaagggcttnnnnnnnnnnnnnnnnnnnntccaatcagttcctgatgcagtannnnnnnnnnnnnnnnnnnnctacgcgtattcttaagcaataa

SEQ ID NO: 912 - DNA, 491 bp, Artificial Sequence
  miRNA-TS
  misc_feature (24)..(43): n is any naturally-occurring nucleotide and up to nineteen of them may be absent
  misc_feature (66)..(85): n is any naturally-occurring nucleotide and up to nineteen of them may be absent
  misc_feature (109)..(128): n is any naturally-occurring nucleotide and up to nineteen of them may be absent
  misc_feature (151)..(170): n is any naturally-occurring nucleotide and up to nineteen of them may be absent
  misc_feature (194)..(213): n is any naturally-occurring nucleotide and up to nineteen of them may be absent
  misc_feature (236)..(255): n is any naturally-occurring nucleotide and up to nineteen of them may be absent
  misc_feature (279)..(298): n is any naturally-occurring nucleotide and up to nineteen of them may be absent
  misc_feature (321)..(340): n is any naturally-occurring nucleotide and up to nineteen of them may be absent
  misc_feature (364)..(383): n is any naturally-occurring nucleotide and up to nineteen of them may be absent
  misc_feature (406)..(425): n is any naturally-occurring nucleotide and up to nineteen of them may be absent
  misc_feature (449)..(468): n is any naturally-occurring nucleotide and up to nineteen of them may be absent
ctacgcgtattcttaagcaataannnnnnnnnnnnnnnnnnnnatgccctttcatcattgcactgnnnnnnnnnnnnnnnnnnnntccaatcagttcctgatgcagtannnnnnnnnnnnnnnnnnnnatgccctttcatcattgcactgnnnnnnnnnnnnnnnnnnnntccaatcagttcctgatgcagtannnnnnnnnnnnnnnnnnnnatgccctttcatcattgcactgnnnnnnnnnnnnnnnnnnnntccaatcagttcctgatgcagtannnnnnnnnnnnnnnnnnnnagccaagctcagacggatccgannnnnnnnnnnnnnnnnnnnctacgcgtattcttaagcaataannnnnnnnnnnnnnnnnnnnatgccctttcatcattgcactgnnnnnnnnnnnnnnnnnnnntccaatcagttcctgatgcagtannnnnnnnnnnnnnnnnnnnctacgcgtattcttaagcaataa

SEQ ID NO: 913 - DNA, 108 bp, Artificial Sequence
  K4 Aptazyme
ggcgcgtcctggattcgtacaaaacataccagatttcgatctggagaggtgaagaatacgaccacctgtacatccagctgatgagtcccaaataggacgaaacgcgct

SEQ ID NO: 914 - DNA, 108 bp, Artificial Sequence
  K7 Aptazyme
ggcgcgtcctggattcgtgcaaaacataccagatttcgatctggagaggtgaagaatacgaccacctgtacatccagctgatgagtcccaaataggacgaaacgcgct

(19) United States
(12) Patent Application Publication
(10) Pub. No.: US 2022/0380735 A1
(43) Pub. Date: Dec. 1, 2022

(54) DUAL VIRUSES AND DUAL ONCOLYTIC VIRUSES AND METHODS OF TREATMENT

(71) Applicant: Oncorus, Inc., Cambridge, MA (US)

(72) Inventors: Edward M. KENNEDY, Cambridge, MA (US); Lorena LERNER, Cambridge, MA (US); Christophe QUEVA, Cambridge, MA (US); Craig A. STRATHDEE, Cambridge, MA (US); Jennifer S. LEE, Cambridge, MA (US)

(21) Appl. No.: 17/767,132

(22) PCT Filed: Oct. 9, 2020
(86) PCT No.: PCT/US2020/055133

§ 371 Date: Apr. 7, 2022

Related U.S. Application Data
(60) Provisional application No. 62/913,514, filed on Oct. 10, 2019.

(51) Int. Cl. C12N 7/00; C12N 15/113; C12N 15/86

(52) U.S. Cl. CPC C12N 7/00; C12N 15/1131; C12N 15/86; C12N 2310/141; C12N 2710/16632; C12N 2710/16644

(57) ABSTRACT

The present disclosure provides dual viruses capable of producing a primary virus and a secondary virus, and dual oncolytic viruses capable of producing a primary oncolytic virus and a secondary oncolytic virus.

### CROSS-REFERENCE TO RELATED APPLICATIONS

This application claims priority to U.S. Provisional Application No. 62/913,514, filed Oct. 10, 2019, the content of which is incorporated herein by reference in its entirety.

### STATEMENT REGARDING SEQUENCE LISTING

The Sequence Listing associated with this application is provided in text format in lieu of a paper copy, and is hereby incorporated by reference into the specification. A computer readable format copy of the Sequence Listing: filename: ONCR-013_01WO_SeqList_ST25.txt, date recorded: Oct. 9, 2020, file size 271 kilobytes.

### BACKGROUND

Oncolytic viruses are designed to preferentially infect and destroy cancer cells (MacLean et al., J. Gen. Virol. 72:630-639 (1991); Robertson et al., J. Gen. Virol. 73:967-970 (1992); Brown et al., J. Gen. Virol. 75:3767-3686 (1994); Chou et al., Science 250:1262-1265 (1990)) and have been used in multiple pre-clinical and clinical studies for cancer treatment. Direct tumor cell lysis results not only in cell death, but also the generation of an adaptive immune response against tumor antigens taken up and presented by local antigen presenting cells. However, robust anti-tumor immune responses are limited by low potency of viral strains as well as potential redirection of the immune response to target the virus itself.

### SUMMARY

The disclosure provides recombinant primary oncolytic viruses comprising a polynucleotide encoding a secondary oncolytic virus. In some embodiments, the primary oncolytic virus and the secondary oncolytic virus are replication-competent. In some embodiments, the primary oncolytic virus and/or the secondary oncolytic virus is/are replication-incompetent. In some embodiments, the polynucleotide encoding a secondary oncolytic virus is operably linked to a regulatable promoter. In some embodiments, the primary oncolytic virus generates an antigen-specific immune response that does not mediate antigen-specific immunity against the secondary oncolytic virus.

The disclosure provides recombinant primary viruses comprising a polynucleotide encoding a secondary virus. In some embodiments, the primary virus and the secondary virus are replication-competent. In some embodiments, the primary virus and/or the secondary virus is/are replication-incompetent. In some embodiments, the polynucleotide encoding the secondary virus is operably linked to a regulatable promoter. In some embodiments, the primary virus generates an antigen-specific immune response that does not mediate antigen-specific immunity against the secondary virus.

In some embodiments, the primary oncolytic virus is a double-stranded DNA (dsDNA) virus. In some embodiments, the primary virus is a double-stranded DNA (dsDNA) virus. In some embodiments, the dsDNA virus is a herpes simplex virus (HSV) or an adenovirus. In some embodiments, the dsDNA virus is a virus of Poxviridae family. In some embodiments, the dsDNA virus is a molluscum contagiosum virus, a myxoma virus, a vaccina virus, a monkeypox virus, or a yatapoxvirus. In some embodiments, the primary oncolytic virus or the primary virus is a RNA virus. In some embodiments, the RNA virus is a paramyxovirus or a rhabdovirus.

In some embodiments, the secondary oncolytic virus is a positive-sense single-stranded RNA (ssRNA) virus, a negative-sense ssRNA virus, or an ambi-sense ssRNA virus. In some embodiments, the secondary virus is a positive-sense single-stranded RNA (ssRNA) virus, a negative-sense ssRNA virus, or an ambi-sense ssRNA virus. In some embodiments, the negative-sense ssRNA virus is a virus of Rrhabdoviridae family, Paramyxoviridae family, or Orthomyxoviridae family. In some embodiments, the virus of Rhabdoviridae family is a vesicular stomatitis virus (VSV) or a maraba virus. In some embodiments, the virus of Paramyxoviridae family is a Newcastle Disease virus, a Sendai virus, or a measles virus. In some embodiments, the virus of Orthomyxoviridae family is an influenza virus. In some embodiments, the positive-sense ssRNA virus is an enterovirus. In some embodiments, the enterovirus is a poliovirus, a Seneca Valley virus (SVV), a coxsackievirus, or an echovirus. In some embodiments, the coxsakivirus is a coxsackievirus A (CVA) or a coxsackievirus B (CVB), In some embodiments, the coxsakivirus is CVA9, CVA21 or CVB3. In some embodiments, the positive-sense ssRNA virus is a Encephalomyocarditis virus (EMCV). In some embodiments, the positive-sense ssRNA virus is a Mengovirus. In some embodiments, the positive-sense ssRNA virus is a virus of Togaviridae family. In some embodiments, the virus of Togaviridae familyis a new world alphavirus or old world alphavirus. In some embodiments, the new world alphavirus or old world alphavirusis is VEEV, WEEV, EEV, Sindbis virus, Semliki Forest virus, Ross River Virus, or Mayaro virus.

In some embodiments, the primary oncolytic virus and/or the secondary oncolytic virus is a chimeric virus. In some embodiments, the primary oncolytic virus and/or the secondary oncolytic virus is a pseudotyped virus. In some embodiments, the secondary oncolytic virus is a pseudotyped virus, and wherein the primary oncolytic virus comprises a coding region for a capsid protein or an envelope protein of the secondary oncolytic virus outside the cording region for the secondary oncolytic virus. In some embodiments, the secondary oncolytic virus is an alphavirus. In some embodiments the secondary virus is a paramyxovirus or a rhabdovirus.

In some embodiments, the primary virus and/or the secondary virus is a chimeric virus. In some embodiments, the primary virus and/or the secondary virus is a pseudotyped virus. In some embodiments, the secondary virus is a pseudotyped virus, and wherein the primary virus comprises a coding region for a capsid protein or an envelope protein of the secondary virus outside the cording region for the secondary virus. In some embodiments, the secondary virus is an alphavirus. In some embodiments the secondary virus is a paramyxovirus or a rhabdovirus.

In some embodiments, the regulatable promoter is selected from a steroid-inducible promoter, a metallothionine promoter, an MX-1 promoter, a GENESWITCH™ hybrid promoter, a cumate-responsive promoter, and a tetracycline-inducible promoter. In some embodiments, the regulatable promoter comprises a constitutive promoter flanked by recombinase recognition sites.

In some embodiments, the primary oncolytic virus of the disclosure further comprises a second polynucleotide encoding a peptide capable of binding to the regulatable promoter. In some embodiments, the primary virus of the disclosure further comprises a second polynucleotide encoding a peptide capable of binding to the regulatable promoter. In some embodiments, the second polynucleotide is operably linked to a constitutive promoter or an inducible promoter. In some embodiments, the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

In some embodiments, the regulatable promoter is a tetracycline (Tet)-dependent promoter and wherein in the peptide is a reverse tetracycline-controlled transactivator (rtTA) peptide. In some embodiments, the regulatable promoter is a tetracycline (Tet)-dependent promoter and wherein in the peptide is a tetracycline-controlled transactivator (tTA) peptide.

In some embodiments, the primary oncolytic virus further comprises a polynucleotide encoding one or more RNA interference (RNAi) molecules. In some embodiments, the polynucleotide encoding one or more RNA interference (RNAi) molecules is operably linked to a second regulatable promoter In some embodiments, the one or more RNAi molecules bind to a target sequence in the genome of the secondary oncolytic virus and inhibits replication of the secondary oncolytic virus. In some embodiments, the RNAi molecule is an siRNA, an miRNA, an shRNA, or an AmiRNA.

In some embodiments, the primary virus further comprises a polynucleotide encoding one or more RNA interference (RNAi) molecules. In some embodiments, the polynucleotide encoding one or more RNA interference (RNAi) molecules is operably linked to a second regulatable promoter. In some embodiments, the one or more RNAi molecules bind to a target sequence in the genome of the secondary virus and inhibits replication of the secondary virus. In some embodiments, the RNAi molecule is an siRNA, an miRNA, an shRNA, or an AmiRNA.

In some embodiments, the polynucleotide encoding the secondary oncolytic virus comprises one or more recombinase recognition sites. In some embodiments, the polynucleotide encoding the secondary oncolytic virus comprises one or more recombinase-responsive cassettes, wherein the recombinase-responsive cassette comprises the one or more recombinase recognition sites.

In some embodiments, the polynucleotide encoding the secondary virus comprises one or more recombinase recognition sites. In some embodiments, the polynucleotide encoding the secondary virus comprises one or more recombinase-responsive cassettes, wherein the recombinase-responsive cassette comprises the one or more recombinase recognition sites.

In some embodiments, the one or more recombinase-responsive cassettes comprise a Recombinase-Responsive Excision Cassette (RREC). In some embodiments, the RREC comprises a transcriptional/translational termination (STOP) element. In some embodiments, the transcriptional/translational termination (STOP) element comprises a sequence having 80% identity to any one of SEQ ID NOS: 854-856. In some embodiments, the one or more recombinase-responsive cassettes comprise a Recombinase-Responsive Inversion Cassette (RRIC). In some embodiments, the RRIC comprises two or more orthogonal Recombinase Recognition Sites on each side of a Central Element. In some embodiments, the RRIC comprises a promoter or a portion of the promoter. In some embodiments, the RRIC comprises a coding region or a portion of the coding region, wherein the coding region encodes the viral genome of the secondary oncolytic virus or the secondary virus. In some embodiments, the RRIC comprises one or more Control Element(s). In some embodiments, the Control Element(s) is/are transcriptional/translational termination (STOP) elements. In some embodiments, the Control Element(s) has/have a sequence having 80% identity to any one of SEQ ID NOS: 854-856. In some embodiments, the Recombinase-Responsive Inversion Cassette (RRIC) further comprises a portion of an intron. In some embodiments, the polynucleotide encoding the secondary oncolytic virus or the secondary virus yields a mature viral genome transcript of the secondary oncolytic virus or the secondary virus without the Recombinase Recognition Site after removal of the intron via mRNA splicing.

In some embodiments, the primary oncolytic virus or the primary virus further comprises a polynucleotide encoding the recombinase. In some embodiments, the primary virus further comprises a polynucleotide encoding the recombinase. In some embodiments, the recombinase is a Flippase (Flp) or a Cre recombinase (Cre). In some embodiments, the coding region of the recombinase comprises an intron. In some embodiments, an expression cassette of the recombinase recombinase comprises one or more mRNA destabilization elements. In some embodiments, the recombinase is a part of a fusion protein comprising an additional polypeptide, and wherein the additional polypeptide regulates the activity and/or cellular localization of the recombinase. In some embodiments, the activity and/or cellular localization of the recombinase is regulated by the presence of a ligand and/or a small molecule. In some embodiments, the additional polypeptide comprises a ligand binding domain of an estrogen receptor protein.

In some embodiments, the one or more recombinase recognition sites are flippase recognition target (FRT) sites.

In some embodiments, the primary oncolytic virus further comprises a polynucleotide encoding a regulatory polypeptide, and wherein the regulatory polypeptide regulates activity of one or more promoters.

In some embodiments, the primary virus further comprises a polynucleotide encoding a regulatory polypeptide, and wherein the regulatory polypeptide regulates activity of one or more promoters.

The disclosure provides recombinant primary oncolytic viruses comprising a first polynucleotide encoding a secondary oncolytic virus and a second polynucleotide encoding one or more RNA interference (RNAi) molecules. In some embodiments, the primary oncolytic virus and the secondary oncolytic viruses are replication-competent. In some embodiments, the first polynucleotide is operably linked to a first regulatable promoter and wherein the second polynucleotide is operably linked to a second regulatable promoter. In some embodiments, the primary oncolytic virus generates an antigen-specific immune response that does not mediate antigen-specific immunity against the secondary oncolytic virus.

In some embodiments, the primary oncolytic virus is a double-stranded DNA (dsDNA) virus. In some embodiments, the dsDNA virus is a herpes simplex virus (HSV), an adenovirus or a virus of Poxviridae family, optionally wherein the virus of virus of Poxviridae family is a molluscum contagiosum virus, a myxoma virus, a vaccina virus, a monkeypox virus, or a yatapoxvirus. In some embodiments, the primary oncolytic virus is a RNA virus. In some embodiments, the RNA virus is a paramyxovirus or a rhabdovirus.

In some embodiments, the secondary oncolytic virus is a positive-sense single-stranded RNA (ssRNA) virus, a negative-sense ssRNA virus, or an ambi-sense ssRNA virus. In some embodiments, the negative-sense ssRNA virus is a virus of Rrhabdoviridae family, Paramyxoviridae family, or Orthomyxoviridae family, optionally wherein the virus of Rhabdoviridae family is a vesicular stomatitis virus (VSV) or a maraba virus; optionally wherein the virus of Paramyxoviridae family is a Newcastle Disease virus, a Sendai virus, or a measles virus; or optionally wherein the virus of Orthomyxoviridae family is an influenza virus. In some embodiments, the positive-sense ssRNA virus is an enterovirus, optionally wherein the enterovirus is a poliovirus, a Seneca Valley virus (SVV), a coxsackievirus, or an echovirus, optionally wherein the coxsakivirus is a coxsackievirus A (CVA) or a coxsackievirus B (CVB), optionally wherein the coxsakivirus is CVA9, CVA21 or CVB3. In some embodiments, the positive-sense ssRNA virus is a Encephalomyocarditis virus (EMCV) or a Mengovirus. In some embodiments, the positive-sense ssRNA virus is a virus of Togaviridae family, optionally wherein the virus of Togaviridae familyis a new world alphavirus or old world alphavirus, and optionally wherein the new world alphavirus or old world alphavirusis is VEEV, WEEV, EEV, Sindbis virus, Semliki Forest virus, Ross River Virus, or Mayaro virus.

In some embodiments, the primary oncolytic virus and/or the secondary oncolytic virus is a chimeric virus. In some embodiments, the primary oncolytic virus and/or the secondary oncolytic virus is a pseudotyped virus.

The disclosure provides recombinant primary viruses comprising a first polynucleotide encoding a secondary virus and a second polynucleotide encoding one or more RNA interference (RNAi) molecules. In some embodiments, the primary virus and the secondary viruses are replication-competent. In some embodiments, the first polynucleotide is operably linked to a first regulatable promoter and wherein the second polynucleotide is operably linked to a second regulatable promoter. In some embodiments, the primary virus generates an antigen-specific immune response that does not mediate antigen-specific immunity against the secondary virus.

In some embodiments, the primary virus is a double-stranded DNA (dsDNA) virus. In some embodiments, the dsDNA virus is a herpes simplex virus (HSV), an adenovirus or a virus of Poxviridae family, optionally wherein the virus of virus of Poxviridae family is a molluscum contagiosum virus, a myxoma virus, a vaccina virus, a monkeypox virus, or a yatapoxvirus. In some embodiments, the primary virus is a RNA virus. In some embodiments, the RNA virus is a paramyxovirus or a rhabdovirus.

In some embodiments, the secondary virus is a positive-sense single-stranded RNA (ssRNA) virus, a negative-sense ssRNA virus, or an ambi-sense ssRNA virus. In some embodiments, the negative-sense ssRNA virus is a virus of Rrhabdoviridae family, Paramyxoviridae family, or Orthomyxoviridae family, optionally wherein the virus of Rhabdoviridae family is a vesicular stomatitis virus (VSV) or a maraba virus; optionally wherein the virus of Paramyxoviridae family is a Newcastle Disease virus, a Sendai virus, or a measles virus; or optionally wherein the virus of Orthomyxoviridae family is an influenza virus. In some embodiments, the positive-sense ssRNA virus is an enterovirus, optionally wherein the enterovirus is a poliovirus, a Seneca Valley virus (SVV), a coxsackievirus, or an echovirus, optionally wherein the coxsakivirus is a coxsackievirus A (CVA) or a coxsackievirus B (CVB), optionally wherein the coxsakivirus is CVA9, CVA21 or CVB3. In some embodiments, the positive-sense ssRNA virus is a Encephalomyocarditis virus (EMCV) or a Mengovirus. In some embodiments, the positive-sense ssRNA virus is a virus of Togaviridae family, optionally wherein the virus of Togaviridae familyis a new world alphavirus or old world alphavirus, and optionally wherein the new world alphavirus or old world alphavirusis is VEEV, WEEV, EEV, Sindbis virus, Semliki Forest virus, Ross River Virus, or Mayaro virus.

In some embodiments, the primary virus and/or the secondary virus is a chimeric virus. In some embodiments, the primary virus and/or the secondary virus is a pseudotyped virus.

In some embodiments, the first and second regulatable promoters are selected from a steroid-inducible promoter, a metallothionine promoter, an MX-1 promoter, a GENESWITCH™ hybrid promoter, a cumate-responsive promoter, and a tetracycline-dependent promoter.

In some embodiments, the primary oncolytic virus or the primary virus of the disclosure further comprises a third polynucleotide encoding a first peptide capable of binding to the first regulatable promoter and a second peptide capable of binding to the second regulatable promoter. In some embodiments, the third polynucleotide is operably linked to a constitutive promoter. In some embodiments, the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

In some embodiments, the first regulatable promoter is a tetracycline (Tet)-inducible promoter and wherein in the first peptide is a reverse tetracycline-controlled transactivator (rtTA) peptide. In some embodiments, the second regulatable promoter is a tetracycline (Tet)-repressible promoter and wherein in the second peptide is a tetracycline-controlled transactivator (tTA) peptide. In some embodiments, the first regulatable promoter is a tetracycline (Tet)-repressible promoter and wherein in the first peptide is a tetracycline-controlled transactivator (tTA) peptide. In some embodiments, the second regulatable promoter is a tetracycline (Tet)-inducible promoter and wherein in the second peptide is a reverse tetracycline-controlled transactivator (rtTA) peptide.

In some embodiments, the one or more RNAi molecules bind to a target sequence in the genome of the secondary oncolytic virus and inhibits replication of the secondary oncolytic virus. In some embodiments, the one or more RNAi molecules bind to a target sequence in the genome of the secondary virus and inhibits replication of the secondary virus. In some embodiments, the RNAi molecule is an siRNA, an miRNA, an shRNA, or an AmiRNA.

In some embodiments, the polynucleotide encoding the secondary oncolytic virus comprises first 3′ ribozyme-encoding sequence and a second 5′ ribozyme encoding sequence. In some embodiments, the first and second ribozyme-encoding sequences encode a Hammerhead ribozyme or a hepatitis delta virus ribozyme.

In some embodiments, the genome of the primary oncolytic virus comprises an miRNA target sequence (miR-TS) cassette comprising one or more miRNA target sequences inserted into one or more viral genes required for replication or inserted into the 3′ or 5′ UTR of the viral genome. In some embodiments, the genome of the secondary oncolytic virus comprises an miRNA target sequence (miR-TS) cassette comprising one or more miRNA target sequences inserted into one or more viral genes required for replication or inserted into the 3′ or 5′ UTR of the viral genome. In some embodiments, the primary oncolytic virus and the secondary oncolytic virus each comprise an miRNA target sequence (miR-TS) cassette comprising one or more miRNA target sequences inserted into one or more viral genes required for replication or inserted into the 3′ or 5′ UTR of the viral genome. In some embodiments, expression of the one or more miRNAs in a cell inhibits replication of the primary and/or secondary oncolytic viruses.

In some embodiments, the polynucleotide encoding the secondary virus comprises first 3′ ribozyme-encoding sequence and a second 5′ ribozyme encoding sequence. In some embodiments, the first and second ribozyme-encoding sequences encode a Hammerhead ribozyme or a hepatitis delta virus ribozyme.

In some embodiments, the genome of the primary virus comprises an miRNA target sequence (miR-TS) cassette comprising one or more miRNA target sequences inserted into one or more viral genes required for replication or inserted into the 3′ or 5′ UTR of the viral genome. In some embodiments, the genome of the secondary virus comprises an miRNA target sequence (miR-TS) cassette comprising one or more miRNA target sequences inserted into one or more viral genes required for replication or inserted into the 3′ or 5′ UTR of the viral genome. In some embodiments, the primary virus and the secondary virus each comprise an miRNA target sequence (miR-TS) cassette comprising one or more miRNA target sequences inserted into one or more viral genes required for replication or inserted into the 3′ or 5′ UTR of the viral genome. In some embodiments, expression of the one or more miRNAs in a cell inhibits replication of the primary and/or secondary viruses.

In some embodiments, the primary oncolytic virus of the disclosure further comprises a polynucleotide sequence encoding at least one exogenous payload protein. In some embodiments, the exogenous payload protein is a fluorescent protein, an enzyme, a cytokine, a chemokine, or an antigen-binding molecule.

In some embodiments, expression of the secondary oncolytic virus is regulated by an exogenous agent. In some embodiments, the exogenous agent is a peptide, a hormone, or a small molecule.

In some embodiments, the primary virus of the disclosure further comprises a polynucleotide sequence encoding at least one exogenous payload protein. In some embodiments, the exogenous payload protein is a fluorescent protein, an enzyme, a cytokine, a chemokine, or an antigen-binding molecule.

In some embodiments, expression of the secondary virus is regulated by an exogenous agent. In some embodiments, the exogenous agent is a peptide, a hormone, or a small molecule.

The disclosure provides compositions comprising the primary oncolytic virus of the disclosure. The disclosure provides compositions comprising the primary virus of the disclosure.

The disclosure provides methods of killing a population of tumor cells comprising administering the primary oncolytic virus of the disclosure or the composition thereof to the population of tumor cells. In some embodiments, a first subpopulation of the tumor cells are infected and killed by the primary oncolytic virus. In some embodiments, a second subpopulation of the tumor cells are infected and killed by the secondary oncolytic virus. In some embodiments, a subpopulation of the tumor cells are infected and killed by both the primary oncolytic virus and the secondary oncolytic virus. In some embodiments, a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone.

In some embodiments, the method of the disclosure further comprises administering one or more exogenous agents to the population of tumor cells, wherein the one or more exogenous agents regulate the production of the secondary oncolytic virus. In some embodiments, the one or more exogenous agents is/are administered at the same time as the primary oncolytic virus, and wherein the presence of the exogenous agent(s) inhibits production of the secondary oncolytic virus. In some embodiments, the one or more exogenous agents is/are administered after the primary oncolytic virus, and wherein the presence of the exogenous agent(s) induces production of the secondary oncolytic virus. In some embodiments, the exogenous agent(s) is/are administered at least 1 day, at least 1 week, or at least 1 month, after administration of the primary oncolytic virus. In some embodiments, no secondary oncolytic virus is detectable prior to the administration of the exogenous agent(s).

The disclosure provides methods of treating a tumor in a subject in need thereof comprising administering the primary oncolytic virus of the disclosure or the composition thereof to the subject. In some embodiments, a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone. In some embodiments, the method leads to greater reduction of tumor size in the subject compared to administration of a reference primary oncolytic virus without the polynucleotide encoding the secondary oncolytic virus or the secondary oncolytic virus alone. In some embodiments, the method induces a stronger immune response against one or more tumor antigens in the subject compared to administering a reference primary oncolytic virus without the polynucleotide encoding the secondary oncolytic virus or administering the secondary oncolytic virus alone. In some embodiments, the method results in a reduced immune response against the primary oncolytic virus in the subject compared to administering a reference primary oncolytic virus without the polynucleotide encoding the secondary oncolytic virus. In some embodiments, the method results in a reduced immune response against the secondary oncolytic virus in the subject compared to administering the secondary oncolytic virus alone. In some embodiments, the method results in preferential/more specific killing of tumor cells in the subject compared to administering a reference primary oncolytic virus without the polynucleotide encoding the secondary oncolytic virus or administering the secondary oncolytic virus alone. In some embodiments, the method results in more persistent production of the primary oncolytic virus in the subject compared to administering a reference primary oncolytic virus without the polynucleotide encoding the secondary oncolytic virus. In some embodiments, the method results in more persistent production of the secondary oncolytic virus in the subject compared to administering the secondary oncolytic virus alone. In some embodiments, the method results in an extended period of tumor inhibition in the subject compared to administering a reference primary oncolytic virus without the polynucleotide encoding the secondary oncolytic virus or the secondary oncolytic virus alone. In some embodiments, the method enables viral infection of more cell types compared to administering a reference primary oncolytic virus without the polynucleotide encoding the secondary oncolytic virus or the secondary oncolytic virus alone. In some embodiments, the method further comprises administering one or more exogenous agents to the population of tumor cells, wherein the one or more exogenous agents regulate the production of the secondary oncolytic virus. In some embodiments, the one or more exogenous agents is/are administered at the same time as the primary oncolytic virus, and wherein the presence of the exogenous agent(s) inhibits production of the secondary oncolytic virus. In some embodiments, the one or more exogenous agents is/are administered after the primary oncolytic virus, and wherein the presence of the exogenous agent(s) induces production of the secondary oncolytic virus. In some embodiments, the exogenous agent(s) is/are administered at least 1 day, at least 1 week, or at least 1 month, after administration of the primary oncolytic virus. In some embodiments, no secondary oncolytic virus is detectable prior to the administration of the exogenous agent(s).

The disclosure provides methods of killing a population of tumor cells comprising administering the primary virus of the disclosure or the composition thereof to the population of tumor cells. In some embodiments, a first subpopulation of the tumor cells are infected and killed by the primary virus. In some embodiments, a second subpopulation of the tumor cells are infected and killed by the secondary virus. In some embodiments, a subpopulation of the tumor cells are infected and killed by both the primary virus and the secondary virus. In some embodiments, a greater number of tumor cells in the population are killed by the primary and secondary viruses compared to the number of tumor cells killed by a reference primary virus without the polynucleotide encoding the secondary virus or the secondary virus alone.

In some embodiments, the method of the disclosure further comprises administering one or more exogenous agents to the population of tumor cells, wherein the one or more exogenous agents regulate the production of the secondary virus. In some embodiments, the one or more exogenous agents is/are administered at the same time as the primary virus, and wherein the presence of the exogenous agent(s) inhibits production of the secondary virus. In some embodiments, the one or more exogenous agents is/are administered after the primary virus, and wherein the presence of the exogenous agent(s) induces production of the secondary virus. In some embodiments, the exogenous agent(s) is/are administered at least 1 day, at least 1 week, or at least 1 month, after administration of the primary virus. In some embodiments, no secondary virus is detectable prior to the administration of the exogenous agent(s).

The disclosure provides methods of treating a tumor in a subject in need thereof comprising administering the primary virus of the disclosure or the composition thereof to the subject. In some embodiments, a greater number of tumor cells in the population are killed by the primary and secondary viruses compared to the number of tumor cells killed by a reference primary virus without the polynucleotide encoding the secondary virus or the secondary virus alone. In some embodiments, the method leads to greater reduction of tumor size in the subject compared to administration of a reference primary virus without the polynucleotide encoding the secondary virus or the secondary virus alone. In some embodiments, the method induces a stronger immune response against one or more tumor antigens in the subject compared to administering a reference primary virus without the polynucleotide encoding the secondary virus or administering the secondary virus alone. In some embodiments, the method results in a reduced immune response against the primary virus in the subject compared to administering a reference primary virus without the polynucleotide encoding the secondary virus. In some embodiments, the method results in a reduced immune response against the secondary virus in the subject compared to administering the secondary virus alone. In some embodiments, the method results in preferential/more specific killing of tumor cells in the subject compared to administering a reference primary virus without the polynucleotide encoding the secondary virus or administering the secondary virus alone. In some embodiments, the method results in more persistent production of the primary virus in the subject compared to administering a reference primary virus without the polynucleotide encoding the secondary virus. In some embodiments, the method results in more persistent production of the secondary virus in the subject compared to administering the secondary virus alone. In some embodiments, the method results in an extended period of tumor inhibition in the subject compared to administering a reference primary virus without the polynucleotide encoding the secondary virus or the secondary virus alone. In some embodiments, the method enables viral infection of more cell types compared to administering a reference primary virus without the polynucleotide encoding the secondary virus or the secondary virus alone. In some embodiments, the method further comprises administering one or more exogenous agents to the population of tumor cells, wherein the one or more exogenous agents regulate the production of the secondary virus. In some embodiments, the one or more exogenous agents is/are administered at the same time as the primary virus, and wherein the presence of the exogenous agent(s) inhibits production of the secondary virus. In some embodiments, the one or more exogenous agents is/are administered after the primary virus, and wherein the presence of the exogenous agent(s) induces production of the secondary virus. In some embodiments, the exogenous agent(s) is/are administered at least 1 day, at least 1 week, or at least 1 month, after administration of the primary virus. In some embodiments, no secondary virus is detectable prior to the administration of the exogenous agent(s).

The disclosure provides polynucleotide encoding the primary oncolytic virus of the disclosure. The disclosure provides polynucleotide encoding the primary virus of the disclosure. The disclosure provides vectors comprising the polynucleotide of the disclosure. The disclosure provides pharmaceutical composition comprising the vector of the disclosure.

### BRIEF DESCRIPTION OF THE FIGURES

FIG. 1A-FIG. 1B show schematics of nested oncolytic viruses and immune responses over time. FIG. 1A illustrates a nested oncolytic viruses, where the genome of the primary oncolytic virus (oV1 genome, grey bar) comprises a polynucleotide encoding the genome of a secondary oncolytic virus (oV2 polynucleotide, white bar), such that two different viruses (oV1 and oV2) are produced from the same construct. FIG. 1B illustrates the relative levels of oV1 and oV2 over time, where production of oV2 is triggered by an inducing stimuli. The corresponding expansion of tumor-specific CD8+ T-cells over time is indicated by the grey dashed line. These procedures may be performed with a virus that is not an oncolytic virus.

FIG. 2 illustrates a nested oncolytic viral construct where the primary virus is a recombinant HSV and the secondary virus is a positive-sense single-stranded RNA virus (lower left) or a negative-sense single-stranded RNA virus (lower right). The expression of the secondary viral genomes are regulated by a tetracycline-responsive Pol II promoter (black arrows) and a RNA polymerase I promoter (grey arrow). The recombinant HSV comprises D285N and A549T mutations in its glycoprotein B (gB: N/T), T128, T219a and T122 miR-target sequences in ICP27, a deletion of the joint region, a US12 mutation, T124, T1 and T143 miR-target sequences in ICP4, and T128, T204 and T219 miR-target sequences in ICP34.5.

FIG. 3 is a schematic depicting control elements for regulating the expression and/or function of virus activating T7 RNA polymerase or recombinase. Transcriptional control can be achieved with a tumor-specific promoter or ligand-inducible promoter. Post-transcriptional control elements include modulating mRNA or the mRNA encoded protein half-life, miRNA target sites, Tet-ON miR-T elements, Tet-OFF ribozymes/aptazymes, and any combination thereof that controls transcript abundance in a ligand dependent or constitutive fashion. Additional control elements can be engineered into the encoded polypeptide (e.g., recombinase) to control its half life, subcellular localization and/or activity.

FIG. 4A-FIG. 4B show the use of site-directed recombination systems to control expression of the secondary oncolytic virus. FIG. 4A shows a scheme for insertion of frameshift/stop codons in the polynucleotide encoding the secondary oncolytic virus that could be excised by a FLP or another recombinase. FIG. 4B shows an inactive inverted promoter that could be rendered active inverted to the correct orientation. These procedures may be performed with a virus that is not an oncolytic virus.

FIG. 5 illustrates exemplary schematics of components that can be inserted into the primary oncolytic viral genome to produce an exemplary nested oncolytic viral construct. Expression of the rtTA peptide is under the control of a constitutively active promoter and expression of the secondary viral genome is under the control of a tetracycline-responsive (TetOn) Pol II promoter, such that transcription of the viral genome occurs in the presence of Tet and the rtTA peptide. Expression of the secondary oncolytic virus transcript is further regulated by an internal TetOff-ribozyme (TetOff-R), such that the transcript is degraded in the absence of Tet. Further, the secondary viral transcript is activated by 5′ and 3′ TetOn-ribozymes (TetOn-R), such that the mRNA transcript is processed on the 5′ and 3′ ends in the presence of Tet. These procedures may be performed with a virus that is not an oncolytic virus.

FIG. 6 illustrates exemplary schematics of components that can be inserted into the primary oncolytic viral genome to produce an exemplary nested oncolytic viral construct. Expression of the rtTA and the tetracycline transactivator (tTA) peptide are under the control of a constitutively active promoter. Expression of the secondary viral genome is under the control of a TetOn Pol II promoter, such that transcription of the viral genome occurs in the presence of Tet and the rtTA peptide. Expression of the secondary oncolytic virus transcript is further regulated by shRNAs specific for target sequences in the mRNA transcript of the secondary virus. Expression of the shRNAs is under the control of a TetOff promoter, such that transcription of the shRNAs occurs in the absence of Tet and the presence of the tTA peptide. Further, the secondary viral transcript is activated by 5′ and 3′ TetOn-R, such that the mRNA transcript is processed on the 5′ and 3′ ends in the presence of Tet. These procedures may be performed with a virus that is not an oncolytic virus.

FIG. 7 illustrates schematics of the components inserted into the primary oncolytic viral genome to produce an exemplary nested viral construct. Expression of the rtTA and the tTA peptides is under the control of a constitutively active promoter. Expression of the secondary viral genome is under the control of a TetOn Pol II promoter, such that transcription of the viral genome occurs in the presence of Tet and the rtTA peptide. Expression of the secondary oncolytic virus transcript is further regulated by shRNAs specific for target sequences in the mRNA transcript of the secondary virus. Expression of the shRNAs is under the control of a TetOff promoter, such that transcription of the shRNAs occurs in the absence of Tet and the presence of the tTA peptide. Further, the secondary viral transcript is activated by cleavage on the 5′-end by an AmiRNA target site and 3′ TetOn-R, such that that the mRNA transcript is processed on the 5′ end in the presence of Tet. These procedures may be performed with a virus that is not an oncolytic virus.

FIG. 8 is a table illustrating multiple levels of control of the recombinase system. Flp is used as a non-limiting exemplary recombinase here.

FIG. 9 is a schematic depicting an exemplary Recombinase-Responsive Excision Cassette (RREC) comprising a STOP element.

FIGS. 10A-10B are schematics depicting exemplary Recombinase-Responsive Inversion Cassettes (RRIC) comprising STOP elements. FIG. 10A depicts a Promoter Inversion design, in which the promoter region and a STOP element is in the inverted orientation in the initial construct as shown by the inverted text. FIG. 10B depicts a Payload Inversion design, in which the cDNA encoding a payload molecule is in the inverted orientation in the initial construct as shown by the inverted text.

FIG. 11 is a schematic depicting an exemplary design of Recombinase-Responsive Inversion Cassettes (RRIC) with intron, which is referred to as Split Intron Inversion design. The inverted elements are depicted by the inverted text.

FIG. 12 is a bar chart showing reporter gene level in HEK293T cells transfected with MND-TetR construct and a construct of mCherry-NLuc reporter gene operably linked to a Tet-dependent promoter.

FIG. 13 is a bar chart showing reporter gene level in HEK293T cells transfected with various constructs.

FIG. 14 is a bar chart showing reporter gene level in HEK293T cells transfected with a combination of 3 different constructs as indicated.

FIG. 15A is a bar chart showing reporter gene level in HEK293T cells transfected with Flp-ERT2 fusion protein constructs with optional intron region and mCherry-NLuc reporter constructs with optional STOP cassette. FIG. 15B is a bar chart showing reporter gene level in HEK293T cells transfected with Flp-ERT2 fusion protein constructs with intron region and optional mRNA destabilization elements and other constructs as indicated.

FIG. 16 is a bar chart showing base line reporter gene level in HEK293T cells transfected with indicated expression construct. The inverted elements are depicted by the inverted text.

FIG. 17 is a bar chart showing reporter gene level in HEK293T cells transfected with indicated expression construct in response to doxycycline and/or 4OHT.

FIG. 18 is a schematic depicting the design of pDEST14 based expression construct for regulating reporter gene expression using multiple control elements. The insertion comprises, from left to right: attB1, SV40 pA, MND-TetR (in reverse orientation), HBP1-TO-FEXPi2, ACTB polyA, attB5, GAPDH polyA, CMV-NLucP (in reverse orientation), HBP2-TO-STOP3-mCherry-Fluc, bGH polyA, attB2.

FIG. 19 is a schematic depicting the design of ONCR222b vector based dual oncolytic viral vector. The 14.1 kb insertion comprises, from left to right: attB1, SV40 pA, MND-TetR (in reverse orientation), HBP1-TO-FEXPi2, ACTB polyA, attB5, GAPDH polyA, CMV (in reverse orientation), HBP2-TO-STOP3-SVV-mCherry, bGH polyA, attB2. The recombinant HSV comprises D285N and A549T mutations in its glycoprotein B (gB: N/T), T128, T219a and T122 miR-target sequences in ICP27, a deletion of the joint region, a US12 mutation, T124, T1 and T143 miR-target sequences in ICP4, and T128, T204 and T219 miR-target sequences in ICP34.5.

FIG. 20A is a graph showing the viral titer of HSV over time after infecting NCI-H1299 cells with the indicated dual oncolytic viral vectors. FIG. 20B is a graph showing the viral titer of SVV over time after infecting NCI-H1299 cells with the indicated dual oncolytic viral vectors.

FIG. 21 is a schematic depicting the design of dual oncolytic viral vectors with SVV viral genome inserted into HSV-1 viral genome. The recombinant HSV comprises D285N and A549T mutations in its glycoprotein B (gB: N/T), UL37 mutations, a deletion of the joint region, a US12 mutation, and T124, T1 and T143 miR-target sequences in ICP4.

FIG. 22 is a series of imaging figures showing 10-fold serial dilutions of viral infections with ONCR-189 or ONCR-190 viruses in Vero or H1299 cells.

FIG. 23 is a series of imaging figures showing 10-fold serial dilutions of viral infections with ONCR-189 and ONCR-190 viruses in H1299 cells.

FIG. 24A is a series of graphs showing an IC50 titer assay of ONCR-189 and ONCR-190 virus infection of H446 cells. FIG. 24B is a table showing the calculated IC50 values according to the experiments shown in FIG. 24A.

FIG. 25 is a bar graph showing a qPCR assay that measures SVV RNA copy numbers in transfected or infected H1299 cells.

FIG. 26A is a graph showing changes of tumor volumes over time for NCI-H1299 xenograft mice treated with oncolytic viruses. FIG. 26B is a graph showing changes in body weight for the same experiment.

FIG. 27 is a bar graph showing a array scanning cytometery assay that evaluates the expression level of mCherry under the control TetOff aptazymes in HEK293 cells.

FIG. 28 is a schematic depicting the design of various components of the dual virus. In some embodiments, the dual virus is a dual oncolytic virus. All italicized letters and/or broken lines indicate optional components. For example, optional RNAi target sequences can be inserted into the coding and/or non-coding region of the polynucleotide as indicated in the figure to control the expression level and/or stability of the corresponding RNA transcript through RNAi, and optional recombinase responsive cassette can be inserted into the polynucleotide as indicated in the figure to allow control of target RNA expression in response to the presence of recombinase.

### DETAILED DESCRIPTION

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers herein to introducing an agent or composition into a subject.

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

The term “effective amount” refers to the amount of an agent or composition that results in a particular physiological effect (e.g., an amount that can increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

The term “oncolytic virus” refers to a virus that has been modified to, or naturally, preferentially infect cancer cells.

The term “pharmaceutically-acceptable” refers to molecular entities and compositions that do not produce an allergic or similar untoward reaction when administered to a subject.

The term “replication-competent virus” refers to a virus capable of replicating in a host cell and producing an infectious viral particle

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

The term “subject” includes animals, such as e.g. mammals including primates and humans. The term includes livestock such as cattle, sheep, goats, cows, swine, and the like; domesticated animals such as dogs and cats; research animals such as rodents (e.g., mice, rats, hamsters), rabbits, primates, or swine such as inbred pigs and the like.

“Treating” as used herein refers to delivering an agent or composition to a subject to affect a physiologic outcome.

The term “vector” is used herein to refer to a nucleic acid molecule capable transferring or transporting another nucleic acid molecule.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

The term “operably linked” refers to a first polynucleotide molecule, such as a promoter, connected with a second transcribable polynucleotide molecule, such as coding sequence of a gene of interest or a viral genome, where the polynucleotide molecules are so arranged that the first polynucleotide molecule affects the function of the second polynucleotide molecule. The two polynucleotide molecules may be part of a single contiguous polynucleotide molecule and may be adjacent. However, polynucleotide molecules need not be contiguous to be operably linked. In some embodiments, the term “operably linked” also refers to two polynucleotide molecules that are operably linked after a recombination (e.g., mediated by a recombinase), but not in the initial arrangement.

### Dual Viruses

Throughout the disclosure, inclusive of all subheadings and all sections, the disclosure and embodiments provided for oncolytic viruses (e.g., dual oncolytic viruses) may be applied to viruses other than oncolytic viruses. In some embodiments, a virus that is not an oncolytic virus may be a non-oncolytic virus.

In some embodiments of the disclosure, a primary virus comprises a polynucleotide encoding a secondary virus. Such embodiments are referred to herein as “dual viruses” or “dual viral constructs” as the viral constructs are capable of producing two different oncolytic viruses from the same construct when introduced into a host cell.

In the context of viral treatment of malignancies, the overall objective is the promotion a tumor-specific immune response by tumor cell lysis. Effective viral therapy requires a virus that is sufficiently immunogenic to stimulate anti-tumor immune responses in the host and sufficiently virulent to mediate tumor cell lysis. At the same time, the immunogenicity and virulence of the virus can redirect the host immune response to the virus itself, thereby limiting the development of the anti-tumor immune response and tumor cell lysis, and instead leading to viral clearance. As such, there is a recognized need in the art for viruses that are able to promote anti-tumor immunity and restrain anti-viral immunity. In some embodiments, the disclosure provides dual viruses for treatment of malignant cancers.

Similar immunogenicity and/or virulence problem may be present for viruses in other applications such as vaccines or gene therapies. In some embodiments, the disclosure provides a vaccine composition comprising a dual virus of the disclose. In some embodiments, the disclosure provides dual viruses of the disclose as gene therapy vectors.

In some embodiments of the disclosure, a primary virus comprising a polynucleotide encoding a secondary virus (i.e., a dual virus). The dual viruses described herein enable production of two different viruses from one viral vector: a primary virus and a secondary virus. In some embodiments, expression of the primary and/or secondary virus is inducible, allowing for temporal control over expression of the primary and/or secondary viruses. In some embodiments, the dual viruses described herein promote viral persistence in the host, enabling increased viral lysis of tumor cells and enhanced development of tumor antigen-specific T cell populations.

### Dual Oncolytic Viruses

In some embodiments, the present disclosure provides a primary oncolytic virus comprising a polynucleotide encoding a secondary oncolytic virus. Such embodiments are referred to herein as “dual oncolytic viruses” or “dual oncolytic viral constructs” as the viral constructs are capable of producing two different oncolytic viruses from the same construct when introduced into a host cell.

In the context of viral treatment of malignancies, the overall objective is the promotion a tumor-specific immune response by tumor cell lysis. Effective oncolytic viral therapy requires a virus that is sufficiently immunogenic to stimulate anti-tumor immune responses in the host and sufficiently virulent to mediate tumor cell lysis. At the same time, the immunogenicity and virulence of the virus can redirect the host immune response to the virus itself, thereby limiting the development of the anti-tumor immune response and tumor cell lysis, and instead leading to viral clearance (Ikeda et al., Nature Medicine (1999) 5:8; 881-887). As such, there is a recognized need in the art for oncolytic viruses that are able to promote anti-tumor immunity and restrain anti-viral immunity (See e.g., Aurelian, Onco Targets Ther (2016) 9; 2627-2637).

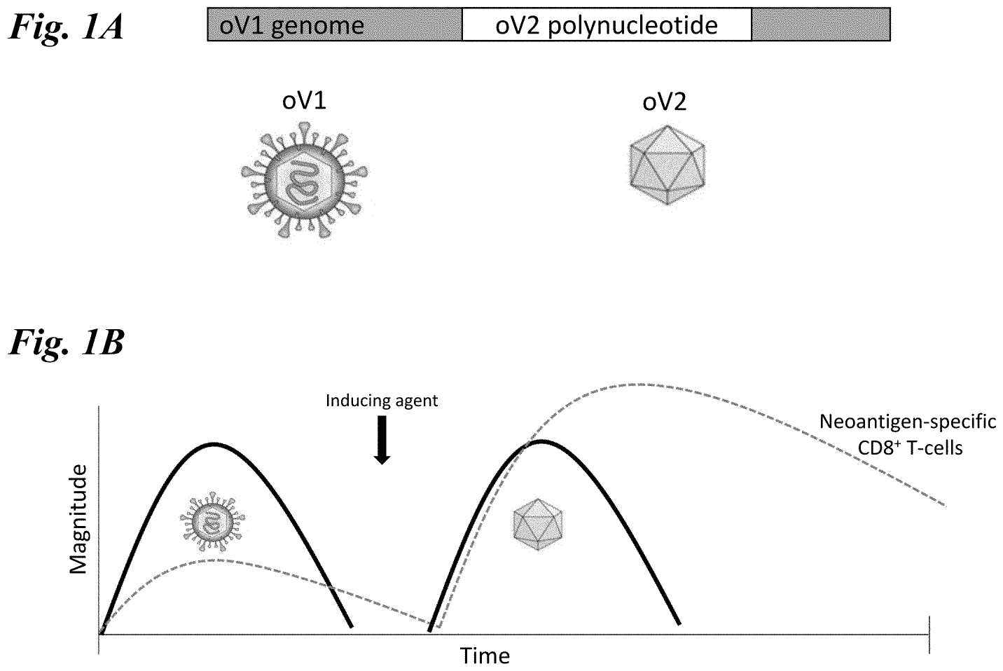

In some embodiments, the present disclosure provides a primary oncolytic virus comprising a polynucleotide encoding a secondary oncolytic virus (i.e., a dual oncolytic virus). The dual oncolytic viruses described herein enable production of two different oncolytic viruses from one viral vector: a primary oncolytic virus and a secondary oncolytic virus. In some embodiments, expression of the primary and/or secondary virus is inducible, allowing for temporal control over expression of the primary and/or secondary viruses. An exemplary illustration of this process is shown in FIG. 1. Briefly, administration of the dual oncolytic virus shown in FIG. 1A results in the initial expression of the primary oncolytic virus (oV1) and viral lysis of tumor cells (FIG. 1B, black line). Tumor cell lysis mediated by oV1 results in the release of tumor neoantigens and the development of a tumor antigen-specific CD8+ T cells, leading to further immune cell-mediated lysis of tumor cells (FIG. 1B, dashed grey line). Transcription of the secondary oncolytic virus (oV2) from the polynucleotide inserted into the genome of oV1 (FIG. 1A) results in the expression of the oV2 and tumor cell lysis mediated by oV2 results in a second release of tumor antigens, providing an antigen boost to the existing population of anti-tumor CD8+ T cells. Transcription and expression of oV2 can optionally be induced by administration of an inducing agent or removal of an inhibitory agent. Because, oV1 and oV2 are different viruses, the anti-viral immune response generated against one will not be effective against the other, thereby mitigating the redirection of the immune response to the viral antigens. Therefore, the dual oncolytic viruses described herein promote viral persistence in the host, enabling increased viral lysis of tumor cells and enhanced development of tumor antigen-specific T cell populations.

Administration

In some embodiments, administration of the dual oncolytic viruses or dual viruses promotes specific immune response against tumor cells or tumor antigens. In some embodiments, administration of the dual oncolytic viruses or dual viruses results in more specific killing of tumor cells in the subject compared to administering the primary oncolytic virus or primary virus alone or administering the secondary oncolytic virus or secondary virus alone. In some embodiments, the infections of the primary oncolytic virus or primary virus and the secondary oncolytic virus or secondary virus to a tumor leads to a focusing of the immune reaction on the common tumor antigens that are released as a result of the infection. In some embodiments, the infections of the primary oncolytic virus or primary virus and the secondary oncolytic virus or secondary virus leads to preferential or specific host immunity against tumor cells or tumor antigens.

In some embodiments, a greater number of tumor cells are killed by administration of the dual oncolytic viruses or dual viruses compared to the number of tumor cells killed by administering the primary oncolytic virus or primary virus alone or the secondary oncolytic virus or secondary virus alone. In some embodiments, at least 10% more, at least 20% more, at least 30% more, at least 50% more, at least 100% more, at least 200% more, or at least 500% more, tumor cells are killed by administration of the dual oncolytic viruses or dual viruses compared to the number of tumor cells killed by administering the same amount/dose of the primary oncolytic virus or primary virus alone or the secondary oncolytic virus or secondary virus alone. In some embodiments, administration of the dual oncolytic viruses or dual viruses leads to greater reduction of tumor size compared to administration of either virus alone (or reduce tumor size in situations where neither virus alone can reduce the tumor size). In some embodiments, administration of the dual oncolytic viruses or dual viruses leads to at least 10%, at least 20%, at least 30%, at least 40%, at least 50%, at least 60%, at least 70%, at least 80%, or at least 90%, additional reduction of tumor size compared to administration of either virus alone.

In some embodiments, administration of the dual oncolytic viruses or dual viruses of the disclosure in a subject results in a stronger immune response against one or more tumor antigens in the subject compared to administering the primary oncolytic virus or primary virus alone or administering the secondary oncolytic virus or secondary virus alone. In some embodiments, the immune response is measured by the number of immune cells (e.g., CD4+ and/or CD8+ T cells) specific for one or more tumor associated antigens. In some embodiments, administration of the dual oncolytic viruses or dual viruses of the disclosure in a subject results in at least 10%, at least 20%, at least 30%, at least 50%, at least 100%, at least 200%, or at least 500% more immune cells (e.g., CD4+ and/or CD8+ T cells) specific for one or more tumor associated antigens, compared to administering the primary oncolytic virus or primary virus alone or administering the secondary oncolytic virus or secondary virus alone. In some embodiments, the immune cells are CD4+ T cells. In some embodiments, the immune cells are CD8+ T cells.

In some embodiments, administration of the dual oncolytic viruses or dual viruses of the disclosure in a subject results in a reduced immune response against the primary oncolytic virus or primary virus in the subject compared to administering the primary oncolytic virus or primary virus alone. In some embodiments, the immune response is measured by the number of immune cells (e.g., CD4+ and/or CD8+ T cells) specific for one or more antigens of the primary oncolytic virus or primary virus. In some embodiments, the immune response is measured by the level of antibodies specific for one or more antigens of the primary oncolytic virus or primary virus. In some embodiments, the immune response is reduced by at least 10%, at least 20%, at least 30%, at least 40%, at least 50%, at least 60%, at least 70%, at least 80%, at least 90%, or at least 95%, compared to administering the primary oncolytic virus or primary virus alone.

In some embodiments, administration of the dual oncolytic viruses or dual viruses of the disclosure in a subject results in a reduced immune response against the secondary oncolytic virus or secondary virus in the subject compared to administering the secondary oncolytic virus or secondary virus alone. In some embodiments, the immune response is measured by the number of immune cells (e.g., CD4+ and/or CD8+ T cells) specific for one or more antigens of the secondary oncolytic virus or secondary virus. In some embodiments, the immune response is measured by the level of antibodies specific for one or more antigens of the secondary oncolytic virus or secondary virus. In some embodiments, the immune response is reduced by at least 10%, at least 20%, at least 30%, at least 40%, at least 50%, at least 60%, at least 70%, at least 80%, at least 90%, or at least 95%, compared to administering the secondary oncolytic virus or secondary virus alone. In some embodiments, administration of the dual oncolytic viruses or dual viruses of the disclosure in a subject does not induce immune response against the secondary oncolytic virus or secondary virus in the subject.

In some embodiments, administration of the dual oncolytic viruses or dual viruses of the disclosure in a subject results in more persistent production of the primary oncolytic virus or primary virus in the subject compared to administering the primary oncolytic virus or primary virus alone. In some embodiments, the persistent production of the primary oncolytic virus or primary virus is measured by the level of the primary oncolytic virus or primary virus in blood circulation or in the tumor site. In some embodiments, administration of the dual oncolytic viruses or dual viruses results in a detectable level of the primary oncolytic virus or primary virus for a longer time in blood circulation or in the tumor site, for example, at least 10% longer, at least 20% longer, at least 30% longer, at least 50% longer, at least 100% longer, at least 200% longer, or at least 500% longer, compared to administering the primary oncolytic virus or primary virus alone.

In some embodiments, administration of the dual oncolytic viruses or dual viruses of the disclosure in a subject results in more persistent production of the secondary oncolytic virus or secondary virus in the subject compared to administering the secondary oncolytic virus or secondary virus alone. In some embodiments, the persistent production of the secondary oncolytic virus or secondary virus is measured by the level of the secondary oncolytic virus or secondary virus in blood circulation or in the tumor site. In some embodiments, administration of the dual oncolytic viruses or dual viruses results in a detectable level of the secondary oncolytic virus or secondary virus for a longer time in blood circulation or in the tumor site, for example, at least 10% longer, at least 20% longer, at least 30% longer, at least 50% longer, at least 100% longer, at least 200% longer, or at least 500% longer, compared to administering the secondary oncolytic virus or secondary virus alone.

In some embodiments, administration of the dual oncolytic viruses or dual viruses of the disclosure in a subject results in an extended period of tumor inhibition in the subject compared to administering the primary oncolytic virus or primary virus alone or the secondary oncolytic virus or secondary virus alone. In some embodiments, the period of tumor inhibition is progression-free period. In some embodiments, the period of tumor inhibition is tumor-free period. In some embodiments, the period of tumor inhibition is the time between initiation of virus administration and cancer remission. In some embodiments, the period of tumor inhibition is metastasis-free period. In some embodiments, the period of tumor inhibition is the time before the tumor grows to its initial size right before the administration of the oncolytic virus or the virus (after a tumor reduction period due to the oncolytic virus treatment or the virus treatment). In some embodiments, administration of the dual oncolytic viruses or dual viruses results in a tumor inhibition period that is at least 10% longer, at least 20% longer, at least 30% longer, at least 50% longer, at least 100% longer, at least 200% longer, at least 500% longer, or at least 1000% longer, compared to administering the primary oncolytic virus or primary virus alone or the secondary oncolytic virus or secondary virus alone.

In some embodiments, the production of the secondary oncolytic virus or secondary virus is regulated by an exogenous agent. In some embodiments, regulation by the exogenous agent offers spatial and/or temporal control of the production of the secondary oncolytic virus or secondary virus. In some embodiments, the exogenous agent is a peptide, a hormone, or a small molecule. In some embodiments, the exogenous agent is a ligand. In some embodiments, the exogenous agent regulates the production of the secondary oncolytic virus or secondary virus through regulating the activity of an promoter, a ribozyme, or RNAi. For example, tetracycline/doxycycline are exemplary exogenous agents for Tet-On or Tet-OFF promoters and/or ribozymes. In some embodiments, the exogenous agent regulates the production of the secondary oncolytic virus or secondary virus through regulating the activity of a recombinase. For example, 4-hydroxytamoxifen is an exemplary exogenous that can regulate the activity/subcellular localization of a recombinase through a modified ligand binding domain of estrogen receptor (ER) fused to the recombinase.

In some embodiments, the exogenous agent is administered systemically. In some embodiments, the exogenous agent is administered locally, for example, intratumorally. In some embodiments, the present disclosure provides a method of administering an exogenous agent to regulates the production of the secondary oncolytic virus or secondary virus. In some embodiments, the presence of the exogenous agent inhibits production of the secondary oncolytic virus or secondary virus. In some embodiments, the presence of the exogenous agent induces production of the secondary oncolytic virus or secondary virus. In some embodiments, no secondary oncolytic virus or secondary virus is detectable in the subject prior to the administration of the exogenous agent. In some embodiments, the exogenous agent is administered at about the same time of or prior to the administration of the dual oncolytic viruses or dual virus. In some embodiments, the exogenous agent is administered after the administration of the dual oncolytic viruses or dual virus. In some embodiments, the exogenous agent is administered at least 1 hour, at least 3 hours, at least 6 hours, at least 12 hours, or at least 24 hours after the administration of the dual oncolytic viruses or dual virus. In some embodiments, the exogenous agent is administered at least 2 days, at least 3 days, at least 4 days, at least 5 days, at least 6 days, at least 1 week, at least 2 weeks, at least 1 month, at least 2 months, at least 3 months, or at least 6 months after administration of the dual oncolytic viruses or dual virus. In some embodiments, the infections of the primary oncolytic virus or primary virus and the secondary virus are temporally separated. In some embodiment, the temporally separated infections of the primary and the secondary oncolytic viruses, or the primary and the secondary viruses, result in focusing of the immune reaction on the tumor cells and/or tumor antigens.

In some embodiments, administration of the dual oncolytic viruses or dual viruses of the disclosure enables viral infection of more cell types compared to administering the primary oncolytic virus or primary virus alone or the secondary oncolytic virus or secondary virus alone. In some embodiments, at least one cell type infected by the dual oncolytic viruses or dual viruses is resistant to the primary oncolytic virus or primary virus alone or the secondary oncolytic virus or secondary virus alone. In some embodiments, at least one cell type infected by the dual oncolytic viruses or dual viruses is resistant to the primary oncolytic virus or primary virus alone. In some embodiments, the cell type resistant to the primary oncolytic virus or primary virus alone or the secondary oncolytic virus or secondary virus alone is a myeloid cells, a macrophage, or a fibroblast cells. In some embodiments, the cell type resistant to the primary oncolytic virus or primary virus alone or the secondary oncolytic virus or secondary virus alone contributes to immune inhibition. In some embodiments, the cell type resistant to the primary oncolytic virus or primary virus alone or the secondary oncolytic virus or secondary virus alone contributes to tumor inhibition.

In some embodiments, “the primary oncolytic virus alone” of the disclosure refers to a reference primary oncolytic virus that does not comprise a polynucleotide encoding a secondary oncolytic virus (i.e., not a dual oncolytic virus). In some embodiments, “the primary virus alone” of the disclosure refers to a reference primary virus that does not comprise a polynucleotide encoding a secondary virus (i.e., not a dual virus).

### Modified Dual Viruses and Modified Dual Oncolytic Viruses

In some embodiments, the disclosure provides viruses (e.g., primary viruses and/or secondary viruses) that are pseudotyped or otherwise engineered. In some embodiments, the viruses are primary oncolytic viruses and/or secondary oncolytic viruses that are pseudotyped or otherwise engineered.

In some embodiments of the disclosure, “Pseudotyped viruses” refer to viruses in which one or more of the viral coat proteins (e.g., envelope proteins) have been replaced or modified. In some embodiments, a pseudotyped virus is capable of infecting a cell or tissue type that the corresponding non-pseudotyped virus is not capable of infecting. In some embodiments, a pseudotyped virus is capable of preferentially infecting a cell or tissue type compared to a non-pseudotyped virus. In some embodiments, a portion of the virus particle (e.g., the envelope or capsid) of the pseudotyped virus comprises heterologous proteins, such as viral proteins derived from a heterologous virus or non-viral proteins. Non-viral proteins may include antibodies and antigen-binding fragments thereof. In some embodiments, a pseudotyped virus is capable of i) altered tropism relative to non-pseudotyped virus, and/or ii) reduction or elimination of a non-beneficial effect. In some embodiments a pseudotyped virus demonstrates reduced toxicity or reduced infection of non-tumor cells or non-tumor tissue as compared to a non-pseudotyped virus.

In general, viruses have natural host cell populations that they infect most efficiently. For example, retroviruses have limited natural host cell ranges, while adenoviruses and adeno-associated viruses are able to efficiently infect a relatively broader range of host cells, although some cell types are refractory to infection by these viruses. The proteins on the surface of a virus (e.g., envelope proteins or capsid proteins) meditate attachment to and entry into a susceptible host cell and thereby determine the tropism of the virus, i.e., the ability of a particular virus to infect a particular cell or tissue type. In some embodiments, the viruses of the disclosure comprise a single types of protein on the surface of the virus. For example, retroviruses and adeno-associated viruses have a single protein coating their membrane. In some embodiments, the viruses of the disclosure comprise more than one type of protein on the surface of the virus. For example, adenoviruses are coated with both an envelope protein and fibers that extend away from the surface of the virus.

In some embodiments, the proteins on the surface of the virus can bind to cell-surface molecules such as heparin sulfate, thereby localizing the virus to the surface of the potential host cell. The proteins on the surface of the virus can also mediate interactions between the virus and specific protein receptors expressed on a host cell that induce structural changes in the viral protein in order to mediate viral entry. In some embodiments, interactions between the proteins on the surface of the virus and cell receptors can facilitate viral internalization into endosomes, wherein acidification of the endosomal lumen induces refolding of the viral coat. In some embodiments, viral entry into potential host cells requires a favorable interaction between at least one molecule on the surface of the virus and at least one molecule on the surface of the cell.

In some embodiments, the viruses of the disclosure comprise a viral coat (e.g., a viral envelope or viral capsid), wherein the proteins present on the surface of the viral coat (e.g., viral envelope proteins or viral capsid proteins) modulate recognition of a potential target cell for viral entry. In some embodiments, this process of determining a potential target cell for entry by a virus is referred to as host tropism. In some embodiments, the host tropism is cellular tropism, wherein viral recognition of a receptor occurs at a cellular level, or tissue tropism, wherein viral recognition of cellular receptors occurs at a tissue level. In some embodiments, the viral coat of a virus recognizes receptors present on a single type of cell. In some embodiments, the viral coat of a virus recognizes receptors present on multiple cell types (e.g., 2, 3, 4, 5, 6 or more different cell types). In some embodiments, the viral coat of a virus recognizes cellular receptors present on a single type of tissue. In some embodiments, the viral coat of a virus recognizes cellular receptors present on multiple tissue types (e.g., 2, 3, 4, 5, 6 or more different tissue types).

In some embodiments, the pseudotyped viruses of the disclosure comprise a viral coat that has been modified to incorporate surface proteins from a different virus in order to facilitate viral entry to a particular cell or tissue type. In some embodiments, a pseudotyped viruses comprises a viral coat wherein the viral coat of a first virus is exchanged with a viral coat of second, wherein the viral coat of the secondary virus allows the pseudotyped virus to infect a particular cell or tissue type. In some embodiments, the viral coat comprises a viral envelope. In some embodiments, the viral envelope comprises a phospholipid bilayer and proteins such as proteins obtained from a host membrane. In some embodiments, the viral envelope further comprises glycoproteins for recognition and attachment to a receptor expressed by a host cell. In some embodiments, the viral coat comprises a capsid. In some instances, the capsid is assembled from oligomeric protein subunits termed protomers. In some embodiments, the capsid is assembled from one type of protomer or protein, or is assembled from two, three, four, or more types of protomers or proteins.

In some embodiments, it is advantageous to limit or expand the range of cells susceptible to transduction by an virus of the disclosure for the purpose of therapy (e.g., cancer therapy). To this end, many viruses have been developed in which the endogenous viral coat proteins (e.g., viral envelope or capsid proteins) proteins have been replaced by viral coat proteins from other viruses or by chimeric proteins. In some embodiments, the chimeric proteins are comprised of parts of a viral protein necessary for incorporation into the virion, as well proteins or nucleic acids designed to interact with specific host cell proteins, such as a targeting moiety.

In some embodiments, the pseudotyped viruses of the disclosure are pseudotyped in order to limit or control the viral tropism (i.e., to reduce the number of cell or tissue types that the pseudotyped virus is capable of infecting). Most strategies adopted to limit tropism have used chimeric viral coat proteins (e.g., envelope proteins) linked antibody fragments. These viruses show great promise for the development of therapies (e.g., cancer therapies). In some embodiments, the pseudotyped viruses of the disclosure are pseudotyped in order to expand the viral tropism (i.e., to increase the number of cell or tissue types that the pseudotyped virus is capable of infecting). One mechanism for expanding the cellular tropism of viruses (e.g., enveloped viruses) is through the formation of phenotypically mixed particles or pseudotypes, a process that commonly occurs during viral assembly in cells infected with two or more viruses. For example, human immunodeficiency virus type 1 (HIV-1). HIV1 infects cells that express CCR4 with an appropriate co-receptor. However, HIV1 forms pseudotypes by the incorporation of heterologous glycoproteins (GPs) through phenotypic mixing, such that the virus can infect cells that do not express the CD4 receptor and/or an appropriate co-receptor, thereby expanding the tropism of the virus. Several studies have demonstrated that wild type HIV-1 produced in cells infected with xenotropic murine leukemia virus (MLV), amphotropic MLV, or herpes simplex virus gives rise to phenotypically mixed virions with an expanded host range, indicating that pseudotyped virions had been produced. Phenotypic mixing of viral GPs has also been shown to occur between HIV-1 and VSV in coinfected cell cultures. These early observations were key to the subsequent design of HIV-1-based lentiviral vectors bearing heterologous GPs.

There is an ever-growing list of alternative GPs for pseudotyping lentiviruses, each with specific advantages and disadvantages. The widespread use of VSV G-proteins (VSV-G) to pseudotype lentiviruses has made this GP in effect the standard against which the usefulness of other viral GPs to form pseudotypes are compared. Additional non-limiting examples of lentivirus pseudotypes include pseudotypes bearing lyssavirus-derived GPs, pseudotyped lentiviruses bearing lymphocytic choriomeningitis virus GPs, lentivirus pseudotypes bearing alphavirus GPs (e.g., lentiviral vectors pseudotyped with the RRV and SFV GPs, lentiviral vectors pseudotyped with sindbis virus GPs), pseudotypes bearing filovirus GPs, and lentiviral vector pseudotypes containing the baculovirus GP64.

In some embodiments, the engineered (e.g., pseudotyped) viruses are capable of binding to a tumor and/or tumor cell, typically by binding to a protein, lipid, or carbohydrate expressed on a tumor cell. In such embodiments, the engineered viruses described herein may comprise a targeting moiety that directs the virus to a particular host cell. In some instances, any cell surface biological material known in the art or yet to be identified that is differentially expressed or otherwise present on a particular cell or tissue type (e.g., a tumor or tumor cell, or tumor associated stroma or stromal cell) may be used as a potential target for the viruses of the disclosure. In some embodiments, the cell surface material is a protein. In some embodiments, the targeting moiety binds cell surface antigens indicative of a disease, such as a cancer (e.g., breast, lung, ovarian, prostate, colon, lymphoma, leukemia, melanoma, and others); an autoimmune disease (e.g. myasthenia gravis, multiple sclerosis, systemic lupus erythmatosis, rheumatoid arthritis, diabetes mellitus, and others); an infectious disease, including infection by HIV, HCV, HBV, CMV, and HPV; and a genetic disease including sickle cell anemia, cystic fibrosis, Tay-Sachs, J3-thalassemia, neurofibromatosis, polycystic kidney disease, hemophilia, etc. In certain embodiments, the targeting moiety targets a cell surface antigen specific to a particular cell or tissue type, e.g., cell-surface antigens present in neural, lung, kidney, muscle, vascular, thyroid, ocular, breast, ovarian, testis, or prostate tissue.

In some embodiments, the virus of the disclosure (primary virus and/or secondary virus) is a chimeric virus (e.g., encode a virus comprising one portion, such as a capsid protein or an IRES, derived from a first virus and another portion, such as a non-structural gene such as a protease or polymerase, derived from a second virus). In some embodiments, the virus is a primary oncolytic virus and/or a secondary oncolytic virus.

### Design of Dual Oncolytic Viruses, Dual Viruses and Control Elements

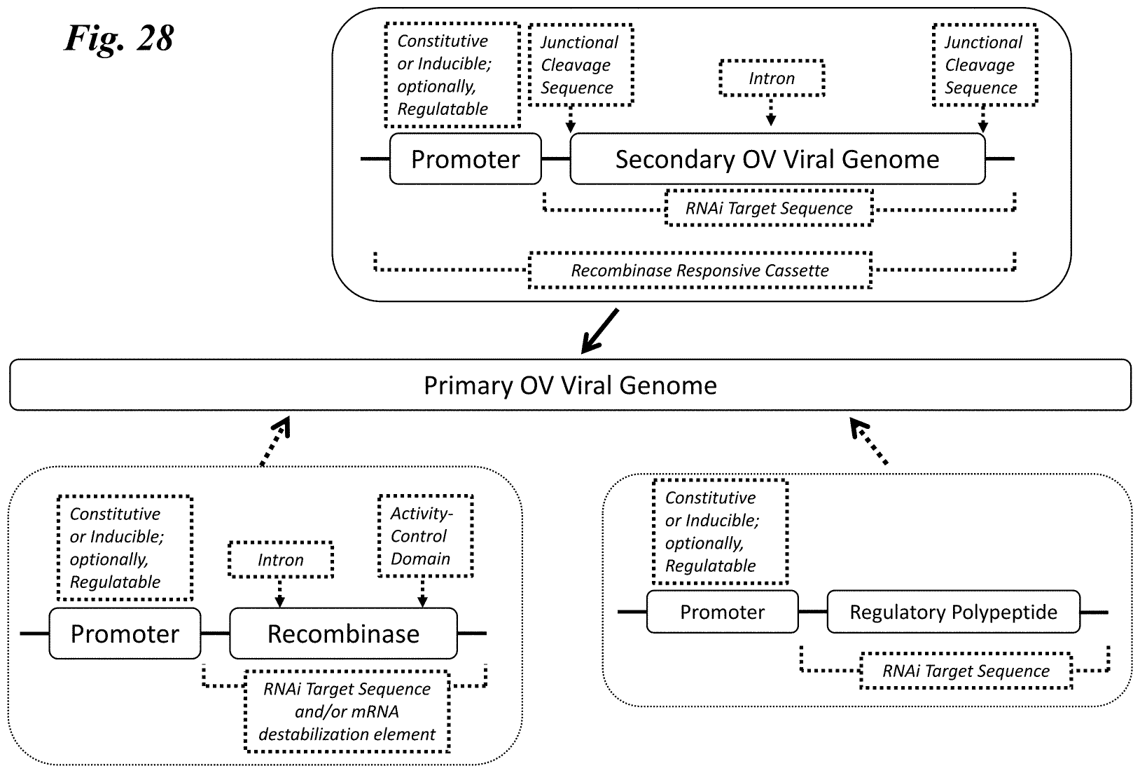

The present disclosure provides a primary oncolytic virus comprising a polynucleotide encoding a secondary oncolytic virus, optionally a polynucleotide encoding a recombinase, and optionally a polynucleotide encoding a regulatory polypeptide, as shown in FIG. 28. In some embodiments, the regulatory polypeptide is capable of binding to a regulatable promoter. In some embodiments, the regulatory polypeptide regulates the function of one or more regulatable promoters shown in FIG. 28. In some embodiments, the regulatory polypeptide is a rtTA protein. In some embodiments, the regulatory polypeptide is a tTA protein.

The present disclosure provides a primary virus comprising a polynucleotide encoding a secondary virus, optionally a polynucleotide encoding a recombinase, and optionally a polynucleotide encoding a regulatory polypeptide, as shown in FIG. 28. In some embodiments, the regulatory polypeptide is capable of binding to a regulatable promoter. In some embodiments, the regulatory polypeptide regulates the function of one or more regulatable promoters shown in FIG. 28. In some embodiments, the regulatory polypeptide is a rtTA protein. In some embodiments, the regulatory polypeptide is a tTA protein.

In some embodiments, the present disclosure provides a primary oncolytic virus comprising a polynucleotide encoding a secondary oncolytic virus, wherein the expression of the secondary oncolytic virus is controlled by a regulatable promoter. In some embodiments, the polynucleotide encoding the secondary oncolytic virus is operably linked to a regulatable promoter. In some embodiments, the regulatable promoter is selected from a steroid-inducible promoter, a metallothionine promoter, an MX-1 promoter, a GENESWITCH™ hybrid promoter, a cumate-responsive promoter, a hormone-responsive promoter (e.g. ponasterone A-inducible promoter), and a tetracycline (Tet)-regulated promoter. In some embodiments, the regulatable promoter is a recombinase recognition site (RRS)-flanked promoter.

In some embodiments, the present disclosure provides a primary virus comprising a polynucleotide encoding a secondary virus, wherein the expression of the secondary virus is controlled by a regulatable promoter. In some embodiments, the polynucleotide encoding the secondary virus is operably linked to a regulatable promoter. In some embodiments, the regulatable promoter is selected from a steroid-inducible promoter, a metallothionine promoter, an MX-1 promoter, a GENESWITCH™ hybrid promoter, a cumate-responsive promoter, a hormone-responsive promoter (e.g. ponasterone A-inducible promoter), and a tetracycline (Tet)-regulated promoter. In some embodiments, the regulatable promoter is a recombinase recognition site (RRS)-flanked promoter.

In some embodiments, the regulatable promoter is a Tet-regulated promoter. Tet-regulated promoters are developed by placing a Tet response element (TRE) upstream of a minimal promoter. A TRE is 7 repeats of a 19 nucleotide tetracycline operator (tetO) sequence, and is recognized by the tetracycline repressor (tetR). In the endogenous bacterial system, if tetracycline, or an analog like doxycycline, is present, the tetR will bind to tetracycline and not to the TRE, thereby permitting transcription. To use Tet as a regulator of gene expression, a tetracycline-controlled transactivator (tTA) was created by fusing tetR with the transcriptional activation domain of virion protein 16 (VP16) (Gossen and Bujard, PNAS (1992) 15:89(12): 5547-5551). In the absence of tetracycline, the tetR portion of the tTA will bind the tetO sequences in the TRE and the VP16 activation domain will promote transcription of the downstream genes. In the presence of tetracycline, tetracycline binds to the tetR domain of tTA, which precludes tTA binding to the tetO sequences and VP16-mediated activation of downstream gene expression. Therefore, in some embodiments, the regulatable promoter is a Tet-regulated promoter wherein transcription of the polynucleotide encoding a secondary oncolytic virus or a secondary virus is active in the presence of a tTA protein and the absence of Tet (or the doxycycline derivative thereof). Such promoters are referred to herein as Tet-OFF promoters, as they are active in the absence of tetracycline. In some embodiments, the tTA polypeptide comprises or consists of an amino acid sequence having at least 70%, at least 75%, at least 80%, at least 85%, at least 90%, at least 95%, or 100% identity to the amino acid sequence encoded by SEQ ID NO: 853.

In some embodiments, the regulatable promoter is a Tet-regulated promoter wherein transcription of the polynucleotide encoding the secondary oncolytic virus or the secondary virus is active in the presence of Tet (or the doxycycline derivative thereof) and a reverse tetracycline-controlled transactivator (rtTA). The rtTA is a fusion protein comprising the VP16 transcriptional activation domain and a tetR domain that has been mutated such that the tetR domain relies on the presence of Tet for binding to the tetO sequences in the promoter. Therefore, transcription of downstream genes is not active absence of tetracycline. However, in the presence of tetracycline, the mutant tetR portion of the rtTA protein will bind to the tetO sequences allowing VP16-mediated transcriptional activation and expression of downstream genes. Such promoters are referred to herein as Tet-ON promoters, as they are active in the presence of tetracycline. In some embodiments, the rtTa protein comprises or consists of an amino acid sequence at least 70%, at least 75%, at least 80%, at least 85%, at least 90%, at least 95%, or 100% identity to the amino acid sequence encoded by SEQ ID NO: 852.

In some embodiments, the primary oncolytic virus or the primary virus comprises a first polynucleotide encoding the secondary oncolytic virus or the secondary virus operably linked to a regulatable promoter, and a second polynucleotide encoding a protein capable of binding to the regulatable promoter. In some embodiments, the regulatable promoter is a Tet-ON promoter and the protein capable of binding to the regulatable promoter is an rtTA protein. In some embodiments, the regulatable promoter is a Tet-OFF promoter and the protein capable of binding to the regulatable promoter is a tTA protein. In some embodiments, the polynucleotide encoding the protein capable of binding to the regulatable promoter is operably linked to a constitutive promoter. Constitutive promoters are known in the art and include, but are not limited to, a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

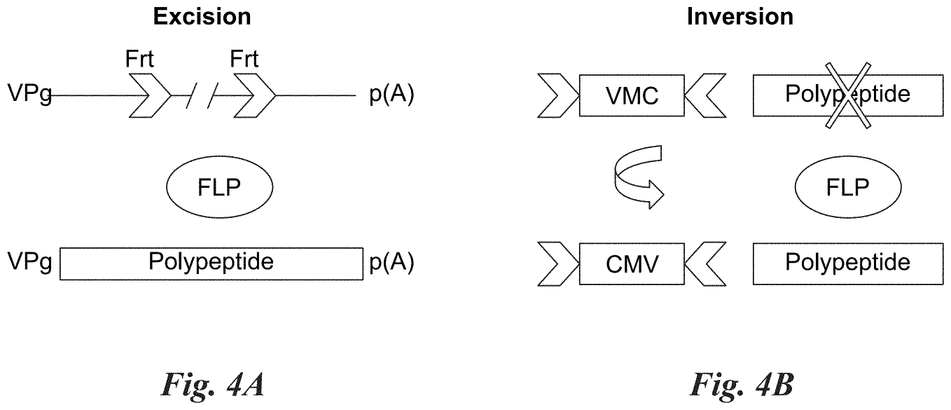

In some embodiments, the regulatable promoter is a recombinase recognition site (RRS)-flanked promoter. An RRS-flanked promoter is generated by flanking a constitutive promoter with recombinase recognition sites. In such embodiments, the primary oncolytic virus or the primary virus comprises a first polynucleotide encoding the secondary oncolytic virus or the secondary virus operably linked to an RRS-flanked promoter, and a second polynucleotide encoding a recombinase protein capable of mediating recombination between the recombinase recognition sites. In some embodiments, expression of the recombinase protein permits transcription of the polynucleotide encoding the secondary oncolytic virus or the secondary virus. For example, in some embodiments, the RRS-flanked promoter comprises an inverted promoter sequence (See e.g., FIG. 4B). In the absence of recombinase expression, the promoter sequence remains inverted and transcription of the polynucleotide encoding the secondary oncolytic virus or the secondary virus does not occur. When the recombinase is expressed, the inverted promoter sequence is flipped, allowing transcription of the polynucleotide encoding the secondary oncolytic virus or the secondary virus.

In some embodiments, the polynucleotide sequence encoding the secondary oncolytic virus or the secondary virus and the polynucleotide sequence encoding the protein capable of binding to the regulatable promoter are comprised in the same polynucleotide. For example, in some embodiments, the polynucleotide sequence encoding the secondary oncolytic virus or the secondary virus and the polynucleotide sequence encoding the protein capable of binding to the regulatable promoter are under the control of a bi-directional promoter. In some embodiments, the polynucleotide sequence encoding the secondary oncolytic virus or the secondary virus and the polynucleotide sequence encoding the protein capable of binding to the regulatable promoter are comprised in the different polynucleotides inserted at different locations in the genome of the primary virus.

In some embodiments, the present disclosure provides a primary oncolytic virus or a primary virus comprising a polynucleotide encoding a secondary oncolytic virus or a secondary virus, wherein expression of the secondary oncolytic virus or the secondary virus is regulated by one or more post-transcriptional control elements. Herein, a “post-transcriptional control element” refers to any element other than a promoter that is capable of modulating the abundance of the secondary oncolytic virus or the secondary virus mRNA transcript. Post-transcriptional control elements control mRNA transcript abundance through a variety of post-transcriptional mechanisms and can be constitutive or inducible elements. Examples of post-transcriptional control elements include ribozymes, aptazymes, target sites for RNAi molecules (e.g., shRNA target sites, microRNA target sites, artificial microRNA (AmiRNA) target sites), and RSS-flanked frame-shift or stop codon insertions.

In some embodiments, the post-transcriptional control element is a ribozyme-encoding sequence that mediates self-cleavage of the mRNA transcript. Exemplary ribozymes include the Hammerhead ribozyme, the Varkud satellite (VS) ribozyme, the hairpin ribozyme, the GIR1 branching ribozyme, the glmS ribozyme, the twister ribozyme, the twister sister ribozyme, the pistol ribozyme, the hatchet ribozyme, and the Hepatitis delta virus ribozyme. In such embodiments, the primary oncolytic virus or the primary virus comprises a first polynucleotide encoding the secondary oncolytic virus or the secondary virus, wherein the genome of the secondary oncolytic virus or the secondary virus comprises one or more internal ribozyme sequences, such that the viral transcript is cleaved internally and thereby preventing expression of the secondary oncolytic virus or the secondary virus.

In some embodiments, the post-transcriptional control element is an aptazyme-encoding sequence. An “aptazyme” is a ribozyme sequences that contain an integrated aptamer domain specific for a ligand to generate a ligand-inducible self-cleaving ribozyme. Ligand binding to the aptamer domain triggers activation of the enzymatic activity of the ribozyme, thereby resulting in cleavage of the RNA transcript. Exemplary aptazymes include theophylline-dependent aptazymes (e.g., hammerhead ribozyme linked to a theophylline-dependent aptamer, described in Auslander et al., Mol BioSyst. (2010) 6, 807-814), tetracycline-dependent aptazymes (e.g., hammerhead ribozyme linked to a Tet-dependent aptamer, described by Zhong et al., eLife 2016; 5:e18858 DOI: 10.7554/eLife.18858; Win and Smolke, PNAS (2007) 104; 14283-14288; Whittmann and Suess, Mol Biosyt (2011) 7; 2419-2427; Xiao et al., Chem & Biol (2008) 15; 125-1137; and Beilstein et al., ACS Syn Biol (2015) 4; 526-534), guanine-dependent aptazymes (e.g., hammerhead ribozyme linked to a guanine-dependent aptamer, described by Nomura et al., Chem Commun., (2012) 48(57); 7215-7217). In such embodiments, the primary oncolytic virus or the primary virus comprises a first polynucleotide encoding the secondary oncolytic virus or the secondary virus, wherein the genome of the secondary oncolytic virus or the secondary virus comprises one or more internal aptazyme sequences, such that the viral transcript is cleaved internally and thereby preventing expression of the secondary oncolytic virus or the secondary virus. In some embodiments, the ribozyme/aptazyme of the disclosure is a TetOff ribozyme/aptazyme. In some embodiments, the TetOff aptazyme comprises or consists of a nucleic acid sequence having at least 70%, at least 75%, at least 80%, at least 85%, at least 90%, at least 95%, or 100% identity to SEQ ID NO: 913. In some embodiments, the TetOff aptazyme comprises or consists of a nucleic acid sequence having at least 70%, at least 75%, at least 80%, at least 85%, at least 90%, at least 95%, or 100% identity to SEQ ID NO: 914. In some embodiments, the ribozyme/aptazyme is located in the 3′ UTR region.

In some embodiments, the post-transcriptional control element is an RNAi target sequence. In such embodiments, the primary oncolytic virus or the primary virus comprises a first polynucleotide encoding the secondary oncolytic virus or the secondary virus, wherein the secondary oncolytic virus or the secondary virus comprises one or more RNAi target sites. “RNA interference molecule” or “RNAi molecule” as used herein refers to an RNA polynucleotide that mediates degradation of a target mRNA sequence through endogenous gene silencing pathways (e.g., Dicer and RNA-induced silencing complex (RISC)). Exemplary RNA interference agents include micro RNAs (miRNAs), artificial microRNA (AmiRNAs), short hair-pin RNAs (shRNAs), and small interfering RNAs (siRNAs).

In some embodiments, the post-transcriptional control element is an miRNA target sequence. An miRNA refers to a naturally-occurring, small non-coding RNA molecule of about 18-25 nucleotides in length that is at least partially complementary to a target mRNA sequence. In animals, genes for miRNAs are transcribed to a primary miRNA (pri-miRNA), which is double stranded and forms a stem-loop structure. Pri-miRNAs are then cleaved in the nucleus by a microprocessor complex comprising the class 2 RNase III, Drosha, and the microprocessor subunit, DCGR8, to form a 70-100 nucleotide precursor miRNA (pre-miRNA). The pre-miRNA forms a hairpin structure and is transported to the cytoplasm where it is processed by the RNase III enzyme, Dicer, into an miRNA duplex of ˜18-25 nucleotides. Although either strand of the duplex may potentially act as a functional miRNA, typically one strand of the miRNA is degraded and only one strand is loaded onto the Argonaute (AGO) nuclease to produce the effector RNA-induced silencing complex (RISC) in which the miRNA and its mRNA target interact (Wahid et al., 1803:11, 2010, 1231-1243).

In some embodiments, the post-transcriptional control element is an siRNA target sequence. siRNAs refer to double stranded RNA molecules typically about 21-23 nucleotides in length. The duplex siRNA molecule is processed in the cytoplasm by the associates with a multi protein complex called the RNA-induced silencing complex (RISC), during which the “passenger” sense strand is enzymatically cleaved from the duplex. The antisense “guide” strand contained in the activated RISC then guides the RISC to the corresponding mRNA by virtue of sequence complementarity and the AGO nuclease cuts the target mRNA, resulting in specific gene silencing. In some embodiments, the siRNA molecule is derived from an shRNA molecule. shRNAs are single stranded artificial RNA molecules 50-70 nucleotides in length that form stem-loop structures. In some embodiments, the shRNAs mimic a pre-miRNA and can bypass Drosha processing and be directly exported for processing by Dicer. In some embodiments, the shRNA is a miRNA-based shRNA. Expression of miRNA-based shRNAs in cells is accomplished by introducing an DNA polynucleotide encoding the miRNA-based shRNA by plasmid or viral vector. The miRNA-based shRNA is then transcribed into a product that mimics the stem-loop structure of a pri-miRNA, and is similarly processed in the nucleus by Drosha to form a single stranded RNA with a hair-pin loop structure. After export of the hair-pin RNA to the cytoplasm, the hair-pin is processed by Dicer to form a duplex siRNA molecule which is then further processed by the RISC to mediate target-gene silencing.

In some embodiments, the post-transcriptional control element is an artificial microRNA (AmiRNA).

In some embodiments, the primary oncolytic virus or the primary virus comprises a first polynucleotide encoding the secondary oncolytic virus or the secondary virus, wherein the secondary oncolytic virus or the secondary virus comprises one or more RNAi target sites, and a second polynucleotide encoding one or more RNAi molecules that bind to the RNAi target sites. In such embodiments, the one or more RNAi molecules bind to the target sequence in the genome of the secondary oncolytic virus or the secondary virus such that expression of the one or more RNAi molecules results in degradation of the secondary oncolytic virus or the secondary virus mRNA transcript, thereby preventing expression of the secondary oncolytic virus or the secondary virus. In some embodiments, the polynucleotide encoding the one or more RNAi molecules is operably linked to a regulatable promoter. In such embodiments, regulated expression of the one or more RNAi molecules can be used to prevent aberrant expression of the secondary oncolytic virus or the secondary virus.

For example, in some embodiments, the first polynucleotide encoding the secondary oncolytic virus or the secondary virus is operably linked to a first regulatable promoter and the second polynucleotide encoding one or more RNAi molecules is operably linked to a second regulatable promoter. In some embodiments, the first regulatable promoter is a Tet-ON promoter (e.g., SEQ ID NO: 844) and the second regulatable promoter is a Tet-OFF promoter (e.g., SEQ ID NO: 845). In such embodiments, the expression of the secondary oncolytic virus or the secondary virus is activated in the presence of Tet, enabling the expression of the secondary oncolytic virus or the secondary virus to be triggered at the desired time. Prior to the administration of Tet (i.e., in the absence of Tet), the RNAi molecules are expressed. Therefore, any RNA transcripts of the secondary oncolytic virus or the secondary virus produced in the absence of Tet will be targeted by the RNAi molecules, thereby preventing aberrant expression of the secondary oncolytic virus or the secondary virus. In some embodiments, the first regulatable promoter is a Tet-OFF promoter and the second regulatable promoter is a Tet-ON promoter. In such embodiments, the primary oncolytic virus or the primary virus can be administered in combination with Tet, such that expression of the secondary oncolytic virus or the secondary virus will be triggered after the Tet has been removed by degradation. While Tet remains present, the RNAi molecules are expressed and target RNA transcripts of the secondary oncolytic virus or the secondary virus produced in the presence of Tet for degradation, thereby preventing aberrant expression of the secondary oncolytic virus or the secondary virus.

In some embodiments, the primary oncolytic virus or the primary virus comprises a first polynucleotide encoding the secondary oncolytic virus or the secondary virus operably linked to a first regulatable promoter; a second polynucleotide encoding one or more RNAi molecules operably linked to a second regulatable promoter; and a third polynucleotide encoding a first protein capable of binding to the first regulatable promoter and/or a second protein capable of binding to the second regulatable promoter. In some embodiments, the first regulatable promoter is a Tet-On promoter and the first protein is rtTA. In some embodiments, the second regulatable promoter is a Tet-On promoter and the second protein is rtTA. In some embodiments, the first regulatable promoter is a Tet-OFF promoter and the first protein is tTA. In some embodiments, the second regulatable promoter is a Tet-OFF promoter and the second protein is tTA. In some embodiments, the first regulatable promoter is a Tet-On promoter and the first protein is rtTA and the second regulatable promoter is a Tet-OFF promoter and the second protein is tTA. In some embodiments, the first regulatable promoter is a Tet-OFF promoter and the first protein is tTA and the second regulatable promoter is a Tet-ON promoter and the second protein is rtTA.

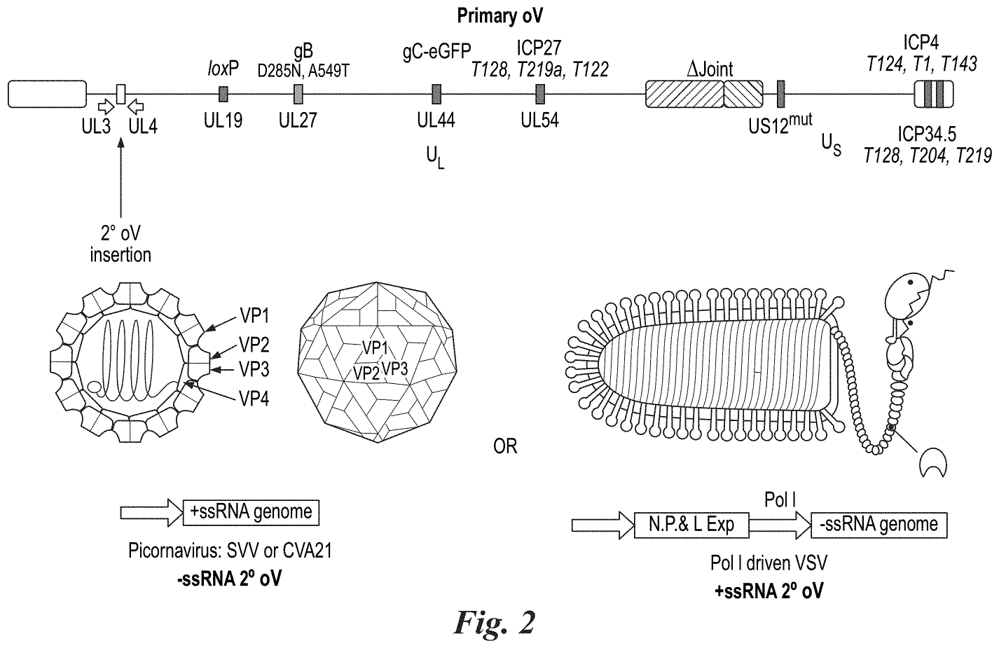

A non-limiting example of a dual oncolytic viral construct is illustrated in FIG. 2. Here, the primary virus is a recombinant HSV and the secondary virus is a positive-sense single-stranded RNA virus or a negative-sense single-stranded RNA virus. In some embodiments, the secondary virus is a positive-sense single-stranded RNA virus selected from SVV and CVA21. In some embodiments, the secondary virus is VSV (a negative-sense RNA virus). The viral genome of the secondary virus is inserted into the polynucleotide encoding the primary HSV virus. In some embodiments, the insertion site is the intergenic region between UL37 and UL38 of HSV. In some embodiments, the expression of the secondary viral genomes are regulated by a tetracycline-responsive Pol II promoter (black arrows) and a RNA polymerase I promoter (grey arrow).

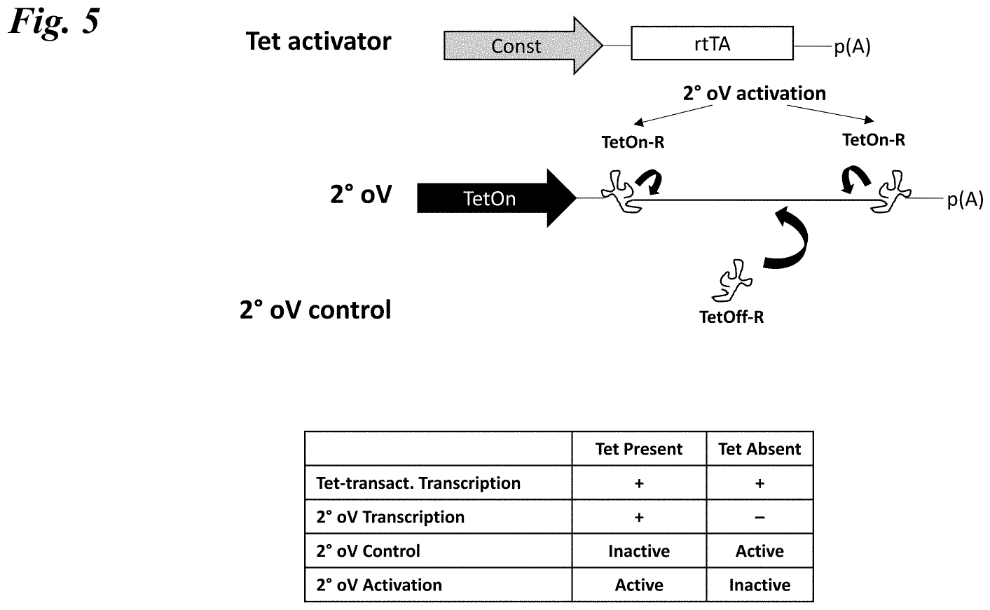

FIGS. 5-7 provide non-limiting examples of controlling the expression, activation and degradation of the viral genome of the secondary oncolytic virus or the secondary virus, using a combination of transcriptional control elements (such as regulatable promoters) and post-transcriptional control element (such as ribozymes and/or RNAi mechanisms). In some embodiments, the transcription of the mRNA encoding the secondary oncolytic viral genome or the secondary viral genome is operably linked to a regulatable promoter (e.g., TetOn promoter). In some embodiments, the polynucleotide encoding the secondary oncolytic viral genome or the secondary viral genome is flanked by TetOn-Ribozyme (TetOn-R) and/or an RNAi target sequence (e.g., AmiRNA) which may remove non-viral RNA from the viral genome transcript. In some embodiments, degradation of the viral genome transcript is controlled by additional regulatory mechanisms (e.g., internal TetOff ribozymes, RNAi molecules) which are also under regulatory control, optionally by the same regulatory mechanism that controls the regulatable promoter.

FIG. 5 illustrates exemplary schematics of components that can be inserted into the primary oncolytic viral genome or the primary viral genome to produce an exemplary nested oncolytic viral construct or nested viral construct. In this non-limiting example, expression of the rtTA peptide is under the control of a constitutively active promoter and expression of the secondary viral genome is under the control of a tetracycline-responsive (TetOn) Pol II promoter, such that transcription of the viral genome occurs in the presence of Tet and the rtTA peptide. Expression of the secondary oncolytic virus or the secondary virus transcript is further regulated by an internal TetOff-ribozyme (TetOff-R), such that the transcript is degraded in the absence of Tet. Further, the secondary viral transcript is activated by 5′ and 3′ TetOn-ribozymes (TetOn-R), such that the mRNA transcript is processed on the 5′ and 3′ ends in the presence of Tet, which creates a RNA transcript of viral genome without flanking extra nucleotides. Therefore, in this example, the presence of Tet turns on the transcription and activation of the secondary oncolytic viral genome or the secondary viral genome and at the same time prevents its degradation.

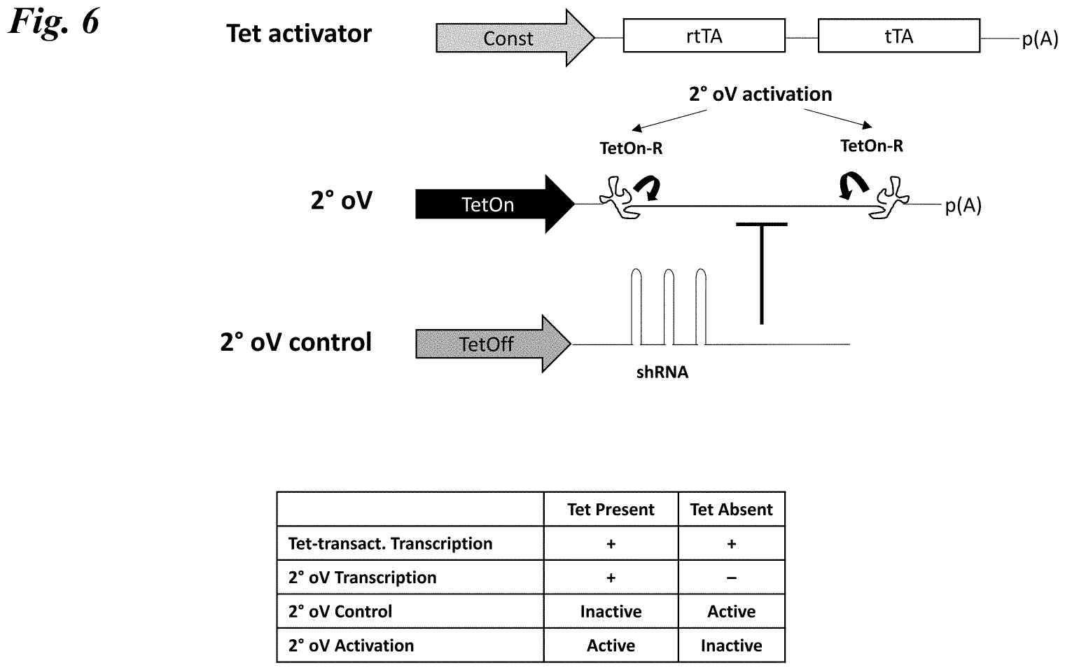

FIG. 6 illustrates exemplary schematics of components that can be inserted into the primary oncolytic viral genome or the primary viral genome to produce an exemplary nested oncolytic viral construct or nested viral construct. Expression of the rtTA and the tetracycline transactivator (tTA) peptide are under the control of a constitutively active promoter. Expression of the secondary viral genome is under the control of a TetOn Pol II promoter, such that transcription of the viral genome occurs in the presence of Tet and the rtTA peptide. Expression of the secondary oncolytic virus or the secondary virus transcript is further regulated by shRNAs specific for target sequences in the mRNA transcript of the secondary virus. Expression of the shRNAs is under the control of a TetOff promoter, such that transcription of the shRNAs occurs in the absence of Tet and the presence of the tTA peptide. Further, the secondary viral transcript is activated by 5′ and 3′ TetOn-R, such that the mRNA transcript is processed on the 5′ and 3′ ends in the presence of Tet, which creates a RNA transcript of viral genome without flanking extra nucleotides. Therefore, in this example, the presence of Tet turns on the transcription and activation of the secondary oncolytic viral genome or the secondary viral genome and at the same time prevents its degradation.

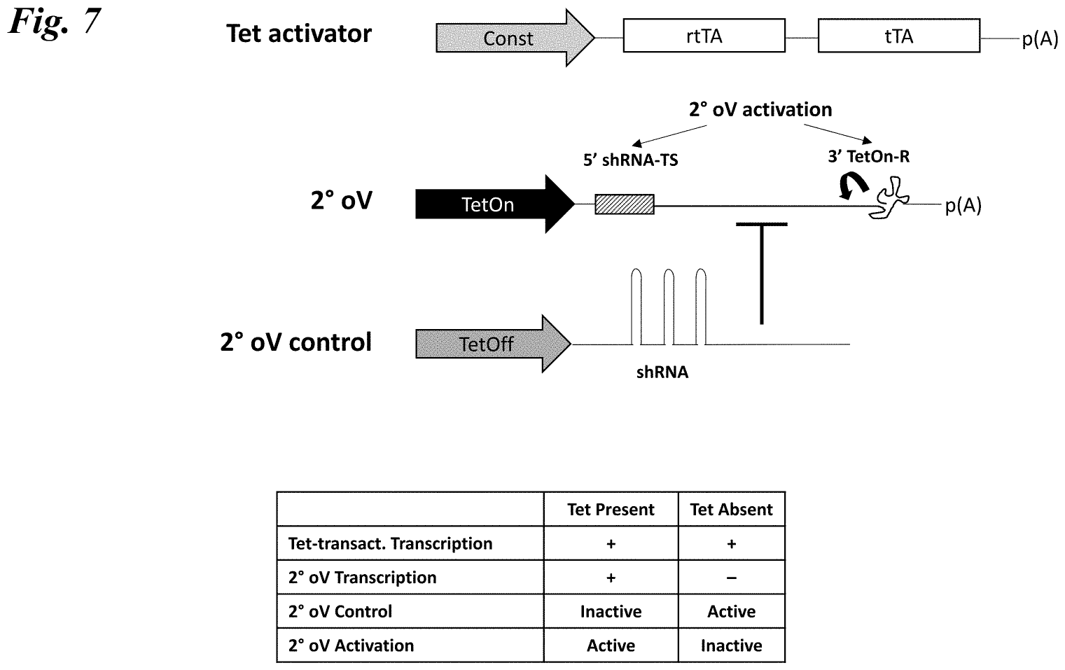

FIG. 7 illustrates exemplary schematics of components that can be inserted into the primary oncolytic viral genome or the primary viral genome to produce an exemplary nested oncolytical viral construct. Expression of the rtTA and the tTA peptides is under the control of a constitutively active promoter. Expression of the secondary viral genome is under the control of a TetOn Pol II promoter, such that transcription of the viral genome occurs in the presence of Tet and the rtTA peptide. Expression of the secondary oncolytic virus or the secondary virus transcript is further regulated by shRNAs specific for target sequences in the mRNA transcript of the secondary virus. Expression of the shRNAs is under the control of a TetOff promoter, such that transcription of the shRNAs occurs in the absence of Tet and the presence of the tTA peptide. Further, the secondary viral transcript is activated by cleavage on the 5′-end by an AmiRNA target site and 3′ TetOn-R, such that that the mRNA transcript is processed on the 5′ end in the presence of Tet.

In some embodiments, site-directed recombination systems are employed to control the expression of the secondary oncolytic virus or the secondary virus. In such embodiments, the primary oncolytic virus or the primary virus comprises a first polynucleotide encoding the secondary oncolytic virus or the secondary virus comprising the recombinase recognition sites and a second polynucleotide encoding the corresponding recombinase protein. The second polynucleotide encoding the recombinase protein can be under the control of an inducible or otherwise regulatable promoter such that expression of the recombinase protein can be temporally controlled. Site-directed recombination systems suitable for use in the present disclosure are known in the art, including the FRT/FLP system comprising flippase recognition target (FRT) sites recognized by the flippase (FLP) recombinase and the Cre/Lox system comprising loxP sites recognized by the Cre recombinase.

In some embodiments, the recombinase is a Flp recombinase. In some embodiments ts, the recombinase recognition sites are FRT sites (e.g., FRT-1 site, FRT-14 site). In some embodiments, the FRT-1 sites comprise or consists of a nucleic acid sequence having at least 90%, at least 95%, or 100% identity to SEQ ID NO: 850 or its complement. In some embodiments, the Recombinase Recognition Sites are FRT-14 sites. In some embodiments, the FRT-14 sites comprise or consists of a nucleic acid sequence having at least 90%, at least 95%, or 100% identity to SEQ ID NO: 851 or its complement.

In some embodiments, the recombinase is a Cre recombinase. In some embodiments, the recombinase is a Dre recombinase. In some embodiments, the recombinase is a ΦC31 (phiC31) recombinase. In some embodiments, the recombinase is a X, integrase. In some embodiments, the recombinase is selected from the Table 1 below:

TABLE 1
List of Recombinase and Exemplary Target Sites/Sequences

| | | | | SEQ |
|---|---|---|---|---|
| | | Exemplary | | ID |
| Recombinase | Origin | Target site | Exemplary Target sequence | NO |
| Flp | S. | FRT | 5′- | 850 |
| | cerevisiae | | GAAGTTCCTATTCTCTAGAAAGTATAGGAAC | |
| | | | TTC-3′ | |
| KD | K. | KDRT | 5′- | 875 |
| | drosophilarum | | AAACGATATCAGACATTTGTCTGATAATGCT | |
| | | | TCATTATCAGACAAATGTCTGATATCGTTT-3′ | |
| B2 | Z. bailii | B2RT | 5′- | 876 |
| | | | GAGTTTCATTAAGGAATAACTAATTCCCTAA | |
| | | | TGAAACTC-3′ | |
| B3 | Z. | B3RT | 5′- | 877 |
| | bisporus | | GGTTGCTTAAGAATAAGTAATTCTTAAGCAA | |
| | | | CC-3′ | |
| R | Z. rouxii | RSRT | 5′- | 878 |
| | | | TTGATGAAAGAATAACGTATTCTTTCATCAA- | |
| | | | 3′ | |
| Cre | Phage P1 | loxP | 5′- | 879 |
| | | | ATAACTTCGTATAGCATACATTATACGAAGT | |
| | | | TAT-3′ | |
| VCre | Vibrio | VloxP | 5′- | 880 |
| | sp. | | TCAATTTCTGAGAACTGTCATTCTCGGAAATT | |
| | | | GA-3′ | |
| SCre | Shewanella | SloxP | 5′- | 881 |
| | sp. | | CTCGTGTCCGATAACTGTAATTATCGGACAT | |
| | | | GAT-3′ | |
| Vika | V. | vox | 5′- | 882 |
| | corallii- | | AATAGGTCTGAGAACGCCCATTCTCAGACGT | |
| | lyticus | | ATT-3′ | |
| Dre | Bacteriophage | rox | 5′- | 883 |
| | D6 | | TAACTTTAAATAATGCCAATTATTTAAAGTT | |
| | | | A-3′ | |
| A-Int | Phage λ | attP | 5′-CAGCTTTTTTATACTAAGTTG-3′ | 884 |
| A-Int | Phage λ | attB | 5′-CTGCTTTTTTATACTAACTTG-3′ | 885 |
| HK022 | Phage | attP | 5′-ATCCTTTAGGTGAATAAGTTG-3′ | 886 |
| | HK022 | | | |
| HK022 | Phage | attB | 5′-GCACTTTAGGTGAAAAAGGTT-3′ | 887 |
| | HK022 | | | |
| φC31 | Phage | attP | 5′- | 888 |
| | φC31 | | CCCCAACTGGGGTAACCTTTGAGTTCTCTCA | |
| | | | GTTGGGG-3′ | |
| φC31 | Phage | attB | 5′- | 889 |
| | φC31 | | GTGCCAGGGCGTGCCCTTGGGCTCCCCGGGC | |
| | | | GCG-3′ | |
| Bxb1 | Phage | attP | 5′- | 890 |
| | Bxb1 | | GGTTTGTCTGGTCAACCACCGCGGTCTCAGT | |
| | | | GGTGTACGGTACAAACC-3′ | |
| Bxb1 | Phage | attB | 5′- | 891 |
| | Bxb1 | | GGCTTGTCGACGACGGCGGTCTCCGTCGTCA | |
| | | | GGATCAT-3′ | |
| Gin | Phage Mu | gix | 5′-TTATCCAAAACCTCGGTTTACAGGAA-3′ | 892 |
| Tn3 | E. coli | res site I | 5′-CGTTCGAAATATTATAAATTATCAGACA-3′ | 893 |

In some embodiments, the expression of the recombinase results in expression of a functional secondary oncolytic virus or a functional secondary virus. For example, in some embodiments, the polynucleotide encoding the secondary virus comprises one or more frame-shift or stop codon insertions flanked by recombinase recognition sites (See e.g., FIG. 4A). In the absence of recombinase expression, the transcribed secondary oncolytic virus or secondary virus comprises the frame-shift or stop codon insertion, which prevents expression of a functional secondary oncolytic virus or a functional secondary virus. When expression of the recombinase protein is activated or induced, the frame-shift or stop codon insertion is excised from the second polynucleotide, allowing expression of a functional secondary oncolytic virus or a functional secondary virus (See e.g., FIG. 4A). In some embodiments, the polynucleotide encoding the secondary oncolytic virus or the secondary virus or a portion thereof is inverted and flanked by recombinase recognition sites. In the absence of recombinase expression, the transcribed secondary oncolytic virus or secondary virus comprises the inverted portion, which prevents expression of a functional secondary oncolytic virus or a functional secondary virus. When expression of the recombinase protein is activated or induced, the inverted portion is flipped to the correct orientation, allowing expression of a functional secondary oncolytic virus or a functional secondary virus (See e.g., FIG. 4B).

In some embodiments, the expression of the recombinase prevents expression of a functional secondary oncolytic virus or a functional secondary virus. For example, in some embodiments, the polynucleotide encoding the secondary virus is flanked by recombinase recognition sites. In the absence of recombinase expression, the polynucleotide is transcribed and produces a functional secondary oncolytic virus or a functional secondary virus. When expression of the recombinase protein is activated or induced, recombination between the recombinase recognition sites can result in inversion of the polynucleotide, preventing expression of the secondary oncolytic virus or the secondary virus. In some embodiments, the promoter controlling transcription of the polynucleotide encoding the secondary oncolytic virus or the secondary virus is flanked by recombinase recognition sites. In the absence of recombinase expression, the promoter remains functional and allows transcription of the secondary oncolytic virus or the secondary virus. When expression of the recombinase protein is activated or induced, recombination between the recombinase recognition sites can result in inversion of the promoter, preventing expression of the secondary oncolytic virus or the secondary virus.

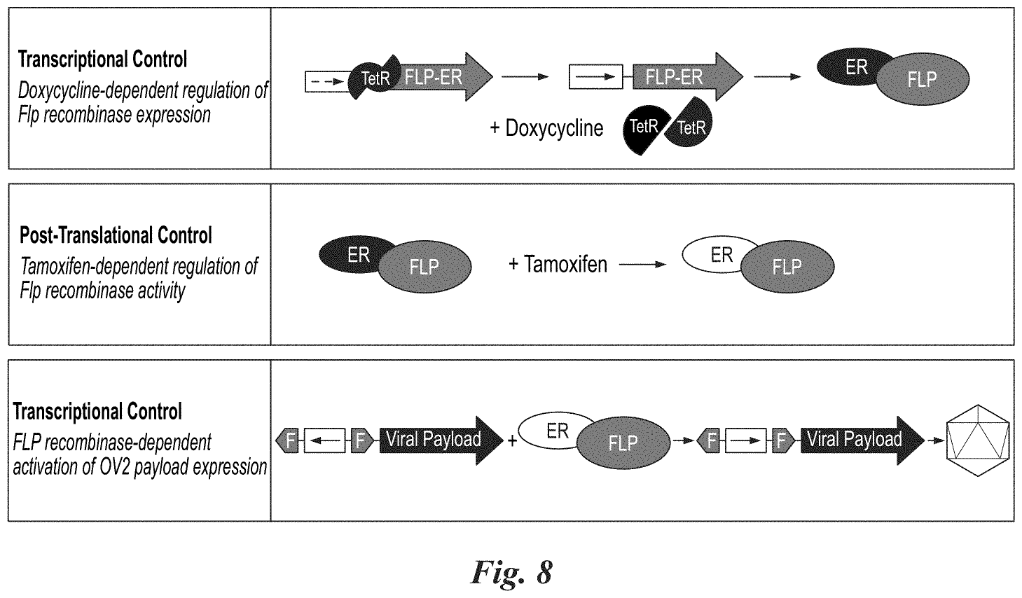

As illustrated in a non-limiting example in FIG. 8, at least three levels of control can be engineered into the recombinase system, which can provide stringent temporal regulation of the expression of the secondary oncolytic virus (OV2) or the secondary virus.

The first level is transcriptional control of the recombinase. In some embodiments, a regulatable promoter is operably linked to the coding region of the recombinase. In some embodiments, the regulatable promoter is a TetOn promoter. In some embodiments, the regulatable promoter allows transcriptional repression by the bacterial TetR repressor. In some embodiments, promoter activity is de-repressed through the addition of doxycycline, which results in expression of the recombinase. In some embodiments, the recombinase is a Flp recombinase or a fusion protein thereof. In some embodiments, the recombinase is a Cre recombinase or a fusion protein thereof.

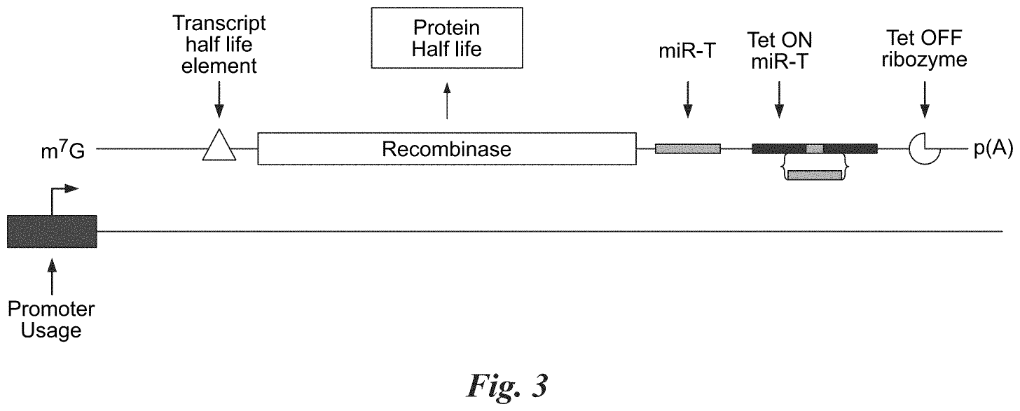

FIG. 3 illustrates exemplary control elements that can be used to regulate the transcription of mRNA encoding the recombinase. This non-limiting example depicts control elements for regulating the expression and/or function of virus activating T7 RNA polymerase or recombinase. In some embodiments, transcriptional control can be achieved with a tumor-specific promoter or regulatable promoter. In some embodiments, the polynucleotide encoding the recombinase is operably linked to a regulatable promoter. In some embodiments, the polynucleotide encoding the recombinase comprises one or more post-transcriptional control elements. Post-transcriptional control elements include modulating mRNA or the mRNA encoded protein half-life, miRNA target sites, Tet-ON miR-T elements, Tet-OFF ribozymes/aptazymes, and any combination thereof that controls transcript abundance in a ligand dependent, tumor cell specific, or constitutive fashion. In some embodiments, additional control elements can be engineered into the encoded polypeptide (e.g., recombinase) to control its half life, subcellular localization and/or activity. Exemplary Tet-On miR-T elements are described in Mou et al, Mol Ther. 2018 May 2; 26(5):1277-1286. Exemplary Tet-On ribozymes/aptazymes are described in Zhong et al., Elife. 2016 Nov. 2; 5:e18858.

In some embodiments, one or more mRNA destabilization elements are inserted into the recombinase expression cassette. In some embodiments, the one or more mRNA destabilization elements can destabilize the mRNA transcript encoding the recombinase and/or increase the mRNA turnover. In some embodiments, the presence of the one or more mRNA destabilization elements can decrease or minimize leaky expression of the recombinase mRNA in the uninduced state, such that there will only be sufficient recombinase available to mediate the intended recombination reaction when the system is induced (e.g., by an exogenous agent). In some embodiments, the mRNA destabilization elements comprise a c-fos coding element. In some embodiments, the c-fos coding element comprises or consists of a nucleic acid sequence having at least 70%, at least 75%, at least 80%, at least 85%, at least 90%, at least 95%, or 100% identity to SEQ ID NO: 894. In some embodiments, the mRNA destabilization elements comprise an AU-rich element from the 3′UTR of c-fos gene. In some embodiments, the AU-rich element from the 3′UTR of c-fos gene comprises or consists of a nucleic acid sequence having at least 70%, at least 75%, at least 80%, at least 85%, at least 90%, at least 95%, or 100% identity to SEQ ID NO: 895. In some embodiments, the mRNA destabilization elements comprise a combination of both FCE and ARE, optionally in tandem. In some embodiments, the combination of both FCE and ARE comprises or consists of a nucleic acid sequence having at least 70%, at least 75%, at least 80%, at least 85%, at least 90%, at least 95%, or 100% identity to SEQ ID NO: 896. In some embodiments, one or more introns are inserted into the recombinase coding region. In some embodiments, the presence of the one or more introns can prevent or minimize undesirable leaky expression of the recombinase in a prokaryotic expression system (e.g., when prokaryotic cells are used to generate the vector encoding the recombinase).

The second level is post-translational control of recombinase activity. In some embodiments, the activity and/or cellular localization of the recombinase is regulatable. In some embodiments, the activity and/or cellular localization of recombinase is regulated by an exogenous agent (e.g., a ligand or a small molecule). In some embodiments, the recombinase is fused to one or more activity control domains. In some embodiments, an exogenous agent (e.g., a ligand or a small molecule) modulates the cellular activity and/or localization of the recombinase though the one or more activity control domains. In some embodiments, the activity control domain is a modified ligand binding domain of estrogen receptor (ER). In the absence of ligand, the fusion protein (recombinase-ER) is retained in the cytoplasm where it cannot catalyze recombination, but the addition of corresponding small molecule (e.g., 4-hydroxytamoxifen) allows the fusion protein to translocate to the nucleus to carry out recombination. In some embodiments, the activity control domain is a progesterone receptor (PR) or a portion thereof. In some embodiments, the activity of the corresponding fusion protein (recombinase-PR) is induced with the progesterone analog RU-486. In some embodiments, the activity control domain is a modified E. coli dihydrofolate reductase (DHFR) protein. In some embodiments, the corresponding fusion protein (recombinase-DHFR) is unstable and is rapidly degraded in proteasomes in the absence of an inducer. In some embodiments, the corresponding inducer is the antibiotic trimethoprim (TMP), and the fusion protein is stabilized, translocates to the nucleus and carry out recombination in the presence of TMP.

In some embodiments, the recombinase is Flp and the activity control domain is a modified ligand binding domain of estrogen receptor (ER). In some embodiments, the Flp-ER fusion protein comprises an amino acid sequence having at least 70%, at least 75%, at least 80%, at least 85%, at least 90%, at least 95%, or 100% identity to the amino acid sequence encoded by SEQ ID NO: 846. In some embodiments, the Flp-ER fusion protein comprises an amino acid sequence having at least 70%, at least 75%, at least 80%, at least 85%, at least 90%, at least 95%, or 100% identity to the amino acid sequence encoded by SEQ ID NO:847. In some embodiments, the fusion protein comprises an RGS linker. In some embodiments, the fusion protein comprises a XTEN linker. In some embodiments, the fusion protein comprises a NLS and/or PEST sequence, optionally at the N-terminus of the fusion protein. In some embodiments, the NLS sequence comprises an amino acid sequence having at least 80%, at least 85%, at least 90%, at least 95%, or 100% identity to SEQ ID NO: 848. In some embodiments, the PEST sequence comprises an amino acid sequence having at least 80%, at least 85%, at least 90%, at least 95%, or 100% identity to SEQ ID NO: 849. In some embodiments, the FLP-RGS-ER fusion polypeptide comprises an amino acid sequence having at least 80%, at least 85%, at least 90%, at least 95%, or 100% identity to SEQ ID NO: 846. In some embodiments, the FLP-XTEN-ERT2 polypeptide comprises an amino acid sequence having at least 80%, at least 85%, at least 90%, at least 95%, or 100% identity to SEQ ID NO: 847.

The third level is transcriptional control of OV2 expression, in which the recombinase mediates excision and/or inversion of a portion of the polynucleotide comprising the promoter and coding region of OV2, resulting in activation or inactivation of the OV2 viral genome transcript expression. In some embodiments, the recombinase mediates excision of a portion of the polynucleotide (e.g., to remove a transcription termination signal). In some embodiments, the recombinase mediates inversion of a portion of the polynucleotide (e.g., to place the coding region of OV2 under the control of the promoter). In some embodiments, the recombinase mediates both excision and inversion. In some embodiments, one or more intron regions are introduced into the polynucleotide. In some embodiments, the intron regions remove the recombinase recognition site from the mature OV2 viral genome transcript.

FIG. 4A-FIG. 4B illustrate exemplary use of site-directed recombination systems to control expression of the secondary oncolytic virus or the secondary virus. FIG. 4A shows a scheme for insertion of frameshift/stop codons in the polynucleotide encoding the secondary oncolytic virus or the secondary virus that could be excised by a FLP or another recombinase. In some embodiments, the viral genome of the secondary oncolytic virus or the secondary virus is rendered inert by insertion of stop codon(s) or polynucleotides that causes frameshift of the coding region, which are flanked by recombination sites (e.g., FRT sites). In the presence of the corresponding recombinase (FLP protein), the stop codon(s) or polynucleotides that causes frameshift of the coding region can be removed which generates a functional viral genome. Similarly, FIG. 4B shows an inactive inverted promoter flanked by recombination sites, which can be rendered active once it is inverted to the correct orientation in the presence of the corresponding recombinase.

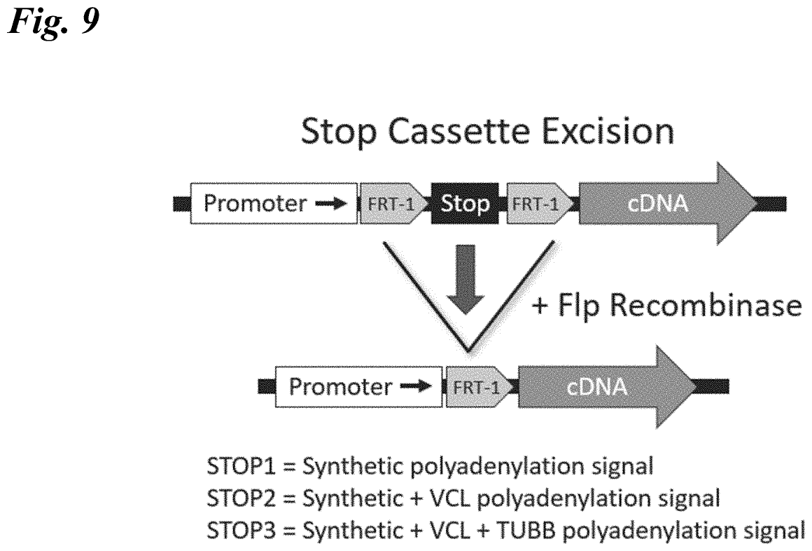

FIGS. 9-11 showcase exemplary recombinase-responsive cassettes that can be used to control the expression of a target polynucleotide.

A Recombinase-Responsive Excision Cassette (RREC) can be used to control the expression of a target polynucleotide (e.g., cDNA). In some embodiments, the RREC comprises a control element in the middle and flanking recombinase recognition sites on each side of the control element.

In some embodiments, a Recombinase-Responsive Excision Cassette (RREC) adopts the following configuration:

### 5′-Recombinase Recognition Site A1-Control Element-Recombinase Recognition Site A2-3′,

wherein the Recombinase Recognition Sites mediate the excision of the control element at the presence of the corresponding recombinase. In some embodiments, the Recombinase Recognition Sites A1 and A2 are in the same orientation. In some embodiments, the Recombinase Recognition Sites A1 and A2 have the same nucleotide sequence. In some embodiments, the Recombinase is a Flp recombinase. In some embodiments, the Recombinase Recognition Sites are FRT sites. In some embodiments, the Recombinase Recognition Sites are FRT-1 sites. In some embodiments, the Recombinase is a Cre recombinase. In some embodiments, the Recombinase Recognition Sites are Lox sites.

In some embodiments, the Control Element comprises or consists of a transcriptional/translational termination element (STOP). In some embodiments, the transcriptional/translational termination element (STOP) comprises or consists of one or more translational stop codons, optionally in each reading frame. In some embodiments, the transcriptional/translational termination element (STOP) comprises or consists of one or more transcriptional termination signals. In some embodiments, the transcriptional/translational termination element (STOP) comprises or consists of a DNA sequence encoding multiple translational stop codons in each reading frame followed by a transcriptional termination signal (e.g., polyadenylation signal). In some embodiments, the Control Element comprises or consists of a frameshift element, which consists of a DNA sequence causing frameshift of the downstream open reading frame. In some embodiments, additional nucleotides are present in between the Control Element and the one or more of the Recombinase Recognition Sites on either or both sides. In some embodiments, the RREC is placed in between a promoter and a coding region (e.g., an Open Reading Frame). In some embodiments, the RREC is placed in the 5′-UTR of a transcript. In some embodiments, the RREC is placed in a promoter region. In some embodiments, the RREC is placed in a coding region (e.g., an Open Reading Frame). In some embodiments, the STOP element comprises or consists of a nucleic acid sequence having at least 70%, at least 75%, at least 80%, at least 85%, at least 90%, at least 95%, or 100% identity to SEQ ID NO: 854. In some embodiments, the STOP element comprises or consists of a nucleic acid sequence having at least 70%, at least 75%, at least 80%, at least 85%, at least 90%, at least 95%, or 100% identity to SEQ ID NO: 855. In some embodiments, the STOP element comprises or consists of a nucleic acid sequence having at least 70%, at least 75%, at least 80%, at least 85%, at least 90%, at least 95%, or 100% identity to SEQ ID NO: 856.

A non-limiting example of RREC—a STOP cassette—is illustrated in FIG. 9, in which the recombinase is Flp, both recombinase recognition sites are FRT-1 sites in the same orientation, and the control element is a transcriptional/translational termination element (STOP). In some embodiments, the The STOP cassette may comprise or consist of a (STOP) element flanked by tandem direct repeats of a minimal FRT element. In some embodiments, the transcriptional/translational termination element comprises or consists of a DNA sequence encoding multiple translational stop codons in each reading frame followed by a polyadenylation signal. In some embodiments, the STOP cassette is inserted between the promoter and the cDNA of interest being regulated such that it is located in the 5′-UTR of the corresponding transcript. In the absence of Flp recombinase the STOP cassette remains stably integrated and functions to terminate transcription and thus prevent expression of the cDNA of interest. When the Flp recombinase is present, it will mediate recombination between the tandem FRT elements and irreversibly excise the STOP element, thus activating expression of the cDNA of interest. In some embodiments, the STOP element contains a single synthetic polyadenylation signal (for example, as in STOP 1, SEQ ID NO: 854). In some embodiments, the STOP element comprises multiple polyadenylation signals, as in STOP2 (SEQ ID NO: 855) and STOP3 (SEQ ID NO: 856). In some embodiments, having multiple polyadenylation signals increases the efficiency of transcriptional termination. In some embodiments, the STOP cassette comprises or consists of a nucleic acid sequence having at least 70%, at least 75%, at least 80%, at least 85%, at least 90%, at least 95%, or 100% identity to SEQ ID NO: 857. In some embodiments, the STOP cassette comprises or consists of a nucleic acid sequence having at least 70%, at least 75%, at least 80%, at least 85%, at least 90%, at least 95%, or 100% identity to SEQ ID NO: 858. In some embodiments, the STOP cassette comprises or consists of a nucleic acid sequence having at least 70%, at least 75%, at least 80%, at least 85%, at least 90%, at least 95%, or 100% identity to SEQ ID NO: 859.

Additional layers of expression control can be achieved via a Recombinase Responsive Inversion Cassette (RRIC). In some embodiments, the RRIC comprises a Central Element in the middle and flanking recombinase recognition sites on each side of the control element.

In some embodiments, a Recombinase Responsive Inversion Cassette (RRIC) adopts the following configuration:

### 5′-Recombinase Recognition Site A1-Central Element-Recombinase Recognition Site A2-3′,

wherein the Recombinase Recognition Sites A1 and A2 mediates the inversion of the Central Element's orientation at the presence of the corresponding recombinase. In some embodiments, the Recombinase Recognition Sites A1 and A2 are in the opposite orientation. In some embodiments, the Recombinase Recognition Sites A1 and A2 have the same nucleotide sequence. In some embodiments, the Recombinase is a Flp recombinase. In some embodiments, the Recombinase Recognition Sites are FRT sites. In some embodiments, the Recombinase Recognition Sites are FRT-1 sites. In some embodiments, the Recombinase is a Cre recombinase. In some embodiments, the Recombinase Recognition Sites are Lox sites.

In some embodiments, the Central Element of the RRIC comprises or consists of a promoter or a portion of the promoter, and such RRIC can optionally be placed upstream of a coding region. In some embodiments, the Central Element of the RRIC comprises or consists of a coding region (e.g., Open Reading Frame) or a portion of the coding region, and such RRIC can optionally be placed downstream of a promoter region. In some embodiments, the coding region encodes the viral genome of the secondary oncolytic virus or the secondary virus. In some embodiments, additional nucleotides are present in between the Central Element and the one or more Recombinase Recognition Sites on either or both sides.

In some embodiments, the RRIC comprises two or more Recombinase Recognition Sites on each side of the Central Element. In some embodiments, the RRIC adopts the following configuration:

### 5′-Recombinase Recognition Site A1-Recombinase Recognition Site B1-Central Element-Recombinase Recognition Site A2-Recombinase Recognition Site B2-3′,

wherein the pair of Recombinase Recognition Sites A1 and A2, and/or the pair of Recombinase Recognition Sites B1 and B2, can mediates the inversion of the Central Element's orientation at the presence of the corresponding recombinase. In some embodiments, these two pairs are orthogonal to each other (i.e., no inversion mediated by one of Recombinase Recognition Sites A and one of Recombinase Recognition Sites B). In some embodiments, the Recombinase Recognition Sites A1 and A2 are in the opposite orientation. In some embodiments, the Recombinase Recognition Sites A1 and A2 have the same nucleotide sequence. In some embodiments, the Recombinase Recognition Sites B1 and B2 are in the opposite orientation. In some embodiments, the Recombinase Recognition Sites B1 and B2 have the same nucleotide sequence. In some embodiments, the Recombinase is a Flp recombinase. In some embodiments, the Recombinase Recognition Sites are FRT sites. In some embodiments, one pair of the Recombinase Recognition Sites (either pair A or pair B) comprises FRT-1 sites, and the other pair comprise FRT-14 sites. In some embodiments, the Recombinase is a Cre recombinase. In some embodiments, the Recombinase Recognition Sites are Lox sites. Additional polynucleotides can be present between these elements in the configuration above.

In some embodiments, inversion mediated by one pair of the Recombinase Recognition Sites (either pair A or pair B) brings the other pair into the same orientation so that the other pair of the Recombinase Recognition Sites can now mediate an excision of part of the RRIC, resulting in the following new polynucleotide configuration:

5′-Recombinase Recognition Site A-Central Element (inverted)-Recombinase Recognition Site B-3′.
In some embodiments, once the excision takes place, the reaction is irreversible. Therefore, one of the benefit of having two pairs of Recombinase Recognition Sites in such a configuration is that the inversion of the Central Element, once carried out by the recombinase, may be irreversible.

Additional elements can be incorporated into the RRIC. As a non-limiting example, one or more Control Elements may be incorporated into the RRIC. In some embodiments, the one or more Control Elements may be incorporated into one or more regions between the Recombinase Recognition Sites. In some embodiments, the Control Element may be a STOP element of the disclosure or other transcriptional/translational termination signal. In some embodiments, introduction of a transcriptional/translational termination signal prevents accidental or leaky expression of a functional payload protein or viral genome due to cryptic promoter region and/or transcriptional initiation signal near the coding region.

Accordingly, in some embodiments, the RRIC adopts the following configuration:

5′-Recombinase Recognition Site A1-Control Element 1 (optional)-Recombinase Recognition Site B1-Central Element-Recombinase Recognition Site A2-Control Element 2 (optional)-Recombinase Recognition Site B2-3′,
wherein one or both Control Elements may be present in the RRIC. In some embodiments, the Control Elements are the same. In some embodiments, the Control Elements are different. In some embodiments, one or more of the Control Elements are STOP elements.

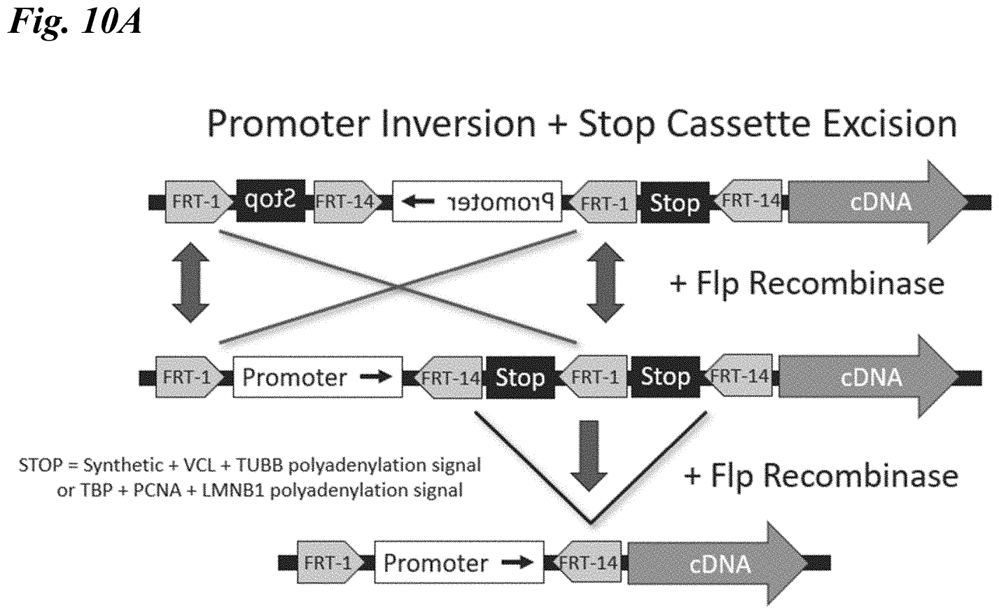

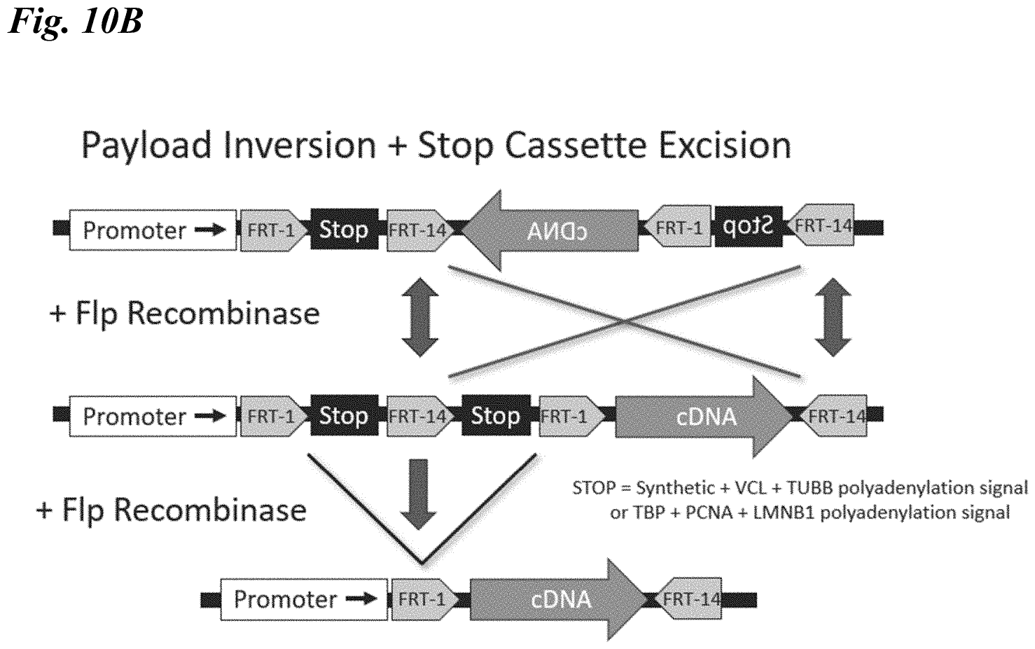

A non-limiting example of the RRIC described above is illustrated in FIG. 10A, wherein the Recombinase is Flp, the Recombinase Recognition Sites are FRT-1 and FRT-14, the Control Elements are STOP3 elements, and the Central Element is a promoter. The promoter is oriented such that it is inverted with respect to the cDNA of interest before inversion occurs, and thus cannot drive cDNA expression in the absence of the Flp recombinase. In the absence of Flp recombinase the STOP cassettes also remain stably integrated and function to terminate transcription and thus preserve the orientation of the inverted promoter element. If Flp recombinase is present it will mediate recombination between one pair of the inverted FRT elements (either FRT-1 or FRT-14) and invert all of the elements located between them. In FIG. 10A this inversion event is shown for the FRT-1 elements, but a similar reaction can occur for FRT-14 elements. Note that the inversion event orients the promoter so that it can potentially drive cDNA expression, and also converts the opposite FRT elements in the other FRT pair from an inverted repeat to a direct repeat orientation. If sufficient FLP is still available then it can mediate a second recombination reaction, either reversing the first reaction and regenerating the original configuration, or recombining the set of direct repeat FRT elements, as shown for FRT-14 in FIG. 10A. This second reaction will irreversibly excise the STOP elements and activate expression of the cDNA of interest. The design in FIG. 10A is sometimes referred to as “Promoter Inversion design” in the disclosure. An exemplary Promoter Inversion design is provided herein as SEQ ID NO: 860. Similarly, FIG. 10B illustrates a non-limiting example of the RRIC with payload coding region as the Central Element that will be inverted at the presence of Flp recombinase, though a process analogous to the promoter inversion element but involves inversion of the cDNA payload instead of the promoter. The design in FIG. 10B is sometimes referred to as “Payload Inversion design” in the disclosure. An exemplary Payload Inversion design is provided herein as SEQ ID NO: 861.

In some embodiments, one or more introns and/or splicing elements are inserted into the cassettes of the disclosure. In some embodiments, one or more introns are inserted into and/or adjacent to the RRIC. In some embodiments, the expression cassette adopts the following configuration:

5′-Recombinase Recognition Site A1-Control Element 1 (optional)-Recombinase Recognition Site B1-Central Element with Intron C1-Recombinase Recognition Site A2-Control Element 2 (optional)-Recombinase Recognition Site B2-Intron C2-3′ coding region-3′,
wherein the Central Element with Intron C1 comprises the following configuration:
5′-Promoter-5′ coding region-Intron C1-3′ (inverted orientation in the cassette),
In some embodiments, the initial orientation of the 5′ coding region in the cassette is opposite to the orientation of the 3′ coding region. In some embodiments, additional nucleotides are present in between any or all of these elements. As in the case of RRIC, the recombinase can mediate the inversion/excision events via the Recombinase Recognition Sites, and the final irreversible recombination product adopts the following configuration:
5′-Recombinase Recognition Site A-Promoter-5′ coding region-Intron C1-Recombinase Recognition Site B-Intron C2-3′ coding region-3′,
wherein the removal of the Intron to Intron′ region (mediated by the Intron C1 and Intron C2 elements) after mRNA transcription generates full coding region without extra nucleotides inside the coding region. In some embodiments, introducing one or more intron regions as described herein prevents accidental or leaky expression of a functional payload protein or viral genome due to the presence of cryptic promoter region and/or transcriptional initiation signal near the coding region.

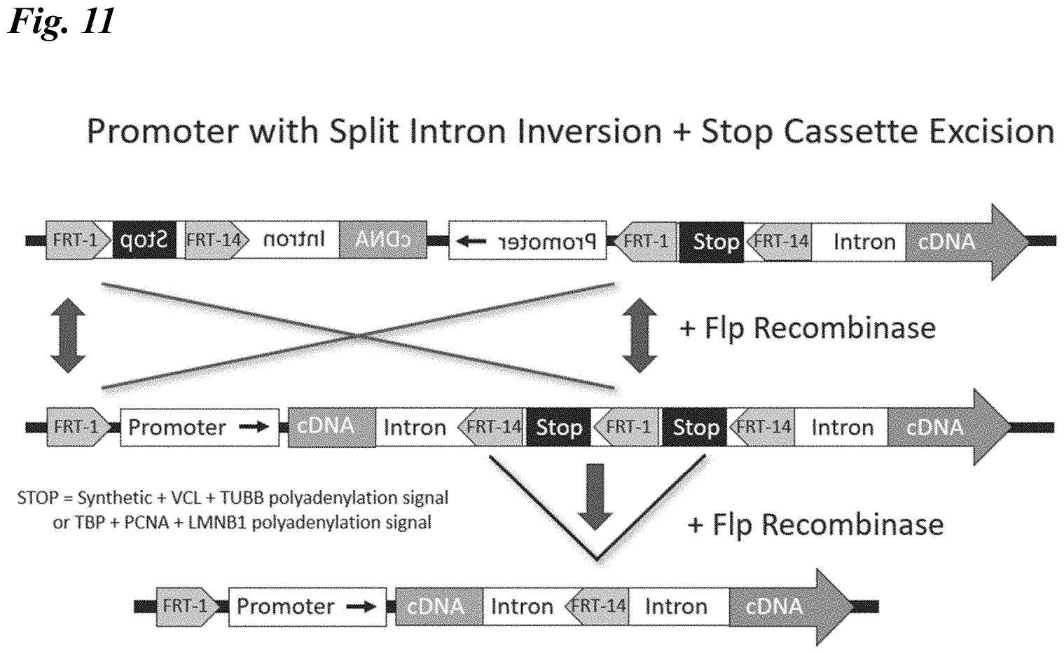

A non-limiting example is illustrated in FIG. 11 (which is sometimes referred to as “Split Intron Inversion design” in the disclosure). An exemplary Split Intron Inversion design is provided herein as SEQ ID NO: 862. The split intron inversion design functions similarly to the Promoter Inversion design. However, a key difference is that the cDNA has been engineered to contain a pair of split introns based on the Intron 3 of the ACTB gene (SEQ ID NO: 863), which disrupt the coding region. The intron was split into a 5′-splice donor and a 3′-splice acceptor fragments, with a BamHI and a EcoRV restriction site delineating the location of the split. The Central Element here comprise the promoter, 5′ cDNA element, one of the introns and 5′-splice donor site which are in the opposite orientation with respect to the 3′ cDNA element. The Central Element is flanked by STOP3 Elements and FRT sites, similar to the Promoter Inversion design. In the absence of Flp recombinase, the two portions of cDNA are split and in opposite orientation and thus cannot drive expression of complete cDNA, and the STOP Element remain stably integrated and function to terminate transcription and thus preserve the orientation of the inverted promoter and 5′ cDNA element. If Flp recombinase is present, it will mediate recombination between one set of the inverted tandem FRT sites and invert all of the elements located between them. In FIG. 11, this inversion event is shown for the FRT-1 sites, but a similar reaction can occur for FRT-14 sites. Note that the inversion event orients the promoter and partial cDNA element so that it can potentially drive cDNA expression, and also converts the other pair of the FRT sites from an inverted repeat to a direct repeat orientation. If sufficient FLP is still available then it can mediate a second recombination reaction, either reversing the first reaction and regenerating the original configuration, or else recombining the set of direct repeat FRT elements, as shown for FRT-14 in FIG. 11. In the second case, the resultant expression cassette comprises a polynucleotide encoding a full cDNA with internal introns which, once transcribed, will form a complete cDNA without intron or FRT site after RNA splicing. Therefore, this second reaction may irreversibly activate expression of the cDNA of interest.

### Payload Molecule

In some embodiments, the primary virus comprises a polynucleotide encoding a secondary oncolytic virus or a secondary virus and a polynucleotide encoding a payload molecule. A “payload molecule” refers to any molecule capable of further enhancing the therapeutic efficacy of the primary and/or secondary oncolytic virus, or the primary and/or secondary virus, including cytokines, chemokines, enzymes, antibodies or antigen-binding fragments thereof, soluble receptors, a ligand for a cell-surface receptor, bi-partite peptides, tri-partite peptides, and cytotoxic peptides.

In some embodiments, the payload molecule is a cytotoxic peptide. A “cytotoxic peptide” refers to a protein capable of inducing cell death in when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribosome inactivating proteins (RIPs) (e.g.; saporin and gelonin), Type II RIPs (e.g., ricin), Shiga-like toxin 1 (Slt1), photosensitive reactive oxygen species (e.g. killer-red). In some embodiments, the cytotoxic peptide is encoded by a suicide gene resulting in cell death through apoptosis, such as a caspase gene.

In some embodiments, the payload molecule is an antibody or antigen binding fragment thereof. In some embodiments, the antibody or antigen binding fragment thereof specifically binds to a cell surface receptor, such as an immune checkpoint receptor (e.g., PD1, PDL1, and CTLA4) or additional cell surface receptors involved in cell growth and activation (e.g., OX40, CD200R, SIRPα, CSF1R, 4-1BB, CD40, and NKG2D). In some embodiments, the payload molecule is a ligand for a cell surface receptor. Exemplary ligands suitable for use as payloads include, but are not limited to, NKG2D ligands, neuropilin ligands, Flt3 ligand, 4-1BBL, CD40L, GITRL, LIGHT, and CD47. In some embodiments, the payload molecule is a soluble receptor. Exemplary soluble receptors suitable for use as payloads include, but are not limited to, soluble receptors such as IL-13R, TGFβR1, TGFβR2, SIRPα, PD-1, IL-35R, IL-15R, IL-2R, IL-12R, and interferon receptors.

In some embodiments, the payload molecule is a cytokine. Exemplary cytokines suitable for use as payloads include, but are not limited to, IL-1, IL-12, IL-15, IL-18, IL-36, TNFα, IFNα, IFNβ, and IFNγ. In some embodiments, the payload molecule is a chemokine. Exemplary chemokines suitable for use as payloads include, but are not limited to, CXCL10, CXCL9, CCL21, CCL4, and CCL5.

In some embodiments, the payload molecule is an enzyme. Exemplary enzymes suitable for use as payloads include, but are not limited to, an adenosine deaminase, 15-Hydroxyprostaglandin Dehydrogenase, a matrix metalloprotease (e.g., MMP9), a collagenase, a hyaluronidase, a gelatinase, and an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450. In some embodiments, the enzyme is capable of inducing or activating cell death pathways in the target cell (e.g., a caspase).

In some embodiments, the payload molecule is a bi-partite peptide comprising a first domain capable of binding a cell surface antigen expressed on a non-cancerous effector cell and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g, a single chain variable fragment (scFv) or an F(ab)) a scorpion polypeptide, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-IG™), a TANDAB®, a bi-specific T cell engager (BITE™); a DUOBODY®, or a dual affinity retargeting (DART) polypeptide. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, or neuropilin.

In some embodiments, the primary oncolytic virus or the primary virus comprises a polynucleotide encoding a secondary oncolytic virus or a secondary virus and a polynucleotide encoding a payload molecule, wherein a target sequence for an RNAi molecule is inserted at one or more locations in the polynucleotide encoding the payload molecule.

In some embodiments, the polynucleotide encoding the payload molecule further comprises one or more internal RNAi target sequences to prevent expression of the payload molecule in a cell or a subject. In some embodiments, the internal RNAi target sequence is a target sequence for an siRNA molecule, an AmiRNA molecule, or an miRNA molecule. In some embodiments, the internal RNAi sequences enable further temporal control over the expression of the payload molecule after introduction of the viral construct to a cell or administration to a subject. In such embodiments, the internal RNAi target sequence is an miRNA target sequence for an miRNA that is endogenously expressed by a cell. For example, in some embodiments, the polynucleotide encoding the payload molecule comprises one or more internal target sequences for an miRNA endogenously expressed by a non-cancerous cell, such that the payload molecule is not expressed in that cell.

In some embodiments, the internal RNAi sequences enable control over the expression of the payload molecule during production of dual viral vector. In some embodiments, the internal RNAi target sequence is a target sequence for an siRNA molecule, an AmiRNA molecule, or an artificial miRNA molecule that is not endogenously expressed by the production cell line or by a cell in a sample or subject.

### Promoter

In some embodiments, the promoter is a tetracycline (Tet)-dependent promoter. In some embodiments, the Tet-dependent promoter comprises a Tet-On element downstream of the promoter element. In some embodiments, the promoter is a CMV promoter (SEQ ID NO: 897), a HSV gB promoter (SEQ ID NO: 900), a HSV gC promoter (SEQ ID NO: 901), a HSV ICP8 promoter (SEQ ID NO: 899), a HSV TK promoter (SEQ ID NO: 898), a HBP1 promoter (hybrid of HSV-ICP8 and -TK promoters) or a HBP2 promoter (hybrid of HSV-TK and -ICP8 promoters). In some embodiments, the promoter is a CMV promoter. An exemplary HBP1-TetOn promoter is provided as SEQ ID NO: 865. An exemplary HBP2-TetOn promoter is provided as SEQ ID NO: 866. In some embodiments, the promoter comprises a nucleic acid sequence having at least 70%, at least 75%, at least 80%, at least 85%, at least 90%, at least 95%, or 100% identity to any one of the sequences according to SEQ ID NOS: 865-866 and 897-901.

### Primary Viruses

In some embodiments, the primary oncolytic virus or the primary virus is a double stranded DNA (dsDNA) virus. Exemplary dsDNA viruses include members of the Myoviridae family, the Podoviridae family, the Siphoviridae family, the Alloherpesviridae family, the Herpesviridae family (e.g., HSV-1, HSV-1, Equine Herpes Virus), the Poxviridae family (e.g., molluscum contagiosum virus, vaccina virus, myxoma virus), and the Adenoviridae family (e.g., an adenovirus). In some embodiments, the primary oncolytic virus or the primary virus is HSV-1 or HSV-2.

In some embodiments, the primary oncolytic virus or the primary virus is a RNA virus. In some embodiments, the primary oncolytic virus or the primary virus is a paramyxovirus or a rhabdovirus.

In some embodiments, the primary oncolytic virus or the primary virus comprises no modifications compared to the wild-type virus other than the insertion of the polynucleotide encoding the secondary oncolytic virus or the secondary virus. In some embodiments, the primary virus is an HSV. HSV viral vectors and methods for their construction are described in, for example, U.S. Pat. Nos. 7,078,029, 6,261,552, 5,998,174, 5,879,934, 5,849,572, 5,849,571, 5,837,532, 5,804,413, and 5,658,724; and International Patent Applications WO 91/02788, WO 96/04394, WO 98/15637, and WO 99/06583, which are incorporated herein by reference in their entireties. The sequence of HSV is published (NCBI Accession No. NC_001806; See also McGoech et al., J. Gen. Virol, 69 (PT 7), 1531-1574 (1988)) and can be used in the design of additional HSV viral vectors.

In some embodiments, the primary virus comprises the polynucleotide encoding the secondary oncolytic virus or the secondary virus and one or more additional modifications compared to a wild-type virus. In some embodiments, the primary virus is a variant of a wild-type HSV. Variant HSV vectors and methods for their construction are described in U.S. Pat. No. 9,593,347; US Patent Application Publication Nos: 2016-0250267, 2017-0036819, 2017-0274025, 2017-0189514, and 2017-0107537; and International PCT Publication Nos: WO 2011/0130749 and WO 2015/066042, which are incorporated herein by reference in their entireties. In some embodiments, the primary virus is a variant HSV and comprises a deletion of the internal repeat (joint) region, which comprises one copy each of the diploid genes ICP0, ICP34.5, LAT, and ICP4 along with the promoter for the ICP47 gene (See e.g., U.S. Pat. Nos. 10,210,575; 10,172,893; and 10,188,686).

In some embodiments, the primary virus comprises a polynucleotide encoding a secondary oncolytic virus or a secondary virus and one or more modifications that enhance entry of the primary virus into cells. In some embodiments, the primary virus comprises a mutation in one or more surface glycoproteins that facilitate viral entry into cells through non-canonical receptors and/or that enhance lateral spread in cells typically resistant to viral lateral spread. In some embodiments, the primary virus comprises a non-native ligand on the surface of the virus, such as an scFv or other antigen binding molecule, that binds to a surface receptor on a target cell. In some embodiments, the surface receptor on the target cell is EGF-R.

In some embodiments, the primary virus is a variant HSV and exhibits enhanced entry into cells. In some embodiments, the primary HSV can directly infect cells through interaction with cell proteins other than typical mediators of HSV infection (e.g., other than nectin-1, HVEM, or heparan sulfate/chondroitin sulfate proteoglycans). In some embodiments, the primary virus is a variant HSV and comprises a mutation of the gB or gH gene that facilitates viral entry through non-canonical receptors. In some embodiments, the primary virus is a variant HSV and comprises mutant gH glycoproteins that exhibit lateral spread in cells typically resistant to HSV lateral spread, such as cells lacking gD receptors.

In some embodiments, the primary virus is a variant HSV and comprises one or more of the mutant gB or gH proteins as described in U.S. Pat. No. 9,593,347, which is incorporated herein by reference in its entirety. Non-limiting mutations of HSV gB or gH glycoproteins include mutations at one or more of the following residues: gB:D285, gB:A549, gB:S668, gH:N753, and gH:A778. In some embodiments, the primary HSV comprises mutations at both gB:D285 and gB:A549, at both gH:N753 and gH:A778, and/or at each of gB:S668, gH:N753, and gH:A778. In some embodiments, the primary HSV comprises mutations at gB:285, gB:549, gH:753, and gH:778. In some embodiments, the primary HSV comprises one or more of the following mutations: gB:D285N, gB:A549T, gB:S668N, gH:N753K, or gH:A778V. In some embodiments, the primary HSV comprises the gB:D285N/gB:A549T double mutation, the gH:N753K/gH:A778V double mutation, or the gB:S668N/gH:N753K/gH:A778V triple mutation. In some embodiments, the primary HSV comprises gB:D285N/gB:A549T/gH:N753K/gH:A778V. The mutations are referred to herein relative to the codon (amino acid) numbering of the gD, gB, and gH genes of the HSV-1 strain KOS derivative K26GFP.

In some embodiments, the primary virus is a variant HSV and comprises one or more mutations in the UL37 gene that reduce HSV infection of neuronal cells, such as those described in International PCT Publication No. WO 2016/141320 and Richard et al., Plos Pathogens, 2017, 13(12), e1006741.

### Secondary Viruses

In some embodiments, the present disclosure provides a primary oncolytic virus or a primary virus comprising a polynucleotide encoding a secondary oncolytic virus or a secondary virus. In some embodiments, the encoded secondary oncolytic virus or the secondary virus is replication competent and are capable of infecting and killing a host cell. In some embodiments, the primary oncolytic virus or the primary virus is replication competent. In some embodiments, both the primary oncolytic virus and the secondary oncolytic virus are replication competent. In some embodiments, both the primary virus and the secondary virus are replication competent.

In some embodiments, the primary oncolytic virus or the primary virus is replication incompetent. In some embodiments, the replication incompetent primary oncolytic virus is an HSV.

In some embodiments, the secondary oncolytic virus or the secondary virus is replication incompetent. In some embodiments, the secondary oncolytic virus or the secondary virus is replication incompetent due to a deletion or mutation in the envelope protein coding region. In some embodiments, the primary oncolytic virus or the primary virus encoding the replication incompetent secondary virus comprises a coding region for the envelope protein of the secondary oncolytic virus or the secondary virus, optionally outside of coding region of the secondary oncolytic virus or the secondary virus. In some embodiments, the replication incompetent secondary oncolytic virus or secondary virus is an alphavirus.

In some embodiments, the secondary oncolytic virus or the secondary virus is an RNA virus. In some embodiments, the secondary oncolytic virus or the secondary virus is a single stranded RNA (ssRNA) virus. In some embodiments, the ssRNA virus is a positive-sense ssRNA (+sense ssRNA) virus or a negative-sense ssRNA (−sense ssRNA) virus. In some embodiments, the secondary oncolytic virus or the secondary virus is a DNA virus. In some embodiments, the secondary oncolytic virus or the secondary virus is a double-stranded RNA (dsRNA) virus or a single-stranded DNA (ssDNA) virus.

In some embodiments, the secondary oncolytic virus or the secondary virus is +sense ssRNA virus. Exemplary +sense ssRNA viruses include members of the Picornaviridae family (e.g. coxsackievirus, poliovirus, and Seneca Valley virus (SVV), including SVV-A), the Coronaviridae family (e.g., Alphacoronaviruses such as HCoV-229E and HCoV-NL63, Betacoronoaviruses such as HCoV-HKU1, HCoV-0C3, and MERS-CoV), the Retroviridae family (e.g., Murine leukemia virus), and the Togaviridae family (e.g., Sindbis virus). Additional exemplary genera of and species of positive-sense, ssRNA viruses are shown below in Table A.

TABLE A
Exemplary Positive-sense ssRNA Viruses

| Family/Subfamily | Genus | Natural Host | Species |
|---|---|---|---|
| Picornaviridae | Cardiovirus | Human | |
| | Cosavirs | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley Virus A |
| Caliciviridae | Sapovirus | Human | |
| | Norovirus | Human | |
| | Nebovirus | Bovine | |
| | Vesivirus | Felines/Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/Coronavirinae | Alphacoronavirus | | HCoV-229E |
| | | | HCoV-NL63 |
| | Betacoronavirus | | HCoV-HKUl |
| | | | HCoV-OC3 |
| | | | MERS-CoV |
| | Deltacoronavirus | | |
| | Gammacoronavirus | | |
| Coronaviridae/Torovirinae | Bafinivirus | | |
| | Torovirus | | |
| Retroviridae | Gammaretrovirus | | Murine leukemia virus |
| Togaviridae | Alphavirus | | Sindbis virus |

In some embodiments, the secondary oncolytic virus or the secondary virus is a Seneca Valley virus (SVV). In some embodiments, the viral genome of the SVV has at least 70%, at least 75%, at least 80%, at least 85%, at least 90%, at least 93%, at least 95%, at least 97%, at least 98%, at least 99%, at least 99.5%, or 100% sequence identity to SEQ ID NO: 842. In some embodiments, the secondary oncolytic virus or the secondary virus comprises a portion of the SVV viral genome having at least 70%, at least 75%, at least 80%, at least 85%, at least 90%, at least 93%, at least 95%, at least 97%, at least 98%, at least 99%, at least 99.5%, or 100% sequence identity to nucleotide 3505 to 7310 according to SEQ ID NO: 842.

In some embodiments, the secondary oncolytic virus or the secondary virus is a coxsackievirus. In some embodiments, the coxsackievirus is selected from CVB3, CVA21, and CVA9. The nucleic acid sequences of exemplary coxsackieviruses are provided GenBank Reference No. M33854.1 (CVB3), GenBank Reference No. KT161266.1 (CVA21), and GenBank Reference No. D00627.1 (CVA9). In some embodiments, the viral genome of the coxsackievirus has at least 70%, at least 75%, at least 80%, at least 85%, at least 90%, at least 93%, at least 95%, at least 97%, at least 98%, at least 99%, at least 99.5%, or 100% sequence identity to SEQ ID NO: 843. In some embodiments, the secondary oncolytic virus or the secondary virus comprises a portion of the coxsackievirus viral genome having at least 70%, at least 75%, at least 80%, at least 85%, at least 90%, at least 93%, at least 95%, at least 97%, at least 98%, at least 99%, at least 99.5%, or 100% sequence identity to nucleotide 3797 to 7435 according to SEQ ID NO: 843.

In some embodiments, the secondary oncolytic virus or the secondary virus is a chimeric virus (e.g., encode a virus comprising one portion, such as a capsid protein or an IRES, derived from a first virus and another portion, such as a non-structural gene such as a protease or polymerase, derived from a second virus). In some embodiments, the secondary oncolytic virus or the secondary virus is a chimeric picornavirus. In some embodiments, the secondary oncolytic virus or the secondary virus is a chimeric SVV. In some embodiments, the secondary oncolytic virus or the secondary virus is a chimeric coxsackievirus.

In some embodiments, the viral genome of the secondary oncolytic virus or the secondary virus comprises a microRNA (miRNA) target sequence (miR-TS) cassette, wherein the miR-TS cassette comprises one or more miRNA target sequences, and wherein expression of one or more of the corresponding miRNAs in a cell inhibits replication of the encoded oncolytic virus or the encoded virus in the cell. In some embodiments, the one or more miRNAs are selected from miR-124, miR-1, miR-143, miR-128, miR-219, miR-219a, miR-122, miR-204, miR-217, miR-137, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the viral genome of the secondary oncolytic virus or the secondary virus comprises one or more miR-TS cassettes incorporated into the 5′ untranslated region (UTR) or 3′ UTR of one or more essential viral genes. In some embodiments, the viral genome of the secondary oncolytic virus or the secondary virus comprises one or more miR-TS cassettes incorporated into the 5′ untranslated region (UTR) or 3′ UTR of one or more non-essential genes. In some embodiments, the viral genome of the secondary oncolytic virus or the secondary virus comprises one or more miR-TS cassettes incorporated 5′ or 3′ of one or more essential viral genes.

The genome of a +sense ssRNA virus comprises an ssRNA molecule in the 5′→3′ orientation which can be directly transcribed from the polynucleotide inserted into the primary oncolytic viral genome or the primary viral genome and directly translated by the host cell to produce the viral proteins. Therefore, primary oncolytic viruses or primary viruses comprising polynucleotides encoding +sense ssRNA viruses are capable of producing the genome of the secondary oncolytic virus or the secondary virus directly from the inserted polynucleotide and do not require the presence of additional viral replication proteins in order to produce the secondary oncolytic virus or the secondary virus.

In some embodiments, the polynucleotide encodes a negative-sense, single-stranded RNA (−sense ssRNA) viral genome. Exemplary −sense ssRNA viruses include members of the Paramyxoviridae family (e.g., measles virus and Newcastle Disease virus), the Rhabdoviridae family (e.g., vesicular stomatitis virus (VSV) and marba virus), the Arenaviridae family (e.g., Lassa virus), and the Orthomyxoviridae family (e.g., influenza viruses such as influenza A, influenza B, influenza C, and influenza D). Additional exemplary genera of and species of positive-sense, ssRNA viruses are shown below in Table B.

TABLE B
Exemplary Negative-sense ssRNA Viruses

| Family/Subfamily | Natural Host | Genus | Species | Virus |
|---|---|---|---|---|
| Bornaviridae | Horses, sheep, | Carbovirus | | |
| | birds, reptiles, and | | | |
| | humans | | | |
| | cattle, rodents, | Orthobornavirus | Mammalian | |
| | | | orthobornavirus | |
| Paramyxoviridae | Vertebrates | Aquaparamyxovirus | | |
| | | Avulavirus | | |
| | | Ferlavirus | | |
| | | Henipavirus | | |
| | | Morbillivirus | | |
| | | Respirovirus | Human | HPIV-1 |
| | | | respirovirus | HPIV-3 |
| | | Rubulavirus | Human | HPIV-2, |
| | | | rubulavirus | HPIV-4a, |
| | | | | HPIV-4b |
| | | | mumps virus | |
| Pneumoviridae | Humans, cattle, and | Metapneumovirus | Human | |
| | rodents | | metapneumovirus | |
| | | Orthopneumovirus | Human | HRSV-A2 |
| | | | orthopneumovirus | HRSV-B1 |
| Rhabdoviridae | Vertebrates | Cytorhabdovirus | | |
| | (mammals and | Dichorhavirus | | |
| | fish), plants, and | Ephemerovirus | | |
| | insects | Lyssavirus | | |
| | | Novirhabdovirus | | |
| | | Nucleorhabdovirus | | |
| | | Perhabdovirus | | |
| | | Sigmavirus | | |
| | | Sprivivirus | | |
| | | Tibrovirus | | |
| | | Tupavirus | | |
| | | Varicosavirus | | |
| | | Vesiculovirus | Alagoas | |
| | | | vesiculovirus | |
| | | | Carajas | |
| | | | vesiculovirus | |
| | | | Chandipura | |
| | | | vesiculovirus | |
| | | | Cocal | |
| | | | vesiculovirus | |
| | | | Indiana | VSV |
| | | | vesiculovirus | |
| | | | Isfahan | |
| | | | vesiculovirus | |
| | | | Maraba | MARAV |
| | | | vesiculovirus | |
| | | | New Jersey | |
| | | | vesiculovirus | |
| | | | Piry vesiculovirus | |
| Arenaviridae | Rodents, humans | Hartmanivirus | | |
| | | Arenavirus | Lassa virus | |
| | | Reptarenavirus | | |
| Orthomyxoviridae | | Influenza virus A | Influenza virus A | |
| | | Influenza virus B | Influenza virus B | |
| Family/Subfamily | Natural Host | Genus | Species | Virus |
| | Humans, pigs, | Influenza virus C | Influenza virus C | |
| | dogs, birds, horses, | Influenza virus D | Influenza virus D | |
| | bats | Isavirus | | |
| | | Thogotovirus | | |
| | | Quaranjavirus | | |
| Unassigned | | Deltavirus | | |

The genome of a −sense ssRNA virus comprises an ssRNA molecule in the 3′→5′ orientation which cannot be directly transcribed from the polynucleotide inserted into the primary oncolytic viral genome or the primary viral genome. Rather, the polynucleotide encoding a −sense ssRNA secondary oncolytic virus or secondary virus is first transcribed into a +sense mRNA, which is then replicated by one or more viral RNA polymerases to produce the −sense ssRNA genome. As such, in some embodiments, the polynucleotide encoding a −sense ssRNA virus inserted into a primary oncolytic virus or a primary virus comprises a first nucleic acid sequence encoding the viral proteins required for replication and a second nucleic acid sequence comprising the anti-genomic sequence of the −sense ssRNA viral genome. In such embodiments, the first nucleic acid sequence encodes a 5′→3′ mRNA transcript that can be directly translated by the host cell into the viral proteins required for replication of the −sense ssRNA genome, and the second nucleic acid sequence encodes a 5′ 4 3′ mRNA transcript of the anti-genomic sequence of the −sense ssRNA genome. The 5′ 4 3′ anti-genomic transcript is then replicated by the viral proteins encoded by the first nucleic acid sequence to produce the −sense ssRNA genome. In some embodiments, the first and second nucleic acid sequences are operably linked to a promoter capable of expression in eukaryotic cells, e.g. a mammalian promoter. In some embodiments, the first and second nucleic acid sequences are operably linked to a bidirectional promoter, such as a bi-directional Pol II promoter.

In some embodiments, the genome of the secondary +sense and/or −sense ssRNA oncolytic virus require discrete 5′ and 3′ ends that are native to the virus. In some embodiments, the genome of the secondary +sense and/or −sense ssRNA virus require discrete 5′ and 3′ ends that are native to the virus. mRNA transcripts produced by mammalian RNA Pol II contain mammalian 5′ and 3′ UTRs and therefore do not contain the discrete, native ends required for production of an infectious ssRNA virus. Therefore, in some embodiments, production of +sense and/or −sense ssRNA viruses requires additional 5′ and 3′ sequences that enable cleavage of the Pol II-encoded viral genome transcript at the junction of the viral ssRNA and the mammalian mRNA sequence such that the non-viral RNA is removed from the transcript in order to maintain the endogenous 5′ and 3′ discrete ends of the viral genome. Such sequences are referred to herein as junctional cleavage sequences. For example, in some embodiments, the polynucleotides encoding the secondary oncolytic viruses or the secondary viruses comprise the following structure:
(a) 5′-Pol II-junctional cleavage-viral genome-junctional cleavage-3′;
(b) 5′-Pol II-junctional cleavage-viral anti-genome-junctional cleavage-3′.

The junctional cleavage sequences can accomplish removal of the non-viral RNA from the viral genome transcript by a variety of methods. For example, in some embodiments, the junctional cleavage sequences are targets for RNAi molecules. Exemplary RNA interference agents include miRNAs, AmiRNAs, shRNAs, and siRNAs. Further, any system for cleaving an RNA transcript at a specific site currently known the art or to be defined the future can be used to generate the discrete ends native to the secondary oncolytic virus or the secondary virus.

In some embodiments, the RNAi molecule is an miRNA and the 5′ and/or 3′ junctional cleavage sequences are miRNA target sequences. In some embodiments, the RNAi molecule is an siRNA molecule and the 5′ and/or 3′ junctional cleavage sequences are siRNA target sequences. In some embodiments, the RNAi molecule is an AmiRNA and the 5′ and/or 3′ junctional cleavage sequences are AmiRNA target sequences.

In some embodiments, the junctional cleavage sequences are guide RNA (gRNA) target sequences. In such embodiments, gRNAs can be designed and introduced with a Cas endonuclease with RNase activity (e.g., Cas13) to mediate cleavage of the viral genome transcript at the precise junctional site. In some embodiments, the 5′ and/or 3′ junctional cleavage sequences are gRNA target sequences. In some embodiments, the junctional cleavage sequences are pri-miRNA-encoding sequences. Upon transcription of the polynucleotide encoding the secondary viral genome, these sequences form the pri-miRNA stem-loop structure which is then cleaved in the nucleus by Drosha to cleave the transcript at the precise junctional site. In some embodiments, the 5′ and/or 3′ junctional cleavage sequences are pri-mRNA target sequences.

In some embodiments, the junctional cleavage sequences are ribozyme-encoding sequences and mediate self-cleavage of the viral transcript to produce the native discrete ends of the secondary oncolytic virus or the secondary virus. In some embodiments, the 5′ and/or 3′ junctional cleavage sequences are ribozyme encoding sequences. In some embodiments, the junctional cleavage sequences are sequences aptazyme-encoding sequences. In some embodiments, the 5′ and/or 3′ junctional cleavage sequences are aptazyme-encoding sequences.

In some embodiments, the junctional cleavage sequences are target sequences for an siRNA molecule, an miRNA molecule, an AmiRNA molecule, or a gRNA molecule. In such embodiments, the target RNA molecule is at least partially complementary to the guide sequence of the RNAi or gRNA molecule. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In some embodiments, the 5′ junctional cleavage sequence and 3′ junctional cleavage sequence are from the same group (e.g., are both RNAi target sequences, both ribozyme-encoding sequences, etc.). For example, in some embodiments, the junctional cleavage sequences are RNAi target sequences (e.g., siRNA, AmiRNA, or miRNA target sequences) and are incorporated into the 5′ and 3′ ends of the polynucleotide encoding the secondary oncolytic virus or the secondary virus. In such embodiments, the 5′ and 3′ RNAi target sequence may be the same (i.e., targets for the same siRNA, AmiRNA, or miRNA) or different (i.e., the 5′ sequence is a target for one siRNA, AmiRNA, or miRNA and the 3′ sequence is a target for another siRNA, AmiRNA, or miRNA). In some embodiments, the junctional cleavage sequences are guide RNA target sequences and are incorporated into the 5′ and 3′ ends of the polynucleotide encoding the secondary oncolytic virus or the secondary virus. In such embodiments, the 5′ and 3′ gRNA target sequences may be the same (i.e., targets for the same gRNA) or different (i.e., the 5′ sequence is a target for one gRNA and the 3′ sequence is a target for another gRNA). In some embodiments, the junctional cleavage sequences are pri-mRNA-encoding sequences and are incorporated into the 5′ and 3′ ends of the polynucleotide encoding the secondary oncolytic virus or the secondary virus. In some embodiments, the junctional cleavage sequences are ribozyme-encoding sequences and are incorporated into the polynucleotide encoding the secondary oncolytic virus or the secondary virus immediately 5′ and 3′ of the polynucleotide sequence encoding the viral genome.

In some embodiments, the 5′ junctional cleavage sequence and 3′ junctional cleavage sequence are from the same group but are different variants or types. For example, in some embodiments, the 5′ and 3′ junctional cleavage sequences may be target sequences for an RNAi molecule, wherein the 5′ junctional cleavage sequence is an siRNA target sequence and the 3′ junctional cleavage sequence is a miRNA target sequence (or vis versa). In some embodiments, the 5′ and 3′ junctional cleavage sequences may be ribozyme-encoding sequences, wherein the 5′ junctional cleavage sequence is a hammerhead ribozyme-encoding sequence and the 3′ junctional cleavage sequence is a hepatitis delta virus ribozyme-encoding sequence.

In some embodiments, the 5′ junctional cleavage sequence and 3′ junctional cleavage sequence are different types. For example, in some embodiments, the 5′ junctional cleavage sequence is an RNAi target sequence (e.g., an siRNA, an AmiRNA, or a miRNA target sequence) and the 3′ junctional cleavage sequence is a ribozyme sequence, an aptazyme sequence, a pri-miRNA sequence, or a gRNA target sequence. In some embodiments, the 5′ junctional cleavage sequence is a ribozyme sequence and the 3′ junctional cleavage sequence is an RNAi target sequence (e.g., an siRNA, an AmiRNA, or a miRNA target sequence), an aptazyme sequence, a pri-miRNA-encoding sequence, or a gRNA target sequence. In some embodiments, the 5′ junctional cleavage sequence is an aptazyme sequence and the 3′ junctional cleavage sequence is an RNAi target sequence (e.g., an siRNA, an AmiRNA, or a miRNA target sequence), a ribozyme sequence, a pri-miRNA sequence, or a gRNA target sequence. In some embodiments, the 5′ junctional cleavage sequence is a pri-miRNA sequence and the 3′ junctional cleavage sequence is an RNAi target sequence (e.g., an siRNA, an AmiRNA, or a miRNA target sequence), a ribozyme sequence, an aptazyme sequence, or a gRNA target sequence. In some embodiments, the 5′ junctional cleavage sequence is a gRNA target sequence and the 3′ junctional cleavage sequence is an RNAi target sequence (e.g., an siRNA, an AmiRNA, or a miRNA target sequence), a ribozyme sequence, a pri-miRNA sequence, or an aptazyme sequence.

Exemplary arrangements of the junctional cleavage sequences relative to the polynucleotides encoding ssRNA secondary oncolytic viruses or secondary viruses are shown below in Tables C1 and C2.

TABLE Cl
Symmetrical Junctional Cleavage Sequence (JSC) Arrangements

| 5′ | JCS | | JCS | 3′ |
|---|---|---|---|---|
| | siRNA TS | 2° oV polynucleotide | siRNA TS | |
| | miR TS | 2° oV polynucleotide | miR TS | |
| | AmiR TS | 2° oV polynucleotide | AmiR TS | |
| | gRNATS | 2° oV polynucleotide | gRNATS | |
| | pri-miR | 2° oV polynucleotide | pri-miR | |
| | ribozyme | 2° oV polynucleotide | ribozyme | |
| | aptazyme | 2° oV polynucleotide | aptazyme | |

TABLE C2
Asymmetrical JCS Arrangments

| 5′ | JCS | | JCS | 3′ |
|---|---|---|---|---|
| | siRNA TS | 2° oV polynucleotide | miR TS | |
| | siRNA TS | 2° oV polynucleotide | AmiR TS | |
| | siRNA TS | 2° oV polynucleotide | gRNA TS | |
| | siRNA TS | 2° oV polynucleotide | pri-miR | |
| | siRNA TS | 2° oV polynucleotide | ribozyme | |
| | siRNA TS | 2° oV polynucleotide | aptazyme | |
| | miR TS | 2° oV polynucleotide | siRNA TS | |
| | miR TS | 2° oV polynucleotide | AmiR TS | |
| | miR TS | 2° oV polynucleotide | gRNA TS | |
| | miR TS | 2° oV polynucleotide | pri-miR | |
| | miR TS | 2° oV polynucleotide | ribozyme | |
| | miR TS | 2° oV polynucleotide | aptazyme | |
| | AmiR TS | 2° oV polynucleotide | siRNA TS | |
| | AmiR TS | 2° oV polynucleotide | miR TS | |
| | AmiR TS | 2° oV polynucleotide | gRNA TS | |
| | AmiR TS | 2° oV polynucleotide | pri-miR | |
| | AmiR TS | 2° oV polynucleotide | ribozyme | |
| | AmiR TS | 2° oV polynucleotide | aptazyme | |
| | gRNA TS | 2° oV polynucleotide | siRNA TS | |
| | gRNA TS | 2° oV polynucleotide | miR TS | |
| | gRNA TS | 2° oV polynucleotide | AmiR TS | |
| | gRNA TS | 2° oV polynucleotide | pri-miR | |
| | gRNA TS | 2° oV polynucleotide | ribozyme | |
| | gRNA TS | 2° oV polynucleotide | aptazyme | |
| | pri-miR | 2° oV polynucleotide | siRNA TS | |
| | pri-miR | 2° oV polynucleotide | miR TS | |
| | pri-miR | 2° oV polynucleotide | AmiR TS | |
| | pri-miR | 2° oV polynucleotide | gRNA TS | |
| | pri-miR | 2° oV polynucleotide | ribozyme | |
| | pri-miR | 2° oV polynucleotide | aptazyme | |
| | ribozyme | 2° oV polynucleotide | siRNA TS | |
| | ribozyme | 2° oV polynucleotide | miR TS | |
| | ribozyme | 2° oV polynucleotide | AmiR TS | |
| | ribozyme | 2° oV polynucleotide | gRNATS | |
| | ribozyme | 2° oV polynucleotide | pri-miR | |
| | ribozyme | 2° oV polynucleotide | aptazyme | |
| | aptazyme | 2° oV polynucleotide | siRNA TS | |
| | aptazyme | 2° oV polynucleotide | miR TS | |
| | aptazyme | 2° oV polynucleotide | AmiR TS | |
| | aptazyme | 2° oV polynucleotide | gRNATS | |
| | aptazyme | 2° oV polynucleotide | pri-miR | |
| | aptazyme | 2° oV polynucleotide | ribozyme | |

In some embodiments, the secondary oncolytic viruses or the secondary viruses further comprise one or more internal RNAi target sequences to prevent expression of the secondary viral genome in a cell or a subject. In some embodiments, the internal RNAi target sequence is a target sequence for an siRNA molecule, an AmiRNA molecule, or an miRNA molecule. In some embodiments, the internal RNAi sequences enable further temporal control over the expression of the secondary oncolytic virus or the secondary virus after introduction of the viral construct to a cell or administration to a subject. In such embodiments, the internal RNAi target sequence is an miRNA target sequence for an miRNA that is endogenously expressed by a cell. In such embodiments, the secondary oncolytic virus or the secondary virus is miRNA-attenuated.

In some embodiments, the internal RNAi sequences enable control over the expression of the secondary oncolytic virus or the secondary virus during production of dual viral vector. In some embodiments, the internal RNAi target sequence is a target sequence for an siRNA molecule, an AmiRNA molecule, or an artificial miRNA molecule that is not endogenously expressed by the production cell line or by a cell in a sample or subject.

miRNA-Attenuation

In some embodiments, the primary and/or secondary viruses comprise one or more of copies of a miRNA target sequence inserted into the locus of one or more essential viral genes. In some embodiments, the primary and/or secondary oncolytic viruses comprise one or more of copies of a miRNA target sequence inserted into the locus of one or more essential viral genes. miRs are differentially expressed in a broad array of disease states, including multiple types of cancer. Importantly, miRNAs are differentially expressed in cancer tissues compared to normal tissues, enabling them to serve as a targeting mechanism in a broad variety of cancers. miRNAs that are associated (either positively or negatively) with carcinogenesis, malignant transformation, or metastasis are known as “oncomiRs”. Examples of oncomiRs and their relation to different cancers are known in the art (See e.g., International PCT Publication No. WO 2017/132552, incorporated herein by reference).

In some embodiments, the primary and/or secondary viruses, or the primary and/or secondary oncolytic viruses, may comprise miRNA target sequences inserted into the locus of at least one, at least two, at least three, at least four, at least five, at least six, at least seven, at least eight, at least nine, or at least ten essential viral genes. miRNAs expressed in normal (non-cancerous) cells can bind to such target sequences and suppress expression of the viral gene containing the miRNA target sequence. By incorporating miRNA target sequences into key genes required for viral replication, viral replication can be conditionally suppressed in normal diploid cells expressing the miRNAs and can proceed normally in cells that do not express the miRNAs. In such embodiments, healthy, non-cancerous cells are protected from the normal cells from lytic effects of infection by the recombinant viral vector. Such recombinant viruses or oncolytic viruses are referred to herein as “miR-attenuated” as they demonstrate reduced or attenuated viral replication in cells that express one or more miRNAs capable of binding to the incorporated miRNA target sequences compared to cells that do not express, or have reduced expression of, the miRNA.

In some aspects, the expression level of a particular oncomiR is positively associated with the development or maintenance of a particular cancer. Such miRs are referred to herein as “oncogenic miRs.” In some embodiments, the expression of an oncogenic miR is increased in cancerous cells or tissues compared to the expression level observed in non-cancerous controls cells (i.e., normal or healthy controls) or is increased compared to the expression level observed in cancerous cells derived from a different cancer type.

In some embodiments, the expression of a particular oncomiR is negatively associated with the development or maintenance of a particular cancer and/or metastasis. Such oncomiRs are referred to herein as “tumor-suppressor miRs” or “tumor-suppressive miRs,” as their expression prevents or suppresses the development of cancer. In some embodiments, the expression of a tumor-suppressor miRNA is decreased in cancerous cells or tissues compared to the expression level observed in non-cancerous control cells (i.e., normal or healthy controls), or is decreased compared to the expression level of the tumor-suppressor miRNA observed in cancerous cells derived from a different cancer type.

In some embodiments, the primary and/or secondary viruses, or the primary and/or secondary oncolytic viruses, comprise one or more miRNA target sequences that are at least 95%, at least 96%, at least 97%, at least 98%, or at least 99% identical to the reverse complement of a sequence selected from SEQ ID NOs: 1-803. In some embodiments, the primary and/or secondary viruses, or the primary and/or secondary oncolytic viruses, comprise one or more miRNA target sequences that comprise or consist of the reverse complement of a sequence selected from SEQ ID NOs: 1-803.

In some embodiments, the miRNA target sequences are inserted into the locus of one or more essential viral genes in the form of a “miR target sequence cassette” or “miR-TS cassette”. A miR-TS cassette refers to a polynucleotide sequence comprising one or more miRNA target sequences and capable of being inserted into a specific locus of a viral gene. In some embodiments, the miR-TS cassettes described herein comprise at least one miRNA target sequence. In some embodiments, the miR-TS cassettes described herein comprise a plurality of miRNA target sequences. For example, in some embodiments, the miR-TS cassettes described herein comprise 2, 3, 4, 5, 6, 7, 8, 9, 10, 11, 12, 13, 14, 15, 16, 17, 18, 19, 20, or more miRNA target sequences.

In some embodiments, the miR-TS cassettes comprise a plurality miRNA target sequences, wherein each miRNA target sequence in the plurality is a target sequence for the same miRNA. For example, the miR-TS cassettes may comprise 2, 3, 4, 5, 6, 7, 8, 9, 10 or more copies of the same miRNA target sequence. In some embodiments, the miR-TS cassettes comprise between 2 to 6 copies of the same miRNA target sequence. In some embodiments, the miR-TS cassettes comprise 3 copies of the same miRNA target sequence. In some embodiments, the miR-TS cassettes comprise 4 copies of the same miRNA target sequence.

In some embodiments, the miR-TS cassettes described herein comprise a plurality of miRNA target sequences, wherein the plurality comprises target sequences that are specific for at least two different miRNAs. For example, in some embodiments, the miR-TS cassette comprises one or more copies of a first miRNA target sequence and one or more copies of a second miRNA target sequence. In some embodiments, the miR-TS cassette comprises one or more copies of a first miRNA target sequence, one or more copies of a second miRNA target sequence, and one or more copies of a third miRNA target sequence. In some embodiments, the miR-TS cassette comprises at least 2, 3, 4, 5, 6, 7, 8, 9, 10, or more copies of a first miRNA target sequence, at least 2, 3, 4, 5, 6, 7, 8, 9, 10, or more copies of a second miRNA target sequence, and at least 2, 3, 4, 5, 6, 7, 8, 9, 10, or more copies of a third miRNA target sequence. In some embodiments, the miR-TS cassette comprises 3 or 4 copies of a first miRNA target sequence, 3 or 4 copies of a second miRNA target sequence, and 3 or 4 copies of a third miRNA target sequence. In some embodiments, the plurality of miRNA target sequences comprises at least 4 different miRNA target sequences. For example, in some embodiments, the miR-TS cassette comprises one or more copies of a first miRNA target sequence, one or more copies of a second miRNA target sequence, one or more copies of a third miRNA target sequence, and one or more copies of a fourth miRNA target sequence. In some embodiments, the miR-TS cassette comprises at least 2, 3, 4, 5, 6, 7, 8, 9, 10, or more copies of a first miRNA target sequence, at least 2, 3, 4, 5, 6, 7, 8, 9, 10, or more copies of a second miR target sequence, at least 2, 3, 4, 5, 6, 7, 8, 9, 10, or more copies of a third miR target sequence, and at least 2, 3, 4, 5, 6, 7, 8, 9, 10, or more copies of a fourth miR target sequence. In some embodiments, the miR-TS cassette comprises 3 or 4 copies of a first miR target sequence, 3 or 4 copies of a second miR target sequence, 3 or 4 copies of a third miR target sequence, and 3 or 4 copies of a fourth miR target sequence.

In some embodiments, the plurality of miRNA target sequences in a miR-TS cassette are interleaved rather than in tandem to one another. In some embodiments, plurality of miRNA target sequences in the miR-TS cassettes are separated by short (e.g., 4-15 nt in length) spacers, resulting in a more compact cassette. In some embodiments, the miR-TS cassettes are free from (or have reduced) RNA secondary structures that inhibit activity of the plurality of miRNA target sequences. In some embodiments, the miR-TS cassettes are free from (or have reduced) seed sequences for miRNAs associated with carcinogenesis, malignant transformation, or metastasis. In some embodiments, the miR-TS cassettes are free from (or have reduced) polyadenylation sites.

In some embodiments, the miR-TS cassettes comprise one or more additional polynucleotide sequences that enable the cassette to be inserted into the locus in the primary and/or secondary viral genomes. For example, a miR-TS cassette may further comprise short polynucleotide sequence on the 5′ and 3′ ends that are complementary to a nucleic acid sequence at a desired location in the viral genome. Such sequences are referred to herein as “homology arms” and facilitate the insertion of a miR-TS cassette into a specific location in the primary and/or secondary viral genomes.

In some embodiments, the primary viral genome comprises at least one miR-TS cassette. In some embodiments, the primary viral genome comprises two or more miR-TS cassettes. In some embodiments, the primary viral genome comprises three or more miR-TS cassettes. In some embodiments, the primary viral genome comprises four or more miR-TS cassettes. In some embodiments, the primary viral genome comprises 5, 6, 7, 8, 9, 10 or more miR-TS cassettes. In some embodiments, the secondary viral genome comprises at least one miR-TS cassette. In some embodiments, the secondary viral genome comprises two or more miR-TS cassettes. In some embodiments, the secondary viral genome comprises three or more miR-TS cassettes. In some embodiments, the secondary viral genome comprises four or more miR-TS cassettes. In some embodiments, the secondary viral genome comprises 5, 6, 7, 8, 9, 10 or more miR-TS cassettes.

In some embodiments, the primary viral genome comprises at least one miR-TS cassette and the secondary viral genome comprises at least one miR-TS cassette. In some embodiments, the primary viral genome comprises at least one miR-TS cassette and the secondary viral genome comprises two or more miR-TS cassettes. In some embodiments, the primary viral genome comprises at least one miR-TS cassette and the secondary viral genome comprises three or more miR-TS cassettes. In some embodiments, the primary viral genome comprises at least one miR-TS cassette and the secondary viral genome comprises four or more miR-TS cassettes. In some embodiments, the primary viral genome comprises at least one miR-TS cassette and the secondary viral genome comprises 5, 6, 7, 8, 9, 10 or more miR-TS cassettes. In some embodiments, the primary viral genome comprises two or more miR-TS cassettes and the secondary viral genome comprises at least one miR-TS cassette. In some embodiments, the primary viral genome comprises three or more miR-TS cassettes and the secondary viral genome comprises at least one miR-TS cassette. In some embodiments, the primary viral genome comprises four or more miR-TS cassettes and the secondary viral genome comprises at least one miR-TS cassette. In some embodiments, the primary viral genome comprises 5, 6, 7, 8, 9, 10 or more miR-TS cassettes and the secondary viral genome comprises at least one miR-TS cassette.

In some embodiments, the primary viral genome comprises at least two miR-TS cassettes and the secondary viral genome comprises at least two miR-TS cassettes. In some embodiments, the primary viral genome comprises at least three miR-TS cassettes and the secondary viral genome comprises at least two miR-TS cassettes. In some embodiments, the primary viral genome comprises at least four miR-TS cassettes and the secondary viral genome comprises at least two miR-TS cassettes. In some embodiments, the primary viral genome comprises 5, 6, 7, 8, 9, 10 or more miR-TS cassettes and the secondary viral genome comprises at least two miR-TS cassettes.

Table D below provides sequences of exemplary miRNAs that can bind to the miRNA target sequences in the primary and/or secondary viruses, or the primary and/or secondary oncolytic viruses. Additional miRNA sequences are provided in SEQ ID NOs: 33-803. In some embodiments, miR-TS cassettes described herein comprise one or more miRNA target sequences that are at least 95%, at least 96%, at least 97%, at least 98%, or at least 99% identical to the reverse complement of a sequence selected from SEQ ID NOs: 1-803. In some embodiments, miR-TS cassettes described herein comprise one or more miRNA target sequences that comprise or consist of the reverse complement of a sequence selected from SEQ ID NOs: 1-803.

TABLE D
Exemplary miRNAs and Target Sequences

| | | SEQ | | SEQ |
|---|---|---|---|---|
| miRNA | miRNA Sequence | ID: | miR-TS | ID: |
| 122-5p | UGGAGUGUGACAAUGGUGUUUG | 1 | CAAACACCATTGTCACACTCCA | 804 |
| 124-3p | UAAGGCACGCGGUGAAUGCC | 2 | GGCATTCACCGCGTGCCTTA | 805 |
| 125a-5p | UCCCUGAGACCCUUUAACCUGUGA | 3 | TCACAGGTTAAAGGGTCTCAGGGA | 806 |
| 126-3p | UCGUACCGUGAGUAAUAAUGCG | 4 | CGCATTATTACTCACGGTACGA | 807 |
| | | | CACATTATTACTCACGGTACGA | 808 |
| 127a-3p | UCGGAUCCGUCUGAGCUUGGCU | 5 | AGCCAAGCTCAGACGGATCCGA | 809 |
| 128-3p | UCACAGUGAACCGGUCUCUUU | 6 | AAAGAGACCGGTTCACTGTGA | 810 |
| | | | AAAGAGACCGGTTCACTGTGG | 811 |
| 129-3p | AAGCCCUUACCCCAAAAAGUAU | 7 | ATACTTTTTGGGGTAAGGGCTT | 812 |
| 129-5p | CUUUUUGCGGUCUGGGCUUGC | 8 | GCAAGCCCAGACCGCAAAAAG | 813 |
| 130b-3p | CAGUGCAAUGAUGAAAGGGCAU | 9 | ATGCCCTTTCATCATTGCACTG | 814 |
| 130b-5p | ACUCUUUCCCUGUUGCACUAC | 10 | GTAGTGCAACAGGGAAAGAGT | 815 |
| 133a-3p | UUUGGUCCCCUUCAACCAGCUG | 11 | CAGCTGGTTGAAGGGGACCAAA | 816 |
| 133b-3p | UUUGGUCCCCUUCAACCAGCUA | 12 | TAGCTGGTTGAAGGGGACCAAA | 817 |
| 134-3p | CCUGUGGGCCACCUAGUCACCAA | 13 | TTGGTGACTAGGTGGCCCACAGG | 818 |
| 137-3p | UUAUUGCUUAAGAAUACGCGUAG | 14 | CTACGCGTATTCTTAAGCAATAA | 819 |
| 1-3p | UGGAAUGUAAAGAAGUAUGUAU | 15 | ATACATACTTCTTTACATTCCA | 820 |
| 143-3p | UGAGAUGAAGCACUGUAGCUC | 16 | GAGCTACAGTGCTTCATCTCA | 821 |
| 145-3p | GGAUUCCUGGAAAUACUGUUCU | 17 | AGAACAGTATTTCCAGGAATCC | 822 |
| 145-5p | GUCCAGUUUUCCCAGGAAUCCCU | 18 | AGGGATTCCTGGGAAAACTGGAC | 823 |
| 184-3p | UGGACGGAGAACUGAUAAGGGU | 19 | ACCCTTATCAGTTCTCCGTCCA | 824 |
| 199a-3p | ACAGUAGUCUGCACAUUGGUUA | 20 | TAACCAATGTGCAGACTACTGT | 825 |
| 199a-5p | CCCAGUGUUCAGACUACCUGUUC | 21 | GAACAGGTAGTCTGAACACTGGG | 826 |
| 204-5p | UUCCCUUUGUCAUCCUAUGCCU | 22 | AGGCATAGGATGACAAAGGGAA | 827 |
| 208b-3p | AUAAGACGAACAAAAGGUUUGU | 23 | ACAAACCTTTTGTTCGTCTTAT | 828 |
| 214-3p | ACAGCAGGCACAGACAGGCAGU | 24 | ACTGCCTGTCTGTGCCTGCTGT | 829 |
| 217-5p | UACUGCAUCAGGAACUGAUUGGA | 25 | TCCAATCAGTTCCTGATGCAGTA | 830 |
| 219a-5p | UGAUUGUCCAAACGCAAUUCU | 26 | AGAATTGCGTTTGGACAATCA | 831 |
| 223-3p | UGUCAGUUUGUCAAAUACCCCA | 27 | TGGGGTATTTGACAAACTGACA | 832 |
| 34a-5p | UGGCAGUGUCUUAGCUGGUUGU | 28 | ACAACCAGCTAAGACACTGCCA | 833 |
| 451a | AAACCGUUACCAUUACUGAGUU | 29 | AACTCAGTAATGGTAACGGTTT | 834 |
| 559-5p | UAAAGUAAAUAUGCACCAAAA | 30 | TTTTGGTGCATATTTACTTTA | 835 |
| Let7a- | UGAGGUAGUAGGUUGUAUAGUU | 31 | AACTATACAACCTACTACCTCA | 836 |
| 5p | | | | |
| 9-5p | UCUUUGGUUAUCUAGCUGUAUGA | 32 | TCATACAGCTAGATAACCAAAGA | 837 |

In some embodiments, the miR-TS cassettes comprise a plurality of miRNA target sequences, wherein the plurality comprises target sequences that are specific for at least two different miRNAs and are selected to protect diverse cell types or organs from viral-mediated cell death. For example, in some embodiments, target sequences for miRNAs that are highly expressed in various types normal non-cancerous cells and are not expressed or are expressed at low levels in cancerous cells are incorporated into an miR-TS cassette (and into the primary and/or secondary viral genome) to prevent viral replication in normal cells while allowing viral replication in cancerous cells.

In some embodiments, the primary and/or secondary virus comprises a first and a second miR-TS cassette, each comprising a plurality of miRNA target sequences. In some embodiments, the first miR-TS cassette comprises one or more copies of a target sequence for miR-124-3p, miR-1-3p, and/or miR-143-3p. In some embodiments, the second miR-TS cassette comprises one or more copies of a target sequence for miR-1-3p, miR-145-5p, miR-199-5p, and/or miR-559. In some embodiments, the second miR-TS cassette comprises one or more copies of a target sequence for miR-219a-5p, miR-122-5p, and/or miR-128-3p. In some embodiments, the second miR-TS cassette comprises one or more copies of a target sequence for miR-122-5p. In some embodiments, the second miR-TS cassette comprises one or more copies of a target sequence for miR-137-3p, miR-208b-3p, and/or miR-126-3p.

In some embodiments, the primary and/or secondary virus comprises a first, a second, and a third miR-TS cassette, each comprising a plurality of miRNA target sequences. In some embodiments, the first miR-TS cassette comprises one or more copies of a target sequence for miR-124-3p, miR-1-3p, and/or miR-143-3p. In some embodiments, the second miR-TS cassette comprises one or more copies of a target sequence for miR-122-3p. In some embodiments, the second miR-TS cassette comprises one or more copies of a target sequence for miR-219a-5p, miR-122-5p, and/or miR-128-3p. In some embodiments, the third miR-TS cassette comprises one or more copies of a target sequence for miR-125-5p. In some embodiments, the third miR-TS cassette comprises one or more copies of a target sequence for miR-137-3p, miR-208b-3p, and/or miR-126-3p.

Exemplary miR-TS cassettes are provided below in Table E. *N or N₁₋₂₀ denotes a linker sequence that may vary in length from between one nucleotide and twenty nucleotides wherein “N” may be any nucleic acid. In some embodiments, the linker sequences are between 1 and 20 nucleic acids. In some embodiments, the linker sequences are between 1 and 8 nucleic acids. In some embodiments, the linker sequences are 1, 2, 3, 4, 5, 6, 7, or 8 nucleic acids. In some embodiments, the linker sequences are 4 nucleic acids. miR-TS cassettes may comprise miRNA TS sequences having at least 70%, 75%, 80%, 85%, 90%, 95%, 97%, 99%, 100% or any percentage in between of identity with one or more sequences shown in Table E. miR-TS cassettes may comprise miRNA TS sequences having at least 70%, 75%, 80%, 85%, 90%, 95%, 97%, 99%, 100% or any percentage in between of identity with one or more sequences shown in Table E, wherein the percentage identity within a seed region is 100%. In some embodiments, the seed region may comprise nucleotides at positions 1-8 of the miRNA TS sequence or complement or a reverse complement thereof.

TABLE E
Exemplary miRNA TS Designs

| miRNA TS | Arrangement | Sequence | SEQ ID: |
|---|---|---|---|
| 124-3p (4x) | (124-3p)-(124-3p)- | | N/A |
| | (124-3p)-(124-3p) | | |
| 1-3p (4x) | (1-3p)-(143-3p)-(1- | CCATATACATACTTCTTTACATTCCAT | 838 |
| 143-3p (4x) | 3p)-(143-3p)-(1- | CCTGAGCTACAGTGCTTCATCTCATTG | |
| | 3p)-(143-3p)-(1- | CATACATACTTCTTTACATTCCAACGT | |
| | 3p)-(143-3p) | GAGCTACAGTGCTTCATCTCATCCGAT | |
| | | ACATACTTCTTTACATTCCACGGCGAG | |
| | | CTACAGTGCTTCATCTCACCTTATACA | |
| | | TACTTCTTTACATTCCAAAAAGAGCTA | |
| | | CAGTGCTTCATCTCACCAT | |
| 1-3p (4x) | (124-3p)-(124-3p)- | | N/A |
| 124-3p (4x) | (124-3p)-(124-3p)- | | |
| 143-3p (4x) | (1-3p)-(143-3p)-(1- | | |
| | 3p)-(143-3p)-(1- | | |
| | 3p)-(143-3p)-(1- | | |
| | 3p)-(143-3p) | | |
| 122-5p (4x) | (219a-5p)-(122-5p)- | CACGAGAATTGCGTTTGGACAATCAG | 839 |
| 128-3p (4x) | (128-3p)-(122-5p)- | ACACAAACACCATTGTCACACTCCAT | |
| 219a-5p (4x) | (219a-5p)-(128-3p)- | CTTAAAGAGACCGGTTCACTGTGGAT | |
| | (122-5p)-(128-3p)- | GTCAAACACCATTGTCACACTCCAAC | |
| | (219a-5p)-(128-3p)- | TTAGAATTGCGTTTGGACAATCAAGG | |
| | (122-5p)-(219a-5p) | GAAAGAGACCGGTTCACTGTGGCCAG | |
| | | CAAACACCATTGTCACACTCCAAAAC | |
| | | AAAGAGACCGGTTCACTGTGGTACGA | |
| | | GAATTGCGTTTGGACAATCAGAAAAA | |
| | | AGAGACCGGTTCACTGTGGAATACAA | |
| | | ACACCATTGTCACACTCCAACAAAGA | |
| | | ATTGCGTTTGGACAATCAGGTT | |
| 128-3p (4x) | (128-3p)-(219a-5p)- | AAGTAAAGAGACCGGTTCACTGTGGA | 840 |
| 204-5p (4x) | (204-5p)-(128-3p)- | ATAAGAATTGCGTTTGGACAATCAAG | |
| 219a-5p (4x) | (219a-5p)-(204-5p)- | GTAGGCATAGGATGACAAAGGGAAC | |
| | (128-3p)-(219a-5p)- | AGCAAAGAGACCGGTTCACTGTGGGG | |
| | (204-5p)-(128-3p)- | CTAGAATTGCGTTTGGACAATCACGT | |
| | (219a-5p)-(204-5p) | AAGGCATAGGATGACAAAGGGAACG | |
| | | AGAAAGAGACCGGTTCACTGTGGGGG | |
| | | AAGAATTGCGTTTGGACAATCATACT | |
| | | AGGCATAGGATGACAAAGGGAATTAG | |
| | | AAAGAGACCGGTTCACTGTGGATTTA | |
| | | GAATTGCGTTTGGACAATCATAGAAG | |
| | | GCATAGGATGACAAAGGGAATTGT | |
| 126-3p (4x) | (208b-3p)-(126-3p)- | TATGCTACGCGTATTCTTAAGCAATAA | 841 |
| 137-3p (4x) | (137-3p)-(208b-3p)- | GACTTCCAATCAGTTCCTGATGCAGTA | |
| 208b-3p (4x) | (137-3p)-(126-3p)- | CGACCACATTATTACTCACGGTACGA | |
| | (208b-3p)-(137-3p)- | AAGCCTACGCGTATTCTTAAGCAATA | |
| | (126-3p)-(137-3p)- | ACCGCCACATTATTACTCACGGTACG | |
| | (126-3p)-(208b-3p) | ATAAATCCAATCAGTTCCTGATGCAG | |
| | | TAATTACTACGCGTATTCTTAAGCAAT | |
| | | AACTATTCCAATCAGTTCCTGATGCAG | |
| | | TACCCCCACATTATTACTCACGGTACG | |
| | | AGAATTCCAATCAGTTCCTGATGCAG | |
| | | TACAGTCACATTATTACTCACGGTACG | |
| | | ATCAACTACGCGTATTCTTAAGCAAT | |
| | | AACCAA | |
| 126-3p (4x) | (137-3p)-(126-3p)- | | N/A |
| 137-3p (4x) | (217-5p)-(126-3p)- | | |
| 217-5p (4x) | (217-5p)-(137-3p)- | | |
| | (217-5p)-(126-3p)- | | |
| | (137-3p)-(126-3p)- | | |
| | (217-5p)-(137-3p) | | |
| 127-3p (4x) | (137-3p)-(127-3p)- | | N/A |
| 137-3p (4x) | (217-5p)-(127-3p)- | | |
| 217-5p (4x) | (217-5p)-(137-3p)- | | |
| | (217-5p)-(127-3p)- | | |
| | (137-3p)-(127-3p)- | | |
| | (217-5p)-(137-3p) | | |
| 128-3p (4x) | (137-3p)-(128-3p)- | | N/A |
| 137-3p (4x) | (217-5p)-(128-3p)- | | |
| 217-5p (4x) | (217-5p)-(137-3p)- | | |
| | (217-5p)-(128-3p)- | | |
| | (137-3p)-(128-3p)- | | |
| | (217-5p)-(137-3p) | | |
| 129-3p (4x) | (137-3p)-(129-3p)- | | N/A |
| 137-3p (4x) | (217-5p)-(129-3p)- | | |
| 217-5p (4x) | (217-5p)-(137-3p)- | | |
| | (217-5p)-(129-3p)- | | |
| | (137-3p)-(129-3p)- | | |
| | (217-5p)-(137-3p) | | |
| 130-3p (4x) | (137-3p)-(130-3p)- | | N/A |
| 137-3p (4x) | (217-5p)-(130-3p)- | | |
| 217-5p (4x) | (217-5p)-(130-3p)- | | |
| | (217-5p)-(127-3p)- | | |
| | (137-3p)-(130-3p)- | | |
| | (217-5p)-(137-3p) | | |
| 124-3p (4x) | (124-3p)-(124-3p)- | GGCATTCACCGCGTGCCTTAN₁₋ | 902 |
| | (124-3p)-(124-3p) | ₂₀GGCATTCACCGCGTGCCTTAN₁₋ | |
| | | ₂₀GGCATTCACCGCGTGCCTTAN₁₋ | |
| | | ₂₀GGCATTCACCGCGTGCCTTA | |
| 1-3p (4x) | (1-3p)-(143-3p)-(1- | ATACATACTTCTTTACATTCCAN₁₋ | 903 |
| 143-3p (4x) | 3p)-(143-3p)-(1- | ₂₀GAGCTACAGTGCTTCATCTCAN₁₋ | |
| | 3p)-(143-3p)-(1- | ₂₀ATACATACTTCTTTACATTCCAN₁₋ | |
| | 3p)-(143-3p) | ₂₀GAGCTACAGTGCTTCATCTCAN₁₋ | |
| | | ₂₀ATACATACTTCTTTACATTCCAN₁₋ | |
| | | ₂₀GAGCTACAGTGCTTCATCTCAN₁₋ | |
| | | ₂₀ATACATACTTCTTTACATTCCAN₁₋ | |
| | | ₂₀GAGCTACAGTGCTTCATCTCA | |
| 1-3p (4x) | (124-3p)-(124-3p)- | GGCATTCACCGCGTGCCITAN₁₋ | 904 |
| 124-3p (4x) | (124-3p)-(124-3p)- | ₂₀GGCATTCACCGCGTGCCTTAN₁₋ | |
| 143-3p (4x) | (1-3p)-(143-3p)-(1- | ₂₀GGCATTCACCGCGTGCCTTAN₁₋ | |
| | 3p)-(143-3p)-(1- | ₂₀GGCATTCACCGCGTGCCTTAN₁₋ | |
| | 3p)-(143-3p)-(1- | ₂₀ATACATACTTCTTTACATTCCAN₁₋ | |
| | 3p)-(143-3p) | ₂₀GAGCTACAGTGCTTCATCTCAN₁₋ | |
| | | ₂₀ATACATACTTCTTTACATTCCAN₁₋ | |
| | | ₂₀GAGCTACAGTGCTTCATCTCAN₁₋ | |
| | | ₂₀ATACATACTTCTTTACATTCCAN₁₋ | |
| | | ₂₀GAGCTACAGTGCTTCATCTCAN₁₋ | |
| | | ₂₀ATACATACTTCTTTACATTCCAN₁₋ | |
| | | ₂₀GAGCTACAGTGCTTCATCTCA | |
| 122-5p (4x) | (219a-5p)-(122-5p)- | AGAATTGCGTTTGGACAATCAN₁₋ | 905 |
| 128-3p (4x) | (128-3p)-(122-5p)- | ₂₀CAAACACCATTGTCACACTCCAN₁₋ | |
| 219a-5p (4x) | (219a-5p)-(128-3p)- | ₂₀AAAGAGACCGGTTCACTGTGRN₁₋ | |
| | (122-5p)-(128-3p)- | ₂₀CAAACACCATTGTCACACTCCAN₁₋ | |
| | (219a-5p)-(128-3p)- | ₂₀AGAATTGCGTTTGGACAATCAN₁₋ | |
| | (122-5p)-(219a-5p) | ₂₀AAAGAGACCGGTTCACTGTGRN₁₋ | |
| | | ₂₀CAAACACCATTGTCACACTCCAN₁₋ | |
| | | ₂₀AAAGAGACCGGTTCACTGTGRN₁₋ | |
| | | ₂₀AGAATTGCGTTTGGACAATCAN₁₋ | |
| | | ₂₀AAAGAGACCGGTTCACTGTGRN₁₋ | |
| | | ₂₀CAAACACCATTGTCACACTCCA₁₋ | |
| | | ₂₀AGAATTGCGTTTGGACAATCA | |
| 128-3p (4x) | (128-3p)-(219a-5p)- | AAAGAGACCGGTTCACTGTGRN₁₋ | 906 |
| 204-5p (4x) | (204-5p)-(128-3p)- | ₂₀AGAATTGCGTTTGGACAATCAN₁₋ | |
| 219a-5p (4x) | (219a-5p)-(204-5p)- | ₂₀AGGCATAGGATGACAAAGGGAAN₁₋ | |
| | (128-3p)-(219a-5p)- | ₂₀AAAGAGACCGGTTCACTGTGRN₁₋ | |
| | (204-5p)-(128-3p)- | ₂₀AGAATTGCGTTTGGACAATCAN₁₋ | |
| | (219a-5p)-(204-5p) | ₂₀AGGCATAGGATGACAAAGGGAAN₁₋ | |
| | | ₂₀AAAGAGACCGGTTCACTGTGRN₁₋ | |
| | | ₂₀AGAATTGCGTTTGGACAATCAN₁₋ | |
| | | ₂₀AGGCATAGGATGACAAAGGGAAN₁₋ | |
| | | ₂₀AAAGAGACCGGTTCACTGTGRN₁₋ | |
| | | ₂₀AGAATTGCGTTTGGACAATCAN₁₋ | |
| | | ₂₀AGGCATAGGATGACAAAGGGAA | |
| 126-3p (4x) | (208b-3p)-(126-3p)- | ACAAACCTTTTGTTCGTCTTATN₁₋ | 907 |
| 137-3p (4x) | (137-3p)-(208b-3p)- | ₂₀CRCATTATTACTCACGGTACGAN₁₋ | |
| 208b-3p (4x) | (137-3p)-(126-3p)- | ₂₀CTACGCGTATTCTTAAGCAATAAN₁₋ | |
| | (208b-3p)-(137-3p)- | ₂₀ACAAACCTTTTGTTCGTCTTATN₁₋ | |
| | (126-3p)-(137-3p)- | ₂₀CTACGCGTATTCTTAAGCAATAAN₁₋ | |
| | (126-3p)-(208b-3p) | ₂₀CRCATTATTACTCACGGTACGAN₁₋ | |
| | | ₂₀ACAAACCTTTTGTTCGTCTTATN₁₋ | |
| | | ₂₀CTACGCGTATTCTTAAGCAATAAN₁₋ | |
| | | ₂₀CRCATTATTACTCACGGTACGAN₁₋ | |
| | | ₂₀CTACGCGTATTCTTAAGCAATAAN₁₋ | |
| | | ₂₀CRCATTATTACTCACGGTACGAN₁₋ | |
| | | ₂₀ACAAACCTTTTGTTCGTCTTAT | |
| 126-3p (4x) | (137-3p)-(126-3p)- | CTACGCGTATTCTTAAGCAATAAN₁₋ | 908 |
| 137-3p (4x) | (217-5p)-(126-3p)- | ₂₀CRCATTATTACTCACGGTACGAN₁₋ | |
| 217-5p (4x) | (217-5p)-(137-3p)- | ₂₀TCCAATCAGTTCCTGATGCAGTAN₁₋ | |
| | (217-5p)-(126-3p)- | ₂₀CRCATTATTACTCACGGTACGAN₁₋ | |
| | (137-3p)-(126-3p)- | ₂₀TCCAATCAGTTCCTGATGCAGTAN₁₋ | |
| | (217-5p)-(137-3p) | ₂₀CTACGCGTATTCTTAAGCAATAAN₁₋ | |
| | | ₂₀TCCAATCAGTTCCTGATGCAGTAN₁₋ | |
| | | ₂₀CRCATTATTACTCACGGTACGAN₁₋ | |
| | | ₂₀CTACGCGTATTCTTAAGCAATAAN₁₋ | |
| | | ₂₀CRCATTATTACTCACGGTACGAN₁₋ | |
| | | ₂₀TCCAATCAGTTCCTGATGCAGTAN₁₋ | |
| | | ₂₀CTACGCGTATTCTTAAGCAATAA | |
| 127-3p (4x) | (137-3p)-(127-3p)- | CTACGCGTATTCTTAAGCAATAAN₁₋ | 909 |
| 137-3p (4x) | (217-5p)-(127-3p)- | ₂₀(AGCCAAGCTCAGACGGATCCGA)N₁₋ | |
| 217-5p (4x) | (217-5p)-(137-3p)- | ₂₀TCCAATCAGTTCCTGATGCAGTAN₁₋ | |
| | (217-5p)-(127-3p)- | ₂₀(AGCCAAGCTCAGACGGATCCGA)N₁₋ | |
| | (137-3p)-(127-3p)- | ₂₀TCCAATCAGTTCCTGATGCAGTAN₁₋ | |
| | (217-5p)-(137-3p) | ₂₀CTACGCGTATTCTTAAGCAATAAN₁₋ | |
| | | ₂₀TCCAATCAGTTCCTGATGCAGTAN₁₋ | |
| | | ₂₀(AGCCAAGCTCAGACGGATCCGA)N₁₋ | |
| | | ₂₀CTACGCGTATTCTTAAGCAATAAN₁₋ | |
| | | ₂₀(AGCCAAGCTCAGACGGATCCGA)N₁₋ | |
| | | ₂₀TCCAATCAGTTCCTGATGCAGTAN₁₋ | |
| | | ₂₀CTACGCGTATTCTTAAGCAATAA | |
| 128-3p (4x) | (137-3p)-(128-3p)- | CTACGCGTATTCTTAAGCAATAAN₁₋ | 910 |
| 137-3p (4x) | (217-5p)-(128-3p)- | ₂₀AAAGAGACCGGTTCACTGTGRN₁₋ | |
| 217-5p (4x) | (217-5p)-(137-3p)- | ₂₀TCCAATCAGTTCCTGATGCAGTAN₁₋ | |
| | (217-5p)-(128-3p)- | ₂₀AAAGAGACCGGTTCACTGTGRN₁₋ | |
| | (137-3p)-(128-3p)- | ₂₀TCCAATCAGTTCCTGATGCAGTAN₁₋ | |
| | (217-5p)-(137-3p) | ₂₀CTACGCGTATTCTTAAGCAATAAN₁₋ | |
| | | ₂₀TCCAATCAGTTCCTGATGCAGTAN₁₋ | |
| | | ₂₀AAAGAGACCGGTTCACTGTGRN₁₋ | |
| | | ₂₀CTACGCGTATTCTTAAGCAATAAN₁₋ | |
| | | ₂₀AAAGAGACCGGTTCACTGTGRN₁₋ | |
| | | ₂₀TCCAATCAGTTCCTGATGCAGTAN₁₋ | |
| | | ₂₀CTACGCGTATTCTTAAGCAATAA | |
| 129-3p (4x) | (137-3p)-(129-3p)- | CTACGCGTATTCTTAAGCAATAAN₁₋ | 911 |
| 137-3p (4x) | (217-5p)-(129-3p)- | ₂₀ATACTTTTTGGGGTAAGGGCTTN₁₋ | |
| 217-5p (4x) | (217-5p)-(137-3p)- | ₂₀TCCAATCAGTTCCTGATGCAGTAN₁₋ | |
| | (217-5p)-(129-3p)- | ₂₀ATACTTTTTGGGGTAAGGGCTTN₁₋ | |
| | (137-3p)-(129-3p)- | ₂₀TCCAATCAGTTCCTGATGCAGTAN₁₋ | |
| | (217-5p)-(137-3p) | ₂₀CTACGCGTATTCTTAAGCAATAAN₁₋ | |
| | | ₂₀TCCAATCAGTTCCTGATGCAGTAN₁₋ | |
| | | ₂₀ATACTTTTTGGGGTAAGGGCTTN₁₋ | |
| | | ₂₀CTACGCGTATTCTTAAGCAATAAN₁₋ | |
| | | ₂₀ATACTTTTTGGGGTAAGGGCTTN₁₋ | |
| | | ₂₀TCCAATCAGTTCCTGATGCAGTAN₁₋ | |
| | | ₂₀CTACGCGTATTCTTAAGCAATAA | |
| 130-3p (4x) | (137-3p)-(130-3p)- | CTACGCGTATTCTTAAGCAATAAN₁₋ | 912 |
| 137-3p (4x) | (217-5p)-(130-3p)- | ₂₀ATGCCCTTTCATCATTGCACTGN₁₋ | |
| 217-5p (4x) | (217-5p)-(130-3p)- | ₂₀TCCAATCAGTTCCTGATGCAGTAN₁₋ | |
| | (217-5p)-(127-3p)- | ₂₀ATGCCCTTTCATCATTGCACTGN₁₋ | |
| | (137-3p)-(130-3p)- | ₂₀TCCAATCAGTTCCTGATGCAGTAN₁₋ | |
| | (217-5p)-(137-3p) | ₂₀ATGCCCTTTCATCATTGCACTGN₁₋ | |
| | | ₂₀TCCAATCAGTTCCTGATGCAGTAN₁₋ | |
| | | ₂₀(AGCCAAGCTCAGACGGATCCGA)N₁₋ | |
| | | ₂₀CTACGCGTATTCTTAAGCAATAAN₁₋ | |
| | | ₂₀ATGCCCTTTCATCATTGCACTGN₁₋ | |
| | | ₂₀TCCAATCAGTTCCTGATGCAGTAN₁₋ | |
| | | ₂₀CTACGCGTATTCTTAAGCAATAA | |

Exemplary miRNAs and related sequences are provided in Table F below.

TABLE F
Exemplary miRNA related Sequences

| | SEQ |
|---|---|
| | ID |
| Sequence Ref. | No. |
| hsa-miR-122-5p | 1 |
| hsa-miR-124-3p | 2 |
| hsa-miR-125a-5p | 3 |
| hsa-miR-126-3p | 4 |
| hsa-miR-127-3p | 5 |
| hsa-miR-128-3p | 6 |
| hsa-miR-129-3p | 7 |
| hsa-miR-129-5p | 8 |
| hsa-miR-130b-3p | 9 |
| hsa-miR-130b-5p | 10 |
| hsa-miR-133a-3p | 11 |
| hsa-miR-133b | 12 |
| hsa-miR-134-3p | 13 |
| hsa-miR-137 | 14 |
| hsa-miR-1-3p | 15 |
| hsa-miR-143-3p | 16 |
| hsa-miR-145-3p | 17 |
| hsa-miR-145-5p | 18 |
| hsa-miR-184 | 19 |
| hsa-miR-199a-3p | 20 |
| hsa-miR-199a-5p | 21 |
| hsa-miR-204-5p | 22 |
| hsa-miR-208b-3p | 23 |
| hsa-miR-214-3p | 24 |
| hsa-miR-217 | 25 |
| hsa-miR-219a-5p | 26 |
| hsa-miR-223-3p | 27 |
| hsa-miR-34a-5p | 28 |
| hsa-miR-451a | 29 |
| hsa-miR-559-5p | 30 |
| hsa-let-7a-5p | 31 |
| hsa-miR-9-5p | 32 |
| hsa-let-7b-5p | 33 |
| hsa-let-7c-5p | 34 |
| hsa-let-7d-5p | 35 |
| hsa-let-7e-5p | 36 |
| hsa-let-7f-5p | 37 |
| hsa-let-7g-5p | 38 |
| hsa-let-7i-5p | 39 |
| hsa-miR-100-5p | 40 |
| hsa-miR-101-3p | 41 |
| hsa-miR-103a-3p | 42 |
| hsa-miR-105-5p | 43 |
| hsa-miR-106a-5p | 44 |
| hsa-miR-106b-5p | 45 |
| hsa-miR-107 | 46 |
| hsa-miR-10a-5p | 47 |
| hsa-miR-10b-5p | 48 |
| hsa-miR-1178-3p | 49 |
| hsa-miR-1180-3p | 50 |
| hsa-miR-1183 | 51 |
| hsa-miR-1185-1-3p | 52 |
| hsa-miR-1185-2-3p | 53 |
| hsa-miR-1185-5p | 54 |
| hsa-miR-1193 | 55 |
| hsa-miR-1197 | 56 |
| hsa-miR-1200 | 57 |
| hsa-miR-1202 | 58 |
| hsa-miR-1203 | 59 |
| hsa-miR-1204 | 60 |
| hsa-miR-1205 | 61 |
| hsa-miR-1206 | 62 |
| hsa-miR-1224-3p | 63 |
| hsa-miR-1224-5p | 64 |
| hsa-miR-1226-3p | 65 |
| hsa-miR-1228-3p | 66 |
| hsa-miR-1233-3p | 67 |
| hsa-miR-1234-3p | 68 |
| hsa-miR-1236-3p | 69 |
| hsa-miR-1244 | 70 |
| hsa-miR-1245a | 71 |
| hsa-miR-1245b-3p | 72 |
| hsa-miR-1245b-5p | 73 |
| hsa-miR-1246 | 74 |
| hsa-miR-1247-5p | 75 |
| hsa-miR-1248 | 76 |
| hsa-miR-1249-3p | 77 |
| hsa-miR-1249-5p | 78 |
| hsa-miR-1250-5p | 79 |
| hsa-miR-1252-5p | 80 |
| hsa-miR-1253 | 81 |
| hsa-miR-1254 | 82 |
| hsa-miR-1255a | 83 |
| hsa-miR-1255b-5p | 84 |
| hsa-miR-1257 | 85 |
| hsa-miR-1258 | 86 |
| hsa-miR-125a-3p | 87 |
| hsa-miR-125b-5p | 88 |
| hsa-miR-1260a | 89 |
| hsa-miR-1260b | 90 |
| hsa-miR-1261 | 91 |
| hsa-miR-1262 | 92 |
| hsa-miR-1264 | 93 |
| hsa-miR-1266-5p | 94 |
| hsa-miR-1268a | 95 |
| hsa-miR-1268b | 96 |
| hsa-miR-1269a | 97 |
| hsa-miR-1269b | 98 |
| hsa-miR-1270 | 99 |
| hsa-miR-1271-3p | 100 |
| hsa-miR-1271-5p | 101 |
| hsa-miR-1272 | 102 |
| hsa-miR-1273c | 103 |
| hsa-miR-1275 | 104 |
| hsa-miR-127-5p | 105 |
| hsa-miR-1276 | 106 |
| hsa-miR-1277-3p | 107 |
| hsa-miR-1278 | 108 |
| hsa-miR-1279 | 109 |
| hsa-miR-1281 | 110 |
| hsa-miR-128-1-5p | 111 |
| hsa-miR-128-2-5p | 112 |
| hsa-miR-1283 | 113 |
| hsa-miR-1285-3p | 114 |
| hsa-miR-1285-5p | 115 |
| hsa-miR-1286 | 116 |
| hsa-miR-1287-3p | 117 |
| hsa-miR-1287-5p | 118 |
| hsa-miR-1288-3p | 119 |
| hsa-miR-1289 | 120 |
| hsa-miR-1290 | 121 |
| hsa-miR-1291 | 122 |
| hsa-miR-129-2-3p | 123 |
| hsa-miR-1293 | 124 |
| hsa-miR-1295a | 125 |
| hsa-miR-1296-3p | 126 |
| hsa-miR-1296-5p | 127 |
| hsa-miR-1297 | 128 |
| hsa-miR-1298-5p | 129 |
| hsa-miR-1299 | 130 |
| hsa-miR-1301-3p | 131 |
| hsa-miR-1302 | 132 |
| hsa-miR-1303 | 133 |
| hsa-miR-1304-3p | 134 |
| hsa-miR-1304-5p | 135 |
| hsa-miR-1305 | 136 |
| hsa-miR-1306-3p | 137 |
| hsa-miR-1306-5p | 138 |
| hsa-miR-1307-3p | 139 |
| hsa-miR-1307-5p | 140 |
| hsa-miR-130a-3p | 141 |
| hsa-miR-130b-3p | 142 |
| hsa-miR-1322 | 143 |
| hsa-miR-1323 | 144 |
| hsa-miR-132-3p | 145 |
| hsa-miR-133a-5p | 146 |
| hsa-miR-134-5p | 147 |
| hsa-miR-135a-5p | 148 |
| hsa-miR-135b-5p | 149 |
| hsa-miR-136-5p | 150 |
| hsa-miR-138-5p | 151 |
| hsa-miR-139-3p | 152 |
| hsa-miR-139-5p | 153 |
| hsa-miR-140-3p | 154 |
| hsa-miR-140-5p | 155 |
| hsa-miR-141-3p | 156 |
| hsa-miR-142-3p | 157 |
| hsa-miR-142-5p | 158 |
| hsa-miR-144-3p | 159 |
| hsa-miR-1469 | 160 |
| hsa-miR-146a-5p | 161 |
| hsa-miR-146b-3p | 162 |
| hsa-miR-146b-5p | 163 |
| hsa-miR-147a | 164 |
| hsa-miR-147b | 165 |
| hsa-miR-148a-3p | 166 |
| hsa-miR-148b-3p | 167 |
| hsa-miR-149-5p | 168 |
| hsa-miR-150-5p | 169 |
| hsa-miR-151a-3p | 170 |
| hsa-miR-151a-5p | 171 |
| hsa-miR-151b | 172 |
| hsa-miR-152-3p | 173 |
| hsa-miR-152-5p | 174 |
| hsa-miR-153-3p | 175 |
| hsa-miR-1537-3p | 176 |
| hsa-miR-154-5p | 177 |
| hsa-miR-155-5p | 178 |
| hsa-miR-15a-5p | 179 |
| hsa-miR-15b-5p | 180 |
| hsa-miR-1-5p | 181 |
| hsa-miR-16-5p | 182 |
| hsa-miR-181a-2-3p | 183 |
| hsa-miR-181a-3p | 184 |
| hsa-miR-181a-5p | 185 |
| hsa-miR-181b-2-3p | 186 |
| hsa-miR-181b-5p | 187 |
| hsa-miR-181c-5p | 188 |
| hsa-miR-181d-3p | 189 |
| hsa-miR-182-3p | 190 |
| hsa-miR-182-5p | 191 |
| hsa-miR-1827 | 192 |
| hsa-miR-183-5p | 193 |
| hsa-miR-185-5p | 194 |
| hsa-miR-186-5p | 195 |
| hsa-miR-187-3p | 196 |
| hsa-miR-188-3p | 197 |
| hsa-miR-188-5p | 198 |
| hsa-miR-18a-5p | 199 |
| hsa-miR-18b-5p | 200 |
| hsa-miR-1908-3p | 201 |
| hsa-miR-1908-5p | 202 |
| hsa-miR-1909-3p | 203 |
| hsa-miR-190a-3p | 204 |
| hsa-miR-190a-5p | 205 |
| hsa-miR-190b | 206 |
| hsa-miR-1910-3p | 207 |
| hsa-miR-1910-5p | 208 |
| hsa-miR-1915-3p | 209 |
| hsa-miR-191-5p | 210 |
| hsa-miR-192-5p | 211 |
| hsa-miR-193a-3p | 212 |
| hsa-miR-193a-5p | 213 |
| hsa-miR-193b-3p | 214 |
| hsa-miR-194-5p | 215 |
| hsa-miR-195-5p | 216 |
| hsa-miR-196a-3p | 217 |
| hsa-miR-196a-5p | 218 |
| hsa-miR-196b-5p | 219 |
| hsa-miR-1972 | 220 |
| hsa-miR-1973 | 221 |
| hsa-miR-197-3p | 222 |
| hsa-miR-197-5p | 223 |
| hsa-miR-1976 | 224 |
| hsa-miR-198 | 225 |
| hsa-miR-199b-5p | 226 |
| hsa-miR-19a-3p | 227 |
| hsa-miR-19b-3p | 228 |
| hsa-miR-200a-3p | 229 |
| hsa-miR-200b-3p | 230 |
| hsa-miR-200c-3p | 231 |
| hsa-miR-202-3p | 232 |
| hsa-miR-203a-3p | 233 |
| hsa-miR-203a-5p | 234 |
| hsa-miR-2053 | 235 |
| hsa-miR-205-5p | 236 |
| hsa-miR-206 | 237 |
| hsa-miR-208a-3p | 238 |
| hsa-miR-208b-5p | 239 |
| hsa-miR-20a-5p | 240 |
| hsa-miR-210-3p | 241 |
| hsa-miR-210-5p | 242 |
| hsa-miR-2110 | 243 |
| hsa-miR-2113 | 244 |
| hsa-miR-211-3p | 245 |
| hsa-miR-211-5p | 246 |
| hsa-miR-2116-5p | 247 |
| hsa-miR-2117 | 248 |
| hsa-miR-212-3p | 249 |
| hsa-miR-215-5p | 250 |
| hsa-miR-21-5p | 251 |
| hsa-miR-216a-5p | 252 |
| hsa-miR-216b-5p | 253 |
| hsa-miR-218-5p | 254 |
| hsa-miR-219a-1-3p | 255 |
| hsa-miR-219a-2-3p | 256 |
| hsa-miR-219b-3p | 257 |
| hsa-miR-221-3p | 258 |
| hsa-miR-221-5p | 259 |
| hsa-miR-222-3p | 260 |
| hsa-miR-22-3p | 261 |
| hsa-miR-224-5p | 262 |
| hsa-miR-2278 | 263 |
| hsa-miR-23a-3p | 264 |
| hsa-miR-23b-3p | 265 |
| hsa-miR-23c | 266 |
| hsa-miR-24-3p | 267 |
| hsa-miR-25-3p | 268 |
| hsa-miR-25-5p | 269 |
| hsa-miR-2682-5p | 270 |
| hsa-miR-26a-5p | 271 |
| hsa-miR-26b-5p | 272 |
| hsa-miR-27a-3p | 273 |
| hsa-miR-27b-3p | 274 |
| hsa-miR-28-3p | 275 |
| hsa-miR-28-5p | 276 |
| hsa-miR-296-3p | 277 |
| hsa-miR-296-5p | 278 |
| hsa-miR-297 | 279 |
| hsa-miR-298 | 280 |
| hsa-miR-299-3p | 281 |
| hsa-miR-299-5p | 282 |
| hsa-miR-29a-3p | 283 |
| hsa-miR-29b-3p | 284 |
| hsa-miR-29c-3p | 285 |
| hsa-miR-300 | 286 |
| hsa-miR-301a-3p | 287 |
| hsa-miR-301a-5p | 288 |
| hsa-miR-301b-3p | 289 |
| hsa-miR-301b-5p | 290 |
| hsa-miR-302a-3p | 291 |
| hsa-miR-302a-5p | 292 |
| hsa-miR-302b-3p | 293 |
| hsa-miR-302c-3p | 294 |
| hsa-miR-302d-3p | 295 |
| hsa-miR-302e | 296 |
| hsa-miR-302f | 297 |
| hsa-miR-3065-3p | 298 |
| hsa-miR-3065-5p | 299 |
| hsa-miR-3074-3p | 300 |
| hsa-miR-30a-3p | 301 |
| hsa-miR-30a-5p | 302 |
| hsa-miR-30b-5p | 303 |
| hsa-miR-30c-5p | 304 |
| hsa-miR-30d-5p | 305 |
| hsa-miR-30e-3p | 306 |
| hsa-miR-30e-5p | 307 |
| hsa-miR-3127-5p | 308 |
| hsa-miR-3130-3p | 309 |
| hsa-miR-3131 | 310 |
| hsa-miR-3136-5p | 311 |
| hsa-miR-3140-3p | 312 |
| hsa-miR-3140-5p | 313 |
| hsa-miR-3144-3p | 314 |
| hsa-miR-3144-5p | 315 |
| hsa-miR-3147 | 316 |
| hsa-miR-3150b-3p | 317 |
| hsa-miR-3151-5p | 318 |
| hsa-miR-3158-3p | 319 |
| hsa-miR-31-5p | 320 |
| hsa-miR-3161 | 321 |
| hsa-miR-3164 | 322 |
| hsa-miR-3168 | 323 |
| hsa-miR-3179 | 324 |
| hsa-miR-3180 | 325 |
| hsa-miR-3180-3p | 326 |
| hsa-miR-3180-5p | 327 |
| hsa-miR-3182 | 328 |
| hsa-miR-3185 | 329 |
| hsa-miR-3190-3p | 330 |
| hsa-miR-3192-5p | 331 |
| hsa-miR-3195 | 332 |
| hsa-miR-3196 | 333 |
| hsa-miR-3202 | 334 |
| hsa-miR-320a | 335 |
| hsa-miR-320b | 336 |
| hsa-miR-320c | 337 |
| hsa-miR-320d | 338 |
| hsa-miR-320e | 339 |
| hsa-miR-323a-3p | 340 |
| hsa-miR-323a-5p | 341 |
| hsa-miR-323b-3p | 342 |
| hsa-miR-323b-5p | 343 |
| hsa-miR-324-3p | 344 |
| hsa-miR-324-5p | 345 |
| hsa-miR-325 | 346 |
| hsa-miR-32-5p | 347 |
| hsa-miR-326 | 348 |
| hsa-miR-328-3p | 349 |
| hsa-miR-328-5p | 350 |
| hsa-miR-329-3p | 351 |
| hsa-miR-329-5p | 352 |
| hsa-miR-330-3p | 353 |
| hsa-miR-330-5p | 354 |
| hsa-miR-331-3p | 355 |
| hsa-miR-331-5p | 356 |
| hsa-miR-335-5p | 357 |
| hsa-miR-337-3p | 358 |
| hsa-miR-337-5p | 359 |
| hsa-miR-338-5p | 360 |
| hsa-miR-339-3p | 361 |
| hsa-miR-339-5p | 362 |
| hsa-miR-33a-5p | 363 |
| hsa-miR-33b-5p | 364 |
| hsa-miR-340-5p | 365 |
| hsa-miR-342-3p | 366 |
| hsa-miR-342-5p | 367 |
| hsa-miR-345-3p | 368 |
| hsa-miR-345-5p | 369 |
| hsa-miR-346 | 370 |
| hsa-miR-34b-3p | 371 |
| hsa-miR-34c-3p | 372 |
| hsa-miR-34c-5p | 373 |
| hsa-miR-3605-3p | 374 |
| hsa-miR-3605-5p | 375 |
| hsa-miR-3613-3p | 376 |
| hsa-miR-3613-5p | 377 |
| hsa-miR-361-3p | 378 |
| hsa-miR-3614-3p | 379 |
| hsa-miR-3614-5p | 380 |
| hsa-miR-3615 | 381 |
| hsa-miR-361-5p | 382 |
| hsa-miR-362-3p | 383 |
| hsa-miR-362-5p | 384 |
| hsa-miR-363-3p | 385 |
| hsa-miR-363-5p | 386 |
| hsa-miR-365a-3p | 387 |
| hsa-miR-365b-5p | 388 |
| hsa-miR-367-3p | 389 |
| hsa-miR-3690 | 390 |
| hsa-miR-369-3p | 391 |
| hsa-miR-369-5p | 392 |
| hsa-miR-370-3p | 393 |
| hsa-miR-370-5p | 394 |
| hsa-miR-371a-5p | 395 |
| hsa-miR-371b-5p | 396 |
| hsa-miR-372-3p | 397 |
| hsa-miR-373-3p | 398 |
| hsa-miR-374a-3p | 399 |
| hsa-miR-374a-5p | 400 |
| hsa-miR-374b-5p | 401 |
| hsa-miR-374c-5p | 402 |
| hsa-miR-375 | 403 |
| hsa-miR-376a-2-5p | 404 |
| hsa-miR-376a-3p | 405 |
| hsa-miR-376b-3p | 406 |
| hsa-miR-376c-3p | 407 |
| hsa-miR-376c-5p | 408 |
| hsa-miR-377-3p | 409 |
| hsa-miR-378b | 410 |
| hsa-miR-378c | 411 |
| hsa-miR-378d | 412 |
| hsa-miR-378e | 413 |
| hsa-miR-378f | 414 |
| hsa-miR-378g | 415 |
| hsa-miR-378h | 416 |
| hsa-miR-378i | 417 |
| hsa-miR-379-5p | 418 |
| hsa-miR-380-3p | 419 |
| hsa-miR-381-3p | 420 |
| hsa-miR-381-5p | 421 |
| hsa-miR-382-3p | 422 |
| hsa-miR-382-5p | 423 |
| hsa-miR-383-5p | 424 |
| hsa-miR-384 | 425 |
| hsa-miR-3916 | 426 |
| hsa-miR-3918 | 427 |
| hsa-miR-3928-3p | 428 |
| hsa-miR-3934-5p | 429 |
| hsa-miR-409-3p | 430 |
| hsa-miR-409-5p | 431 |
| hsa-miR-410-3p | 432 |
| hsa-miR-411-5p | 433 |
| hsa-miR-412-3p | 434 |
| hsa-miR-421 | 435 |
| hsa-miR-422a | 436 |
| hsa-miR-423-3p | 437 |
| hsa-miR-423-5p | 438 |
| hsa-miR-424-5p | 439 |
| hsa-miR-425-5p | 440 |
| hsa-miR-4284 | 441 |
| hsa-miR-4286 | 442 |
| hsa-miR-429 | 443 |
| hsa-miR-431-5p | 444 |
| hsa-miR-432-5p | 445 |
| hsa-miR-433-3p | 446 |
| hsa-miR-433-5p | 447 |
| hsa-miR-4421 | 448 |
| hsa-miR-4425 | 449 |
| hsa-miR-4431 | 450 |
| hsa-miR-4435 | 451 |
| hsa-miR-4443 | 452 |
| hsa-miR-4448 | 453 |
| hsa-miR-4451 | 454 |
| hsa-miR-4454 | 455 |
| hsa-miR-4455 | 456 |
| hsa-miR-4458 | 457 |
| hsa-miR-4461 | 458 |
| hsa-miR-448 | 459 |
| hsa-miR-4485-3p | 460 |
| hsa-miR-4488 | 461 |
| hsa-miR-449a | 462 |
| hsa-miR-449b-5p | 463 |
| hsa-miR-449c-5p | 464 |
| hsa-miR-450a-1-3p | 465 |
| hsa-miR-450a-2-3p | 466 |
| hsa-miR-450a-5p | 467 |
| hsa-miR-450b-3p | 468 |
| hsa-miR-450b-5p | 469 |
| hsa-miR-4516 | 470 |
| hsa-miR-4521 | 471 |
| hsa-miR-4524a-5p | 472 |
| hsa-miR-452-5p | 473 |
| hsa-miR-4531 | 474 |
| hsa-miR-4532 | 475 |
| hsa-miR-4536-3p | 476 |
| hsa-miR-4536-5p | 477 |
| hsa-miR-454-3p | 478 |
| hsa-miR-455-3p | 479 |
| hsa-miR-455-5p | 480 |
| hsa-miR-4647 | 481 |
| hsa-miR-4707-3p | 482 |
| hsa-miR-4707-5p | 483 |
| hsa-miR-4741 | 484 |
| hsa-miR-4755-5p | 485 |
| hsa-miR-4787-3p | 486 |
| hsa-miR-4787-5p | 487 |
| hsa-miR-4792 | 488 |
| hsa-miR-483-3p | 489 |
| hsa-miR-483-5p | 490 |
| hsa-miR-484 | 491 |
| hsa-miR-485-3p | 492 |
| hsa-miR-485-5p | 493 |
| hsa-miR-486-3p | 494 |
| hsa-miR-487a-3p | 495 |
| hsa-miR-487b-3p | 496 |
| hsa-miR-487b-5p | 497 |
| hsa-miR-488-3p | 498 |
| hsa-miR-489-3p | 499 |
| hsa-miR-490-3p | 500 |
| hsa-miR-490-5p | 501 |
| hsa-miR-491-3p | 502 |
| hsa-miR-491-5p | 503 |
| hsa-miR-492 | 504 |
| hsa-miR-493-3p | 505 |
| hsa-miR-494-3p | 506 |
| hsa-miR-494-5p | 507 |
| hsa-miR-495-3p | 508 |
| hsa-miR-495-5p | 509 |
| hsa-miR-496 | 510 |
| hsa-miR-497-5p | 511 |
| hsa-miR-498 | 512 |
| hsa-miR-499a-3p | 513 |
| hsa-miR-499a-5p | 514 |
| hsa-miR-499b-3p | 515 |
| hsa-miR-499b-5p | 516 |
| hsa-miR-5001-3p | 517 |
| hsa-miR-5001-5p | 518 |
| hsa-miR-500a-5p | 519 |
| hsa-miR-5010-3p | 520 |
| hsa-miR-5010-5p | 521 |
| hsa-miR-501-3p | 522 |
| hsa-miR-502-3p | 523 |
| hsa-miR-502-5p | 524 |
| hsa-miR-503-3p | 525 |
| hsa-miR-503-5p | 526 |
| hsa-miR-504-3p | 527 |
| hsa-miR-504-5p | 528 |
| hsa-miR-505-3p | 529 |
| hsa-miR-506-3p | 530 |
| hsa-miR-506-5p | 531 |
| hsa-miR-507 | 532 |
| hsa-miR-508-3p | 533 |
| hsa-miR-508-5p | 534 |
| hsa-miR-509-3-5p | 535 |
| hsa-miR-509-3p | 536 |
| hsa-miR-509-5p | 537 |
| hsa-miR-510-3p | 538 |
| hsa-miR-510-5p | 539 |
| hsa-miR-511-5p | 540 |
| hsa-miR-512-3p | 541 |
| hsa-miR-512-5p | 542 |
| hsa-miR-513a-3p | 543 |
| hsa-miR-513a-5p | 544 |
| hsa-miR-513b-5p | 545 |
| hsa-miR-513c-3p | 546 |
| hsa-miR-513c-5p | 547 |
| hsa-miR-514a-3p | 548 |
| hsa-miR-514a-5p | 549 |
| hsa-miR-514b-3p | 550 |
| hsa-miR-514b-5p | 551 |
| hsa-miR-515-3p | 552 |
| hsa-miR-515-5p | 553 |
| hsa-miR-516a-3p | 554 |
| hsa-miR-516a-5p | 555 |
| hsa-miR-516b-5p | 556 |
| hsa-miR-517a-3p | 557 |
| hsa-miR-517b-3p | 558 |
| hsa-miR-517c-3p | 559 |
| hsa-miR-518b | 560 |
| hsa-miR-518c-3p | 561 |
| hsa-miR-518d-3p | 562 |
| hsa-miR-518e-3p | 563 |
| hsa-miR-518f-3p | 564 |
| hsa-miR-5196-3p | 565 |
| hsa-miR-5196-5p | 566 |
| hsa-miR-519b-3p | 567 |
| hsa-miR-519b-5p | 568 |
| hsa-miR-519c-3p | 569 |
| hsa-miR-519d-3p | 570 |
| hsa-miR-519e-3p | 571 |
| hsa-miR-520a-3p | 572 |
| hsa-miR-520a-5p | 573 |
| hsa-miR-520b | 574 |
| hsa-miR-520c-3p | 575 |
| hsa-miR-520d-3p | 576 |
| hsa-miR-520d-5p | 577 |
| hsa-miR-520e | 578 |
| hsa-miR-520f-3p | 579 |
| hsa-miR-520g-3p | 580 |
| hsa-miR-520h | 581 |
| hsa-miR-521 | 582 |
| hsa-miR-522-3p | 583 |
| hsa-miR-523-3p | 584 |
| hsa-miR-524-3p | 585 |
| hsa-miR-525-3p | 586 |
| hsa-miR-525-5p | 587 |
| hsa-miR-526a | 588 |
| hsa-miR-526b-5p | 589 |
| hsa-miR-532-3p | 590 |
| hsa-miR-532-5p | 591 |
| hsa-miR-539-3p | 592 |
| hsa-miR-539-5p | 593 |
| hsa-miR-541-3p | 594 |
| hsa-miR-542-3p | 595 |
| hsa-miR-542-5p | 596 |
| hsa-miR-543 | 597 |
| hsa-miR-544a | 598 |
| hsa-miR-545-3p | 599 |
| hsa-miR-548a-3p | 600 |
| hsa-miR-548a-5p | 601 |
| hsa-miR-548aa | 602 |
| hsa-miR-548ad-3p | 603 |
| hsa-miR-548ah-5p | 604 |
| hsa-miR-548ai | 605 |
| hsa-miR-548ak | 606 |
| hsa-miR-548al | 607 |
| hsa-miR-548ar-3p | 608 |
| hsa-miR-548ar-5p | 609 |
| hsa-miR-548b-3p | 610 |
| hsa-miR-548c-5p | 611 |
| hsa-miR-548d-3p | 612 |
| hsa-miR-548d-5p | 613 |
| hsa-miR-548e-3p | 614 |
| hsa-miR-548e-5p | 615 |
| hsa-miR-548g-3p | 616 |
| hsa-miR-548h-5p | 617 |
| hsa-miR-548i | 618 |
| hsa-miR-548j-3p | 619 |
| hsa-miR-548j-5p | 620 |
| hsa-miR-548k | 621 |
| hsa-miR-548l | 622 |
| hsa-miR-548m | 623 |
| hsa-miR-548n | 624 |
| hsa-miR-548o-3p | 625 |
| hsa-miR-548q | 626 |
| hsa-miR-548v | 627 |
| hsa-miR-548y | 628 |
| hsa-miR-548z | 629 |
| hsa-miR-549a | 630 |
| hsa-miR-550a-5p | 631 |
| hsa-miR-551a | 632 |
| hsa-miR-551b-3p | 633 |
| hsa-miR-552-3p | 634 |
| hsa-miR-553 | 635 |
| hsa-miR-554 | 636 |
| hsa-miR-555 | 637 |
| hsa-miR-556-3p | 638 |
| hsa-miR-556-5p | 639 |
| hsa-miR-561-3p | 640 |
| hsa-miR-561-5p | 641 |
| hsa-miR-562 | 642 |
| hsa-miR-563 | 643 |
| hsa-miR-564 | 644 |
| hsa-miR-566 | 645 |
| hsa-miR-567 | 646 |
| hsa-miR-568 | 647 |
| hsa-miR-570-3p | 648 |
| hsa-miR-571 | 649 |
| hsa-miR-572 | 650 |
| hsa-miR-573 | 651 |
| hsa-miR-574-3p | 652 |
| hsa-miR-574-5p | 653 |
| hsa-miR-575 | 654 |
| hsa-miR-576-3p | 655 |
| hsa-miR-576-5p | 656 |
| hsa-miR-577 | 657 |
| hsa-miR-578 | 658 |
| hsa-miR-579-3p | 659 |
| hsa-miR-579-5p | 660 |
| hsa-miR-580-3p | 661 |
| hsa-miR-582-3p | 662 |
| hsa-miR-582-5p | 663 |
| hsa-miR-584-3p | 664 |
| hsa-miR-584-5p | 665 |
| hsa-miR-585-3p | 666 |
| hsa-miR-587 | 667 |
| hsa-miR-589-5p | 668 |
| hsa-miR-590-3p | 669 |
| hsa-miR-590-5p | 670 |
| hsa-miR-591 | 671 |
| hsa-miR-592 | 672 |
| hsa-miR-593-3p | 673 |
| hsa-miR-595 | 674 |
| hsa-miR-596 | 675 |
| hsa-miR-597-5p | 676 |
| hsa-miR-598-3p | 677 |
| hsa-miR-599 | 678 |
| hsa-miR-600 | 679 |
| hsa-miR-601 | 680 |
| hsa-miR-603 | 681 |
| hsa-miR-604 | 682 |
| hsa-miR-605-5p | 683 |
| hsa-miR-606 | 684 |
| hsa-miR-607 | 685 |
| hsa-miR-608 | 686 |
| hsa-miR-610 | 687 |
| hsa-miR-612 | 688 |
| hsa-miR-613 | 689 |
| hsa-miR-614 | 690 |
| hsa-miR-615-3p | 691 |
| hsa-miR-615-5p | 692 |
| hsa-miR-616-3p | 693 |
| hsa-miR-617 | 694 |
| hsa-miR-619-3p | 695 |
| hsa-miR-620 | 696 |
| hsa-miR-624-3p | 697 |
| hsa-miR-625-5p | 698 |
| hsa-miR-626 | 699 |
| hsa-miR-627-3p | 700 |
| hsa-miR-627-5p | 701 |
| hsa-miR-628-3p | 702 |
| hsa-miR-628-5p | 703 |
| hsa-miR-629-5p | 704 |
| hsa-miR-630 | 705 |
| hsa-miR-631 | 706 |
| hsa-miR-637 | 707 |
| hsa-miR-638 | 708 |
| hsa-miR-639 | 709 |
| hsa-miR-640 | 710 |
| hsa-miR-641 | 711 |
| hsa-miR-642a-3p | 712 |
| hsa-miR-642a-5p | 713 |
| hsa-miR-643 | 714 |
| hsa-miR-644a | 715 |
| hsa-miR-648 | 716 |
| hsa-miR-649 | 717 |
| hsa-miR-650 | 718 |
| hsa-miR-6503-3p | 719 |
| hsa-miR-6503-5p | 720 |
| hsa-miR-6511a-3p | 721 |
| hsa-miR-6511a-5p | 722 |
| hsa-miR-651-3p | 723 |
| hsa-miR-651-5p | 724 |
| hsa-miR-652-3p | 725 |
| hsa-miR-652-5p | 726 |
| hsa-miR-654-3p | 727 |
| hsa-miR-654-5p | 728 |
| hsa-miR-655-3p | 729 |
| hsa-miR-656-3p | 730 |
| hsa-miR-660-3p | 731 |
| hsa-miR-660-5p | 732 |
| hsa-miR-661 | 733 |
| hsa-miR-663a | 734 |
| hsa-miR-664a-3p | 735 |
| hsa-miR-664b-3p | 736 |
| hsa-miR-664b-5p | 737 |
| hsa-miR-665 | 738 |
| hsa-miR-671-3p | 739 |
| hsa-miR-671-5p | 740 |
| hsa-miR-6720-3p | 741 |
| hsa-miR-6721-5p | 742 |
| hsa-miR-6724-5p | 743 |
| hsa-miR-675-5p | 744 |
| hsa-miR-708-5p | 745 |
| hsa-miR-744-5p | 746 |
| hsa-miR-758-3p | 747 |
| hsa-miR-758-5p | 748 |
| hsa-miR-7-5p | 749 |
| hsa-miR-760 | 750 |
| hsa-miR-761 | 751 |
| hsa-miR-764 | 752 |
| hsa-miR-765 | 753 |
| hsa-miR-766-3p | 754 |
| hsa-miR-766-5p | 755 |
| hsa-miR-767-3p | 756 |
| hsa-miR-767-5p | 757 |
| hsa-miR-769-3p | 758 |
| hsa-miR-769-5p | 759 |
| hsa-miR-770-5p | 760 |
| hsa-miR-802 | 761 |
| hsa-miR-873-3p | 762 |
| hsa-miR-873-5p | 763 |
| hsa-miR-874-3p | 764 |
| hsa-miR-874-5p | 765 |
| hsa-miR-875-3p | 766 |
| hsa-miR-876-3p | 767 |
| hsa-miR-876-5p | 768 |
| hsa-miR-877-5p | 769 |
| hsa-miR-885-3p | 770 |
| hsa-miR-885-5p | 771 |
| hsa-miR-887-3p | 772 |
| hsa-miR-887-5p | 773 |
| hsa-miR-888-5p | 774 |
| hsa-miR-889-3p | 775 |
| hsa-miR-890 | 776 |
| hsa-miR-891a-5p | 777 |
| hsa-miR-891b | 778 |
| hsa-miR-892a | 779 |
| hsa-miR-892b | 780 |
| hsa-miR-922 | 781 |
| hsa-miR-924 | 782 |
| hsa-miR-92a-1-5p | 783 |
| hsa-miR-92a-3p | 784 |
| hsa-miR-92b-3p | 785 |
| hsa-miR-933 | 786 |
| hsa-miR-934 | 787 |
| hsa-miR-935 | 788 |
| hsa-miR-93-5p | 789 |
| hsa-miR-936 | 790 |
| hsa-miR-937-3p | 791 |
| hsa-miR-939-5p | 792 |
| hsa-miR-940 | 793 |
| hsa-miR-941 | 794 |
| hsa-miR-942-3p | 795 |
| hsa-miR-942-5p | 796 |
| hsa-miR-944 | 797 |
| hsa-miR-95-3p | 798 |
| hsa-miR-96-5p | 799 |
| hsa-miR-98-3p | 800 |
| hsa-miR-98-5p | 801 |
| hsa-miR-99a-5p | 802 |
| hsa-miR-99b-5p | 803 |
| hsa-miR-122-5p-TS | 804 |
| hsa-miR-124-3p-TS | 805 |
| hsa-miR-125a-5p-TS | 806 |
| hsa-miR-126-3p-TS | 807 |
| hsa-miR-126-3p-TSM | 808 |
| hsa-miR-127-3p-TS | 809 |
| hsa-miR-128-3p-TS | 810 |
| hsa-miR-128-3p-TSM | 811 |
| hsa-miR-129-3p-TS | 812 |
| hsa-miR-129-5p-TS | 813 |
| hsa-miR-130b-3p-TS | 814 |
| hsa-miR-130b-5p-TS | 815 |
| hsa-miR-133a-3p-TS | 816 |
| hsa-miR-133b-TS | 817 |
| hsa-miR-134-3p-TS | 818 |
| hsa-miR-137-TS | 819 |
| hsa-miR-1-3p-TS | 820 |
| hsa-miR-143-3p-TS | 821 |
| hsa-miR-145-3p-TS | 822 |
| hsa-miR-145-5p-TS | 823 |
| hsa-miR-184-TS | 824 |
| hsa-miR-199a-3p-TS | 825 |
| hsa-miR-199a-5p-TS | 826 |
| hsa-miR-204-5p-TS | 827 |
| hsa-miR-208b-3p-TS | 828 |
| hsa-miR-214-3p-TS | 829 |
| hsa-miR-217-TS | 830 |
| hsa-miR-219a-5p-TS | 831 |
| hsa-miR-223-3p-TS | 832 |
| hsa-miR-34a-5p-TS | 833 |
| hsa-miR-451a-TS | 834 |
| hsa-miR-559-5p-TS | 835 |
| hsa-let-7a-5p-TS | 836 |
| hsa-miR-9-5p-TS | 837 |
| miRT-l-143_1736 | 838 |
| miRT-128m-122-219_6793 | 839 |
| miRT-128m-204-219_9304 | 840 |
| miRT-217-137-126m_3163 | 841 |

### Exemplary Dual Oncolytic Virus Constructs or Dual Virus Constructs

In some embodiments, the present disclosure provides a primary oncolytic virus or a primary virus comprising a polynucleotide encoding a secondary oncolytic virus or a secondary virus. In some embodiments, the primary and secondary viruses or oncolytic viruses are replication-competent. In some embodiments, the present disclosure provides a primary oncolytic virus or a primary virus comprising (i) a first polynucleotide encoding a secondary oncolytic virus or a secondary virus operably linked to a regulatable promoter and (ii) a second polynucleotide encoding a protein capable of binding to the regulatable promoter and operably linked to a constitutive promoter.

In some embodiments, the primary oncolytic virus or the primary virus comprises (i) a first polynucleotide operably linked to a Tet-OFF promoter and encoding a secondary oncolytic virus or a secondary virus and (ii) a second polynucleotide operably linked to a constitutive promoter and encoding a tTA protein capable of binding to the Tet-OFF promoter and regulating transcription of the polynucleotide encoding the secondary oncolytic virus or the secondary virus. In such embodiments, the primary oncolytic virus or the primary virus is expressed in a cell in the presence or absence of tetracycline, while the secondary virus is only expressed in the absence of tetracycline. In such embodiments, the 5′ and 3′ junctional cleavage sequences flanking the polynucleotide encoding the secondary virus can be any of: ribozymes, non-tetracycline activated aptazymes, pre-miRNA sequences, miRNA target sequence, gRNA target sequences, or AmiRNA target sequences. The primary and secondary viruses can be further miR-attenuated as described above.

In some embodiments, the primary oncolytic virus or the primary virus comprises (i) a first polynucleotide operably linked to a Tet-OFF promoter and encoding a secondary oncolytic virus or a secondary virus; (ii) a second polynucleotide operably linked to a Tet-ON promoter and encoding an RNAi molecule targeting a sequence in the secondary viral genome; and (ii) a third polynucleotide operably linked to a constitutive promoter and encoding a tTA protein capable of binding to the Tet-OFF promoter and regulating transcription of the polynucleotide encoding the secondary oncolytic virus or the secondary virus and an rtTA protein capable of binding to the Tet-ON promoter and regulating transcription of the polynucleotide encoding the RNAi molecule. In such embodiments, the primary oncolytic virus or the primary virus is expressed in a cell in the presence or absence of tetracycline, while the secondary virus is only expressed in the absence of tetracycline. Aberrant expression of the secondary oncolytic virus or the secondary virus in the presence of tetracycline is prevented by the expression of the safety RNAi molecule in the presence of tetracycline, which recognizes a target sequence in the secondary viral genome and mediates degradation of the secondary viral transcript. In such embodiments, the 5′ and 3′ junctional cleavage sequences flanking the polynucleotide encoding the secondary virus can be any of: ribozymes, non-tetracycline activated aptazymes, pre-miRNA sequences, miRNA target sequence, gRNA target sequences, or AmiRNA target sequences. The primary and secondary viruses can be further miR-attenuated as described above.

In some embodiments, the primary oncolytic virus or the primary virus comprises (i) a first polynucleotide operably linked to a Tet-ON promoter and encoding a secondary oncolytic virus or a secondary virus and (ii) a second polynucleotide operably linked to a constitutive promoter and encoding an rtTA protein capable of binding to the Tet-ON promoter and regulating transcription of the polynucleotide encoding the secondary oncolytic virus or the secondary virus. In such embodiments, the primary oncolytic virus or the primary virus is expressed in a cell in the presence or absence of tetracycline, while the secondary virus is only expressed in the presence of tetracycline. In such embodiments, the 5′ and 3′ junctional cleavage sequences flanking the polynucleotide encoding the secondary virus can be any of: ribozymes, aptazymes (including tetracycline activated aptazymes), pre-miRNA sequences, miRNA target sequence, gRNA target sequences, or AmiRNA target sequences. The primary and secondary viruses can be further miR-attenuated as described above.

In some embodiments, the primary oncolytic virus or the primary virus comprises (i) a first polynucleotide operably linked to a Tet-ON promoter and encoding a secondary oncolytic virus or a secondary virus; (ii) a second polynucleotide operably linked to a Tet-OFF promoter and encoding an RNAi molecule targeting a sequence in the secondary viral genome; and (ii) a third polynucleotide operably linked to a constitutive promoter and encoding an rtTA protein capable of binding to the Tet-ON promoter and regulating transcription of the polynucleotide encoding the secondary oncolytic virus or the secondary virus and an tTA protein capable of binding to the Tet-OFF promoter and regulating transcription of the polynucleotide encoding the RNAi molecule. In such embodiments, the primary oncolytic virus or the primary virus is expressed in a cell in the presence or absence of tetracycline, while the secondary virus is only expressed in the presence of tetracycline. Aberrant expression of the secondary oncolytic virus or the secondary virus in the absence of tetracycline is prevented by the expression of the safety RNAi molecule in the absence of tetracycline, which recognizes a target sequence in the secondary viral genome and mediates degradation of the secondary viral transcript. In such embodiments, the 5′ and 3′ junctional cleavage sequences flanking the polynucleotide encoding the secondary virus can be any of: ribozymes, aptazymes (including tetracycline activated aptazymes), pre-miRNA sequences, miRNA target sequence, gRNA target sequences, or AmiRNA target sequences. The primary and secondary viruses can be further miR-attenuated as described above.

In some embodiments, the primary oncolytic virus or the primary virus comprises a polynucleotide encoding a secondary oncolytic virus or a secondary virus. In some embodiments, the polynucleotide encoding the secondary oncolytic virus or the secondary virus comprises one or more recombinase recognition sites. In some embodiments, the polynucleotide encoding the secondary oncolytic virus or the secondary virus comprises one or more recombinase-responsive cassettes. Exemplary recombinase-responsive cassettes include RREC and RRIC (optionally comprising a portion of an intron) of the disclosure. In some embodiments, the primary oncolytic virus or the primary virus comprises a polynucleotide encoding a recombinase. In some embodiments, the polynucleotide encoding the recombinase comprises an intron (or a portion thereof). In some embodiments, the polynucleotide encoding the recombinase is operably linked to a regulatable promoter.

In some embodiments, the primary oncolytic virus comprises a polynucleotide encoding a secondary oncolytic virus, wherein the primary oncolytic virus is HSV and the secondary oncolytic virus is a Picornavirus. In some embodiments, the dual oncolytic viruses comprise a primary oncolytic virus comprising a polynucleotide encoding a secondary oncolytic virus, wherein the primary oncolytic virus is HSV and the secondary oncolytic virus is SVV. In some embodiments, the dual oncolytic viruses comprise a primary oncolytic virus comprising a polynucleotide encoding a secondary oncolytic virus, wherein the primary oncolytic virus is HSV and the secondary oncolytic virus is CVA. In some embodiments, the primary virus comprises a polynucleotide encoding a secondary virus, wherein the primary virus is HSV and the secondary virus is a Picornavirus. In some embodiments, the dual viruses comprise a primary virus comprising a polynucleotide encoding a secondary virus, wherein the primary virus is HSV and the secondary virus is SVV. In some embodiments, the dual viruses comprise a primary virus comprising a polynucleotide encoding a secondary virus, wherein the primary virus is HSV and the secondary virus is CVA.

### Production of Dual Oncolytic Virus Constructs or Dual Virus Constructs

In some embodiments, the present disclosure provides methods of producing the dual oncolytic viruses or dual viruses described herein.

In some embodiments, the present disclosure provides viral stocks of the dual oncolytic viruses or dual viruses described herein. In some embodiments, the viral stock is a homogeneous stock. The preparation and analysis of viral stocks is well known in the art. For example, a viral stock can be manufactured in roller bottles containing cells transduced with the viral vector. The viral stock can then be purified on a continuous nycodenze gradient, and aliquotted and stored until needed. Viral stocks vary considerably in titer, depending largely on viral genotype and the protocol and cell lines used to prepare them. In some embodiments, the titer of a viral stock contemplated herein is at least about 10⁵ plaque-forming units (pfu), such as at least about 10⁶ pfu or at least about 10¹⁰ pfu. In certain embodiments, the titer can be at least about 10⁸ pfu, or at least about 10⁹ pfu, at least about 10¹⁰ pfu, or at least about 10¹¹ pfu.

### Therapeutic Compositions

In some embodiments, the present disclosure provides compositions comprising the dual oncolytic viruses or dual viruses described herein. In some embodiments, the compositions further comprise a pharmaceutically acceptable carrier. The term “composition” as used herein refers to a formulation of one or more dual oncolytic viruses or dual viruses described herein that is capable of being administered or delivered to a subject and/or a cell. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” is a composition of one or more agents capable of being administered or delivered to a patient and/or subject and/or cell for the treatment of a particular disease or disorder.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like that are physiologically compatible, including pharmaceutically acceptable cell culture media and/or emulsifier which has been approved by the United States Food and Drug Administration as being acceptable for use in humans and/or domestic animals. The use of such media and agents for pharmaceutical active substances is well known in the art. Except insofar as any conventional media or agent is incompatible with the active ingredient, its use in the therapeutic compositions is contemplated. Supplement

In one embodiment, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with a viral vector or nucleic acid molecule, use thereof in the pharmaceutical compositions of the disclosure is contemplated.

The compositions of the disclosure may comprise one or more polypeptides, polynucleotides, vectors comprising same, infected cells, etc., as described herein, formulated in pharmaceutically-acceptable or physiologically-acceptable solutions for administration to a cell or an animal, either alone, or in combination with one or more other modalities of therapy. It will also be understood that, if desired, the compositions of the disclosure may be administered in combination with other agents as well, such as, e.g., cytokines, growth factors, hormones, small molecules or various pharmaceutically-active agents. There is virtually no limit to other components that may also be included in the compositions, provided that the additional agents do not adversely affect the ability of the composition to deliver the intended therapy.

In the pharmaceutical compositions of the disclosure, formulation of pharmaceutically-acceptable excipients and carrier solutions is well-known to those of skill in the art, as is the development of suitable dosing and treatment regimens for using the particular compositions described herein in a variety of treatment regimens. Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Center for Biologics Evaluation and Research standards. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration.

In certain circumstances it will be desirable to deliver the compositions, recombinant viral vectors, and nucleic acid molecules disclosed herein parenterally, intravenously, intramuscularly, or even intraperitoneally as described, for example, in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabenes, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain embodiments, the compositions may be delivered by intranasal sprays, inhalation, and/or other aerosol delivery vehicles. Methods for delivering polynucleotides and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

### Methods of Use

In some embodiments, the present disclosure provides methods of killing a cancerous cell, comprising exposing the cancerous cell to a dual-oncolytic virus described herein or compositions. In some embodiments, the dual-oncolytic virus replicates within the cancerous cell and produces a secondary oncolytic virus. In some embodiments, the secondary oncolytic virus infects and replicates within another cancerous cell. Therefore, in some embodiments, the dual-oncolytic viruses of the present disclosure are capable of killing a plurality of cancerous cells. In such embodiments, a first subset of the plurality of cancer cells may be killed by the first oncolytic virus and a second subset of the plurality of cancer cells may be killed by the secondary oncolytic virus. In some embodiments, a cancerous cells are in vivo. In certain embodiments, the cancerous cells are within a tumor.

In some embodiments, the present disclosure provides methods of killing a cancerous cell, comprising exposing the cancerous cell to a dual virus described herein or compositions. In some embodiments, the dual virus replicates within the cancerous cell and produces a secondary virus. In some embodiments, the secondary virus infects and replicates within another cancerous cell. Therefore, in some embodiments, the dual viruses of the present disclosure are capable of killing a plurality of cancerous cells. In such embodiments, a first subset of the plurality of cancer cells may be killed by the first virus and a second subset of the plurality of cancer cells may be killed by the secondary virus. In some embodiments, a cancerous cells are in vivo. In certain embodiments, the cancerous cells are within a tumor.

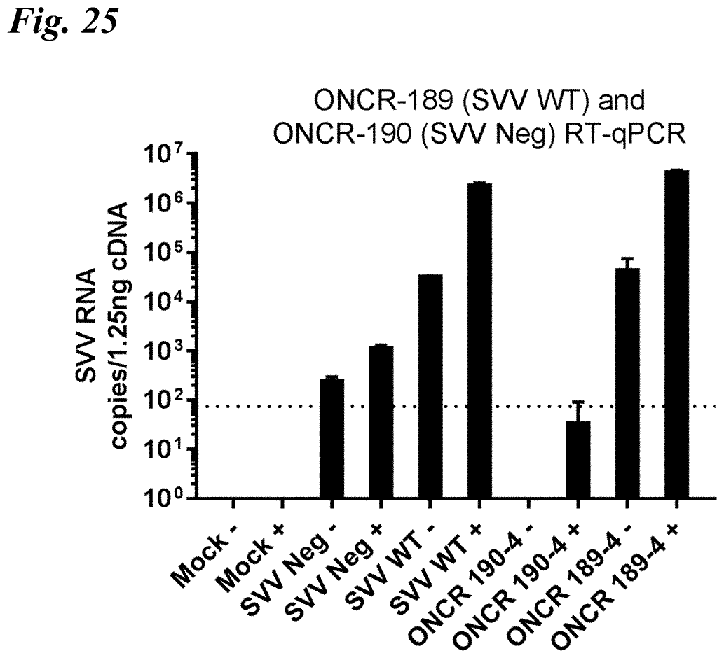

Production of the primary and secondary oncolytic viruses from a dual-oncolytic viral vector, or production of the primary and secondary viruses from a dual viral vector, can be measured by means known in the art, including RT-PCR for viral RNA and/or DNA sequences. For example, FIG. 25 shows a aPCR assay of the production of a secondary oncolytic SVV. H1299 cells were transfected with in vitro transcribed SVV-neg or SVV-wildtype (WT) positive strand RNA, or infected with oncolytic HSV-1 virus comprising polynucleotide encoding either a replication competent SVV (ONCR-189) or replication incompetent (ONCR-190) SVV viral genome. RNA was extracted and subjected to qRT-PCR for both the positive and negative sense SVV RNA strands. As shown, comparable levels of positive and negative sense RNAs were detected in both the ONCR-189 and the control transfection with SVV wildtype RNA (SVV WT), where as the SVV RNA leves are much lower in the ONCR-190 control, indicating that SVV viral replication is initiated from infection of HSV-1 comprising polynucleotide encoding a replication competent SVV. High levels of both strands are indicative of an active SVV infection induced or expressed by an oncolytic HSV, and this exemplifies initiation of infection of positive sense RNA virus from an oHSV infection.

In some embodiments, the present disclosure provides methods of treating a cancer in a subject in need thereof, comprising administering a dual-oncolytic virus or a dual virus described herein or composition thereof to the subject. A “subject,” as used herein, includes any animal that exhibits a symptom of a disease, disorder, or condition that can be treated with the recombinant viral vectors, compositions, and methods disclosed herein. Suitable subjects (e.g., patients) include laboratory animals (such as mouse, rat, rabbit, or guinea pig), farm animals (such as horse or cow), and domestic animals or pets (such as cat or dog). Non-human primates and, preferably, human patients, are included.

“Administration” refers herein to introducing a dual-oncolytic virus or a dual virus described herein or composition thereof to a subject or contacting a dual-oncolytic virus or a dual virus described herein or composition thereof with a cell and/or tissue. Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

The term “treating” and “treatment” as used herein refers to administering to a subject a therapeutically effective amount of a dual-oncolytic virus or a dual virus described herein or composition thereof so that the subject has an improvement in a disease or condition, or a symptom of the disease or condition. The improvement is any improvement or remediation of the disease or condition, or symptom of the disease or condition. The improvement is an observable or measurable improvement, or may be an improvement in the general feeling of well-being of the subject. Thus, one of skill in the art realizes that a treatment may improve the disease condition, but may not be a complete cure for the disease. A “prophylactically effective amount” refers to an amount of a dual-oncolytic virus or a dual virus described herein or composition thereof effective to achieve the desired prophylactic result. As used herein, “prophylaxis” can mean complete prevention of the symptoms of a disease, a delay in onset of the symptoms of a disease, or a lessening in the severity of subsequently developed disease symptoms. Typically, but not necessarily, since a prophylactic dose is used in subjects prior to or at an earlier stage of disease, the prophylactically effective amount is less than the therapeutically effective amount.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer.

In certain embodiments, a dual-oncolytic virus or a dual virus described herein or composition thereof is used to treat a cancer selected from lung cancer (e.g., small cell lung cancer or non-small cell lung cancer), breast cancer, ovarian cancer, cervical cancer, prostate cancer, testicular cancer, colorectal cancer, colon cancer, pancreatic cancer, liver cancer (e.g., hepatocellular carcinoma (HCC)), gastric cancer, head and neck cancer, thyroid cancer, malignant glioma, glioblastoma, melanoma, B-cell chronic lymphocytic leukemia, diffuse large B-cell lymphoma (DLBCL), and marginal zone lymphoma (MZL)

### EXAMPLES

### Example 1: Characterization of Doxycycline-Responsive Promoters

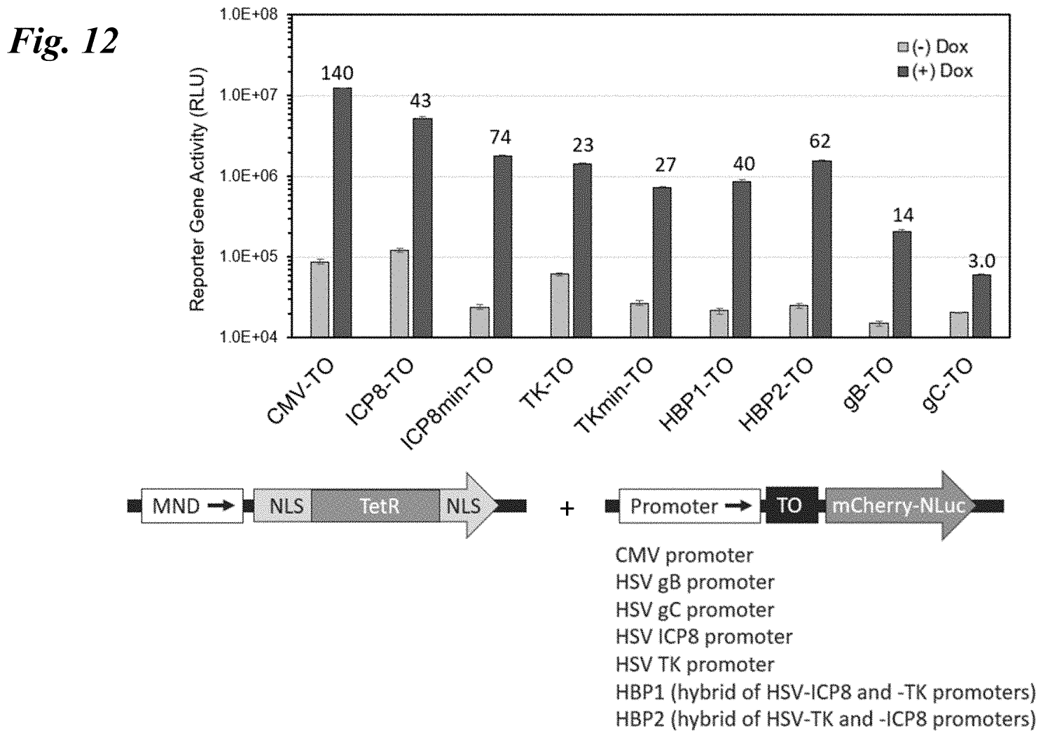

We tested the ability of various tetracycline (Tet)-dependent promoters to induce the expression of a report gene. Each Tet-dependent promoter comprising a Tet-On element downstream of the promoter was operably linked to a mCherry-NLuc reporter gene in a report construct (FIG. 12, right panel). HEK293T cells were transfected with the MND-TetR construct and one of the report construct using LipofectAMINE 2000 (ThermoFisher Scientific) according to standard methods. Following growth overnight, gene expression was induced by adding doxycycline to 200 nM to one set of replicate wells. Cells were incubated overnight and relative reporter gene activity was determined using a homogeneous assay for Nano luciferase activity (NanoGlo, Promega) (FIG. 12 bar graph; the numeric value above each bar shows the fold change of RLU after addition of doxycycline). The result showed that addition of doxycycline significantly increased the expression of the reporter gene. Among the Tet-dependent promoters tested, the CMV promoter showed the largest fold-change upon addition of doxycycline.

### Example 2: Characterization of Flp-Responsive STOP Cassette

HEK293T cells were co-transfected with:
a) MND-TetR construct (the NLS-TetR-NLS polypeptide is encoded by SEQ ID NO: 864);
b) Optionally, CMV-TetOn(TO)-Flp recombinase constructs (+Flp); negative control is indicated as (−Flp); and
c) Either CMV-TO-mCherry-NLuc without STOP cassette (NLuc) or CMV-TO-mCherry-NLuc construct with Flp-responsive STOP cassette (FSF-NLuc),
using LipofectAMINE 2000 (ThermoFisher Scientific) according to standard methods. Following growth overnight, gene expression was induced by adding doxycycline to 200 nM to one set of replicate wells. Cells were incubated overnight and relative reporter gene activity was determined using a homogeneous assay for Nano luciferase activity (NanoGlo, Promega).

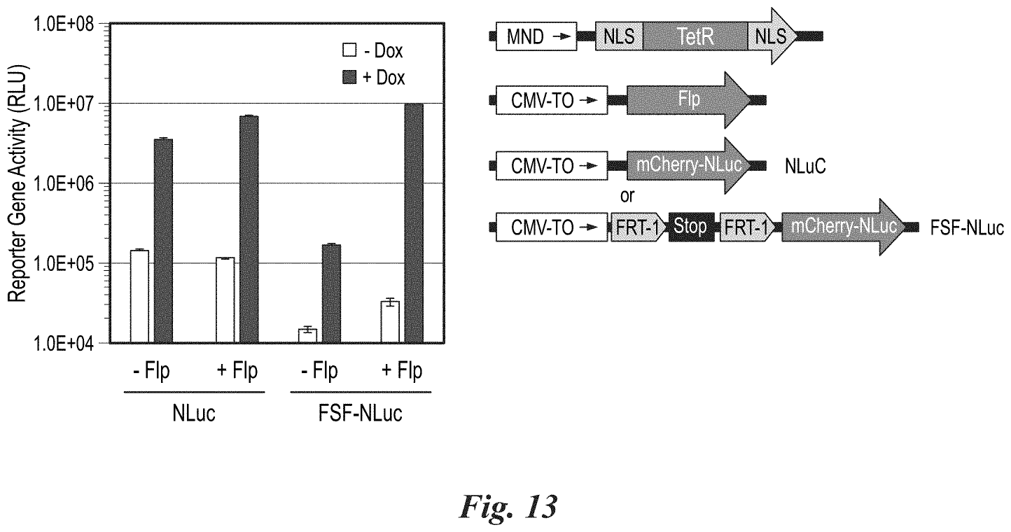

The results (FIG. 13) show that incorporation of the Flp-responsive STOP cassette (FSF-NLuc) resulted in greater control of reporter gene expression by doxycycline. In the absence of doxycycline, the baseline expression of reporter gene decreases in the FSF-NLuc groups (FIG. 13 right side of the bar graph). Addition of Flp recombinase expression construct, which is also controlled by Tet-On element, increases report gene expression overall for the FSF-NLuc construct compared to the FSF-NLuc group without Flp recombinase. On the contrary, for the reporter gene construct without Flp-responsive STOP cassette (NLuc; FIG. 13 left side of the bar graph), there was a higher baseline expression of reporter gene in the absence of doxycycline, and the expression level was not affected by the presence or absence of Flp recombinase expression construct.

### Example 3: Design of Flp-ERT2 Fusion Proteins for Post-Translational Control of Flp Activity

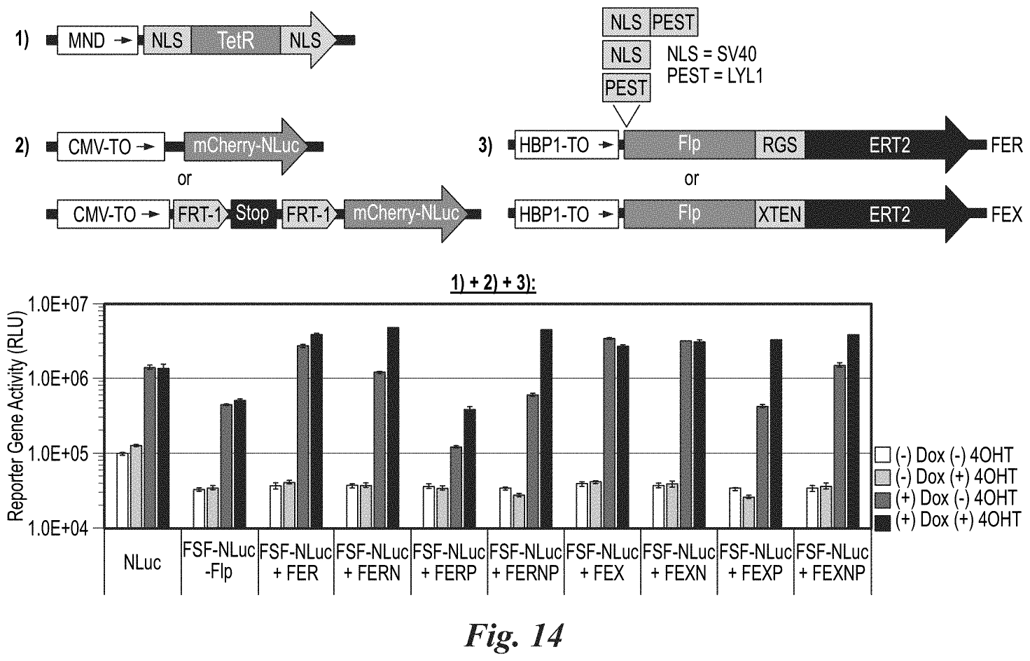

A number of Flp-ERT2 fusion proteins were tested for their ability to control Flp activity using Tamoxifen (FIG. 14).

Each Flp-ERT2 fusion proteins comprise Flp fused to a mutated estrogen receptor (ERT2) via a linker region. ERT2 only becomes activated and then translocates into the nucleus upon binding of the active tamoxifen metabolite 4-hydroxytamoxifen (4OHT). Accordingly, the Flp activity of fusion protein in the nucleus can be controlled by 4OHT.

A number of Flp-ERT2 fusion proteins were constructed. Fusion proteins that contains RGS linker region is denoted “FER”, whereas fusion proteins that contains XTEN linker region is denoted “FEX”. The N, P, and NP variants of the FER and FEX constructs refer to variants where the indicated NLS (N) and PEST (P) domains are engineered into the N-terminus of each recombinant protein. Each expression construct of the Flp-ERT2 fusion protein comprise a “HBP1 promoter-TetOn” region operably linked to the coding region of Flp-ERT2 fusion protein.

An exemplary FLP-RGS-ERT2 polypeptide as shown in FIG. 14 has an amino acid sequence encoded by SEQ ID NO: 846. An exemplary FLP-XTEN-ERT2 polypeptide as shown in FIG. 14 has an amino acid sequence encoded by SEQ ID NO: 847. An exemplary NLS sequence has an amino acid sequence encoded by SEQ ID NO: 848. An exemplary PEST sequence has an amino acid sequence encoded by SEQ ID NO: 849.

We tested the ability of each fusion protein construct to control gene expression. HEK293T cells were co-transfected with
a) the MND-TetR construct;
b) either the CMV-TO-mCherry-NLuc or CMV-TO-FSF-mCherry-NLuc construct; and
c) either a HBP1-TO-FER or HBP1-TO-FEX expression construct,
using LipofectAMINE 2000 (ThermoFisher Scientific) according to standard methods. Following growth overnight, gene expression was induced by adding 200 nM doxycycline and/or 1 uM 4-hydroxytamoxifen to sets of replicate wells. Cells were incubated overnight and relative reporter gene activity was determined using a homogeneous assay for Nano luciferase activity (NanoGlo, Promega).

According to the results (FIG. 14), several Flp-ERT2 fusion protein constructs showed 4OHT dependent Flp activity, including FERP, FERNPA, FEXP and FEXNP.

### Example 4: Effects of STOP Cassettes, Intron in the Flp Coding Region, and mRNA Destabilization Elements on Expression Control

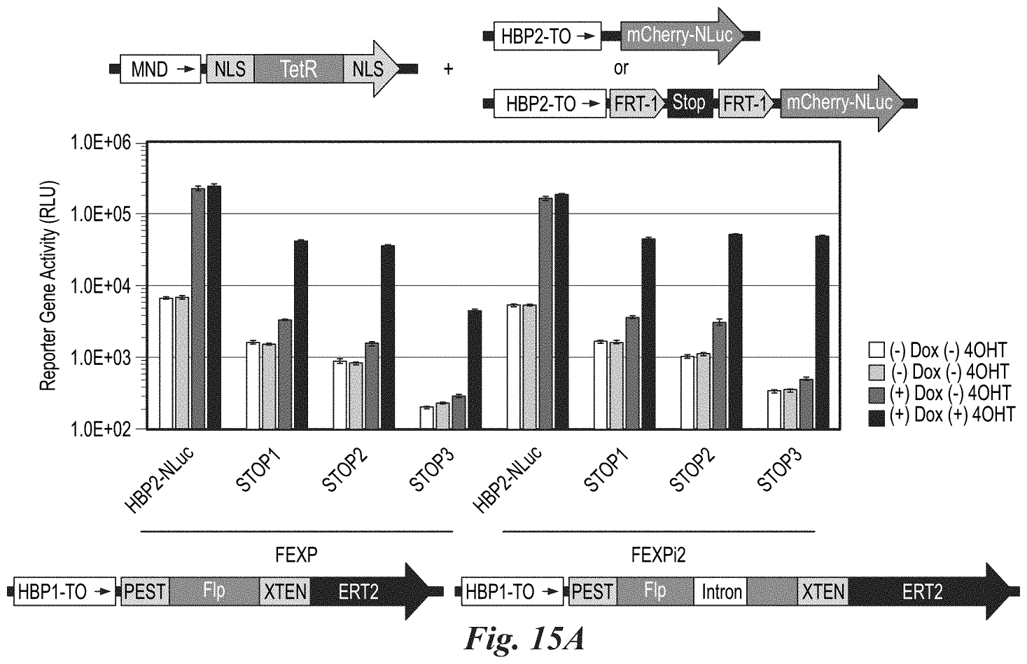

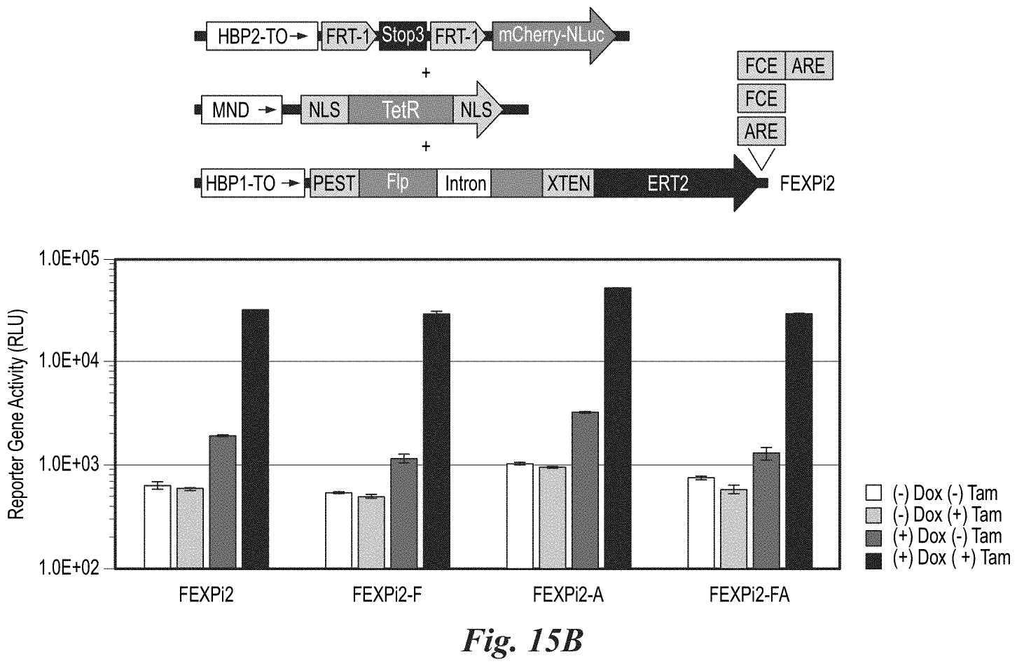

To further control Flp activity, we tested additional designs of Flp expression construct with insertion of intron element (FIG. 15A).

We used the FEXP construct (SEQ ID NO: 867) in the previous example as template and inserted an intron region (intron 2 of the ACTB gene, with necessary splice donor/acceptor elements) into the Flp coding region. The resultant construct is denoted “FEXPi2” (SEQ ID NO: 868). Therefore, The FEXP and FEXPi2 constructs differ by the insertion of intron 2 of the ACTB gene into the FLP coding region.

HEK293T cells were transfected with
a) MND-TetR construct;
b) either HBP2-TO-mCherry-NLuc or a HBP2-TO-FSF-mCherry-NLuc construct; and
c) either the HBP1-TO-FEXP or HBP1-TO-FEXPi2 (SEQ ID NO: 869) constructs
using LipofectAMINE 2000 (ThermoFisher Scientific) according to standard methods.
   Three versions of the HBP2-TO-FSF-mCherry-NLuc were used, each contain a STOP1 (SEQ ID NO: 854), STOP2 (SEQ ID NO: 855), or STOP3 (SEQ ID NO: 856) variants of the Flp-responsive STOP cassette, respectively. The STOP1, STOP2 and STOP3 variants differ by the number of tandem polyadenylaton signals in the cassette. Following growth overnight, gene expression was induced by adding 200 nM doxycycline and/or 1 uM 4-hydroxytamoxifen to sets of replicate wells. Cells were incubated overnight and relative reporter gene activity was determined using a homogeneous assay for Nano luciferase activity (NanoGlo, Promega).

According to the results (FIG. 15A), among the Flp responsive STOP cassettes, the STOP3 cassette with a longer tandem polyadenylation signal was more effective at suppressing baseline expression, whereas the presence of intron in the Flp coding region helped to maintain the maximal activity of Flp in the presence of both doxycycline and 4OHT and the expression level of the reporter gene when it was combined with an expression construct comprising STOP3 cassette.

We also tested additional designs of Flp expression construct with insertion of mRNA destabilization elements (FIG. 15B). Different mRNA destabilization elements were inserted into the FEXPi2 construct. The mRNA destabilization elements used were a c-fos coding element (FCE, SEQ ID NO: 894), an AU-rich element from the 3′UTR of c-fos gene (ARE, SEQ ID NO: 895), and the combination of both FCE and ARE in tandem (SEQ ID NO: 896). The FEXPi2 construct with FCE inserted is denoted FEXPi2-F, the FEXPi2 construct with ARE inserted is denoted FEXPi2-A, and the FEXPi2 construct with both FCE and ARE inserted is denoted FEXPi2-FA.

We transfected HEK293T cells with the MND-TetR and HBP2-TO-FSF-mCherry-NLuc constructs and variants of the HBP1-TO-FEXPi2 expression constructs using LipofectAMINE 2000 (ThermoFisher Scientific) according to standard methods. Following growth overnight, gene expression was induced by adding 200 nM doxycycline and/or 1 uM 4-hydroxytamoxifen to sets of replicate wells. Cells were incubated overnight and relative reporter gene activity was determined using a homogeneous assay for Nano luciferase activity (NanoGlo, Promega). The result (FIG. 15B) showed that incorporation of mRNA destabilization elements may affect the expression of the Flp recombinase.

### Example 5: Control of Target Gene Expression with Various Expression Construct Design

Various expression construct designs were tested for their effect on controlling gene expression.

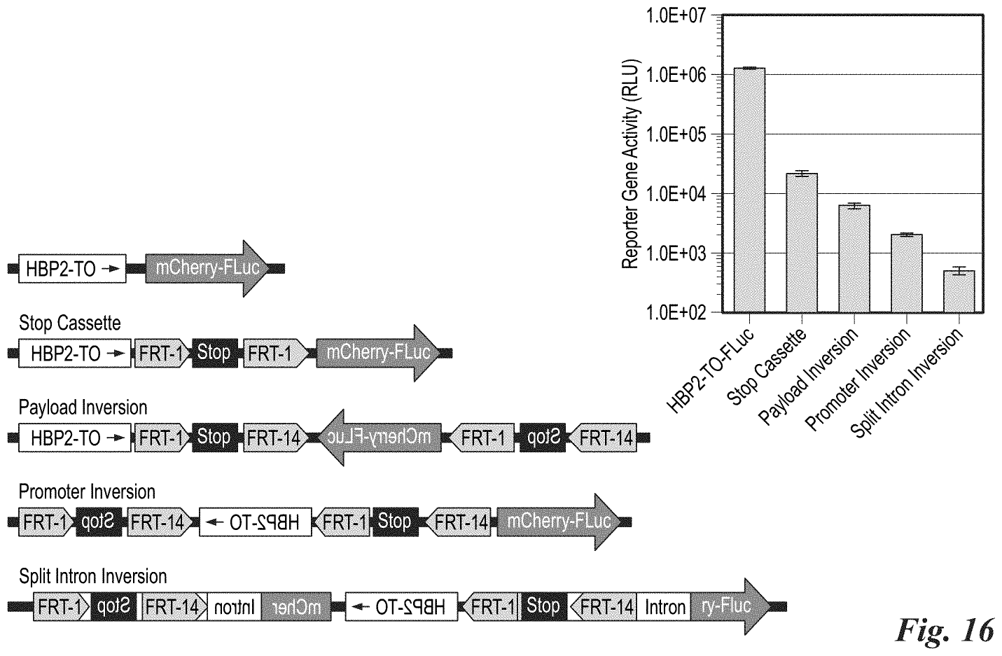

As shown in FIG. 16, the designs included:
1) STOP cassette: a construct comprising a STOP cassette inserted between promoter-TetOn region and reporter gene coding region;
2) Payload Inversion: a construct comprising two STOP cassettes; and the reporter gene coding region is inverted and placed under control of orthogonal Flp recognition sites (SEQ ID NO: 861);
3) Promoter Inversion: a construct comprising two STOP cassettes; and the promoter region is inverted and placed under control of orthogonal Flp recognition sites (SEQ ID NO: 860);
4) Split Intron Inversion: a construct comprising two STOP cassettes; the promoter region and part of the reporter gene coding region is inverted and placed under control of orthogonal Flp recognition sites; and intron regions are operably linked to the reporter gene coding regions (SEQ ID NO: 862).

HEK293T cells were transfected with the indicated expression constructs using LipofectAMINE 2000 (ThermoFisher Scientific) according to standard methods. Cells were incubated for two days and relative reporter gene activity was determined using a homogeneous assay for Firefly luciferase activity (ONE-Glo, Promega). The results (FIG. 16) showed that these designs resulted in decreasing baseline expression of reporter gene, among which the Split Intron Inversion design achieved lowest baseline (leaky) expression.

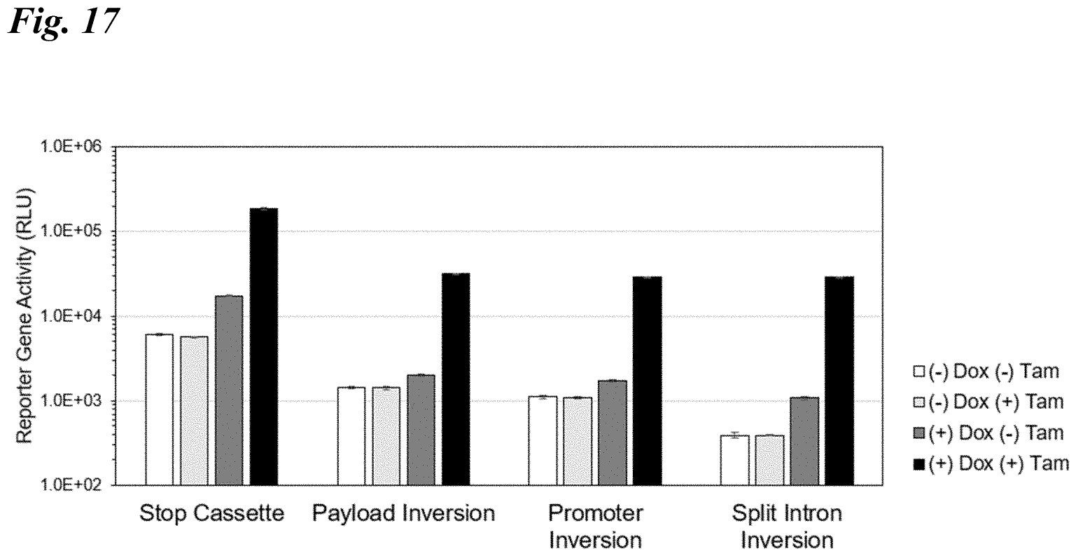

All these designs were also tested for their responsiveness to doxycycline and 4OHT. HEK293T cells were transfected with the indicated expression constructs using LipofectAMINE 2000 (ThermoFisher Scientific) according to standard methods. Following growth overnight, gene expression was induced by adding 200 nM doxycycline and/or 1 uM 4-hydroxytamoxifen to sets of replicate wells. Cells were incubated overnight and relative reporter gene activity was determined using a homogeneous assay for Firefly luciferase activity (ONE-Glo, Promega). The results (FIG. 17) showed that all these designs showed responsiveness to doxycycline and 40H and need both drugs for maximal expression.

### Example 6: Engineering of Transcriptional Control, Translational Control, and Payload Components in a Single Construct

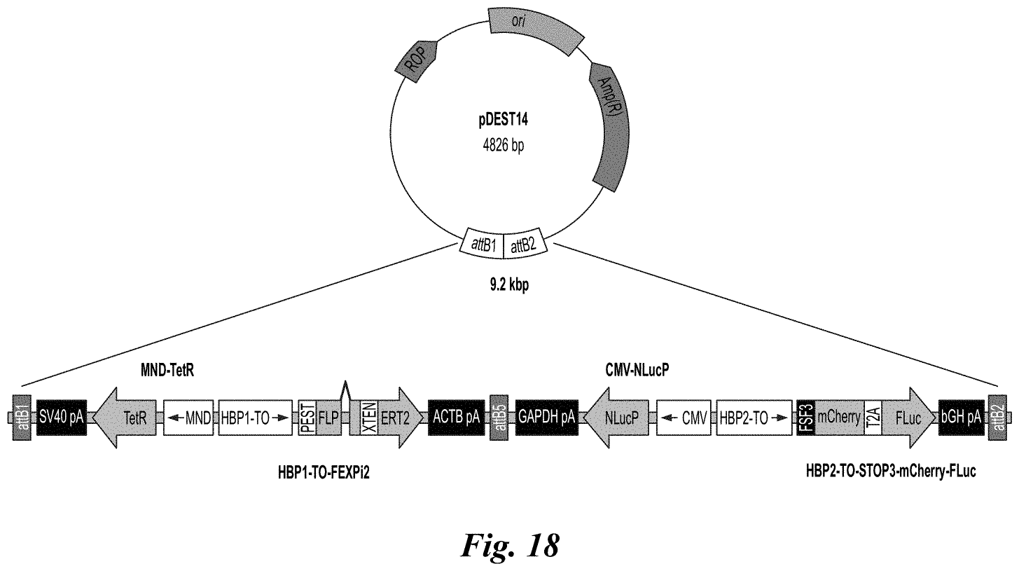

To construct a vector that integrates the transcriptional control, translational control, and payload components, Gateway attL sites were engineered into the various constructs to facilitate LR Clonase-mediated assembly of each component into the pDEST14 vector using the Multi Site Gateway system (ThermoFisher Scientific). The construct shown in FIG. 18 utilizes the STOP3 cassette as shown in the previous example (HBP2-TO-STOP3-mCherry-fLuc, SEQ ID NO: 870). Additional constructs utilizing the Payload Inversion design, the Promoter Inversion design, and the Split Intron Inversion design according to the previous example were also generated.

### Example 7: Engineering of Transcriptional Control, Translational Control, and OV2 Payload Components into an HSV Vector

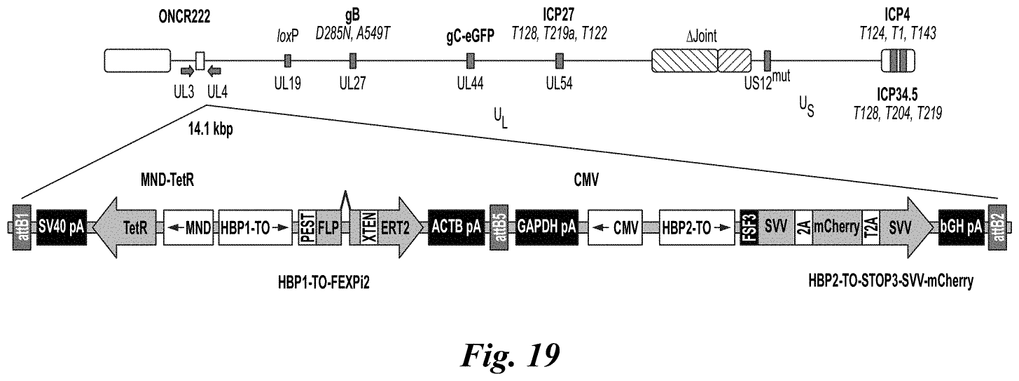

We constructs various dual oncolytic viral vectors that integrates transcriptional control, translational control, and secondary oncolytic viral genome components in a single HSV based oncolytic viral vector. Gateway attL sites were engineered into the various constructs to facilitate LR Clonase-mediated assembly of each component into the ONCR222b vector using the MultiSite Gateway system (ThermoFisher Scientific). One exemplary construct is shown in FIG. 19, which integrates the HBP1_promoter-TetOn-FEXPi2 cassette (SEQ ID NO: 869) for Flp recombinase expression, and a HBP2_promoter-TetOn-STOP3-SVV-mCherry (SEQ ID NO: 871) cassette to enable transcription and translation of SVV viral genome as well as mCherry reporter gene once the STOP3 element is excised by the Flp recombinase. The full 14.1 kb insertion sequence is provided in SEQ ID NO: 872. While the recombinase responsive STOP3 cassette is used to control the secondary oncolytic virus in FIG. 19, we also constructed similar dual oncolytic viral vectors that uses the Payload Inversion design, the Promoter Inversion design, and the Split Intron Inversion design, respectively, to control the secondary oncolytic virus. Each of the HSV based dual oncolytic viral vector was reconstituted by transfection into Vero-SF cells using Fugene HD (Promega) and viral stocks were expanded and tittered according to standard methods.

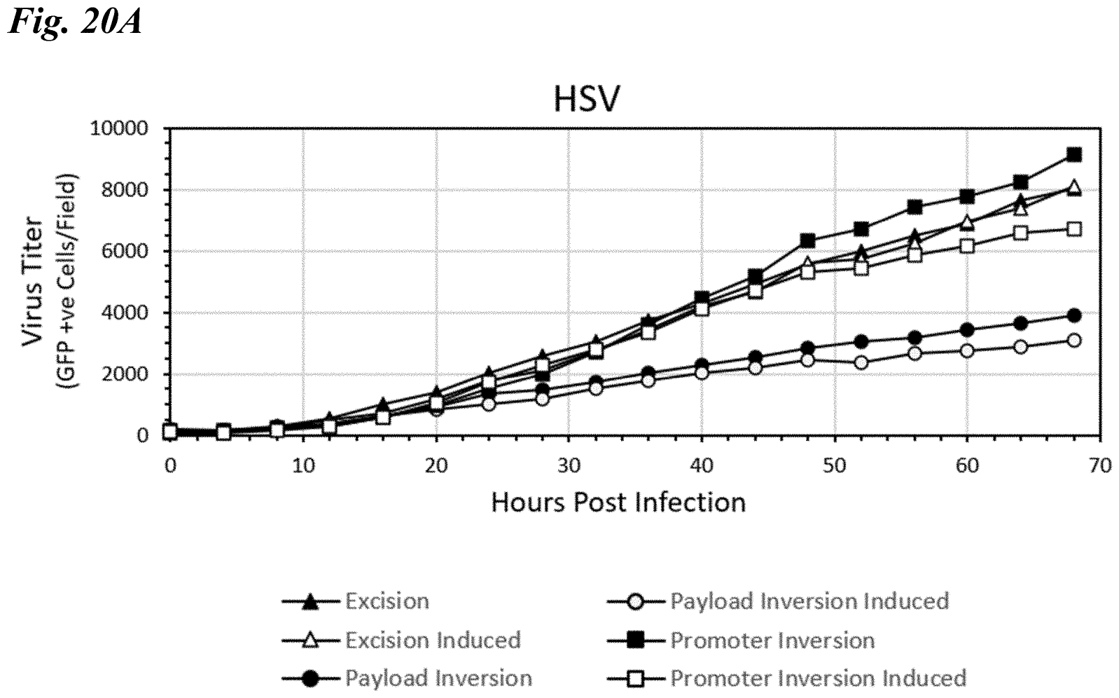

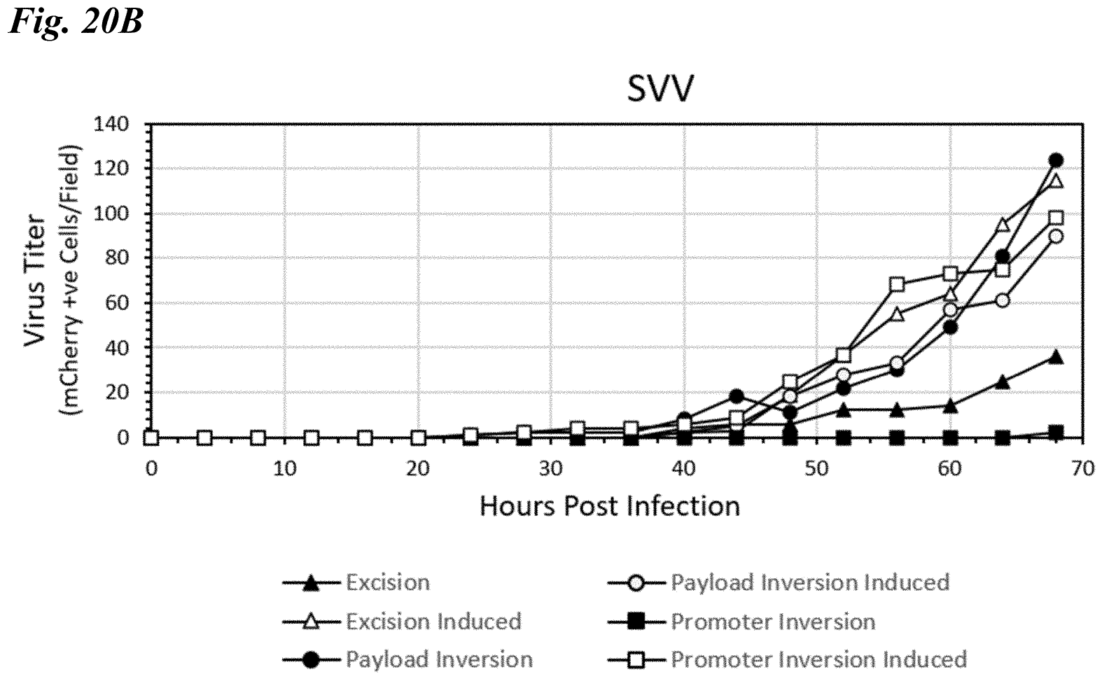

NCI-H1299 cells were infected with the indicated ONCR-222 based dual oncolytic viral vector at a multiplicity of infection of 0.1 pfu/cell, and SVV-mCherry replication was induced by adding 200 nM doxycycline and 1 uM 4-hydroxytamoxifen to replicate wells. Viral replication was assayed every 2 hours for 3 days using an automated inverted fluorescent microscope (IncuCyte S3) to screen for GFP expression from HSV and mCherry expression from SVV. The data was plotted as the total number of GFP and mCherry cells/microscope field as indication of virus titer for HSV (FIG. 20A) or SVV (FIG. 20B). The results showed that, for all the dual oncolytic viral vectors tested, virus titer of HSV was not significantly affected by the presence of doxycycline and 4-hydroxytamoxifen (FIG. 20A). On the other hand, for the dual oncolytic vectors with STOP3 Excision cassette design or the Promoter Inversion design, the virus titer of SVV increased significantly in the presence of doxycycline and 4-hydroxytamoxifen (FIG. 20B). Especially, for the dual oncolytic viral vector using the Promoter Inversion design in the SVV secondary oncolytic virus (SVV) expression cassette, there was minimal basal production of SVV in the absence of doxycyucline and 4-hydroxytamoxifen but high SVV production after induction with these small molecules.

### Example 8: Dual Oncolytic Virus Displays More Potent Anti-Tumor Effect In Vivo

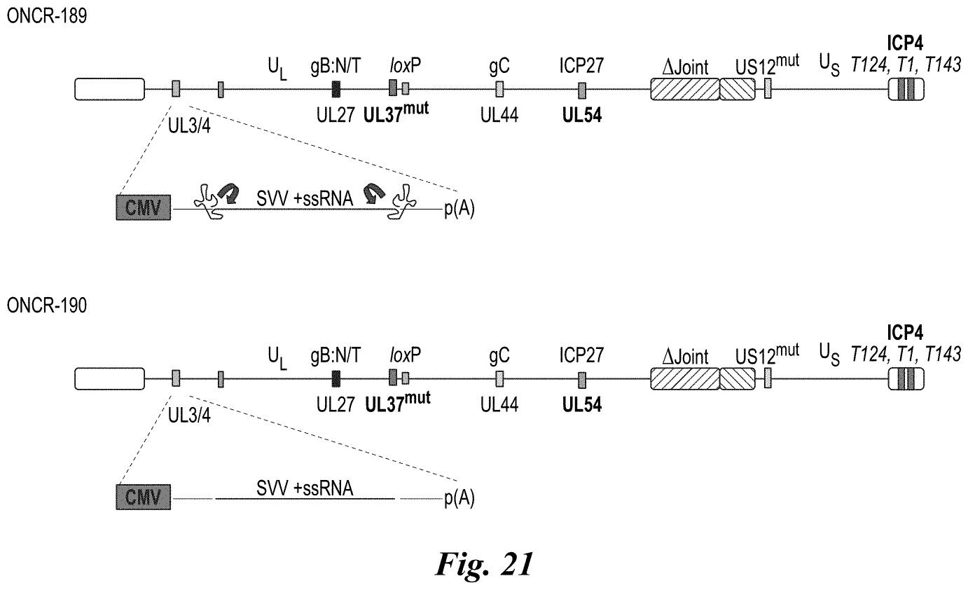

We constructed dual oncolytic viral vectors by inserting a SVV viral genome into an oncolytic HSV backbone vector ONCR-142 (FIG. 21). Expression of SVV +ssRNA is controlled by the CMV promoter within the gateway cassette. In ONCR-189 (SEQ ID NO: 873), after the transcription of RNA encoding SVV viral genome, the self cleaving ribozymes flanking the SVV viral genome serve as junctional cleavage sequences to remove the non-viral RNA form the transcript, which in turn liberates the SVV RNA to allow production of infectious SVV virus. In the control vector ONCR-190 (SEQ ID NO: 874), there is no such ribozymes, and therefore infectious SVV viral RNA is not produced. Therefore, ONCR-189 is SVV replication competent, whereas ONCR-190 is SVV replication incompetent.

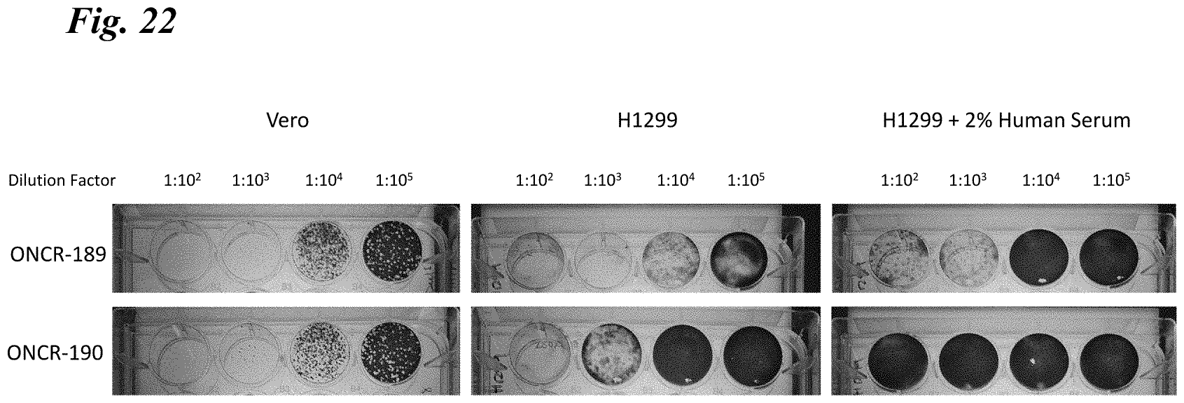

Viral stocks from ONCR-189 and ONCR-190 were produced and titer in Vero cells. We tested the lytic activity of ONCR-189 or ONCR-190 virus stock by infecting Vero or H1299 cells (FIG. 22). 10-fold serial dilutions of each virus stock were used to infect Vero or H1299 cells, and cells were stained with crystal violet to visualize lytic cell death and monolayer clearance. ONCR-189 and ONCR-190 demonstrated equivalent cell killing in Vero cells, which are sensitive to HSV but resistant to SVV infection. On the other hand, H1299 cells are sensitive to both HSV and SVV infection, and ONCR-189 cleared monolayers of H1299 cells at a higher dilution than ONCR-190. Further, human serum neutralizes HSV but not SVV, and when viral infection was conducted in the presence of 2% human serum, cell lysis is inhibited in ONCR-190 control but not ONCR-189 infection. These experiments demonstrated that ONCR-189 can effectively generate functional SVV secondary oncolytic viruses.

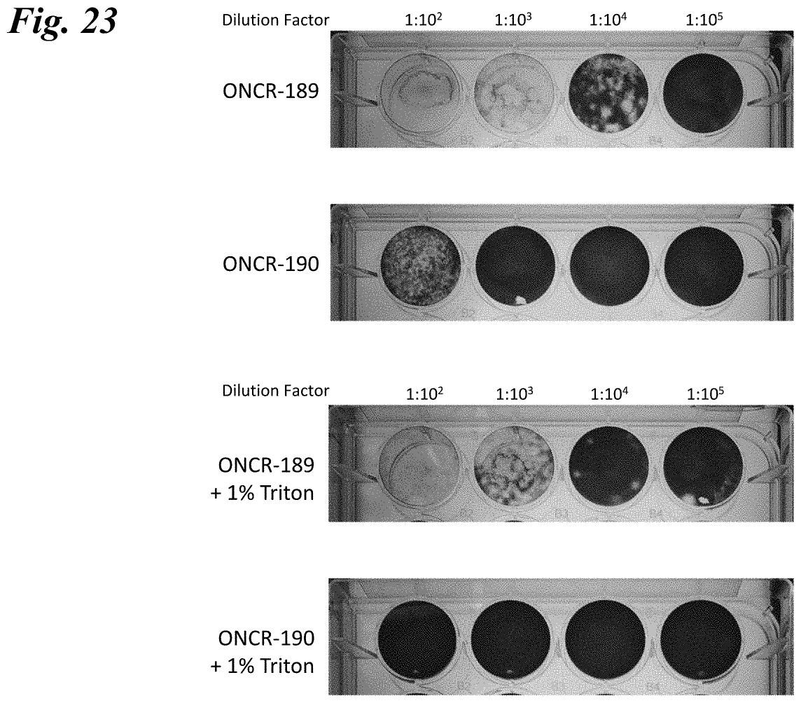

We then tested whether 1% Triton would affect the ability of ONCR-189 or ONCR-190 virus stock to infect H1299 cells (FIG. 23). 10-fold serial dilutions of each virus stock were used to infect H1299 cells, and cells were stained with crystal violet to visualize lytic cell death and monolayer clearance. H1299 cells are sensitive to both HSV and SVV induced cell lysis. ONCR-189 clears monolayers at a higher dilution compared to ONCR-190 when infected with the same HSV-1 MOI (due to the presence of HSV and SVV virions in the stock). 1% Triton disrupts the envelope of HSV and inactivates infectious HSV virions, but does not affect the non-enveloped SVV. Thus cell lysis is inhibited at the presence of 1% Triton for ONCR-190, but not for ONCR-189 stock (only HSV infection was inactivated, but since ONCR-189 viral stock contained HSV and SVV virions, and SVV is not inactivated by 1% Triton, SVV virions lysed H1299 cells).

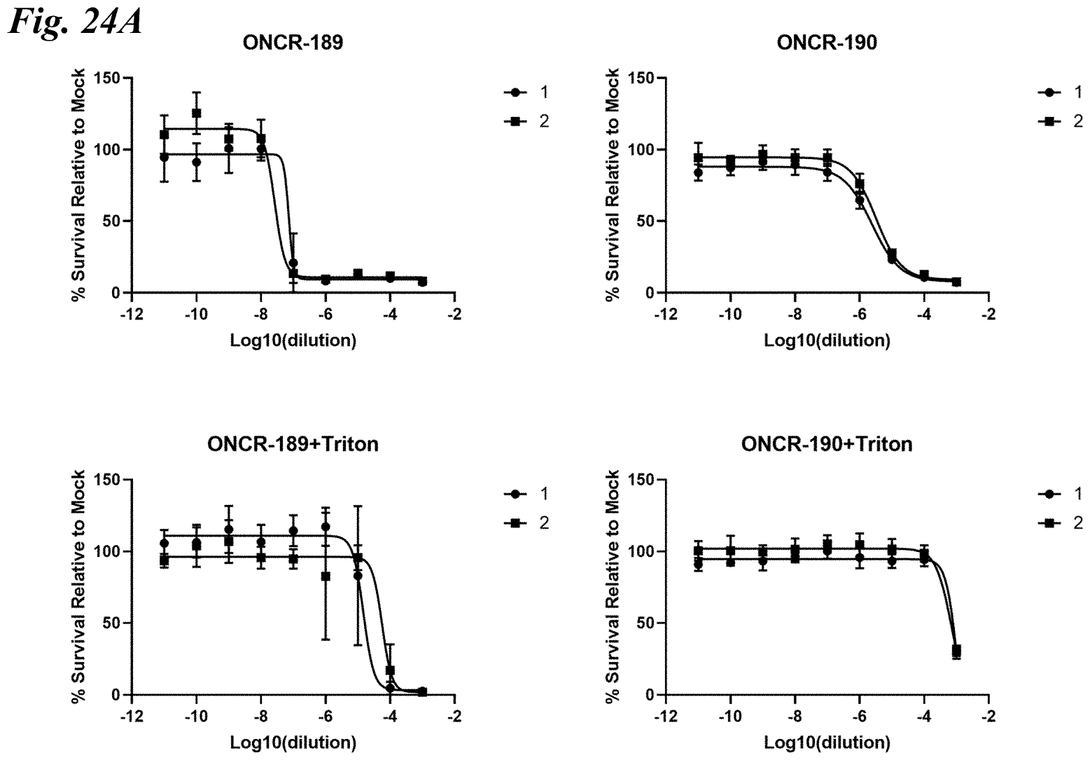

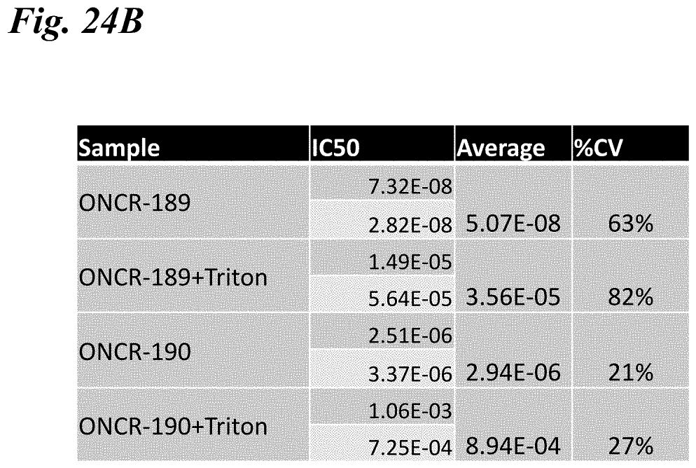

An IC50 titer assay was performed for ONCR-189 and ONCR-190 virus infection of H446 cells (FIG. 24A). H446 cells that are sensitive to both HSV and SVV infections. The results showed that the IC50 titer of ONCR-189 was lower as compared to ONCR-190. That is, ONCR-189 is more potent at killing H446 cells. Infection in the presence of 1% Triton disrupts the envelope of HSV and inactivates infectious HSV virions but does not affect SVV, thus cell lysis was inhibited in ONCR-190 but not ONCR-189 infection when 1% Triton was added. The IC50 values are summarized in FIG. 24B.

In another set of experiments, H1299 cells were transfected with in vitro transcribed SVV-neg or SVV-wt positive strand RNA or infected with ONCR-189 and ONCR-190 oHSV. RNA sample from each test group was extracted and subject to a RT qPCR assay (FIG. 25). The results showed that transfection of SVV-WT positive strand RNA resulted in high copy of both positive and negative strand RNA indicating viral replication. Levels of SVV positive and negative strand RNA in the ONCR-189 infected cells were similar to SVV-WT transfected cells, and were much higher than the SVV RNA levels in the ONCR-190 infected cells, indicating successful SVV viral replication in ONCR-189 oHSV infected cells.

### Example 9: Dual Oncolytic Virus Shows Anti-Tumor Effect In Vivo

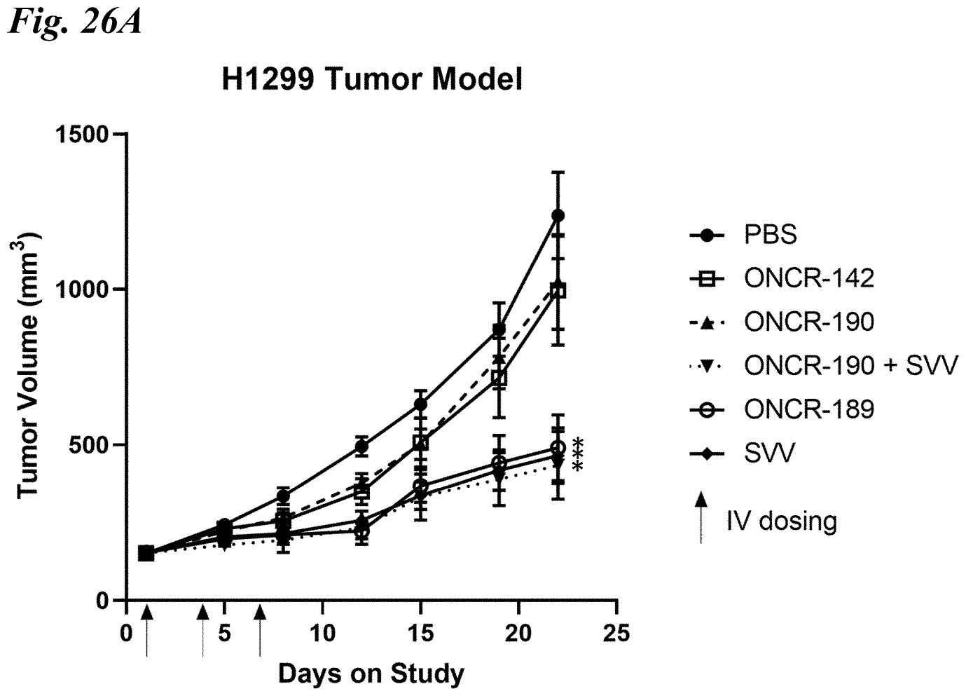

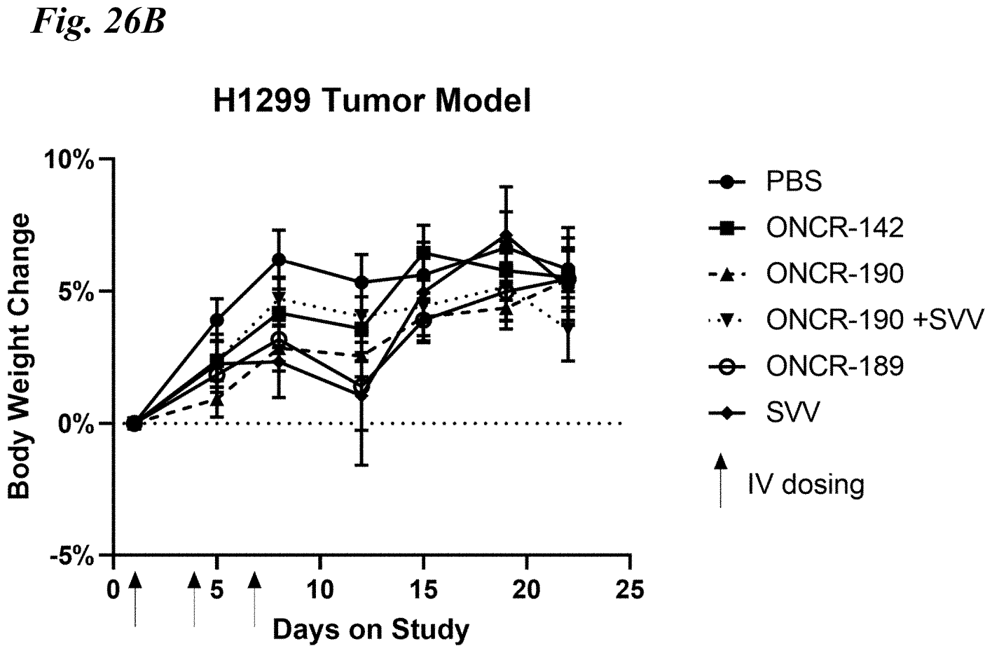

Evaluation of the in vivo efficacy of dual oncolytic virus was carried out in nude mice bearing subcutaneous xenograft tumors (NCI-H1299). Each oHSV was administered intravenously (IV) at IV doses of 1×10⁷ PFU, and SVV was administered at IV doses of 1×10⁴ PFU. NCI-H1299 bearing mice were cohort when tumors reached 150 mm³ (n=7 per group) and dosed intravenously on days 1, 4, and 7. Tumor growth (FIG. 26A) and body weight (FIG. 26B) were measured twice weekly.

As shown in FIG. 26A, oHSV backbone vector (ONCR-142) and oHSV encoding SVV without ribozymes (ONCR-190) had no anti-tumor effect in this tumor model. On the other hand, mice treated with SVV virions or ONCR-190 combined with SVV virions showed significant tumor growth inhibition. Similar tumor growth inhibition was observed for mice dosed IV with ONCR-189, suggesting that ONCR-189 can effectively produce functional SVV virions once it is delivered in vivo which are able to inhibitor tumor growth. No adverse effects on animal body weight were observed for any of the treatments tested in this study (FIG. 26B). For group statistical comparisons, a Two-way ANOVA (Bonferroni's multiple comparisons test) was used. P values are relative to PBS control, where * indicates P<0.05.

### Example 10: Control of Gene Expression Using a TetOFF Ribozyme

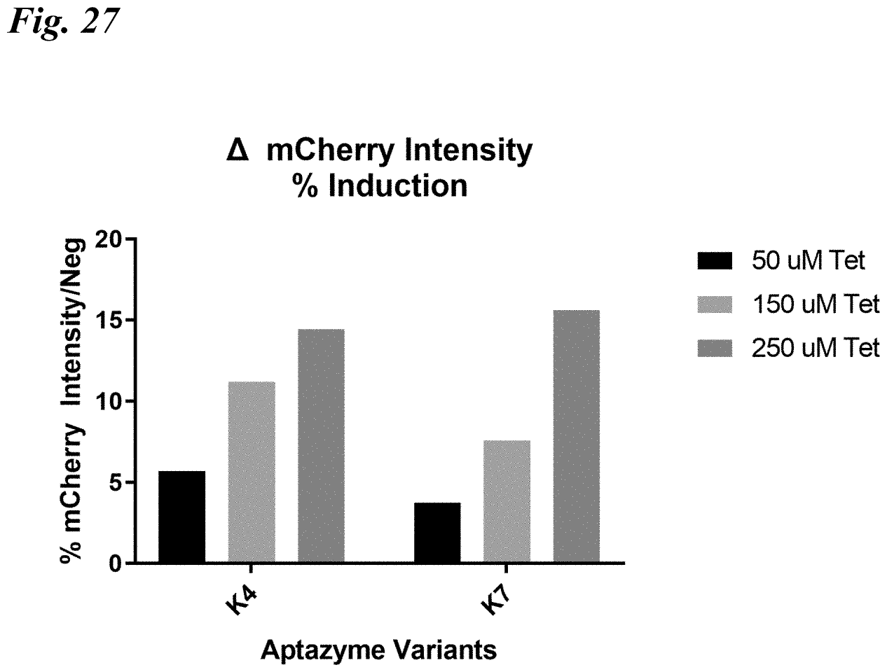

HEK293 cells were transiently transfected with mCherry reporter vector plasmid expressing a transcript containing the K4 aptazyme (SEQ ID NO: 913) or the K7 aptazyme (SEQ ID NO: 914) in the 3′ UTR. The expression level of mCherry were assessed by array scanning cytometery on a Spectramax Minimax at 48 hours post transfection upon addition of indicated concentration of tetracycline. The result (FIG. 27) showed that tetracycline represses aptazyme cleavage of transcript to induce gene expression.

### Example 11: Dual Oncolytic Virus Shows Sustained Anti-Tumor Effect In Vivo

Evaluation of the in vivo efficacy of dual oncolytic virus will be carried out in nude mice bearing subcutaneous xenograft tumors partially sensitive to the primary oncolytic virus. Viruses will be administered intravenously (IV) at IV. Tumor bearing mice will be cohorted when tumors reached 150 mm³ (n=7-10 per group) and dosed three times intravenously. Tumor growth will be measured twice weekly.

Primary oncolytic virus encoding a non-replication competent secondary oncolytic virus will have a partial anti-tumor effect in this tumor model, where tumors will relapse after a certain amount of time. On the other hand, mice treated primary oncolytic virus encoding a replication-competent secondary oncolytic virus that can effectively produce functional secondary virions will inhibit tumor growth to a greater extent, and for a more extended period.

### FURTHER NUMBERED EMBODIMENTS

Further embodiments of the instant disclosure are provided in the numbered embodiments below:

Embodiment 1. A recombinant primary oncolytic virus, comprising: a polynucleotide encoding a secondary oncolytic virus.

Embodiment 2. A recombinant primary virus, comprising: a polynucleotide encoding a secondary virus.

Embodiment 3. The virus of Embodiment 1, wherein the primary oncolytic virus and the secondary oncolytic virus are replication-competent.

Embodiment 4. The virus of Embodiment 2, wherein the primary virus and the secondary virus are replication-competent.

Embodiment 5. The virus of Embodiment 1, wherein the primary oncolytic virus and/or the secondary oncolytic virus is/are replication-incompetent.

Embodiment 6. The virus of Embodiment 2, wherein the primary virus and/or the secondary virus is/are replication-incompetent.

Embodiment 7. The virus of any one of Embodiments 1, 3, and 5, wherein the polynucleotide encoding the secondary oncolytic virus is operably linked to a regulatable promoter.

Embodiment 8. The virus of any one of Embodiments 2, 4, and 6, wherein the polynucleotide encoding the secondary virus is operably linked to a regulatable promoter.

Embodiment 9. The virus of any one of Embodiments 1, 3, 5, and 7, wherein the primary oncolytic virus generates an antigen-specific immune response that does not mediate antigen-specific immunity against the secondary oncolytic virus.

Embodiment 10. The virus of any one of Embodiments 2, 4, 6, and 8, wherein the primary virus generates an antigen-specific immune response that does not mediate antigen-specific immunity against the secondary virus.

Embodiment 11. The virus of any one of Embodiments 1, 3, 5, 7, and 9, wherein the primary oncolytic virus is a double-stranded DNA (dsDNA) virus.

Embodiment 12. The virus of any one of Embodiments 2, 4, 6, 8, and 10, wherein the primary virus is a double-stranded DNA (dsDNA) virus.

Embodiment 13. The virus of Embodiment 11 or 12, wherein the dsDNA virus is a herpes simplex virus (HSV) or an adenovirus.

Embodiment 14. The virus of Embodiment 11 or 12, wherein the dsDNA virus is a virus of Poxviridae family.

Embodiment 15. The virus of Embodiment 14, wherein the dsDNA virus is a molluscum contagiosum virus, a myxoma virus, a vaccina virus, a monkeypox virus, or a yatapoxvirus.

Embodiment 16. The virus of any one of Embodiments 1, 3, 5, 7, and 9, wherein the primary oncolytic virus is a RNA virus.

Embodiment 17. The virus of any one of Embodiments 2, 4, 6, 8, and 10, wherein the primary virus is a RNA virus.

Embodiment 18. The virus of Embodiment 16 or 17, wherein the RNA virus is a paramyxovirus or a rhabdovirus.

Embodiment 19. The virus of any one of Embodiments 1, 3, 5, 7, 9, 11, 13-16, and 18, wherein the secondary oncolytic virus is a positive-sense single-stranded RNA (ssRNA) virus, a negative-sense ssRNA virus, or an ambi-sense ssRNA virus.

Embodiment 20. The virus of any one of Embodiments 2, 4, 6, 8, 10, 12-15, and 17-18, wherein the secondary virus is a positive-sense single-stranded RNA (ssRNA) virus, a negative-sense ssRNA virus, or an ambi-sense ssRNA virus.

Embodiment 21. The virus of Embodiment 19 or 20, wherein the secondary oncolytic virus or the secondary virus is a negative-sense ssRNA virus of Rrhabdoviridae family, Paramyxoviridae family, or Orthomyxoviridae family.

Embodiment 22. The virus of Embodiment 21, wherein the virus of Rhabdoviridae family is a vesicular stomatitis virus (VSV) or a maraba virus.

Embodiment 23. The virus of Embodiment 21, wherein the virus of Paramyxoviridae family is a Newcastle Disease virus, a Sendai virus, or a measles virus.

Embodiment 24. The virus of Embodiment 21, wherein the virus of Orthomyxoviridae family is an influenza virus.

Embodiment 25. The virus of Embodiment 19 or 20, wherein the secondary oncolytic virus or the secondary virus is the positive-sense ssRNA virus, and wherein the positive-sense ssRNA virus is an enterovirus.

Embodiment 26. The virus of Embodiment 25, wherein the enterovirus is a poliovirus, a Seneca Valley virus (SVV), a coxsackievirus, or an echovirus.

Embodiment 27. The virus of Embodiment 26, wherein the coxsakivirus is a coxsackievirus A (CVA) or a coxsackievirus B (CVB),

Embodiment 28. The virus of Embodiment 27, wherein the coxsakivirus is CVA9, CVA21 or CVB3.

Embodiment 29. The virus of Embodiment 19 or 20, wherein the secondary oncolytic virus or the secondary virus is the positive-sense ssRNA virus, and wherein the positive-sense ssRNA virus is a Encephalomyocarditis virus (EMCV).

Embodiment 30. The virus of Embodiment 19 or 20, wherein the secondary oncolytic virus or the secondary virus is the positive-sense ssRNA virus, and wherein the positive-sense ssRNA virus is a Mengovirus.

Embodiment 31. The virus of Embodiment 19 or 20, wherein the secondary oncolytic virus or the secondary virus is the positive-sense ssRNA virus, and wherein the positive-sense ssRNA virus is a virus of Togaviridae family.

Embodiment 32. The virus of Embodiment 31, wherein the virus of Togaviridae familyis a new world alphavirus or old world alphavirus.

Embodiment 33. The virus of Embodiment 32, wherein the new world alphavirus or old world alphavirusis is VEEV, WEEV, EEV, Sindbis virus, Semliki Forest virus, Ross River Virus, or Mayaro virus.

Embodiment 34. The virus of any one of Embodiments 1, 3, 5, 7, 9, 11, 13-16, 18-19, and 21-33, wherein the primary oncolytic virus and/or the secondary oncolytic virus is a chimeric virus.

Embodiment 35. The virus of any one of Embodiments 2, 4, 6, 8, 10, 12-15, 17-18, and 20-33, wherein the primary virus and/or the secondary virus is a chimeric virus.

Embodiment 36. The virus of any one of Embodiments 1, 3, 5, 7, 9, 11, 13-16, 18-19, and 21-34, wherein the primary oncolytic virus and/or the secondary oncolytic virus is a pseudotyped virus.

Embodiment 37. The virus of any one of Embodiments 2, 4, 6, 8, 10, 12-15, 17-18, 20-33, and 35, wherein the primary virus and/or the secondary virus is a pseudotyped virus.

Embodiment 38. The virus of Embodiment 36, wherein the secondary oncolytic virus is a pseudotyped virus, and wherein the primary oncolytic virus comprises a coding region for a capsid protein or an envelope protein of the secondary oncolytic virus outside the cording region for the secondary oncolytic virus.

Embodiment 39. The virus of Embodiment 38, wherein the secondary oncolytic virus is an alphavirus, a paramyxovirus or a rhabdovirus.

Embodiment 40. The virus of Embodiment 37, wherein the secondary virus is a pseudotyped virus, and wherein the primary virus comprises a coding region for a capsid protein or an envelope protein of the secondary virus outside the cording region for the secondary virus.

Embodiment 41. The virus of Embodiment 40, wherein the secondary virus is an alphavirus, a paramyxovirus or a rhabdovirus.

Embodiment 42. The virus of any one of Embodiments 7-41, wherein the regulatable promoter is selected from a steroid-inducible promoter, a metallothionine promoter, an MX-1 promoter, a GENESWITCH™ hybrid promoter, a cumate-responsive promoter, and a tetracycline-inducible promoter.

Embodiment 43. The virus of any one of Embodiments 7-41, wherein the regulatable promoter comprises a constitutive promoter flanked by recombinase recognition sites.

Embodiment 44. The virus of any one of Embodiments 1-43, further comprising a second polynucleotide encoding a peptide capable of binding to the regulatable promoter.

Embodiment 45. The virus of Embodiment 44, wherein the second polynucleotide is operably linked to a constitutive promoter or an inducible promoter.

Embodiment 46. The virus of Embodiment 45, wherein the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

Embodiment 47. The virus of any one of Embodiments 44-46, wherein the regulatable promoter is a tetracycline (Tet)-dependent promoter and wherein in the peptide is a reverse tetracycline-controlled transactivator (rtTA) peptide.

Embodiment 48. The virus of any one of Embodiments 44-46, wherein the regulatable promoter is a tetracycline (Tet)-dependent promoter and wherein in the peptide is a tetracycline-controlled transactivator (tTA) peptide.

Embodiment 49. The virus of any one of Embodiments 1-48, wherein the primary oncolytic virus or the primary virus further comprises a polynucleotide encoding one or more RNA interference (RNAi) molecules.

Embodiment 50. The virus of Embodiment 49, wherein the polynucleotide encoding one or more RNA interference (RNAi) molecules is operably linked to a second regulatable promoter.

Embodiment 51. The virus of Embodiment 49 or 50, wherein the one or more RNAi molecules bind to a target sequence in the genome of the secondary oncolytic virus or the secondary virus and inhibits replication of the secondary oncolytic virus or the secondary virus.

Embodiment 52. The virus of any one of Embodiments 49-51, wherein the RNAi molecule is an siRNA, an miRNA, an shRNA, or an AmiRNA.

Embodiment 53. The virus of any one of Embodiments 1, 3, 5, 7, 9, 11, 13-16, 18-19, 21-34, 36, 38-39, and 42-52, wherein the polynucleotide encoding the secondary oncolytic virus comprises one or more recombinase recognition sites.

Embodiment 54. The virus of any one of Embodiments 2, 4, 6, 8, 10, 12-15, 17-18, 20-33, 35, 37, and 40-52, wherein the polynucleotide encoding the secondary virus comprises one or more recombinase recognition sites.

Embodiment 55. The virus of any one of Embodiments 1, 3, 5, 7, 9, 11, 13-16, 18-19, 21-34, 36, 38-39, and 42-53, wherein the polynucleotide encoding the secondary oncolytic virus comprises one or more recombinase-responsive cassettes, wherein the recombinase-responsive cassette comprises the one or more recombinase recognition sites.

Embodiment 56. The virus of any one of Embodiments 2, 4, 6, 8, 10, 12-15, 17-18, 20-33, 35, 37, 40-52, and 54, wherein the polynucleotide encoding the secondary virus comprises one or more recombinase-responsive cassettes, wherein the recombinase-responsive cassette comprises the one or more recombinase recognition sites.

Embodiment 57. The virus of Embodiment 55 or 56, wherein the one or more recombinase-responsive cassettes comprise a Recombinase-Responsive Excision Cassette (RREC).

Embodiment 58. The virus of Embodiment 57, wherein the RREC comprises a transcriptional/translational termination (STOP) element.

Embodiment 59. The virus of the Embodiment 58, wherein the transcriptional/translational termination (STOP) element comprises a sequence having 80% identity to any one of SEQ ID NOS: 854-856.

Embodiment 60. The virus of any one of Embodiments 55-59, wherein the one or more recombinase-responsive cassettes comprise a Recombinase-Responsive Inversion Cassette (RRIC).

Embodiment 61. The virus of the Embodiment 60, wherein the RRIC comprises two or more orthogonal Recombinase Recognition Sites on each side of a Central Element.

Embodiment 62. The virus of Embodiment 60 or 61, wherein the RRIC comprises a promoter or a portion of the promoter.

Embodiment 63. The virus of Embodiment 60 or 61, wherein the RRIC comprises a coding region or a portion of the coding region, wherein the coding region encodes the viral genome of the secondary oncolytic virus or the secondary virus.

Embodiment 64. The virus of any one of Embodiments 60-63, wherein the RRIC comprises one or more Control Element(s).

Embodiment 65. The virus of Embodiment 64, wherein the Control Element(s) is/are transcriptional/translational termination (STOP) elements.

Embodiment 66. The virus of Embodiment 65, wherein the Control Element(s) has/have a sequence having 80% identity to any one of SEQ ID NOS: 854-856.

Embodiment 67. The virus of any one of Embodiments 60-66, wherein the Recombinase-Responsive Inversion Cassette (RRIC) further comprises a portion of an intron.

Embodiment 68. The virus of Embodiment 67, wherein the polynucleotide encoding the secondary oncolytic virus or the secondary virus yields a mature viral genome transcript of the secondary oncolytic virus or the secondary virus without the Recombinase Recognition Site after removal of the intron via mRNA splicing.

Embodiment 69. The virus of any one of Embodiments 1-68, wherein the primary oncolytic virus or the primary virus further comprises a polynucleotide encoding the recombinase.

Embodiment 70. The virus of Embodiment 69, wherein the recombinase is a Flippase (Flp) or a Cre recombinase (Cre).

Embodiment 71. The virus of Embodiment 69 or 70, wherein the coding region of the recombinase comprises an intron.

Embodiment 72. The virus of any one of Embodiments 69-71, wherein an expression cassette of the recombinase recombinase comprises one or more mRNA destabilization elements.

Embodiment 73. The virus of any one of Embodiments 69-72, wherein the recombinase is a part of a fusion protein comprising an additional polypeptide, and wherein the additional polypeptide regulates the activity and/or cellular localization of the recombinase.

Embodiment 74. The virus of Embodiment 73, wherein the activity and/or cellular localization of the recombinase is regulated by the presence of a ligand and/or a small molecule.

Embodiment 75. The virus of Embodiment 73 or 74, wherein the additional polypeptide comprises a ligand binding domain of an estrogen receptor protein.

Embodiment 76. The virus Embodiment of any one of Embodiments 53-75, wherein the one or more recombinase recognition sites are flippase recognition target (FRT) sites.

Embodiment 77. The virus of any one of Embodiments 1, 3, 5, 7, 9, 11, 13-16, 18-19, 21-34, 36, 38-39, 42-53, 55, and 57-76, wherein the primary oncolytic virus further comprises a polynucleotide encoding a regulatory polypeptide, and wherein the regulatory polypeptide regulates activity of one or more promoters.

Embodiment 78. The virus of any one of Embodiments 2, 4, 6, 8, 10, 12-15, 17-18, 20-33, 35, 37, 40-52, 54, and 56-76, wherein the primary virus further comprises a polynucleotide encoding a regulatory polypeptide, and wherein the regulatory polypeptide regulates activity of one or more promoters.

Embodiment 79. A recombinant primary oncolytic virus comprising:

a first polynucleotide encoding a secondary oncolytic virus; and

a second polynucleotide encoding one or more RNA interference (RNAi) molecules.

Embodiment 80. A recombinant primary virus comprising:

a first polynucleotide encoding a secondary virus; and

a second polynucleotide encoding one or more RNA interference (RNAi) molecules.

Embodiment 81. The virus of Embodiment 79, wherein the primary oncolytic virus and the secondary oncolytic viruses are replication-competent.

Embodiment 82. The virus of Embodiment 80, wherein the primary virus and the secondary viruses are replication-competent.

Embodiment 83. The virus of any one of Embodiments 79-82, wherein the first polynucleotide is operably linked to a first regulatable promoter and wherein the second polynucleotide is operably linked to a second regulatable promoter.

Embodiment 84. The virus of any one of Embodiments 79, 81,a and 83, wherein the primary oncolytic virus generates an antigen-specific immune response that does not mediate antigen-specific immunity against the secondary oncolytic virus.

Embodiment 85. The virus of any one of Embodiments 80, 82, and 83, wherein the primary virus generates an antigen-specific immune response that does not mediate antigen-specific immunity against the secondary virus.

Embodiment 86. The virus of any one of Embodiments 79, 81, 83, and 84, wherein the primary oncolytic virus is a double-stranded DNA (dsDNA) virus.

Embodiment 87. The virus of any one of Embodiments 80, 82, 83, and 85, wherein the primary virus is a double-stranded DNA (dsDNA) virus.

Embodiment 88. The virus of Embodiment 86 or 87, wherein the dsDNA virus is a herpes simplex virus (HSV), an adenovirus or a virus of Poxviridae family, optionally wherein the virus of virus of Poxviridae family is a molluscum contagiosum virus, a myxoma virus, a vaccina virus, a monkeypox virus, or a yatapoxvirus.

Embodiment 89. The virus of any one of Embodiments 79, 81, 83, and 84, wherein the primary oncolytic virus is a RNA virus.

Embodiment 90. The virus of any one of Embodiments 80, 82, 83, and 85, wherein the primary virus is a RNA virus.

Embodiment 91. The virus of Embodiment 89 or 90, wherein the RNA virus is a paramyxovirus or a rhabdovirus.

Embodiment 92. The virus of any one of Embodiments 79, 81, 83, 84, 86, 88, 89, and 91, wherein the secondary oncolytic virus is a positive-sense single-stranded RNA (ssRNA) virus, a negative-sense ssRNA virus, or an ambi-sense ssRNA virus.

Embodiment 93. The virus of any one of Embodiments 80, 82, 83, 85, 87, 88, and 90-91 wherein the secondary virus is a positive-sense single-stranded RNA (ssRNA) virus, a negative-sense ssRNA virus, or an ambi-sense ssRNA virus.

Embodiment 94. The virus of Embodiment 92 or 93, wherein the secondary oncolytic virus or the secondary virus is the negative-sense ssRNA virus, and

wherein the negative-sense ssRNA virus is a virus of Rrhabdoviridae family, Paramyxoviridae family, or Orthomyxoviridae family, optionally:

wherein the virus of Rhabdoviridae family is a vesicular stomatitis virus (VSV) or a maraba virus;

wherein the virus of Paramyxoviridae family is a Newcastle Disease virus, a Sendai virus, or a measles virus; or

wherein the virus of Orthomyxoviridae family is an influenza virus.

Embodiment 95 The virus of Embodiment 92 or 93, wherein the secondary oncolytic virus or the secondary virus is the positive-sense ssRNA virus, and wherein the positive-sense ssRNA virus is an enterovirus, optionally wherein the enterovirus is a poliovirus, a Seneca Valley virus (SVV), a coxsackievirus, or an echovirus, optionally wherein the coxsakivirus is a coxsackievirus A (CVA) or a coxsackievirus B (CVB), optionally wherein the coxsakivirus is CVA9, CVA21 or CVB3.

Embodiment 96. The virus of Embodiment 92 or 93, wherein the secondary oncolytic virus or the secondary virus is the positive-sense ssRNA virus, and wherein the positive-sense ssRNA virus is a Encephalomyocarditis virus (EMCV) or a Mengovirus.

Embodiment 97. The virus of Embodiment 92 or 93, wherein the secondary oncolytic virus or the secondary virus is the positive-sense ssRNA virus, and wherein the positive-sense ssRNA virus is a virus of Togaviridae family, optionally wherein the virus of Togaviridae familyis a new world alphavirus or old world alphavirus, and optionally wherein the new world alphavirus or old world alphavirusis is VEEV, WEEV, EEV, Sindbis virus, Semliki Forest virus, Ross River Virus, or Mayaro virus.

Embodiment 98. The virus of any one of Embodiments 79, 81, 83, 84, 86, 88, 89, 91-92, and 94-97, wherein the primary oncolytic virus and/or the secondary oncolytic virus is a chimeric virus.

Embodiment 99. The virus of any one of Embodiments 80, 82, 83, 85, 87, 88, 90-91, and 93-97, wherein the primary virus and/or the secondary virus is a chimeric virus.

Embodiment 100. The virus of any one of Embodiments 79, 81, 83, 84, 86, 88, 89, 91-92, and 94-98, wherein the primary oncolytic virus and/or the secondary oncolytic virus is a pseudotyped virus.

Embodiment 101. The virus of any one of Embodiments 80, 82, 83, 85, 87, 88, 90-91, 93-97, and 99, wherein the primary virus and/or the secondary virus is a pseudotyped virus.

Embodiment 102. The virus of any one of Embodiments 79-101, wherein the first and second regulatable promoters are selected from a steroid-inducible promoter, a metallothionine promoter, an MX-1 promoter, a GENESWITCH™ hybrid promoter, a cumate-responsive promoter, and a tetracycline-dependent promoter.

Embodiment 103. The virus of any one of Embodiments 79-102, further comprising a third polynucleotide encoding a first peptide capable of binding to the first regulatable promoter and a second peptide capable of binding to the second regulatable promoter.

Embodiment 104. The virus of Embodiment 103, wherein the third polynucleotide is operably linked to a constitutive promoter.

Embodiment 105. The virus of Embodiment 104, wherein the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

Embodiment 106. The virus of any one of Embodiments 103-105, wherein the first regulatable promoter is a tetracycline (Tet)-inducible promoter and wherein in the first peptide is a reverse tetracycline-controlled transactivator (rtTA) peptide.

Embodiment 107. The virus of any one of Embodiments 103-106, wherein the second regulatable promoter is a tetracycline (Tet)-repressible promoter and wherein in the second peptide is a tetracycline-controlled transactivator (tTA) peptide.

Embodiment 108. The virus of any one of Embodiments 103-106, wherein the first regulatable promoter is a tetracycline (Tet)-repressible promoter and wherein in the first peptide is a tetracycline-controlled transactivator (tTA) peptide.

Embodiment 109. The virus of any one of Embodiments 103-108, wherein the second regulatable promoter is a tetracycline (Tet)-inducible promoter and wherein in the second peptide is a reverse tetracycline-controlled transactivator (rtTA) peptide.

Embodiment 110. Thevirus of any one of Embodiments 79, 81, 83, 84, 86, 88, 89, 91-92, 94-98, 100, and 102-109, wherein the one or more RNAi molecules bind to a target sequence in the genome of the secondary oncolytic virus and inhibits replication of the secondary oncolytic virus.

Embodiment 111. The virus of any one of Embodiments 80, 82, 83, 85, 87, 88, 90-91, 93-97, 99, and 101-109, wherein the one or more RNAi molecules bind to a target sequence in the genome of the secondary virus and inhibits replication of the secondary virus.

Embodiment 112. The virus of Embodiment 110 or 111, wherein the RNAi molecule is an siRNA, an miRNA, an shRNA, or an AmiRNA.

Embodiment 113. The virus of any one of Embodiments 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, and 112, wherein the polynucleotide encoding the secondary oncolytic virus comprises first 3′ ribozyme-encoding sequence and a second 5′ ribozyme encoding sequence.

Embodiment 114. The virus of any one of Embodiments 2, 4, 6, 8, 10, 12-15, 17-18, 20-33, 35, 37, 40-52, 54, 56-76, 78, 80, 82, 83, 85, 87, 88, 90-91, 93-97, 99, 101-109, and 111-112, wherein the polynucleotide encoding the secondary virus comprises first 3′ ribozyme-encoding sequence and a second 5′ ribozyme encoding sequence.

Embodiment 115. The virus of Embodiment 113 or 114, wherein the first and second ribozyme-encoding sequences encode a Hammerhead ribozyme or a hepatitis delta virus ribozyme.

Embodiment 116. The virus of any one of Embodiments 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, and 115, wherein the genome of the primary oncolytic virus comprises an miRNA target sequence (miR-TS) cassette comprising one or more miRNA target sequences inserted into one or more viral genes required for replication or inserted into the 3′ or 5′ UTR of the viral genome.

Embodiment 117. The virus of any one of Embodiments 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114, and 115, wherein the genome of the primary virus comprises an miRNA target sequence (miR-TS) cassette comprising one or more miRNA target sequences inserted into one or more viral genes required for replication or inserted into the 3′ or 5′ UTR of the viral genome.

Embodiment 118. The virus of any one of Embodiments 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, and 115-116, wherein the genome of the secondary oncolytic virus comprises an miRNA target sequence (miR-TS) cassette comprising one or more miRNA target sequences inserted into one or more viral genes required for replication or inserted into the 3′ or 5′ UTR of the viral genome.

Embodiment 119. The virus of any one of Embodiments 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, and 117, wherein the genome of the secondary virus comprises an miRNA target sequence (miR-TS) cassette comprising one or more miRNA target sequences inserted into one or more viral genes required for replication or inserted into the 3′ or 5′ UTR of the viral genome.

Embodiment 120. The virus of any one of Embodiments 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, 115-116, and 118, wherein the primary oncolytic virus and the secondary oncolytic virus each comprise an miRNA target sequence (miR-TS) cassette comprising one or more miRNA target sequences inserted into one or more viral genes required for replication or inserted into the 3′ or 5′ UTR of the viral genome.

Embodiment 121. The virus of any one of Embodiments 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, 117, and 119, wherein the primary virus and the secondary virus each comprise an miRNA target sequence (miR-TS) cassette comprising one or more miRNA target sequences inserted into one or more viral genes required for replication or inserted into the 3′ or 5′ UTR of the viral genome.

Embodiment 122. The virus of any one of Embodiments 116, 118, and 120, wherein expression of the one or more miRNAs in a cell inhibits replication of the primary and/or secondary oncolytic viruses.

Embodiment 123. The virus of any one of Embodiments 117, 119, and 121, wherein expression of the one or more miRNAs in a cell inhibits replication of the primary and/or secondary viruses.

Embodiment 124. The virus of any one of Embodiments 1-123, further comprising a polynucleotide sequence encoding at least one exogenous payload protein.

Embodiment 125. The virus of Embodiment 124, wherein the exogenous payload protein is a fluorescent protein, an enzyme, a cytokine, a chemokine, or an antigen-binding molecule.

Embodiment 126. The virus of any one of Embodiments 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, 115-116, 118, 120, 122, and 124-125, wherein expression of the secondary oncolytic virus is regulated by an exogenous agent.

Embodiment 127. The virus of any one of Embodiments 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, 117, 119, 121, and 123-125, wherein expression of the secondary virus is regulated by an exogenous agent.

Embodiment 128. The virus of Embodiment 126 or 127, wherein the exogenous agent is a peptide, a hormone, or a small molecule.

Embodiment 129. A composition comprising the virus of any one of Embodiments 1-128.

Embodiment 130. A method of killing a population of tumor cells comprising administering the virus of any one of Embodiments 1-128 or the composition of Embodiment 129 to the population of tumor cells.

Embodiment 131. The method of Embodiment 130, wherein a first subpopulation of the tumor cells are infected and killed by the primary oncolytic virus.

Embodiment 132. The method of Embodiment 130 or 131, wherein a second subpopulation of the tumor cells are infected and killed by the secondary oncolytic virus.

Embodiment 133. The method of any one of Embodiments 130-132, wherein a subpopulation of the tumor cells are infected and killed by both the primary oncolytic virus and the secondary oncolytic virus.

Embodiment 134. The method of any one of Embodiments 130-133, wherein a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone.

Embodiment 135. The method of any one of Embodiments 130-134, further comprising administering one or more exogenous agents to the population of tumor cells, wherein the one or more exogenous agents regulate the production of the secondary oncolytic virus.

Embodiment 136. The method of Embodiment 135, wherein the one or more exogenous agents is/are administered at the same time as the primary oncolytic virus, and wherein the presence of the exogenous agent(s) inhibits production of the secondary oncolytic virus.

Embodiment 137. The method of Embodiment 135, wherein the one or more exogenous agents is/are administered after the primary oncolytic virus, and wherein the presence of the exogenous agent(s) induces production of the secondary oncolytic virus.

Embodiment 138. The method of Embodiment 137, wherein the exogenous agent(s) is/are administered at least 1 day, at least 1 week, or at least 1 month, after administration of the primary oncolytic virus.

Embodiment 139. The method of any one of the Embodiments 135-138, wherein no secondary oncolytic virus is detectable prior to the administration of the exogenous agent(s).

Embodiment 140. The method of Embodiment 130, wherein a first subpopulation of the tumor cells are infected and killed by the primary virus.

Embodiment 141. The method of Embodiment 130 or 140, wherein a second subpopulation of the tumor cells are infected and killed by the secondary virus.

Embodiment 142. The method of any one of Embodiments 130, 140, and 141, wherein a subpopulation of the tumor cells are infected and killed by both the primary virus and the secondary virus.

Embodiment 143. The method of any one of Embodiments 130 and 140-142, wherein a greater number of tumor cells in the population are killed by the primary and secondary viruses compared to the number of tumor cells killed by a reference primary virus without the polynucleotide encoding the secondary virus or the secondary virus alone.

Embodiment 144. The method of any one of Embodiments 130 and 140-143, further comprising administering one or more exogenous agents to the population of tumor cells, wherein the one or more exogenous agents regulate the production of the secondary virus.

Embodiment 145. The method of Embodiment 144, wherein the one or more exogenous agents is/are administered at the same time as the primary virus, and wherein the presence of the exogenous agent(s) inhibits production of the secondary virus.

Embodiment 146. The method of Embodiment 145, wherein the one or more exogenous agents is/are administered after the primary virus, and wherein the presence of the exogenous agent(s) induces production of the secondary virus.

Embodiment 147. The method of Embodiment 146, wherein the exogenous agent(s) is/are administered at least 1 day, at least 1 week, or at least 1 month, after administration of the primary virus.

Embodiment 148. The method of any one of the Embodiments 144-147, wherein no secondary virus is detectable prior to the administration of the exogenous agent(s).

Embodiment 149. A method of treating a tumor in a subject in need thereof comprising administering the virus of any one of Embodiments 1-128 or the composition of Embodiment 129 to the subject.

Embodiment 150. The method of Embodiment 149, wherein a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone.

Embodiment 151. The method of Embodiment 149 or 150, wherein the method leads to greater reduction of tumor size in the subject compared to administration of a reference primary oncolytic virus without the polynucleotide encoding the secondary oncolytic virus or the secondary oncolytic virus alone.

Embodiment 152. The method of any one of Embodiments 149-151, wherein the method induces a stronger immune response against one or more tumor antigens in the subject compared to administering a reference primary oncolytic virus without the polynucleotide encoding the secondary oncolytic virus or administering the secondary oncolytic virus alone.

Embodiment 153. The method of any one of Embodiments 149-152, wherein the method results in a reduced immune response against the primary oncolytic virus in the subject compared to administering a reference primary oncolytic virus without the polynucleotide encoding the secondary oncolytic virus.

Embodiment 154. The method of any one of Embodiments 149-153, wherein the method results in a reduced immune response against the secondary oncolytic virus in the subject compared to administering the secondary oncolytic virus alone.

Embodiment 155. The method of any one of Embodiments 149-154, wherein the method results in preferential/more specific killing of tumor cells in the subject compared to administering a reference primary oncolytic virus without the polynucleotide encoding the secondary oncolytic virus or administering the secondary oncolytic virus alone.

Embodiment 156. The method of any one of Embodiments 149-155, wherein the method results in more persistent production of the primary oncolytic virus in the subject compared to administering a reference primary oncolytic virus without the polynucleotide encoding the secondary oncolytic virus.

Embodiment 157. The method of any one of Embodiments 149-156, wherein the method results in more persistent production of the secondary oncolytic virus in the subject compared to administering the secondary oncolytic virus alone.

Embodiment 158. The method of any one of Embodiments 149-157, wherein the method results in an extended period of tumor inhibition in the subject compared to administering a reference primary oncolytic virus without the polynucleotide encoding the secondary oncolytic virus or the secondary oncolytic virus alone.

Embodiment 159. The method of any one of Embodiments 149-158, wherein the method enables viral infection of more cell types compared to administering a reference primary oncolytic virus without the polynucleotide encoding the secondary oncolytic virus or the secondary oncolytic virus alone.

Embodiment 160. The method of any one of Embodiments 149-159, further comprising administering one or more exogenous agents to the population of tumor cells, wherein the one or more exogenous agents regulate the production of the secondary oncolytic virus.

Embodiment 161. The method of Embodiment 160, wherein the one or more exogenous agents is/are administered at the same time as the primary oncolytic virus, and wherein the presence of the exogenous agent(s) inhibits production of the secondary oncolytic virus.

Embodiment 162. The method of Embodiment 160, wherein the one or more exogenous agents is/are administered after the primary oncolytic virus, and wherein the presence of the exogenous agent(s) induces production of the secondary oncolytic virus.

Embodiment 163. The method of Embodiment 162, wherein the exogenous agent(s) is/are administered at least 1 day, at least 1 week, or at least 1 month, after administration of the primary oncolytic virus.

Embodiment 164. The method of any one of the Embodiments 160-163, wherein no secondary oncolytic virus is detectable prior to the administration of the exogenous agent(s).

Embodiment 165. The method of Embodiment 149, wherein a greater number of tumor cells in the population are killed by the primary and secondary viruses compared to the number of tumor cells killed by a reference primary virus without the polynucleotide encoding the secondary virus or the secondary virus alone.

Embodiment 166. The method of Embodiment 149 or 165, wherein the method leads to greater reduction of tumor size in the subject compared to administration of a reference primary virus without the polynucleotide encoding the secondary virus or the secondary virus alone.

Embodiment 167. The method of any one of Embodiments 149, 165, and 166, wherein the method induces a stronger immune response against one or more tumor antigens in the subject compared to administering a reference primary virus without the polynucleotide encoding the secondary virus or administering the secondary virus alone.

Embodiment 168. The method of any one of Embodiments 149 and 165-167, wherein the method results in a reduced immune response against the primary virus in the subject compared to administering a reference primary virus without the polynucleotide encoding the secondary virus.

Embodiment 169. The method of any one of Embodiments 149 and 165-168, wherein the method results in a reduced immune response against the secondary virus in the subject compared to administering the secondary virus alone.

Embodiment 170. The method of any one of Embodiments 149 and 165-169, wherein the method results in preferential/more specific killing of tumor cells in the subject compared to administering a reference primary virus without the polynucleotide encoding the secondary virus or administering the secondary virus alone.

Embodiment 171. The method of any one of Embodiments 149 and 165-170, wherein the method results in more persistent production of the primary virus in the subject compared to administering a reference primary virus without the polynucleotide encoding the secondary virus.

Embodiment 172. The method of any one of Embodiments 149 and 165-171, wherein the method results in more persistent production of the secondary virus in the subject compared to administering the secondary virus alone.

Embodiment 173. The method of any one of Embodiments 149 and 165-172, wherein the method results in an extended period of tumor inhibition in the subject compared to administering a reference primary virus without the polynucleotide encoding the secondary virus or the secondary virus alone.

Embodiment 174. The method of any one of Embodiments 149 and 165-173, wherein the method enables viral infection of more cell types compared to administering a reference primary virus without the polynucleotide encoding the secondary virus or the secondary virus alone.

Embodiment 175. The method of any one of Embodiments 149 and 165-174, further comprising administering one or more exogenous agents to the population of tumor cells, wherein the one or more exogenous agents regulate the production of the secondary virus.

Embodiment 176. The method of Embodiment 175, wherein the one or more exogenous agents is/are administered at the same time as the primary virus, and wherein the presence of the exogenous agent(s) inhibits production of the secondary virus.

Embodiment 177. The method of Embodiment 175, wherein the one or more exogenous agents is/are administered after the primary virus, and wherein the presence of the exogenous agent(s) induces production of the secondary virus.

Embodiment 178. The method of Embodiment 177, wherein the exogenous agent(s) is/are administered at least 1 day, at least 1 week, or at least 1 month, after administration of the primary virus.

Embodiment 179. The method of any one of the Embodiments 175-178, wherein no secondary virus is detectable prior to the administration of the exogenous agent(s).

Embodiment 180. A polynucleotide encoding the virus of Embodiments 1-128.

Embodiment 181. A vector comprising the polynucleotide of Embodiment 180.

Embodiment 182. A pharmaceutical composition comprising the vector of Embodiment 181.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A recombinant primary oncolytic virus, comprising:
a polynucleotide encoding a secondary oncolytic virus.

2. A recombinant primary virus, comprising:
a polynucleotide encoding a secondary virus.

3. The virus of claim 1, wherein the primary oncolytic virus and the secondary oncolytic virus are replication-competent.

4. The virus of claim 2, wherein the primary virus and the secondary virus are replication-competent.

5. The virus of claim 1, wherein the primary oncolytic virus and/or the secondary oncolytic virus is/are replication-incompetent.

6. The virus of claim 2, wherein the primary virus and/or the secondary virus is/are replication-incompetent.

7. The virus of any one of claims 1, 3, and 5, wherein the polynucleotide encoding the secondary oncolytic virus is operably linked to a regulatable promoter.

8. The virus of any one of claims 2, 4, and 6, wherein the polynucleotide encoding the secondary virus is operably linked to a regulatable promoter.

9. The virus of any one of claims 1, 3, 5, and 7, wherein the primary oncolytic virus generates an antigen-specific immune response that does not mediate antigen-specific immunity against the secondary oncolytic virus.

10. The virus of any one of claims 2, 4, 6, and 8, wherein the primary virus generates an antigen-specific immune response that does not mediate antigen-specific immunity against the secondary virus.

11. The virus of any one of claims 1, 3, 5, 7, and 9, wherein the primary oncolytic virus is a double-stranded DNA (dsDNA) virus.

12. The virus of any one of claims 2, 4, 6, 8, and 10, wherein the primary virus is a double-stranded DNA (dsDNA) virus.

13. The virus of claim 11 or 12, wherein the dsDNA virus is a herpes simplex virus (HSV) or an adenovirus.

14. The virus of claim 11 or 12, wherein the dsDNA virus is a virus of Poxviridae family.

15. The virus of claim 14, wherein the dsDNA virus is a molluscum contagiosum virus, a myxoma virus, a vaccina virus, a monkeypox virus, or a yatapoxvirus.

16. The virus of any one of claims 1, 3, 5, 7, and 9, wherein the primary oncolytic virus is a RNA virus.

17. The virus of any one of claims 2, 4, 6, 8, and 10, wherein the primary virus is a RNA virus.

18. The virus of claim 16 or 17, wherein the RNA virus is a paramyxovirus or a rhabdovirus.

19. The virus of any one of claims 1, 3, 5, 7, 9, 11, 13-16, and 18, wherein the secondary oncolytic virus is a positive-sense single-stranded RNA (ssRNA) virus, a negative-sense ssRNA virus, or an ambi-sense ssRNA virus.

20. The virus of any one of claims 2, 4, 6, 8, 10, 12-15, and 17-18, wherein the secondary virus is a positive-sense single-stranded RNA (ssRNA) virus, a negative-sense ssRNA virus, or an ambi-sense ssRNA virus.

21. The virus of claim 19 or 20, wherein the secondary oncolytic virus or the secondary virus is a negative-sense ssRNA virus of Rrhabdoviridae family, Paramyxoviridae family, or Orthomyxoviridae family.

22. The virus of claim 21, wherein the virus of Rhabdoviridae family is a vesicular stomatitis virus (VSV) or a maraba virus.

23. The virus of claim 21, wherein the virus of Paramyxoviridae family is a Newcastle Disease virus, a Sendai virus, or a measles virus.

24. The virus of claim 21, wherein the virus of Orthomyxoviridae family is an influenza virus.

25. The virus of claim 19 or 20, wherein the secondary oncolytic virus or the secondary virus is the positive-sense ssRNA virus, and wherein the positive-sense ssRNA virus is an enterovirus.

26. The virus of claim 25, wherein the enterovirus is a poliovirus, a Seneca Valley virus (SVV), a coxsackievirus, or an echovirus.

27. The virus of claim 26, wherein the coxsakivirus is a coxsackievirus A (CVA) or a coxsackievirus B (CVB),

28. The virus of claim 27, wherein the coxsakivirus is CVA9, CVA21 or CVB3.

29. The virus of claim 19 or 20, wherein the secondary oncolytic virus or the secondary virus is the positive-sense ssRNA virus, and wherein the positive-sense ssRNA virus is a Encephalomyocarditis virus (EMCV).

30. The virus of claim 19 or 20, wherein the secondary oncolytic virus or the secondary virus is the positive-sense ssRNA virus, and wherein the positive-sense ssRNA virus is a Mengovirus.

31. The virus of claim 19 or 20, wherein the secondary oncolytic virus or the secondary virus is the positive-sense ssRNA virus, and wherein the positive-sense ssRNA virus is a virus of Togaviridae family.

32. The virus of claim 31, wherein the virus of Togaviridae family is a new world alphavirus or old world alphavirus.

33. The virus of claim 32, wherein the new world alphavirus or old world alphavirusis is VEEV, WEEV, EEV, Sindbis virus, Semliki Forest virus, Ross River Virus, or Mayaro virus.

34. The virus of any one of claims 1, 3, 5, 7, 9, 11, 13-16, 18-19, and 21-33, wherein the primary oncolytic virus and/or the secondary oncolytic virus is a chimeric virus.

35. The virus of any one of claims 2, 4, 6, 8, 10, 12-15, 17-18, and 20-33, wherein the primary virus and/or the secondary virus is a chimeric virus.

36. The virus of any one of claims 1, 3, 5, 7, 9, 11, 13-16, 18-19, and 21-34, wherein the primary oncolytic virus and/or the secondary oncolytic virus is a pseudotyped virus.

37. The virus of any one of claims 2, 4, 6, 8, 10, 12-15, 17-18, 20-33, and 35, wherein the primary virus and/or the secondary virus is a pseudotyped virus.

38. The virus of claim 36, wherein the secondary oncolytic virus is a pseudotyped virus, and wherein the primary oncolytic virus comprises a coding region for a capsid protein or an envelope protein of the secondary oncolytic virus outside the cording region for the secondary oncolytic virus.

39. The virus of claim 38, wherein the secondary oncolytic virus is an alphavirus, a paramyxovirus or a rhabdovirus.

40. The virus of claim 37, wherein the secondary virus is a pseudotyped virus, and wherein the primary virus comprises a coding region for a capsid protein or an envelope protein of the secondary virus outside the cording region for the secondary virus.

41. The virus of claim 40, wherein the secondary virus is an alphavirus, a paramyxovirus or a rhabdovirus.

42. The virus of any one of claims 7-41, wherein the regulatable promoter is selected from a steroid-inducible promoter, a metallothionine promoter, an MX-1 promoter, a GENESWITCH™ hybrid promoter, a cumate-responsive promoter, and a tetracycline-inducible promoter.

43. The virus of any one of claims 7-41, wherein the regulatable promoter comprises a constitutive promoter flanked by recombinase recognition sites.

44. The virus of any one of claims 1-43, further comprising a second polynucleotide encoding a peptide capable of binding to the regulatable promoter.

45. The virus of claim 44, wherein the second polynucleotide is operably linked to a constitutive promoter or an inducible promoter.

46. The virus of claim 45, wherein the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

47. The virus of any one of claims 44-46, wherein the regulatable promoter is a tetracycline (Tet)-dependent promoter and wherein in the peptide is a reverse tetracycline-controlled transactivator (rtTA) peptide.

48. The virus of any one of claims 44-46, wherein the regulatable promoter is a tetracycline (Tet)-dependent promoter and wherein in the peptide is a tetracycline-controlled transactivator (tTA) peptide.

49. The virus of any one of claims 1-48, wherein the primary oncolytic virus or the primary virus further comprises a polynucleotide encoding one or more RNA interference (RNAi) molecules.

50. The virus of claim 49, wherein the polynucleotide encoding one or more RNA interference (RNAi) molecules is operably linked to a second regulatable promoter.

51. The virus of claim 49 or 50, wherein the one or more RNAi molecules bind to a target sequence in the genome of the secondary oncolytic virus or the secondary virus and inhibits replication of the secondary oncolytic virus or the secondary virus.

52. The virus of any one of claims 49-51, wherein the RNAi molecule is an siRNA, an miRNA, an shRNA, or an AmiRNA.

53. The virus of any one of claims 1, 3, 5, 7, 9, 11, 13-16, 18-19, 21-34, 36, 38-39, and 42-52, wherein the polynucleotide encoding the secondary oncolytic virus comprises one or more recombinase recognition sites.

54. The virus of any one of claims 2, 4, 6, 8, 10, 12-15, 17-18, 20-33, 35, 37, and 40-52, wherein the polynucleotide encoding the secondary virus comprises one or more recombinase recognition sites.

55. The virus of any one of claims 1, 3, 5, 7, 9, 11, 13-16, 18-19, 21-34, 36, 38-39, and 42-53, wherein the polynucleotide encoding the secondary oncolytic virus comprises one or more recombinase-responsive cassettes, wherein the recombinase-responsive cassette comprises the one or more recombinase recognition sites.

56. The virus of any one of claims 2, 4, 6, 8, 10, 12-15, 17-18, 20-33, 35, 37, 40-52, and 54, wherein the polynucleotide encoding the secondary virus comprises one or more recombinase-responsive cassettes, wherein the recombinase-responsive cassette comprises the one or more recombinase recognition sites.

57. The virus of claim 55 or 56, wherein the one or more recombinase-responsive cassettes comprise a Recombinase-Responsive Excision Cassette (RREC).

58. The virus of claim 57, wherein the RREC comprises a transcriptional/translational termination (STOP) element.

59. The virus of the claim 58, wherein the transcriptional/translational termination (STOP) element comprises a sequence having 80% identity to any one of SEQ ID NOS: 854-856.

60. The virus of any one of claims 55-59, wherein the one or more recombinase-responsive cassettes comprise a Recombinase-Responsive Inversion Cassette (RRIC).

61. The virus of the claim 60, wherein the RRIC comprises two or more orthogonal Recombinase Recognition Sites on each side of a Central Element.

62. The virus of claim 60 or 61, wherein the RRIC comprises a promoter or a portion of the promoter.

63. The virus of claim 60 or 61, wherein the RRIC comprises a coding region or a portion of the coding region, wherein the coding region encodes the viral genome of the secondary oncolytic virus or the secondary virus.

64. The virus of any one of claims 60-63, wherein the RRIC comprises one or more Control Element(s).

65. The virus of claim 64, wherein the Control Element(s) is/are transcriptional/translational termination (STOP) elements.

66. The virus of claim 65, wherein the Control Element(s) has/have a sequence having 80% identity to any one of SEQ ID NOS: 854-856.

67. The virus of any one of claims 60-66, wherein the Recombinase-Responsive Inversion Cassette (RRIC) further comprises a portion of an intron.

68. The virus of claim 67, wherein the polynucleotide encoding the secondary oncolytic virus or the secondary virus yields a mature viral genome transcript of the secondary oncolytic virus or the secondary virus without the Recombinase Recognition Site after removal of the intron via mRNA splicing.

69. The virus of any one of claims 1-68, wherein the primary oncolytic virus or the primary virus further comprises a polynucleotide encoding the recombinase.

70. The virus of claim 69, wherein the recombinase is a Flippase (Flp) or a Cre recombinase (Cre).

71. The virus of claim 69 or 70, wherein the coding region of the recombinase comprises an intron.

72. The virus of any one of claims 69-71, wherein an expression cassette of the recombinase comprises one or more mRNA destabilization elements.

73. The virus of any one of claims 69-72, wherein the recombinase is a part of a fusion protein comprising an additional polypeptide, and wherein the additional polypeptide regulates the activity and/or cellular localization of the recombinase.

74. The virus of claim 73, wherein the activity and/or cellular localization of the recombinase is regulated by the presence of a ligand and/or a small molecule.

75. The virus of claim 73 or 74, wherein the additional polypeptide comprises a ligand binding domain of an estrogen receptor protein.

76. The virus claim of any one of claims 53-75, wherein the one or more recombinase recognition sites are flippase recognition target (FRT) sites.

77. The virus of any one of claims 1, 3, 5, 7, 9, 11, 13-16, 18-19, 21-34, 36, 38-39, 42-53, 55, and 57-76, wherein the primary oncolytic virus further comprises a polynucleotide encoding a regulatory polypeptide, and wherein the regulatory polypeptide regulates activity of one or more promoters.

78. The virus of any one of claims 2, 4, 6, 8, 10, 12-15, 17-18, 20-33, 35, 37, 40-52, 54, and 56-76, wherein the primary virus further comprises a polynucleotide encoding a regulatory polypeptide, and wherein the regulatory polypeptide regulates activity of one or more promoters.

79. A recombinant primary oncolytic virus comprising:
a first polynucleotide encoding a secondary oncolytic virus; and
a second polynucleotide encoding one or more RNA interference (RNAi) molecules.

80. A recombinant primary virus comprising:
a first polynucleotide encoding a secondary virus; and
a second polynucleotide encoding one or more RNA interference (RNAi) molecules.

81. The virus of claim 79, wherein the primary oncolytic virus and the secondary oncolytic viruses are replication-competent.

82. The virus of claim 80, wherein the primary virus and the secondary viruses are replication-competent.

83. The virus of any one of claims 79-82, wherein the first polynucleotide is operably linked to a first regulatable promoter and wherein the second polynucleotide is operably linked to a second regulatable promoter.

84. The virus of any one of claims 79, 81, a and 83, wherein the primary oncolytic virus generates an antigen-specific immune response that does not mediate antigen-specific immunity against the secondary oncolytic virus.

85. The virus of any one of claims 80, 82, and 83, wherein the primary virus generates an antigen-specific immune response that does not mediate antigen-specific immunity against the secondary virus.

86. The virus of any one of claims 79, 81, 83, and 84, wherein the primary oncolytic virus is a double-stranded DNA (dsDNA) virus.

87. The virus of any one of claims 80, 82, 83, and 85, wherein the primary virus is a double-stranded DNA (dsDNA) virus.

88. The virus of claim 86 or 87, wherein the dsDNA virus is a herpes simplex virus (HSV), an adenovirus or a virus of Poxviridae family, optionally wherein the virus of virus of Poxviridae family is a molluscum contagiosum virus, a myxoma virus, a vaccina virus, a monkeypox virus, or a yatapoxvirus.

89. The virus of any one of claims 79, 81, 83, and 84, wherein the primary oncolytic virus is a RNA virus.

90. The virus of any one of claims 80, 82, 83, and 85, wherein the primary virus is a RNA virus.

91. The virus of claim 89 or 90, wherein the RNA virus is a paramyxovirus or a rhabdovirus.

92. The virus of any one of claims 79, 81, 83, 84, 86, 88, 89, and 91, wherein the secondary oncolytic virus is a positive-sense single-stranded RNA (ssRNA) virus, a negative-sense ssRNA virus, or an ambi-sense ssRNA virus.

93. The virus of any one of claims 80, 82, 83, 85, 87, 88, and 90-91 wherein the secondary virus is a positive-sense single-stranded RNA (ssRNA) virus, a negative-sense ssRNA virus, or an ambi-sense ssRNA virus.

94. The virus of claim 92 or 93, wherein the secondary oncolytic virus or the secondary virus is the negative-sense ssRNA virus, and wherein the negative-sense ssRNA virus is a virus of Rrhabdoviridae family, Paramyxoviridae family, or Orthomyxoviridae family, optionally:
wherein the virus of Rhabdoviridae family is a vesicular stomatitis virus (VSV) or a maraba virus;
wherein the virus of Paramyxoviridae family is a Newcastle Disease virus, a Sendai virus, or a measles virus; or
wherein the virus of Orthomyxoviridae family is an influenza virus.

95. The virus of claim 92 or 93, wherein the secondary oncolytic virus or the secondary virus is the positive-sense ssRNA virus, and wherein the positive-sense ssRNA virus is an enterovirus, optionally wherein the enterovirus is a poliovirus, a Seneca Valley virus (SVV), a coxsackievirus, or an echovirus, optionally wherein the coxsakivirus is a coxsackievirus A (CVA) or a coxsackievirus B (CVB), optionally wherein the coxsakivirus is CVA9, CVA21 or CVB3.

96. The virus of claim 92 or 93, wherein the secondary oncolytic virus or the secondary virus is the positive-sense ssRNA virus, and wherein the positive-sense ssRNA virus is a Encephalomyocarditis virus (EMCV) or a Mengovirus.

97. The virus of claim 92 or 93, wherein the secondary oncolytic virus or the secondary virus is the positive-sense ssRNA virus, and wherein the positive-sense ssRNA virus is a virus of Togaviridae family, optionally wherein the virus of Togaviridae familyis a new world alphavirus or old world alphavirus, and optionally wherein the new world alphavirus or old world alphavirusis is VEEV, WEEV, EEV, Sindbis virus, Semliki Forest virus, Ross River Virus, or Mayaro virus.

98. The virus of any one of claims 79, 81, 83, 84, 86, 88, 89, 91-92, and 94-97, wherein the primary oncolytic virus and/or the secondary oncolytic virus is a chimeric virus.

99. The virus of any one of claims 80, 82, 83, 85, 87, 88, 90-91, and 93-97, wherein the primary virus and/or the secondary virus is a chimeric virus.

100. The virus of any one of claims 79, 81, 83, 84, 86, 88, 89, 91-92, and 94-98, wherein the primary oncolytic virus and/or the secondary oncolytic virus is a pseudotyped virus.

101. The virus of any one of claims 80, 82, 83, 85, 87, 88, 90-91, 93-97, and 99, wherein the primary virus and/or the secondary virus is a pseudotyped virus.

102. The virus of any one of claims 79-101, wherein the first and second regulatable promoters are selected from a steroid-inducible promoter, a metallothionine promoter, an MX-1 promoter, a GENESWITCH™ hybrid promoter, a cumate-responsive promoter, and a tetracycline-dependent promoter.

103. The virus of any one of claims 79-102, further comprising a third polynucleotide encoding a first peptide capable of binding to the first regulatable promoter and a second peptide capable of binding to the second regulatable promoter.

104. The virus of claim 103, wherein the third polynucleotide is operably linked to a constitutive promoter.

105. The virus of claim 104, wherein the constitutive promoter is selected from a cytomegalovirus (CMV) promoter, a simian virus 40 (SV40) promoter, a Moloney murine leukemia virus (MoMLV) LTR promoter, a Rous sarcoma virus (RSV) LTR promoter, an elongation factor 1-alpha (EF1a) promoter, an early growth response 1 (EGR1) promoter, a ferritin H (FerH) promoter, a ferritin L (FerL) promoter, a glyceraldehyde 3-phosphate dehydrogenase (GAPDH) promoter, a eukaryotic translation initiation factor 4A1 (EIF4A1) promoter, a ubiquitin C promoter (UBC) promoter, a phosphoglycerate kinase-1 (PGK) promoter, and a cytomegalovirus enhancer/chicken β-actin (CAG) promoter.

106. The virus of any one of claims 103-105, wherein the first regulatable promoter is a tetracycline (Tet)-inducible promoter and wherein in the first peptide is a reverse tetracycline-controlled transactivator (rtTA) peptide.

107. The virus of any one of claims 103-106, wherein the second regulatable promoter is a tetracycline (Tet)-repressible promoter and wherein in the second peptide is a tetracycline-controlled transactivator (tTA) peptide.

108. The virus of any one of claims 103-106, wherein the first regulatable promoter is a tetracycline (Tet)-repressible promoter and wherein in the first peptide is a tetracycline-controlled transactivator (tTA) peptide.

109. The virus of any one of claims 103-108, wherein the second regulatable promoter is a tetracycline (Tet)-inducible promoter and wherein in the second peptide is a reverse tetracycline-controlled transactivator (rtTA) peptide.

110. The virus of any one of claims 79, 81, 83, 84, 86, 88, 89, 91-92, 94-98, 100, and 102-109, wherein the one or more RNAi molecules bind to a target sequence in the genome of the secondary oncolytic virus and inhibits replication of the secondary oncolytic virus.

111. The virus of any one of claims 80, 82, 83, 85, 87, 88, 90-91, 93-97, 99, and 101-109, wherein the one or more RNAi molecules bind to a target sequence in the genome of the secondary virus and inhibits replication of the secondary virus.

112. The virus of claim 110 or 111, wherein the RNAi molecule is an siRNA, an miRNA, an shRNA, or an AmiRNA.

113. The virus of any one of claims 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, and 112, wherein the polynucleotide encoding the secondary oncolytic virus comprises first 3′ ribozyme-encoding sequence and a second 5′ ribozyme encoding sequence.

114. The virus of any one of claims 2, 4, 6, 8, 10, 12-15, 17-18, 20-33, 35, 37, 40-52, 54, 56-76, 78, 80, 82, 83, 85, 87, 88, 90-91, 93-97, 99, 101-109, and 111-112, wherein the polynucleotide encoding the secondary virus comprises first 3′ ribozyme-encoding sequence and a second 5′ ribozyme encoding sequence.

115. The virus of claim 113 or 114, wherein the first and second ribozyme-encoding sequences encode a Hammerhead ribozyme or a hepatitis delta virus ribozyme.

116. The virus of any one of claims 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, and 115, wherein the genome of the primary oncolytic virus comprises an miRNA target sequence (miR-TS) cassette comprising one or more miRNA target sequences inserted into one or more viral genes required for replication or inserted into the 3′ or 5′ UTR of the viral genome.

117. The virus of any one of claims 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114, and 115, wherein the genome of the primary virus comprises an miRNA target sequence (miR-TS) cassette comprising one or more miRNA target sequences inserted into one or more viral genes required for replication or inserted into the 3′ or 5′ UTR of the viral genome.

118. The virus of any one of claims 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, and 115-116, wherein the genome of the secondary oncolytic virus comprises an miRNA target sequence (miR-TS) cassette comprising one or more miRNA target sequences inserted into one or more viral genes required for replication or inserted into the 3′ or 5′ UTR of the viral genome.

119. The virus of any one of claims 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, and 117, wherein the genome of the secondary virus comprises an miRNA target sequence (miR-TS) cassette comprising one or more miRNA target sequences inserted into one or more viral genes required for replication or inserted into the 3′ or 5′ UTR of the viral genome.

120. The virus of any one of claims 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, 115-116, and 118, wherein the primary oncolytic virus and the secondary oncolytic virus each comprise an miRNA target sequence (miR-TS) cassette comprising one or more miRNA target sequences inserted into one or more viral genes required for replication or inserted into the 3′ or 5′ UTR of the viral genome.

121. The virus of any one of claims 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, 117, and 119, wherein the primary virus and the secondary virus each comprise an miRNA target sequence (miR-TS) cassette comprising one or more miRNA target sequences inserted into one or more viral genes required for replication or inserted into the 3′ or 5′ UTR of the viral genome.

122. The virus of any one of claims 116, 118, and 120, wherein expression of the one or more miRNAs in a cell inhibits replication of the primary and/or secondary oncolytic viruses.

123. The virus of any one of claims 117, 119, and 121, wherein expression of the one or more miRNAs in a cell inhibits replication of the primary and/or secondary viruses.

124. The virus of any one of claims 1-123, further comprising a polynucleotide sequence encoding at least one exogenous payload protein.

125. The virus of claim 124, wherein the exogenous payload protein is a fluorescent protein, an enzyme, a cytokine, a chemokine, or an antigen-binding molecule.

126. The virus of any one of claims 1, 3, 5, 7, 9, 11, 13-16, 18-19, 21-34, 36, 38-39, 42-53, 55, 57-77, 79, 81, 83, 84, 86, 88, 89, 91-92, 94-98, 100, 102-109, 110, 112-113, 115-116, 118, 120, 122, and 124-125, wherein expression of the secondary oncolytic virus is regulated by an exogenous agent.

127. The virus of any one of claims 2, 4, 6, 8, 10, 12-15, 17-18, 20-33, 35, 37, 40-52, 54, 56-76, 78, 80, 82, 83, 85, 87, 88, 90-91, 93-97, 99, 101-109, 111-112, 114-115, 117, 119, 121, and 123-125, wherein expression of the secondary virus is regulated by an exogenous agent.

128. The virus of claim 126 or 127, wherein the exogenous agent is a peptide, a hormone, or a small molecule.

129. A composition comprising the virus of any one of claims 1-128.

130. A method of killing a population of tumor cells comprising administering the virus of any one of claims 1-128 or the composition of claim 129 to the population of tumor cells.

131. The method of claim 130, wherein a first subpopulation of the tumor cells are infected and killed by the primary oncolytic virus.

132. The method of claim 130 or 131, wherein a second subpopulation of the tumor cells are infected and killed by the secondary oncolytic virus.

133. The method of any one of claims 130-132, wherein a subpopulation of the tumor cells are infected and killed by both the primary oncolytic virus and the secondary oncolytic virus.

134. The method of any one of claims 130-133, wherein a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone.

135. The method of any one of claims 130-134, further comprising administering one or more exogenous agents to the population of tumor cells, wherein the one or more exogenous agents regulate the production of the secondary oncolytic virus.

136. The method of claim 135, wherein the one or more exogenous agents is/are administered at the same time as the primary oncolytic virus, and wherein the presence of the exogenous agent(s) inhibits production of the secondary oncolytic virus.

137. The method of claim 135, wherein the one or more exogenous agents is/are administered after the primary oncolytic virus, and wherein the presence of the exogenous agent(s) induces production of the secondary oncolytic virus.

138. The method of claim 137, wherein the exogenous agent(s) is/are administered at least 1 day, at least 1 week, or at least 1 month, after administration of the primary oncolytic virus.

139. The method of any one of the claims 135-138, wherein no secondary oncolytic virus is detectable prior to the administration of the exogenous agent(s).

140. The method of claim 130, wherein a first subpopulation of the tumor cells are infected and killed by the primary virus.

141. The method of claim 130 or 140, wherein a second subpopulation of the tumor cells are infected and killed by the secondary virus.

142. The method of any one of claims 130, 140, and 141, wherein a subpopulation of the tumor cells are infected and killed by both the primary virus and the secondary virus.

143. The method of any one of claims 130 and 140-142, wherein a greater number of tumor cells in the population are killed by the primary and secondary viruses compared to the number of tumor cells killed by a reference primary virus without the polynucleotide encoding the secondary virus or the secondary virus alone.

144. The method of any one of claims 130 and 140-143, further comprising administering one or more exogenous agents to the population of tumor cells, wherein the one or more exogenous agents regulate the production of the secondary virus.

145. The method of claim 144, wherein the one or more exogenous agents is/are administered at the same time as the primary virus, and wherein the presence of the exogenous agent(s) inhibits production of the secondary virus.

146. The method of claim 145, wherein the one or more exogenous agents is/are administered after the primary virus, and wherein the presence of the exogenous agent(s) induces production of the secondary virus.

147. The method of claim 146, wherein the exogenous agent(s) is/are administered at least 1 day, at least 1 week, or at least 1 month, after administration of the primary virus.

148. The method of any one of the claims 144-147, wherein no secondary virus is detectable prior to the administration of the exogenous agent(s).

149. A method of treating a tumor in a subject in need thereof comprising administering the virus of any one of claims 1-128 or the composition of claim 129 to the subject.

150. The method of claim 149, wherein a greater number of tumor cells in the population are killed by the primary and secondary oncolytic viruses compared to the number of tumor cells killed by a reference primary oncolytic virus without the polynucleotide encoding the secondary oncolytic virus or the secondary oncolytic virus alone.

151. The method of claim 149 or 150, wherein the method leads to greater reduction of tumor size in the subject compared to administration of a reference primary oncolytic virus without the polynucleotide encoding the secondary oncolytic virus or the secondary oncolytic virus alone.

152. The method of any one of claims 149-151, wherein the method induces a stronger immune response against one or more tumor antigens in the subject compared to administering a reference primary oncolytic virus without the polynucleotide encoding the secondary oncolytic virus or administering the secondary oncolytic virus alone.

153. The method of any one of claims 149-152, wherein the method results in a reduced immune response against the primary oncolytic virus in the subject compared to administering a reference primary oncolytic virus without the polynucleotide encoding the secondary oncolytic virus.

154. The method of any one of claims 149-153, wherein the method results in a reduced immune response against the secondary oncolytic virus in the subject compared to administering the secondary oncolytic virus alone.

155. The method of any one of claims 149-154, wherein the method results in preferential/more specific killing of tumor cells in the subject compared to administering a reference primary oncolytic virus without the polynucleotide encoding the secondary oncolytic virus or administering the secondary oncolytic virus alone.

156. The method of any one of claims 149-155, wherein the method results in more persistent production of the primary oncolytic virus in the subject compared to administering a reference primary oncolytic virus without the polynucleotide encoding the secondary oncolytic virus.

157. The method of any one of claims 149-156, wherein the method results in more persistent production of the secondary oncolytic virus in the subject compared to administering the secondary oncolytic virus alone.

158. The method of any one of claims 149-157, wherein the method results in an extended period of tumor inhibition in the subject compared to administering a reference primary oncolytic virus without the polynucleotide encoding the secondary oncolytic virus or the secondary oncolytic virus alone.

159. The method of any one of claims 149-158, wherein the method enables viral infection of more cell types compared to administering a reference primary oncolytic virus without the polynucleotide encoding the secondary oncolytic virus or the secondary oncolytic virus alone.

160. The method of any one of claims 149-159, further comprising administering one or more exogenous agents to the population of tumor cells, wherein the one or more exogenous agents regulate the production of the secondary oncolytic virus.

161. The method of claim 160, wherein the one or more exogenous agents is/are administered at the same time as the primary oncolytic virus, and wherein the presence of the exogenous agent(s) inhibits production of the secondary oncolytic virus.

162. The method of claim 160, wherein the one or more exogenous agents is/are administered after the primary oncolytic virus, and wherein the presence of the exogenous agent(s) induces production of the secondary oncolytic virus.

163. The method of claim 162, wherein the exogenous agent(s) is/are administered at least 1 day, at least 1 week, or at least 1 month, after administration of the primary oncolytic virus.

164. The method of any one of the claims 160-163, wherein no secondary oncolytic virus is detectable prior to the administration of the exogenous agent(s).

165. The method of claim 149, wherein a greater number of tumor cells in the population are killed by the primary and secondary viruses compared to the number of tumor cells killed by a reference primary virus without the polynucleotide encoding the secondary virus or the secondary virus alone.

166. The method of claim 149 or 165, wherein the method leads to greater reduction of tumor size in the subject compared to administration of a reference primary virus without the polynucleotide encoding the secondary virus or the secondary virus alone.

167. The method of any one of claims 149, 165, and 166, wherein the method induces a stronger immune response against one or more tumor antigens in the subject compared to administering a reference primary virus without the polynucleotide encoding the secondary virus or administering the secondary virus alone.

168. The method of any one of claims 149 and 165-167, wherein the method results in a reduced immune response against the primary virus in the subject compared to administering a reference primary virus without the polynucleotide encoding the secondary virus.

169. The method of any one of claims 149 and 165-168, wherein the method results in a reduced immune response against the secondary virus in the subject compared to administering the secondary virus alone.

170. The method of any one of claims 149 and 165-169, wherein the method results in preferential/more specific killing of tumor cells in the subject compared to administering a reference primary virus without the polynucleotide encoding the secondary virus or administering the secondary virus alone.

171. The method of any one of claims 149 and 165-170, wherein the method results in more persistent production of the primary virus in the subject compared to administering a reference primary virus without the polynucleotide encoding the secondary virus.

172. The method of any one of claims 149 and 165-171, wherein the method results in more persistent production of the secondary virus in the subject compared to administering the secondary virus alone.

173. The method of any one of claims 149 and 165-172, wherein the method results in an extended period of tumor inhibition in the subject compared to administering a reference primary virus without the polynucleotide encoding the secondary virus or the secondary virus alone.

174. The method of any one of claims 149 and 165-173, wherein the method enables viral infection of more cell types compared to administering a reference primary virus without the polynucleotide encoding the secondary virus or the secondary virus alone.

175. The method of any one of claims 149 and 165-174, further comprising administering one or more exogenous agents to the population of tumor cells, wherein the one or more exogenous agents regulate the production of the secondary virus.

176. The method of claim 175, wherein the one or more exogenous agents is/are administered at the same time as the primary virus, and wherein the presence of the exogenous agent(s) inhibits production of the secondary virus.

177. The method of claim 175, wherein the one or more exogenous agents is/are administered after the primary virus, and wherein the presence of the exogenous agent(s) induces production of the secondary virus.

178. The method of claim 177, wherein the exogenous agent(s) is/are administered at least 1 day, at least 1 week, or at least 1 month, after administration of the primary virus.

179. The method of any one of the claims 175-178, wherein no secondary virus is detectable prior to the administration of the exogenous agent(s).

180. A polynucleotide encoding the virus of claims 1-128.

181. A vector comprising the polynucleotide of claim 180.

182. A pharmaceutical composition comprising the vector of claim 181.
